(19) United States
(12) Patent Application Publication
(10) Pub. No.: US 2023/0192705 A1
(43) Pub. Date: Jun. 22, 2023

(54) SHP2 INHIBITORS, COMPOSITIONS AND USES THEREOF

(71) Applicant: Betta Pharmaceuticals Co., Ltd, Zhejiang (CN)

(72) Inventors: Bang FU, Beijing (CN); Zhongxin SUN, Beijing (CN); Xiaofeng XU, Beijing (CN); Wei REN, Beijing (CN); Yinlong LI, Beijing (CN); Ling LI, Zhejiang (CN); Lieming DING, Zhejiang (CN); Jiabing WANG, Beijing (CN)

(73) Assignee: Betta Pharmaceuticals Co., Ltd., Zhejiang (CN)

(21) Appl. No.: 18/009,277

(22) PCT Filed: Jun. 10, 2021
(86) PCT No.: PCT/CN2021/099275

§ 371 Date: Dec. 8, 2022

(30) Foreign Application Priority Data
Jun. 11, 2020 (WO) PCT/CN2020/095674
Nov. 26, 2020 (WO) PCT/CN2020/131791
May 25, 2021 (CN) 202110574621.0

(51) Int. Cl. C07D 487/04; C07D 487/14; C07D 519/00; C07F 9/6561; C07D 491/107; A61P 35/00; A61P 11/00; A61P 35/02

(52) U.S. Cl. CPC C07D 487/04; C07D 487/14; C07D 519/00; C07F 9/6561; C07D 491/107; A61P 35/00; A61P 11/00; A61P 35/02

(57) ABSTRACT

Provided are compounds of Formula (I), methods of using the compounds as SHP2 inhibitors, and pharmaceutical compositions comprising such compounds. The compounds are useful in treating SHP2-mediated diseases.

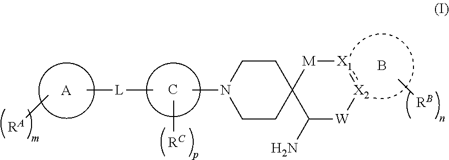

### FIELD OF THE INVENTION

The present invention relates to series of compounds as inhibitors of Src homologyregion 2-containing protein tyrosine phosphatase 2 (SHP2), methods and pharmaceutical compositions thereof. The present invention also relates to the use of the compounds or pharmaceutical compositions thereof for the treatment of SHP2-mediated diseases.

### BACKGROUND OF THE INVENTION

Src homologyregion 2-containing protein tyrosine phosphatase 2, SHP2 is a non-receptor type protein tyrosine phosphatase encoded by the PTPN11 gene. PTPN11 is the first recognized recognition-oncogene that encodes a tyrosine phosphatase (Chan R J et al. Blood, 2007, 109:862-867). The encoded SHP2 protein comprises an N-terminal SHP2 Structure domain (N-SHP2), a C-terminal SHP2 Structure domain (C-SHP2), and a protein phosphatase catalytic Structure domain (PTP), two C-terminal tyrosine residues (Y542 and Y580) and a proline (Pro) rich mold.

Recently, the Ras/ERK pathway is considered to be the most important signal transduction pathway for SHP2, and its mechanism (Dance M et al. The molecular functions of Shp2 in the RAS/mitogen-activated protein kinase (ERK1/2) pathway. Cell Signal, 2008, 20:453-459) is approximately: after activation of the growth factor receptor, its tyrosine residues are phosphorylated autologously to provide a stop site for Grb2 and SHP2 (adaptor protein containing the SH2 structure domain) phosphotyrosine binding region SH2. Binding of Grb2 to the phosphorylated growth factor receptor leads to the aggregation of SOS proteins in the cell membrane. SOS, as a guanine nucleotide exchange factor (GEF), can catalyze the conversion of membrane-bound protein Ras from inactive Ras-GDP to active Ras-GTP. The Ras-GTP is further associated with a downstream signal system to activate the Ser/Thr kinase Raf1 and the like, thereby activating the ERK under the action of regulating the kinase MEK, and directly acting on the target molecule of the cytoplasmic or transferring same to intracellular regulatory gene transcription after activation of the ERK to proliferate or differentiate the cells. This process may also be affected by SHP2 binding proteins and substrates (SHP substrate-1, SHPS-1), Ras-GTPase activating proteins (Ras-GAP), and other Src members.

The SHP2 protein not only modulates the Ras/ERK signaling path, but also reports that it also modulates a plurality of signaling paths such as JAK-STAT3, NF-κB, PI3K/Akt, RHO and NFAT, thereby regulating the physiological functions such as cell proliferation, differentiation, migration and apoptosis.

SHP2 has been proved to be related to a variety of diseases, and about 50% of Noonan syndrome patients were found to have missense mutations of PTPN11. In addition, PTPN11 mutation was found to be an important cause of JMML and multiple leukaemia (Tartaglia m et al. NAT genet, 2003, 34:148-150; LOH ml et al. Blood, 2004, 103:2325-2331; Tartaglia m et al. Br J Haematol, 2005, 129:333-339; Xu R et al. Blood, 2005, 106:3142-3149). With the development of the study on PTPN11/SHP2, it was found that PTPN11/SHP2 is related to the occurrence of lung cancer, gastric cancer, colon cancer, melanoma, thyroid cancer and other cancers (Tang Chunlan et al. China Journal of lung cancer, 2010, 13:98-101; Higuchi m et al. Cancer SCI, 2004, 95:442-447; bentires alj m et al. Cancer Res, 2004, 64:8816-8820; Martinelli s et al. Cancer gene cycle et al, 2006, 166:124-129).

Currently, SHP2 inhibitors have been more and more concerned as potential treatment for cancer. There are a plurality of SHP2 inhibitors under development, such as TNO155 developed by Novartis enters a Phase I clinical trial for the treatment of solid tumors in 2017. JAB-3068, developed by Jacobio Pharm, formally obtained the U. S. FDA New Drug Clinical Experiments in January 2018. RMC-4630, developed by Revolution, performed a first human clinical trial in half the year 2018. However, there is no drug for this target in the domestic and extraneous markets.

In WO2019183367 patent published on Sep. 26, 2019, Compound 178 structure was disclosed as below. And it was recorded that IC₅₀ of Compound 178 in the SHP2 allosteric inhibition test is greater than 10 uM, which is considered inactive in the skilled art.

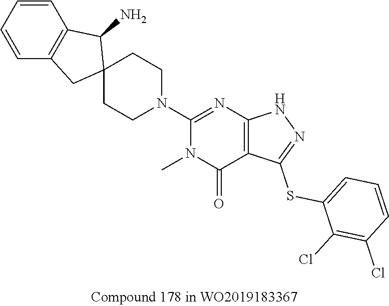 [SMILES: Cn1c(N2CCC3(CC2)Cc2ccccc2[C@H]3N)nc2[nH]nc(Sc3cccc(Cl)c3Cl)c2c1=O]

And therefore it is important to develop small molecule drugs capable of targeting and inhibiting the activity of SHP2, to provide SHP2 inhibitors with excellent pharmacodynamic properties, good safety and superior pharmacokinetics properties.

### SUMMARY OF INVENTION

The present invention relates to compounds that are used as Src homologyregion 2-containing protein tyrosine phosphatase 2 (SHP2) inhibitors. The SHP2 inhibitors are useful in the treatment of cancers.

A compound of Formula I, or a pharmaceutically acceptable salt, isomeride, stereoisomer, prodrug, chelate, non-covalent complex, or solvate thereof,

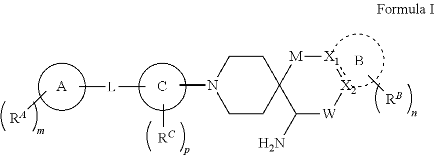

wherein,

[symbol] is a single bond or a double bond;

ring A is 6- to 14-membered aryl, 5- to 14-membered heteroaryl, 5- to 15-membered partially unsaturated heterocyclic ring, or 5- to 15-membered partially unsaturated carbocyclic ring; wherein the heteroaryl, the heterocyclic ring having 1-4 heteroatoms independently selected from N, O, and S;

ring B is absent, 6- to 10-membered aryl, 5- to 10-membered heteroaryl or 5- to 10-membered partially unsaturated heterocyclic ring;

provided that if ring B is 6- to 10-membered aryl, 5- to 10-membered heteroaryl or 5- to 10-membered partially unsaturated heterocyclic ring, then X₁ and X₂ are independently selected from C and N; or if ring B is absent, then X₁ and X₂ are independently selected from O, S, NR¹⁰⁰ and CR¹⁰⁰R¹⁰¹;

R¹⁰⁰ and R¹⁰¹ are independently selected from absent, hydrogen, halo, hydroxy, —C₁₋₆ alkyl and —C₁₋₆ alkoxy;

ring C is 5- to 14-membered heteroaryl or 5- to 14-membered partially unsaturated heterocyclic ring;

M is selected from absent, CH₂, O, NH and S;

W is absent or —CR³¹R³²—.

L is a single bond, —CR¹R²—, 3- to 6-membered monocyclic carbocyclic ring, 3- to 6-membered monocyclic heterocyclic ring, 7- to 12-membered bicyclic carbocyclic ring or 7- to 12-membered bicyclic heterocyclic ring; wherein, the 3- to 6-membered monocyclic carbocyclic ring, 3- to 6-membered monocyclic heterocyclic ring, 7- to 12-membered bicyclic carbocyclic ring and 7- to 12-membered bicyclic heterocyclic ring are optionally substituted with one to four substituents independently selected from R^{L};

each R^{A} is independently selected from hydrogen, halogen, —CN, —NO₂, ═O, C₁₋₆ alkyl, 3- to 14-membered saturated or partially unsaturated carbocyclic ring, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —P(═O)R¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or

two R^{A} together with the atoms to which they are attached to form a 3- to 6-membered carbocyclic ring or 3- to 6-membered heterocyclic ring, wherein the 5- to 6-membered carbocyclic ring and 3- to 6-membered heterocyclic ring is optionally substituted with one to four substituents independently selected from R³⁰;

each R^{B}, R^{C} and R^{L} are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸; R¹ and R² are independently selected from hydrogen, halogen, —CN, —NO₂, and C₁₋₆ alkyl; wherein the C₁₋₆ alkyl is optionally substituted with one or more substituents independently selected from halogen, —CN, —NO₂, —OR⁶, —NR⁷R⁸; wherein, R¹ and R² are not simultaneously hydrogen; and provided that if R¹ is hydrogen, R² is not methyl;

each R³⁰ is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;

R³¹ and R³² are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;

R³, R⁴, R⁵, R¹³, R²⁶, R²⁷ and R²⁹ are independently selected from hydrogen, halogen, —CN, —NO₂, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OR⁶, —NR⁷R⁸, —SR⁹, —C(═O)OR¹⁰, —C(═O)NR¹¹R¹², —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;

R⁶, R⁷, s, R⁹, R¹⁰, R¹¹, R¹², R¹⁴, R¹, R¹⁶, R¹⁷, R¹⁸, R¹⁹, R²⁰, R²¹, R²², R²³, R²⁴, R²⁵ and R²⁸ are independently selected from hydrogen, hydroxyl, halogen, —CN, —NO₂, ═O, C₁₋₈ alkyl, C₁₋₆ alkoxy, C₁₋₆haloalkyl, C₃₋₈ cycloalkyl, 6- to 14-membered aryl, 5- to 14-membered heteroaryl, and 3- to 8-membered saturated or partially unsaturated heterocyclic ring;

m is selected from 0, 1, 2, 3, 4, 5 and 6;

n is selected from 0, 1, 2, 3 and 4;

p is selected from 0, 1, 2, 3 and 4;

r is selected from 1, 2, 3 and 4;

s is selected from 1, 2, 3 and 4.

In some embodiments of Formula I, wherein L is a single bond.

In some embodiments of Formula I, wherein L is —CR¹R²—.

In some embodiments of Formula I, wherein R¹ and R² are independently selected from hydrogen, halogen and C₁₋₆ alkyl.

In some embodiments of Formula I, wherein R¹ and R² are independently selected from F, Cl, Br, methyl and ethyl.

In some embodiments of Formula I, wherein L is 3- to 6-membered monocyclic carbocyclic ring.

In some embodiments of Formula I, wherein L is cyclopropyl, cyclobutyl, cyclopentyl or cyclohexyl.

In some embodiments of Formula I, wherein L is

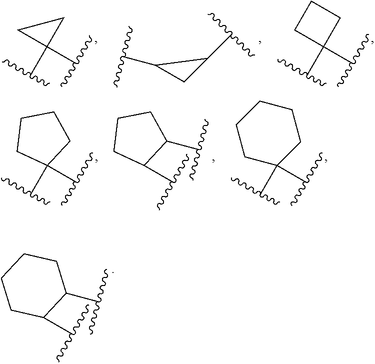

In some embodiments of Formula I, wherein L is 3- to 6-membered monocyclic heterocyclic ring.

In some embodiments of Formula I, wherein L is

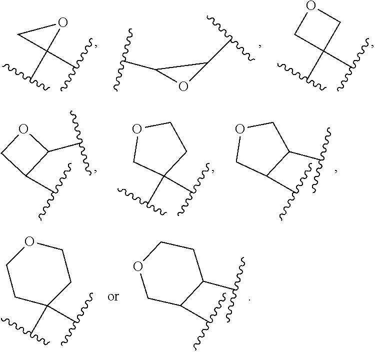

In some embodiments of Formula I, wherein L is 7- to 12-membered bicyclic carbocyclic ring.

In some embodiments of Formula I, wherein L is

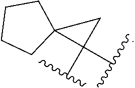 [SMILES: *C1(*)CC12CCCC2]

In some embodiments of Formula I, wherein L is 7- to 12-membered bicyclic heterocyclic ring.

In some embodiments of Formula I, wherein ring B is absent.

In some embodiments of Formula I, wherein X₁ and X₂ are independently selected from O, S, CH₂, and CHCH₃.

In some embodiments of Formula I, wherein X₁ is selected from O and CH₂.

In some embodiments of Formula I, wherein X₂ is selected from CH₂ and CHCH₃.

In some embodiments of Formula I, wherein ring B is 6- to 10-membered aryl or 5- to 10-membered heteroaryl.

In some embodiments of Formula I, wherein ring B is

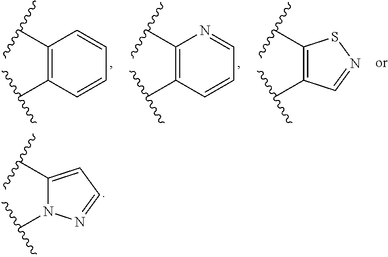

In some embodiments of Formula I, wherein ring C is 5- to 8-membered heteroaryl or 5- to 8-membered partially unsaturated heterocyclic ring.

In some embodiments of Formula I, wherein ring C is

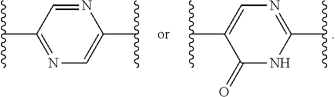 [SMILES: *c1cnc(*)cn1 ; *c1ncc(*)c(=O)[nH]1]

In some embodiments of Formula I, wherein ring C is 9- to 10-membered bicyclic heteroaryl or 9- to 14-membered partially unsaturated bicyclic heterocyclic ring.

In some embodiments of Formula I, wherein ring C is

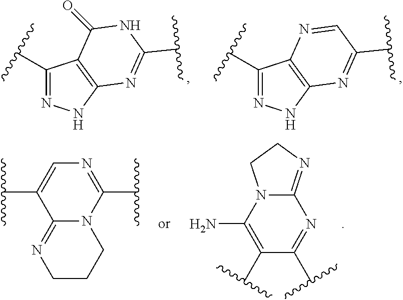

In some embodiments of Formula I, wherein ring C is 12- to 14-membered tricyclic heteroaryl or 12- to 14-membered partially unsaturated tricyclic heterocyclic ring.

In some embodiments of Formula I, wherein ring C is

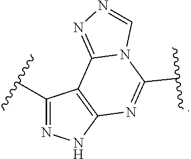 [SMILES: *c1n[nH]c2nc(*)n3cnnc3c12]

In some embodiments of Formula I, wherein M is absent or CH₂.

In some embodiments of Formula I, wherein W is absent.

In some embodiments of Formula I, wherein each R^{B}, R^{C} and R^{L} is independently selected from hydrogen, halogen, —CN, —NO₂, ═O, C₁₋₆ alkyl, —S—C₁₋₆ alkyl and C₁₋₆ alkoxy.

In some embodiments of Formula I, wherein each R^{B}, R^{C} and R^{L} is independently selected from hydrogen, F, Cl, Br, ═O, methyl, ethyl, —S—CH₃ and methoxy.

In some embodiments of Formula I, wherein ring A is 6- to 14-membered aryl, 5- to 14-membered heteroaryl, 5- to 8-membered partially unsaturated monocyclic heterocyclic ring, 9- to 12-membered partially unsaturated bicyclic carbocyclic ring, 9- to 12-membered partially unsaturated bicyclic heterocyclic ring, 11- to 15-membered partially unsaturated tricyclic carbocyclic ring, or 11- to 15-membered partially unsaturated tricyclic heterocyclic ring.

In some embodiments of Formula I, wherein ring A is 6- to 14-membered aryl.

In some embodiments of Formula I, wherein ring A is

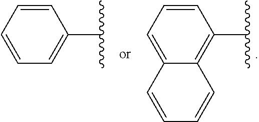

In some embodiments of Formula I, wherein ring A is 5- to 14-membered heteroaryl.

In some embodiments of Formula I, wherein ring A is

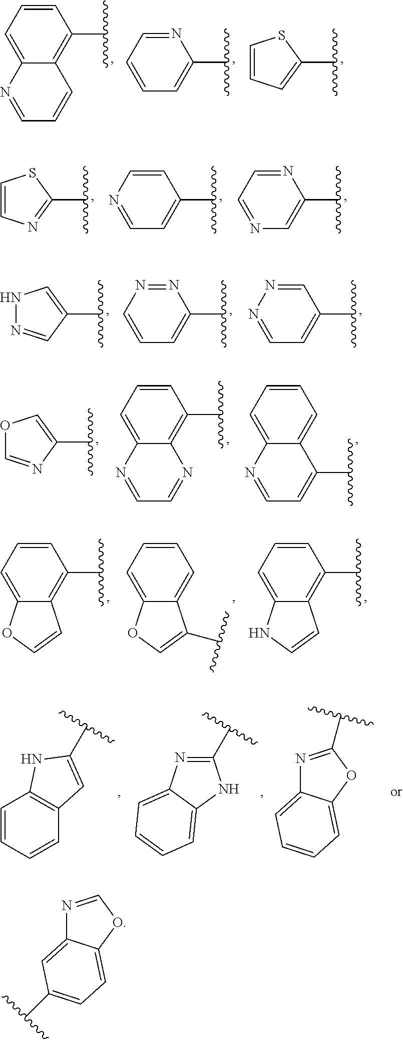

In some embodiments of Formula I, wherein ring A is 5- to 8-membered partially unsaturated monocyclic heterocyclic ring.

In some embodiments of Formula I, wherein ring A is 9- to 11-membered partially unsaturated bicyclic carbocyclic ring.

In some embodiments of Formula I, wherein ring A is

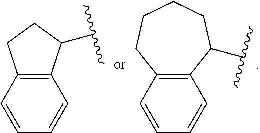

In some embodiments of Formula I, wherein ring A is 9- to 11-membered partially unsaturated bicyclic heterocyclic ring.

In some embodiments of Formula I, wherein ring A is

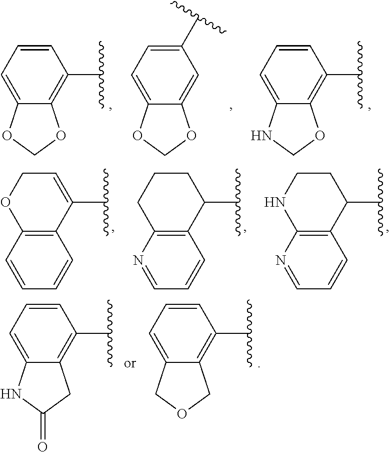

In some embodiments of Formula I, wherein ring A is

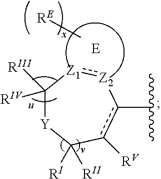

wherein,

[symbol] is a single bond or a double bond;

ring E is 6- to 14-membered aryl, 5- to 14-membered heteroaryl, 5- to 14-membered partially unsaturated heterocyclic ring; wherein the heteroaryl, the heterocyclic ring having 1-4 heteroatoms independently selected from N, O, and S;

Z₁ and Z₂ are independently selected from C and N;

Y is absent, O, NR^{Y}, C(═O), C(═O)O, C(═O)NR^{Y}, S, S(═O), S(═O)₂, S(═O)O, S(═O)NR^{Y}, S(═O)₂O or S(═O)₂NR^{Y};

each R^{E} is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or

two R^{E} together with the atoms to which they are attached to form a 3- to 6-membered carbocyclic ring or 3- to 6-membered heterocyclic ring, wherein the 5- to 6-membered carbocyclic ring and 3- to 6-membered heterocyclic ring is optionally substituted with one to four substituents independently selected from R³⁰;

each R^{I}, R^{II}, R^{III}, R^{IV} and R^{V} are independently selected from hydrogen, halogen, —CN, —NO₂, C₁₋₆ alkyl, C₃₋₈ cycloalkyl, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or

R^{I} together with R^{II} to form ═O; or

R^{I} and R^{II} together with the atoms to which they are attached can form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰; or

R^{III} and R^{IV} together with the atoms to which they are attached can form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰; or

R^{III} together with R^{IV} can form ═O;

u is selected from 0, 1, 2 and 3;

v is selected from 0, 1, 2 and 3.

In some embodiments of Formula I, wherein ring A is

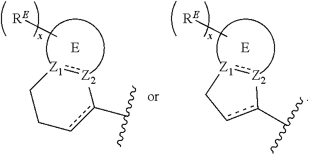

In some embodiments of Formula I, wherein ring A is

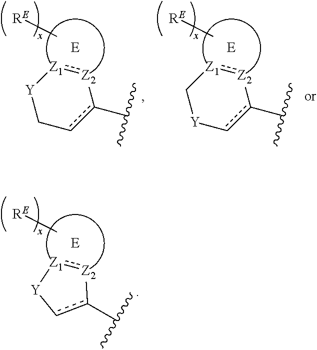

wherein, Y is O, NR^{Y}, C(═O), C(═O)O, C(═O)NR^{Y}, S, S(═O), S(═O)₂, S(═O)O, S(═O)NR^{Y}, S(═O)₂O or S(═O)₂NR^{Y}.

In some embodiments of Formula I, wherein ring A is

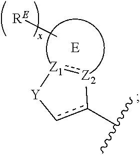

wherein, Y is C(═O), C(═O)O, C(═O)NR^{Y}, S(═O), S(═O)₂, S(═O)O, S(═O)NR^{Y}, S(═O)₂O or S(═O)₂NR^{Y}.

In some embodiments of Formula I, wherein ring A is

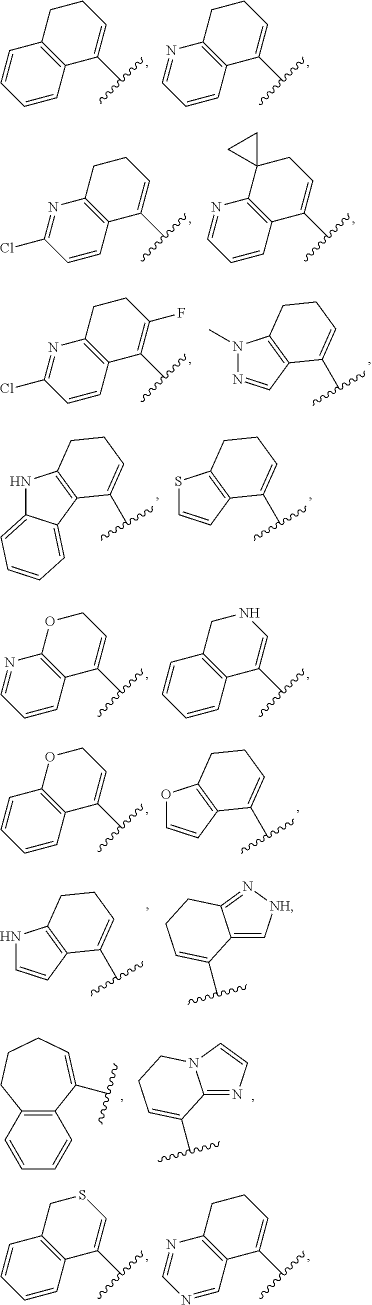

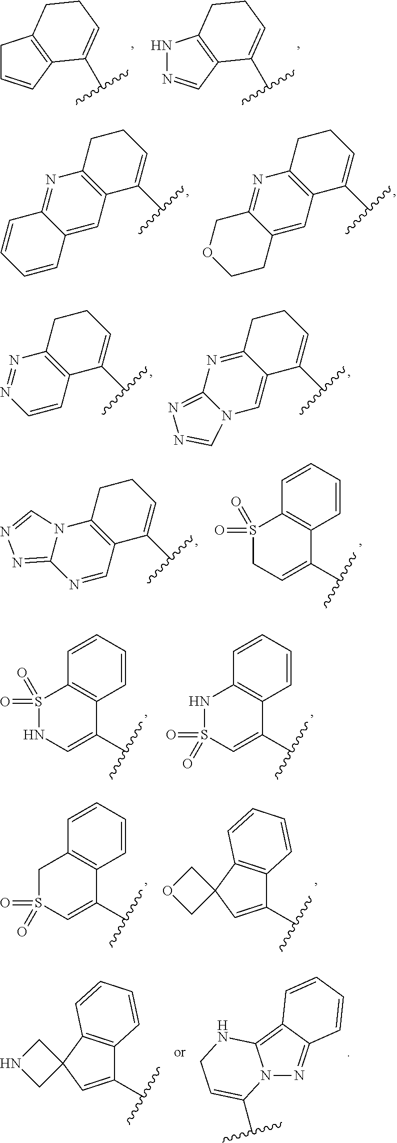

In some embodiments of Formula I, wherein each R^{A} is independently selected from hydrogen, halogen, —CN, —NO₂, ═O, C₁₋₆ alkyl, 6- to 10-membered aryl, 5- to 10-membered heteroaryl, —C(═O)R⁵, —OR⁶, —NR⁷R⁸, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁵C(═O)R²⁶; wherein C₁₋₆ alkyl, 6- to 10-membered aryl, and 5- to 10-membered heteroaryl are optionally substituted with one to four substituents independently selected from R³⁰.

In some embodiments of Formula I, wherein each R^{A} is independently selected from CH₃, F, CHF₂, CF₃, Cl, OCF₃, OCH₃, NH₂, CN, NH(CO)CH₂CH₃, OH, OCH₂CH₂OCH₃, OCHF₂, N(CH₃)₂, COCH₃, CH(CH₃)OH,

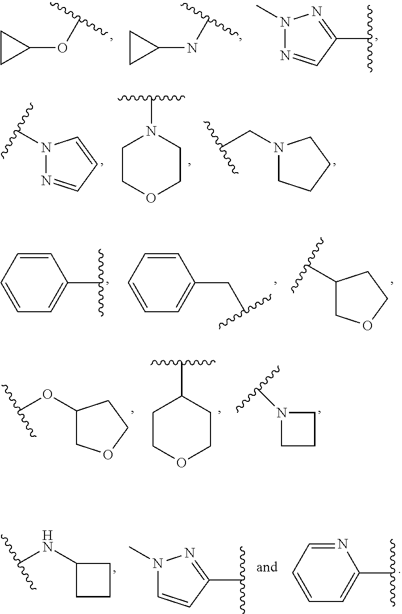

In some embodiments of Formula I, wherein each R³⁰ is independently selected from hydrogen, halogen, —CN, —NO₂, ═O and C₁₋₆ alkyl.

In some embodiments of Formula I, wherein each R³⁰ is independently selected from hydrogen, F, Cl, Br, ═O, methyl and ethyl.

In some embodiments of Formula I, wherein m is selected from 0, 1, 2 and 3.

In some embodiments of Formula I, wherein n is selected from 0, 1 and 2.

In some embodiments of Formula I, wherein p is selected from 0, 1 and 2.

In some embodiments of Formula I, wherein the compound is of Formula II, or a pharmaceutically acceptable salt, isomeride, stereoisomer, prodrug, chelate, non-covalent complex, or solvate thereof,

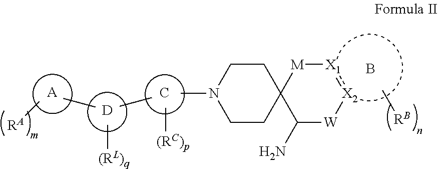

wherein,

[symbol] is a single bond or a double bond;

ring A is 6- to 14-membered aryl, 5- to 14-membered heteroaryl, 5- to 15-membered partially unsaturated heterocyclic ring, or 5- to 15-membered partially unsaturated carbocyclic ring; wherein the heteroaryl, the heterocyclic ring having 1-4 heteroatoms independently selected from N, O, and S;

ring B is absent, 6- to 10-membered aryl, 5- to 10-membered heteroaryl or 5- to 10-membered partially unsaturated heterocyclic ring;

provided that if ring B is 6- to 10-membered aryl, 5- to 10-membered heteroaryl or 5- to 10-membered partially unsaturated heterocyclic ring, then X₁ and X₂ are independently selected from C and N; or if ring B is absent, then X₁ and X₂ are independently selected from O, S, NR¹⁰⁰ and CR¹⁰⁰R¹⁰¹;

R¹⁰⁰ and R¹⁰¹ are independently selected from absent, hydrogen, halo, hydroxy, —C₁₋₆ alkyl and —C₁₋₆ alkoxy;

ring C is 5- to 14-membered heteroaryl or 5- to 14-membered partially unsaturated heterocyclic ring;

ring D is 3- to 6-membered monocyclic carbocyclic ring, 3- to 6-membered monocyclic heterocyclic ring, 7- to 12-membered bicyclic carbocyclic ring or 7- to 12-membered bicyclic heterocyclic ring;

M is selected from absent, CH₂, O, NH and S;

W is absent or —CR³¹R³²—.

each R^{A} is independently selected from hydrogen, halogen, —CN, —NO₂, ═O, C₁₋₆ alkyl, 3- to 14-membered saturated or partially unsaturated carbocyclic ring, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —P(═O)R¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰;

two R^{A} together with the atoms to which they are attached to form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰;

each R^{B}, R^{C} and R^{L} are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;

each R³⁰ is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;

R³¹ and R³² are independently selected from hydrogen, halogen, —CN, —NO₂, ═O, C₁₋₆ alkyl, —OR⁶, —NR⁷R⁸, and —SR⁹; wherein C₁₋₆ alkyl is optionally substituted with one or more substituents independently selected from halogen, —CN, —NO₂, —OR⁶, and —NR⁷R⁸;

R³, R⁴, R⁵, R¹³, R²⁶, R²⁷ and R²⁹ are independently selected from hydrogen, halogen, —CN, —NO₂, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OR⁶, —NR⁷R⁸, —SR⁹, —C(═O)OR¹⁰, —C(═O)NR¹¹R¹², —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;

R⁶, R⁷, R⁸, R⁹, R¹⁰, R¹¹, R¹², R¹⁴, R¹, R¹⁶, R¹⁷, R¹⁸, R¹⁹, R²⁰, R²¹, R²², R²³, R²⁴, R²⁵ and R²⁸ are independently selected from hydrogen, hydroxyl, halogen, —CN, —NO₂, ═O, C₁₋₆ alkyl, C₁₋₆ alkoxy, C₁₋₆haloalkyl, C₃₋₈ cycloalkyl, 6- to 14-membered aryl, 5- to 14-membered heteroaryl, and 3- to 8-membered saturated or partially unsaturated heterocyclic ring;

m is selected from 0, 1, 2, 3, 4, 5 and 6;

n is selected from 0, 1, 2, 3 and 4;

p is selected from 0, 1, 2, 3 and 4;

q is selected from 1, 2, 3 and 4;

r is selected from 1, 2, 3 and 4;

s is selected from 1, 2, 3 and 4.

In some embodiments of Formula II, wherein ring D is 3- to 6-membered monocyclic carbocyclic ring.

In some embodiments of Formula II, wherein ring D is cyclopropyl, cyclobutyl, cyclopentyl or cyclohexyl.

In some embodiments of Formula II, wherein ring D is

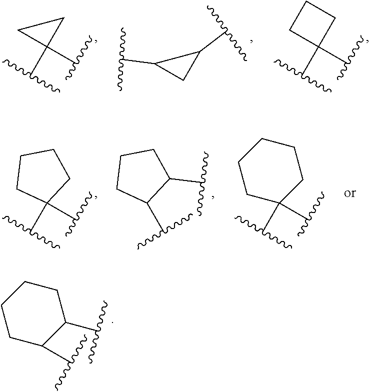

In some embodiments of Formula II, wherein ring D is 3- to 6-membered monocyclic heterocyclic ring.

In some embodiments of Formula II, wherein ring D is

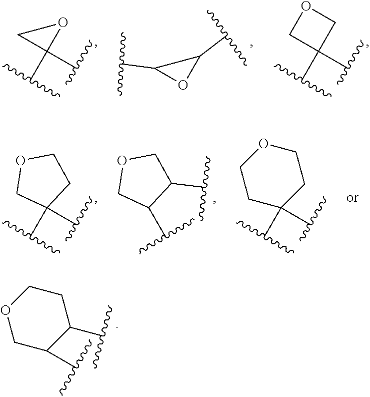

In some embodiments of Formula II, wherein ring D is 7- to 12-membered bicyclic carbocyclic ring.

In some embodiments of Formula II, wherein ring D is

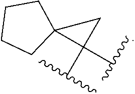 [SMILES: *C1(*)CC12CCCC2]

In some embodiments of Formula II, wherein ring B is absent.

In some embodiments of Formula II, wherein X₁ and X₂ are independently selected from O, S, CH₂, and CHCH₃.

In some embodiments of Formula II, wherein X₁ is selected from O and CH₂.

In some embodiments of Formula II, wherein X₂ is selected from CH₂ and CHCH₃.

In some embodiments of Formula II, wherein ring B is 6- to 10-membered aryl or 5- to 10-membered heteroaryl.

In some embodiments of Formula II, wherein ring B is

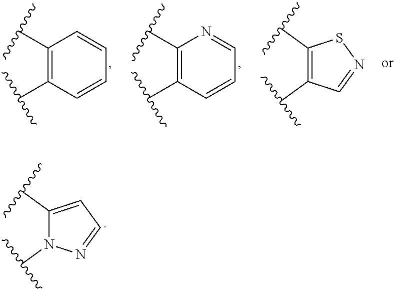

In some embodiments of Formula II, wherein ring C is 9- to 10-membered bicyclic heteroaryl or 9- to 14-membered partially unsaturated bicyclic heterocyclic ring.

In some embodiments of Formula II, wherein ring C is

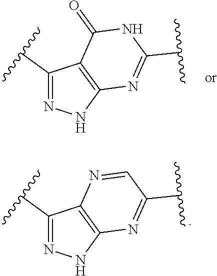 [SMILES: *c1nc2[nH]nc(*)c2c(=O)[nH]1 ; *c1cnc2c(*)n[nH]c2n1]

In some embodiments of Formula II, wherein ring C is 12- to 14-membered tricyclic heteroaryl or 12- to 14-membered partially unsaturated tricyclic heterocyclic ring.

In some embodiments of Formula II, wherein ring C is

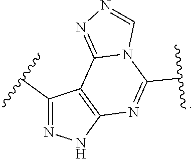 [SMILES: *c1n[nH]c2nc(*)n3cnnc3c12]

In some embodiments of Formula II, wherein each R^{B}, R^{C} and R^{L} is independently selected from hydrogen, halogen, —CN, —NO₂, ═O, and C₁₋₆ alkyl.

In some embodiments of Formula II, wherein each R^{B}, R^{C} and R^{L} is independently selected from hydrogen, F, Cl, Br, ═O, methyl and ethyl.

In some embodiments of Formula II, wherein M is absent or CH₂.

In some embodiments of Formula II, wherein W is absent.

In some embodiments of Formula II, wherein ring A is 6- to 14-membered aryl, 5- to 14-membered heteroaryl, 5- to 8-membered partially unsaturated monocyclic heterocyclic ring, 9- to 12-membered partially unsaturated bicyclic carbocyclic ring, 9- to 12-membered partially unsaturated bicyclic heterocyclic ring, 11- to 15-membered partially unsaturated tricyclic carbocyclic ring, or 11- to 15-membered partially unsaturated tricyclic heterocyclic ring.

In some embodiments of Formula II, wherein ring A is 6- to 14-membered aryl.

In some embodiments of Formula II, wherein ring A is

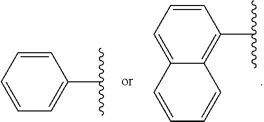 [SMILES: *c1ccccc1 ; *c1cccc2ccccc12]

In some embodiments of Formula II, wherein, ring A is 5- to 14-membered heteroaryl.

In some embodiments of Formula II, wherein ring A is

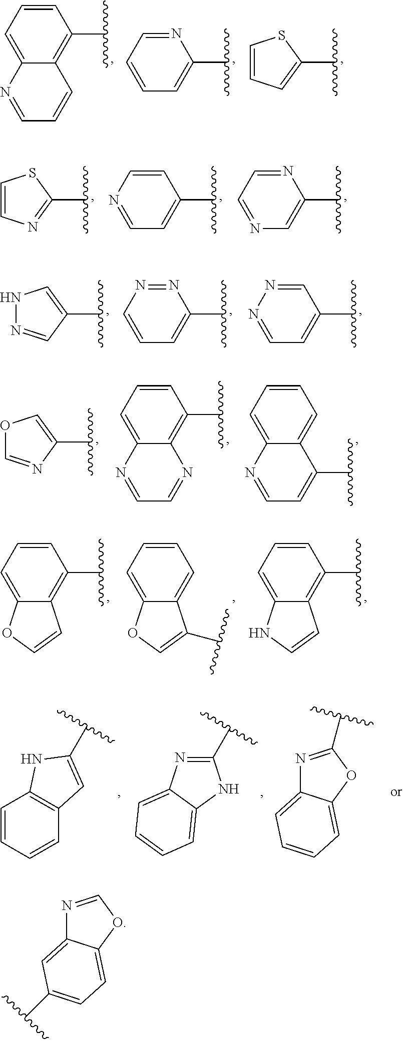

In some embodiments of Formula II, wherein ring A is 9- to 11-membered partially unsaturated bicyclic carbocyclic ring.

In some embodiments of Formula II, wherein ring A is

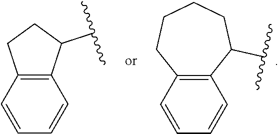 [SMILES: *C1CCc2ccccc21 ; *C1CCCCc2ccccc21]

In some embodiments of Formula II, wherein ring A is 9- to 11-membered partially unsaturated bicyclic heterocyclic ring.

In some embodiments of Formula II, wherein ring A is

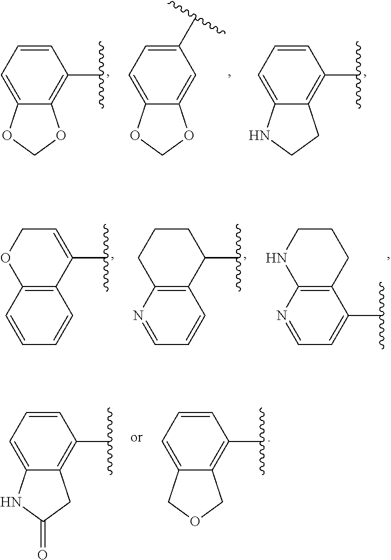

In some embodiments of Formula II, wherein each R^{A} is independently selected from hydrogen, halogen, —CN, —NO₂, ═O, C₁₋₆ alkyl, 6- to 10-membered aryl, 5- to 10-membered heteroaryl, —C(═O)R⁵, —OR⁶, —NR⁷R⁸, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁵C(═O)R²⁶; wherein C₁₋₆ alkyl, 6- to 10-membered aryl, and 5- to 10-membered heteroaryl are optionally substituted with one to four substituents independently selected from R³⁰.

In some embodiments of Formula II, wherein each R^{A} is independently selected from CH₃, CHF₂, F, CF₃, Cl, OCF₃, OCH₃, NH₂, CN, NH(CO)CH₂CH₃, OH, OCH₂CH₂OCH₃, OCHF₂, N(CH₃)₂, COCH₃, CH(CH₃)OH,

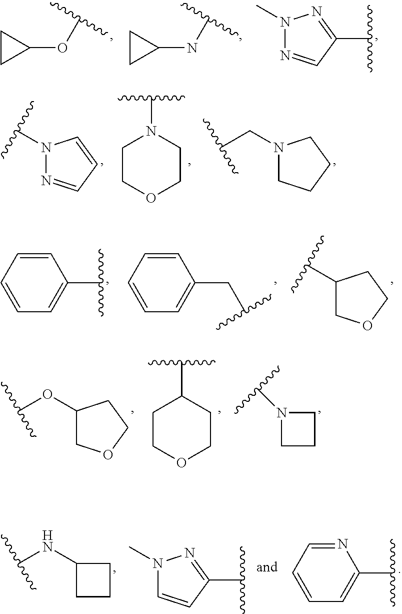

In some embodiments of Formula II, wherein each R³⁰ is independently selected from hydrogen, halogen, —CN, —NO₂, ═O, and C₁₋₆ alkyl.

In some embodiments of Formula II, wherein each R³⁰ is independently selected from hydrogen, F, Cl, Br, ═O, methyl and ethyl.

In some embodiments of Formula II, wherein m is selected from 0, 1, 2 and 3.

In some embodiments of Formula II, wherein n is selected from 0, 1 and 2.

In some embodiments of Formula II, wherein p is selected from 0, 1 and 2.

In some embodiments of Formula II, wherein q is selected from 0, 1 and 2.

In some embodiments of Formula I, wherein the compound is of Formula III, or a pharmaceutically acceptable salt, isomeride, stereoisomer, prodrug, chelate, non-covalent complex, or solvate thereof,

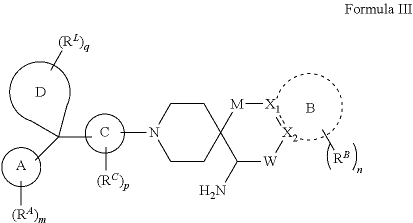

wherein,

[symbol] is a single bond or a double bond;

ring A is 6- to 14-membered aryl, 5- to 14-membered heteroaryl, 5- to 15-membered partially unsaturated heterocyclic ring, or 5- to 15-membered partially unsaturated carbocyclic ring; wherein the heteroaryl, the heterocyclic ring having 1-4 heteroatoms independently selected from N, O, and S;

ring B is absent, 6- to 10-membered aryl, 5- to 10-membered heteroaryl or 5- to 10-membered partially unsaturated heterocyclic ring;

provided that if ring B is 6- to 10-membered aryl, 5- to 10-membered heteroaryl or 5- to 10-membered partially unsaturated heterocyclic ring, then X₁ and X₂ are independently selected from C and N; or if ring B is absent, then X₁ and X₂ are independently selected from O, S, NR¹⁰⁰ and CR¹⁰⁰R¹⁰¹;

R¹⁰⁰ or R¹⁰¹ are independently selected from absent, hydrogen, halo, hydroxy, —C₁₋₆ alkyl and —C₁₋₆ alkoxy;

ring C is 5- to 14-membered heteroaryl or 5- to 14-membered partially unsaturated heterocyclic ring;

ring D is 3- to 6-membered monocyclic carbocyclic ring, 3- to 6-membered monocyclic heterocyclic ring, 7- to 12-membered bicyclic carbocyclic ring or 7- to 12-membered bicyclic heterocyclic ring;

M is selected from absent, CH₂, O, NH and S;

W is absent or —CR³¹R³²—.

each R^{A} is independently selected from hydrogen, halogen, —CN, —NO₂, ═O, C₁₋₆ alkyl, 3- to 14-membered saturated or partially unsaturated carbocyclic ring, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —P(═O)R¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or

two R^{A} together with the atoms to which they are attached to form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰;

each R^{B}, R^{C} and R^{L} are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;

R³¹ and R³² are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;

each R³⁰ is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;

R³, R⁴, R⁵, R¹³, R²⁶, R²⁷ and R²⁹ are independently selected from hydrogen, halogen, —CN, —NO₂, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OR⁶, —NR⁷R⁸, —SR⁹, —C(═O)OR¹⁰, —C(═O)NR¹¹R¹², —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;

R⁶, R⁷, R⁸, R⁹, R¹⁰, R¹¹, R¹², R¹⁴, R¹, R¹⁶, R¹⁷, R¹⁸, R¹⁹, R²⁰, R²¹, R²², R²³, R²⁴, R²⁵ and R²⁸ are independently selected from hydrogen, hydroxyl, halogen, —CN, —NO₂, ═O, C₁₋₆ alkyl, C₁₋₆ alkoxy, C₁₋₆haloalkyl, C₃₋₈ cycloalkyl, 6- to 14-membered aryl, 5- to 14-membered heteroaryl, and 3- to 8-membered saturated or partially unsaturated heterocyclic ring;

m is selected from 0, 1, 2, 3, 4, 5 and 6;

n is selected from 0, 1, 2, 3 and 4;

p is selected from 0, 1, 2, 3 and 4;

q is selected from 0, 1, 2, 3 and 4;

r is selected from 1, 2, 3 and 4;

s is selected from 1, 2, 3 and 4.

In some embodiments of Formula III, wherein ring D is 3- to 6-membered monocyclic carbocyclic ring.

In some embodiments of Formula III, wherein ring D is cyclopropyl, cyclobutyl, cyclopentyl or cyclohexyl.

In some embodiments of Formula III, wherein ring D is

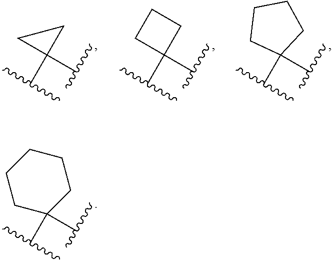 [SMILES: *C1(*)CC1 ; *C1(*)CCC1 ; *C1(*)CCCC1 ; *C1(*)CCCCC1]

In some embodiments of Formula III, wherein ring D is 3- to 6-membered monocyclic heterocyclic ring.

In some embodiments of Formula III, wherein ring D is

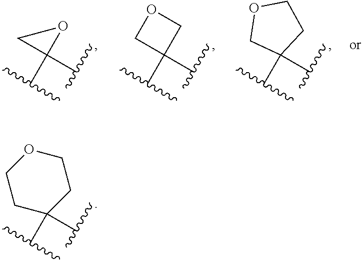 [SMILES: *C1(*)CO1 ; *C1(*)COC1 ; *C1(*)CCOC1 ; *C1(*)CCOCC1]

In some embodiments of Formula III, wherein ring D is 7- to 12-membered bicyclic carbocyclic ring.

In some embodiments of Formula III, wherein ring D is

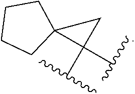 [SMILES: *C1(*)CC12CCCC2]

In some embodiments of Formula III, wherein ring B is absent.

In some embodiments of Formula III, wherein X₁ and X₂ are independently selected from O, S, CH₂, and CHCH₃.

In some embodiments of Formula III, wherein X₁ is selected from O and CH₂.

In some embodiments of Formula III, wherein X₂ is selected from CH₂ and CHCH₃.

In some embodiments of Formula III, wherein ring B is 6- to 10-membered aryl or 5- to 10-membered heteroaryl.

In some embodiments of Formula III, wherein ring B is

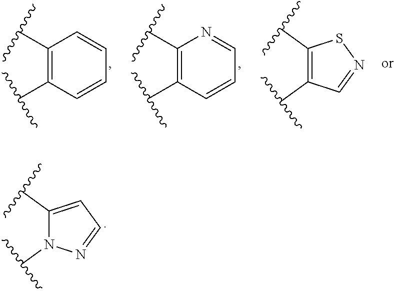

In some embodiments of Formula III, wherein ring C is 9- to 10-membered bicyclic heteroaryl or 9- to 14-membered partially unsaturated bicyclic heterocyclic ring.

In some embodiments of Formula III, wherein ring C is

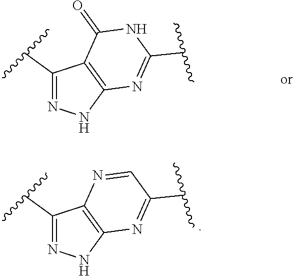 [SMILES: *c1nc2[nH]nc(*)c2c(=O)[nH]1 ; *c1cnc2c(*)n[nH]c2n1]

In some embodiments of Formula III, wherein ring C is 12- to 14-membered tricyclic heteroaryl or 12- to 14-membered partially unsaturated tricyclic heterocyclic ring.

In some embodiments of Formula III, wherein ring C is

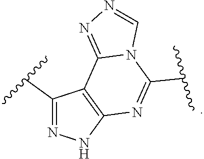 [SMILES: *c1n[nH]c2nc(*)n3cnnc3c12]

In some embodiments of Formula III, wherein each R^{B}, R^{C} and R^{L} is independently selected from hydrogen, halogen, —CN, —NO₂, ═O, and C₁₋₆ alkyl.

In some embodiments of Formula III, wherein each R^{B}, R^{C} and R^{L} is independently selected from hydrogen, F, Cl, Br, ═O, methyl and ethyl.

In some embodiments of Formula III, wherein M is absent or CH₂.

In some embodiments of Formula III, wherein W is absent.

In some embodiments of Formula III, wherein ring A is 6- to 14-membered aryl.

In some embodiments of Formula III, wherein ring A is

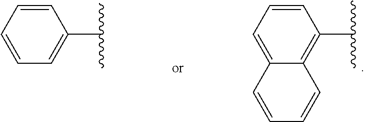 [SMILES: *c1ccccc1 ; *c1cccc2ccccc12]

In some embodiments of Formula III, wherein ring A is 5- to 14-membered heteroaryl.

In some embodiments of Formula III, wherein ring A is

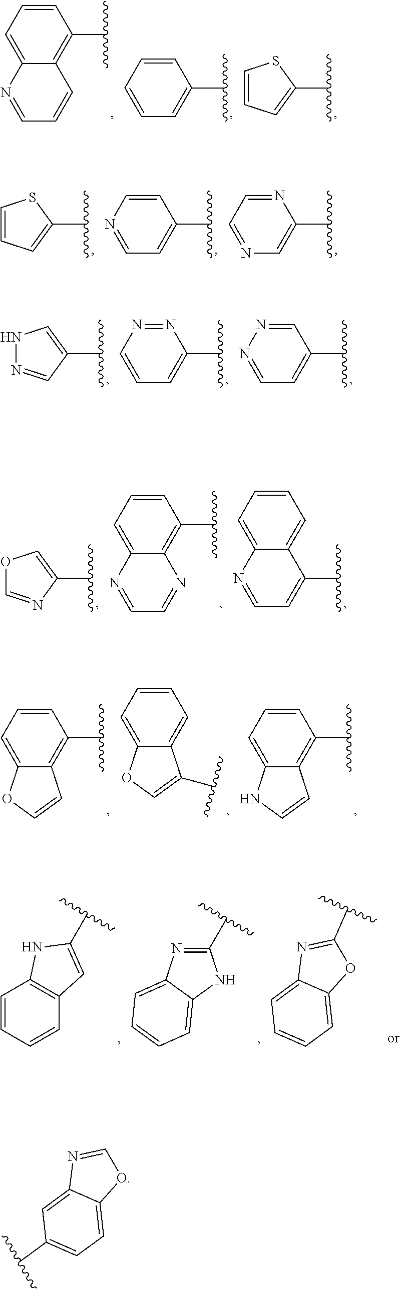

In some embodiments of Formula III, wherein ring A is 9- to 11-membered partially unsaturated bicyclic carbocyclic ring.

In some embodiments of Formula III, wherein ring A is

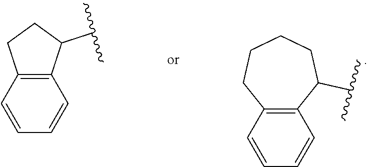 [SMILES: *C1CCc2ccccc21 ; *C1CCCCc2ccccc21]

In some embodiments of Formula III, wherein ring A is 9- to 11-membered partially unsaturated bicyclic heterocyclic ring.

In some embodiments of Formula III, wherein ring A is

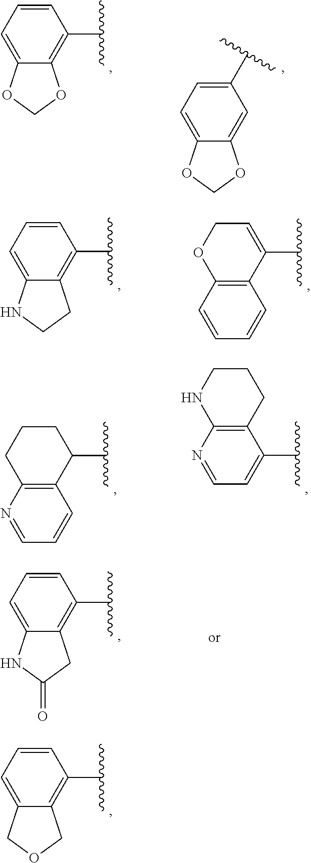

In some embodiments of Formula III, wherein each R^{A} is independently selected from hydrogen, halogen, —CN, —NO₂, ═O, C₁₋₆ alkyl, 6- to 10-membered aryl, 5- to 10-membered heteroaryl, —C(═O)R⁵, —OR⁶, —NR⁷R⁸, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁵C(═O)R²⁶; wherein C₁₋₆ alkyl, 6- to 10-membered aryl, and 5- to 10-membered heteroaryl are optionally substituted with one to four substituents independently selected from R³⁰.

In some embodiments of Formula III, wherein each R^{A} is independently selected from CH₃, F, CHF₂, CF₃, Cl, OCF₃, OCH₃, NH₂, CN, NH(CO)CH₂CH₃, OH, OCH₂CH₂OCH₃, OCHF₂, N(CH₃)₂, COCH₃, CH(CH₃)OH,

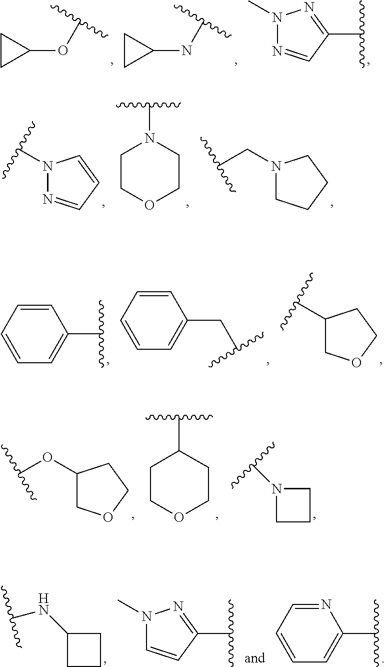

In some embodiments of Formula III, wherein each R³⁰ is independently selected from hydrogen, halogen, —CN, —NO₂, ═O, and C₁₋₆ alkyl.

In some embodiments of Formula III, wherein each R³⁰ is independently selected from hydrogen, F, Cl, Br, ═O, methyl and ethyl.

In some embodiments of Formula III, wherein m is selected from 0, 1, 2 and 3.

In some embodiments of Formula III, wherein n is selected from 0, 1 and 2.

In some embodiments of Formula III, wherein p is selected from 0, 1 and 2.

In some embodiments of Formula III, wherein q is selected from 0, 1 and 2.

Surprisingly, for the compounds of Formula III, when ring C is

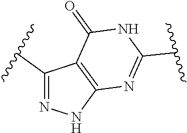 [SMILES: *c1nc2[nH]nc(*)c2c(=O)[nH]1]

and p is 0, at least the following effects is obtained:

1. The inhibitory activity on SHP2 enzyme, MV-4-11 cell and NCI-H358 cell is greatly improved;

2. Significant improvement in hERG;

3. Significant improvement in liver microsomal stability.

These improvements suggest excellent pharmacodynamic properties, good safety and superior pharmacokinetics properties.

In some embodiments of Formula I, wherein the compound is of Formula IV, or a pharmaceutically acceptable salt, isomeride, stereoisomer, prodrug, chelate, non-covalent complex, or solvate thereof,

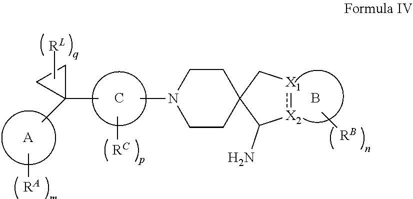

wherein,

ring A is 6- to 14-membered aryl, 5- to 14-membered heteroaryl, 5- to 15-membered partially unsaturated heterocyclic ring, or 5- to 15-membered partially unsaturated carbocyclic ring; wherein the heteroaryl, the heterocyclic ring having 1-4 heteroatoms independently selected from N, O, and S;

ring C is 5- to 14-membered heteroaryl or 5- to 14-membered partially unsaturated heterocyclic ring;

X₁ and X₂ are independently selected from C and N;

each R^{A} is independently selected from hydrogen, halogen, —CN, —NO₂, ═O, C₁₋₆ alkyl, 3- to 14-membered saturated or partially unsaturated carbocyclic ring, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —P(═O)R¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or

two R^{A} together with the atoms to which they are attached form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰;

each R^{B}, R^{C} and R^{L} is independently selected from hydrogen, halogen, —CN, —NO₂, ═O, C₁₋₆ alkyl, —OR⁶, —NR⁷R⁸, and —SR⁹; wherein C₁₋₆ alkyl is optionally substituted with one or more substituents independently selected from halogen, —CN, —NO₂, —OR⁶, and —NR⁷R⁸;

each R³⁰ is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;

R³, R⁴, R⁵, R¹³, R²⁶, R²⁷ and R²⁹ are independently selected from hydrogen, halogen, —CN, —NO₂, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OR⁶, —NR⁷R⁸, —SR⁹, —C(═O)OR¹⁰, —C(═O)NR¹¹R¹², —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;

R⁶, R⁷, R⁸, R⁹, R¹⁰, R¹¹, R¹², R¹⁴, R¹⁵, R¹⁶, R¹⁷, R¹⁸, R¹⁹, R²⁰, R²¹, R²², R²³, R²⁴, R²⁵ and R²⁸ are independently selected from hydrogen, hydroxyl, halogen, —CN, —NO₂, ═O, C₁₋₆ alkyl, C₁₋₆ alkoxy, C₁₋₆haloalkyl, C₃₋₈ cycloalkyl, 6- to 14-membered aryl, 5- to 14-membered heteroaryl, and 3- to 8-membered saturated or partially unsaturated heterocyclic ring;

m is selected from 0, 1, 2, 3, 4, 5 and 6;

n is selected from 0, 1, 2, 3 and 4;

p is selected from 0, 1, 2, 3 and 4;

q is selected from 0, 1, 2, 3 and 4;

r is selected from 1, 2, 3 and 4;

s is selected from 1, 2, 3 and 4.

In some embodiments of Formula IV, wherein ring B is 5- to 6-membered aryl or 5- to 6-membered heteroaryl.

In some embodiments of Formula IV, wherein ring B is

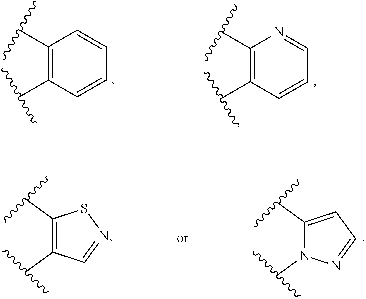 [SMILES: *c1ccccc1* ; *c1cccnc1* ; *c1cnsc1* ; *c1ccnn1*]

In some embodiments of Formula IV, wherein ring B is

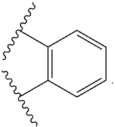 [SMILES: *c1ccccc1*]

In some embodiments of Formula IV, wherein ring C is 9- to 10-membered bicyclic heteroaryl or 9- to 14-membered partially unsaturated bicyclic heterocyclic ring.

In some embodiments of Formula IV, wherein ring C is dihydropyrazolo[3,4-d]pyrimidin-one or pyrazolo[3,4-b]pyrazine.

In some embodiments of Formula IV, wherein ring C is

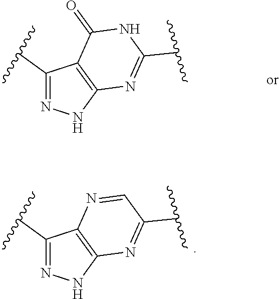 [SMILES: *c1nc2[nH]nc(*)c2c(=O)[nH]1 ; *c1cnc2c(*)n[nH]c2n1]

In some embodiments of Formula IV, wherein ring C is

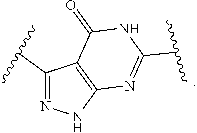 [SMILES: *c1nc2[nH]nc(*)c2c(=O)[nH]1]

In some embodiments of Formula IV, wherein each R^{B}, R^{C} and R^{L} is independently selected from hydrogen, halogen, —CN, —NO₂, ═O, C₁₋₆ alkyl, —S—C₁₋₆ alkyl and C₁₋₆ alkoxy.

In some embodiments of Formula IV, wherein R^{B} is H.

In some embodiments of Formula IV, wherein R^{C} is H or —CH₃.

In some embodiments of Formula IV, wherein R^{C} is H.

In some embodiments of Formula IV, wherein R^{L} is H, F, or Cl.

In some embodiments of Formula IV, wherein R^{L} is H.

In some embodiments of Formula IV, wherein ring A is 6- to 14-membered aryl, 5- to 14-membered heteroaryl, 5- to 8-membered partially unsaturated monocyclic heterocyclic ring, 9- to 12-membered partially unsaturated bicyclic carbocyclic ring, 9- to 12-membered partially unsaturated bicyclic heterocyclic ring, 11- to 15-membered partially unsaturated tricyclic carbocyclic ring, or 11- to 15-membered partially unsaturated tricyclic heterocyclic ring.

In some embodiments of Formula IV, wherein ring A is 6- to 14-membered aryl.

In some embodiments of Formula IV, wherein ring A is or

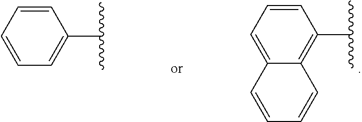 [SMILES: *c1ccccc1 ; *c1cccc2ccccc12]

In some embodiments of Formula IV, wherein ring A is

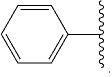 [SMILES: *c1ccccc1]

In some embodiments of Formula IV, wherein ring A is 5- to 14-membered heteroaryl.

In some embodiments of Formula IV, wherein ring A is

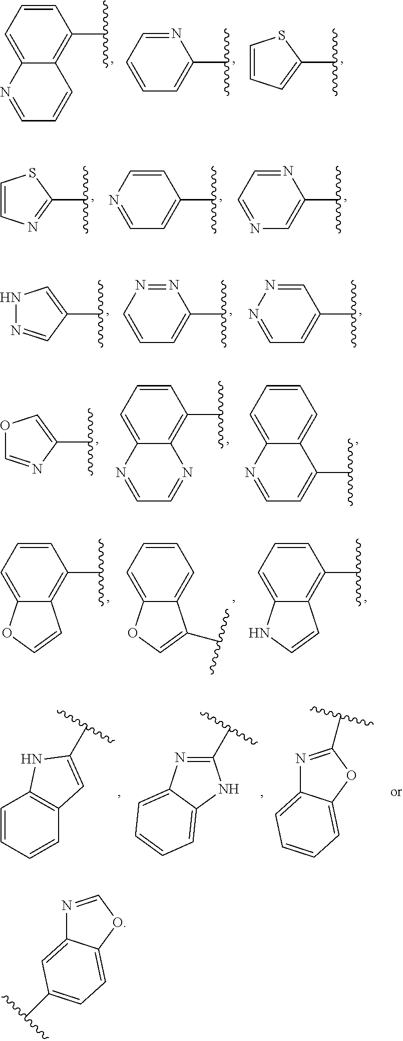

In some embodiments of Formula IV, wherein ring A is

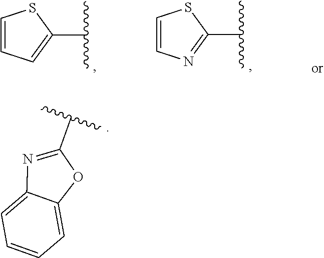 [SMILES: *c1cccs1 ; *c1nccs1 ; *c1nc2ccccc2o1]

In some embodiments of Formula IV, wherein ring A is

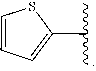 [SMILES: *c1cccs1]

In some embodiments of Formula IV, wherein ring A is 9- to 11-membered partially unsaturated bicyclic heterocyclic ring.

In some embodiments of Formula IV, wherein ring A is

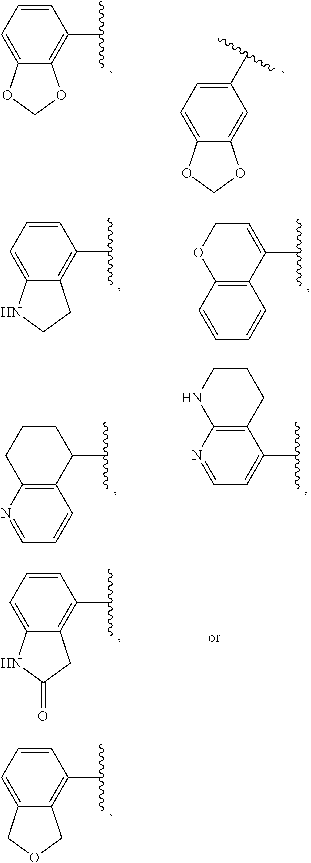

In some embodiments of Formula IV, wherein ring A is

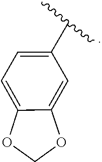 [SMILES: *c1ccc2c(c1)OCO2]

In some embodiments of Formula IV, wherein R^{A} is independently selected from hydrogen, halogen, —CN, —NO₂, ═O, C₁₋₆ alkyl, 6- to 10-membered aryl, 5- to 10-membered heteroaryl, —C(═O)R⁵, —OR⁶, —NR⁷R⁸, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁵C(═O)R²⁶.

In some embodiments of Formula IV, wherein R^{A} is independently selected from hydrogen, CH₃, F, CHF₂, CF₃, Cl, OCF₃, OCH₃, NH₂, CN, NH(CO)CH₂CH₃, OH, OCH₂CH₂OCH₃, OCHF₂, N(CH₃)₂, COCH₃, CH(CH₃)OH,

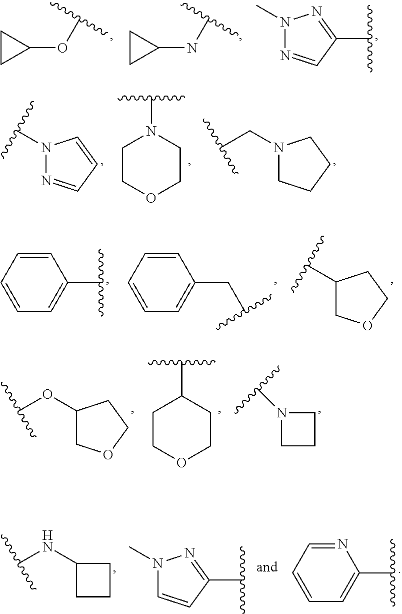

In some embodiments of Formula IV, wherein R^{A} is independently selected from hydrogen, CH₃, F, CF₃, Cl, Br, OCH₃, NH₂, CN, OH, COCH₃ and

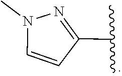 [SMILES: *c1ccn(C)n1]

In some embodiments of Formula IV, wherein R³⁰ are independently selected from H, F, Cl, Br, —CH₃ and —CH₂CH₃.

In some embodiments of Formula IV, wherein R³⁰ is independently selected from H.

In some embodiments of Formula IV, wherein m is selected from 0, 1 and 2.

In some embodiments of Formula IV, wherein n is selected from 0, 1 and 2.

In some embodiments of Formula IV, wherein p is selected from 0, 1 and 2.

In some embodiments of Formula IV, wherein q is selected from 0, 1 and 2.

In some embodiments of Formula I, wherein the compound is of Formula V, or a pharmaceutically acceptable salt, isomeride, stereoisomer, prodrug, chelate, non-covalent complex, or solvate thereof,

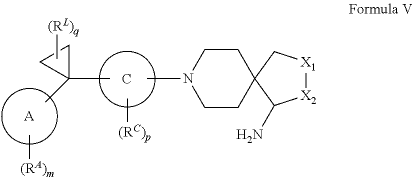

wherein,

ring A is 6- to 14-membered aryl, 5- to 14-membered heteroaryl, 5- to 15-membered partially unsaturated heterocyclic ring, or 5- to 15-membered partially unsaturated carbocyclic ring; wherein the heteroaryl, the heterocyclic ring having 1-4 heteroatoms independently selected from N, O, and S;

ring C is 5- to 14-membered heteroaryl or 5- to 14-membered partially unsaturated heterocyclic ring;

X₁ and X₂ are independently selected from O, S, NR¹⁰⁰ and CR¹⁰⁰R¹⁰¹;

R¹⁰⁰ and R¹⁰¹ are independently selected from absent, hydrogen, halo, hydroxy, —C₁₋₆ alkyl and —C₁₋₆ alkoxy;

each R^{A} is independently selected from hydrogen, halogen, —CN, —NO₂, ═O, C₁₋₆ alkyl, 3- to 14-membered saturated or partially unsaturated carbocyclic ring, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —P(═O)R¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or

two R^{A} together with the atoms to which they are attached form a 3- to 6-membered carbocyclic ring or a 3- to 6-membered heterocyclic ring, wherein the 5- to 6-membered carbocyclic ring and the 3- to 6-membered heterocyclic ring are optionally substituted with one to four substituents independently selected from R³⁰;

R^{C} and R^{L} are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;

each R³⁰ is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;

R³, R⁴, R⁵, R¹³, R²⁶, R²⁷ and R²⁹ are independently selected from hydrogen, halogen, —CN, —NO₂, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OR⁶, —NR⁷R⁸, —SR⁹, —C(═O)OR¹⁰, —C(═O)NR¹¹R¹², —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;

R⁶, R⁷, s, R⁹, R¹⁰, R¹¹, R¹², R¹⁴, R¹⁵, R¹⁶, R¹⁷, R¹⁸, R¹⁹, R²⁰, R²¹, R²², R²³, R²⁴, R²⁵ and R²⁸ are independently selected from hydrogen, hydroxyl, halogen, —CN, —NO₂, ═O, C₁₋₆ alkyl, C₁₋₆ alkoxy, C₁₋₆haloalkyl, C₃₋₈ cycloalkyl, 6- to 14-membered aryl, 5- to 14-membered heteroaryl, and 3- to 8-membered saturated or partially unsaturated heterocyclic ring;

m is selected from 0, 1, 2, 3, 4, 5 and 6;

p is selected from 0, 1, 2, 3 and 4;

q is selected from 0, 1, 2, 3 and 4;

r is selected from 1, 2, 3 and 4;

s is selected from 1, 2, 3 and 4.

In some embodiments of Formula V, wherein ring C is 5- to 6-membered monocyclic heterocyclic ring, 5- to 6-membered monocyclic heteroaryl ring, 9- to 10-membered bicyclic heteroaryl or 9- to 14-membered partially unsaturated bicyclic heterocyclic ring.

In some embodiments of Formula V, wherein ring C is

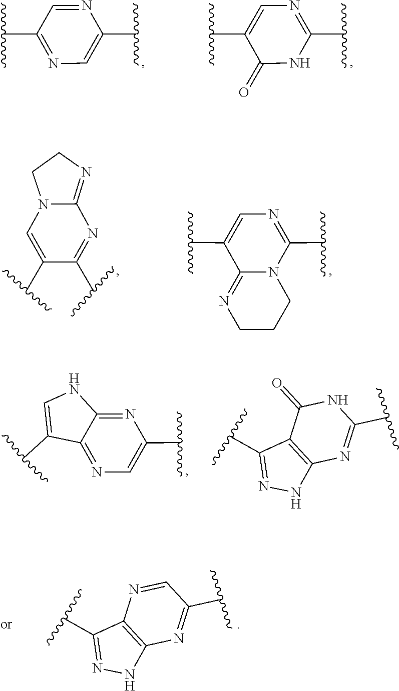

In some embodiments of Formula V, wherein X₁ is selected from O, NH, CHCH₃ and CH₂.

In some embodiments of Formula V, wherein X₂ is selected from O, NH, CHCH₃ and CH₂.

In some embodiments of Formula V, wherein R^{C} and R^{L} are independently selected from hydrogen, halogen, —CN, —NO₂, ═O, and C₁₋₆ alkyl.

In some embodiments of Formula V, wherein R^{C} and R^{L} are independently selected from hydrogen, F, Cl, Br, ═O, methyl and ethyl.

In some embodiments of Formula V, wherein ring A is 6- to 14-membered aryl, 5- to 14-membered heteroaryl, 5- to 8-membered partially unsaturated monocyclic heterocyclic ring, 9- to 12-membered partially unsaturated bicyclic carbocyclic ring, 9- to 12-membered partially unsaturated bicyclic heterocyclic ring, 11- to 15-membered partially unsaturated tricyclic carbocyclic ring, or 11- to 15-membered partially unsaturated tricyclic heterocyclic ring.

In some embodiments of Formula V, wherein ring A is 6- to 14-membered aryl, 5- to 14-membered heteroaryl or 5- to 15-membered partially unsaturated heterocyclic ring.

In some embodiments of Formula V, wherein ring A is

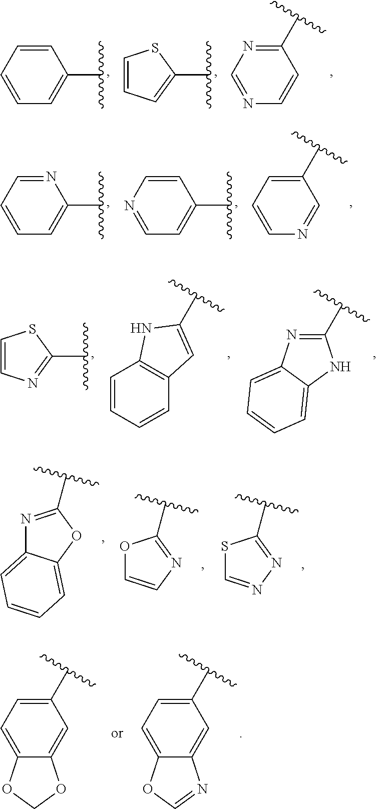

In some embodiments of Formula V, wherein each R^{A} is independently selected from hydrogen, halogen, —CN, —NO₂, ═O, C₁₋₆ alkyl, 6- to 10-membered aryl, 5- to 10-membered heteroaryl, —C(═O)R⁵, —OR⁶, —NR⁷R⁸, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁵C(═O)R²⁶; wherein C₁₋₆ alkyl, 6- to 10-membered aryl, and 5- to 10-membered heteroaryl are optionally substituted with one to four substituents independently selected from R³⁰.

In some embodiments of Formula V, wherein each R^{A} is independently selected from CH₃, F, CHF₂, CF₃, Cl, OCF₃, OCH₃, NH₂, CN, NH(CO)CH₂CH₃, OH, OCH₂CH₂OCH₃, OCHF₂, N(CH₃)₂, COCH₃, CH(CH₃)OH,

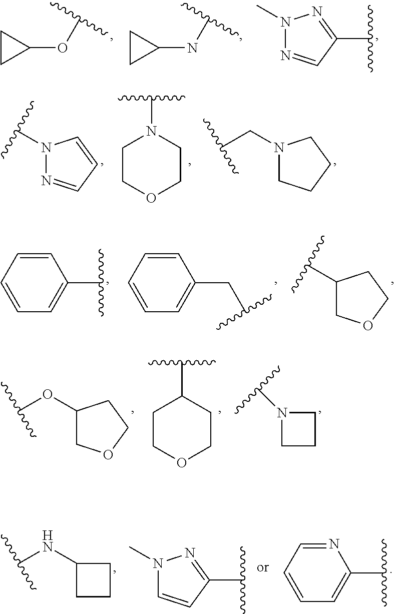

In some embodiments of Formula V, wherein each R³⁰ is independently selected from hydrogen, halogen, —CN, —NO₂, ═O, and C₁₋₆ alkyl.

In some embodiments of Formula V, wherein each R³⁰ is independently selected from hydrogen, F, Cl, Br, ═O, methyl and ethyl.

In some embodiments of Formula V, wherein m is selected from 0, 1, 2 and 3.

In some embodiments of Formula V, wherein p is selected from 0, 1 and 2.

In some embodiments of Formula V, wherein q is selected from 0, 1 and 2.

In some embodiments of Formula I, wherein the compound is of Formula VI, or a pharmaceutically acceptable salt, isomeride, stereoisomer, prodrug, chelate, non-covalent complex, or solvate thereof,

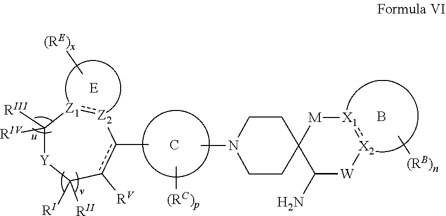

wherein,

[symbol] is a single bond or a double bond;

ring B is 6- to 10-membered aryl, 5- to 10-membered heteroaryl or 5- to 10-membered partially unsaturated heterocyclic ring;

ring C is 5- to 14-membered heteroaryl or 5- to 14-membered partially unsaturated heterocyclic ring;

ring E is 6- to 14-membered aryl, 5- to 14-membered heteroaryl, 5- to 14-membered partially unsaturated heterocyclic ring; wherein the heteroaryl, the heterocyclic ring having 1-4 heteroatoms independently selected from N, O, and S;

X₁ and X₂ are independently selected from C and N;

Z₁ and Z₂ are independently selected from C and N;

M is selected from CH₂, O, NH and S;

W is absent or —CR³¹R³²—.

Y is absent, O, NR^{Y}, C(═O), C(═O)O, C(═O)NR^{Y}, S, S(═O), S(═O)₂, S(═O)O, S(═O)NR^{Y}, S(═O)₂O or S(═O)₂NR^{Y};

each R^{E} is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or

two R^{E} together with the atoms to which they are attached to form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰;

each R^{I}, R^{II}, R^{III}, R^{IV}, R^{V} and R^{Y} is independently selected from hydrogen, halogen, —CN, —NO₂, C₁₋₆ alkyl, C₃₋₈ cycloalkyl, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, or —NR²⁸S(═O)₂R²⁹; wherein C₁₋₆ alkyl, C₃₋₈ cycloalkyl, 6- to 14-membered aryl, 5- to 14-membered heteroaryl and 3- to 8-membered saturated or partially unsaturated heterocyclic ring are optionally substituted with one to four substituents independently selected from R³⁰; or

R^{I} and R^{II} together with the atoms to which they are attached form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰; or

R^{I} together with R^{II} form ═O; or

R^{III} and R^{IV} together with the atoms to which they are attached to form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰; or

R^{III} together with R^{IV} to form ═O;

each R^{B} and R^{C} is independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;

R³¹ and R³² are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;

each R³⁰ is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷ and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring and —C₁₋₆ alkyl;

R³, R⁴, R⁵, R¹³, R²⁶, R²⁷ and R²⁹ are independently selected from hydrogen, halogen, —CN, —NO₂, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OR⁶, —NR⁷R⁸, —SR⁹, —C(═O)OR¹⁰, —C(═O)NR¹¹R¹², —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴ and —NR²⁸S(═O)₂R²⁹; wherein C₁₋₆ alkyl, C₃₋₈ cycloalkyl, 6- to 14-membered aryl, 5- to 14-membered heteroaryl and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring and —C₁₋₆ alkyl;

R⁶, R⁷, R⁸, R⁹, R¹⁰, R¹¹, R¹², R¹⁴, R¹, R¹⁶, R¹⁷, R¹⁸, R¹⁹, R²⁰, R²¹, R²², R²³, R²⁴, R²⁵ and R²⁸ are independently selected from hydrogen, halogen, —CN, —NO₂, ═O, C₁₋₆ alkyl, C₃₋₈ cycloalkyl, 6- to 14-membered aryl, 5- to 14-membered heteroaryl, and 3- to 8-membered saturated or partially unsaturated heterocyclic ring; wherein C₁₋₆ alkyl, C₃₋₈ cycloalkyl, 6- to 14-membered aryl, 5- to 14-membered heteroaryl and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, —OC(═O)R³, —S(═O)R⁴, —C(═O)R⁵, —C(═O)OR¹⁰, —C(═O)NR¹¹R¹², —S(═O)₂R¹³, —S(═O)₂OR¹⁴, —S(═O)₂NR¹⁵R¹⁶, C₃₋₈ cycloalkyl, 3- to 8-membered saturated or partially unsaturated heterocyclic ring, and —C₁₋₆ alkyl;

n is selected from 0, 1, 2, 3 and 4;

p is selected from 0, 1, 2, 3 and 4;

r is selected from 1, 2, 3 and 4;

s is selected from 1, 2, 3 and 4;

x is selected from 0, 1, 2 and 3;

u is selected from 0, 1, 2 and 3;

v is selected from 0, 1, 2 and 3.

In some embodiments of Formula VI, wherein ring E is 6- to 14-membered aryl, 5- to 14-membered heteroaryl or 9- to 14-membered partially unsaturated heterocyclic ring.

In some embodiments of Formula VI, wherein ring E is

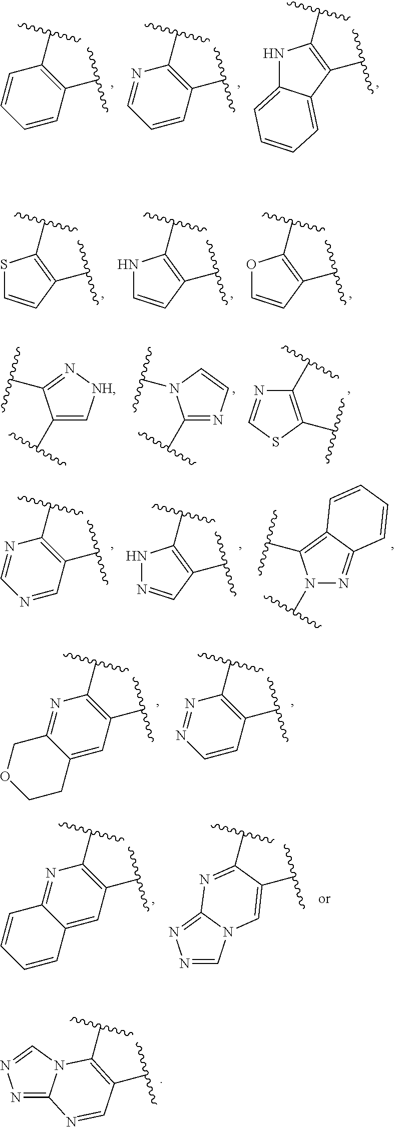

In some embodiments of Formula VI, wherein

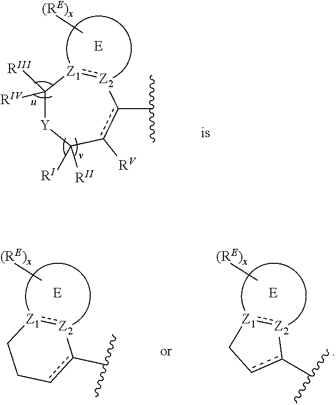

In some embodiments of Formula VI, wherein

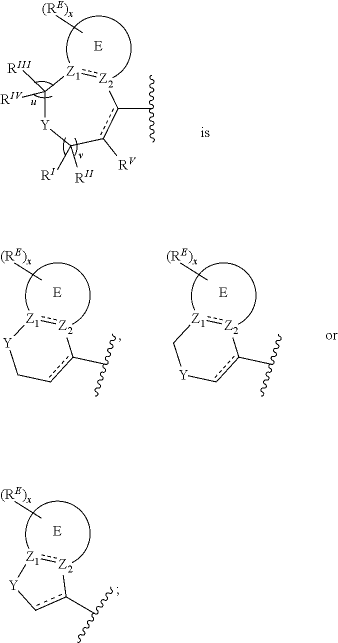

wherein, Y is O, NR, C(═O), C(═O)O, C(═O)NR, S, S(═O), S(═O)₂, S(═O)O, S(═O)NR^{Y}, S(═O)₂O or S(═O)₂NR^{Y}.

In some embodiments of Formula VI, wherein

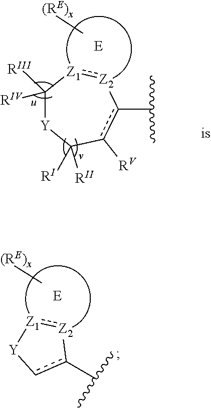

wherein, Y is C(═O), C(═O)O, C(═O)NR^{Y}, S(═O), S(═O)₂, S(═O)O, S(═O)NR^{Y}, S(═O)₂O or S(═O)₂NR^{Y}.

In some embodiments of Formula VI, wherein

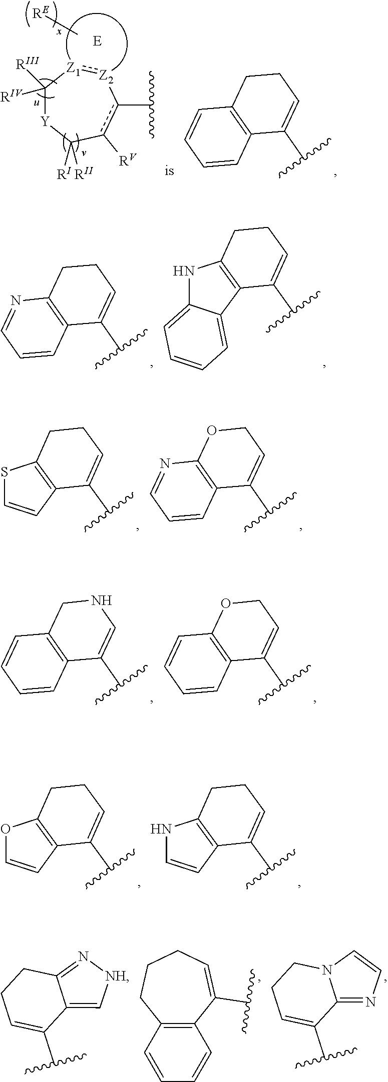

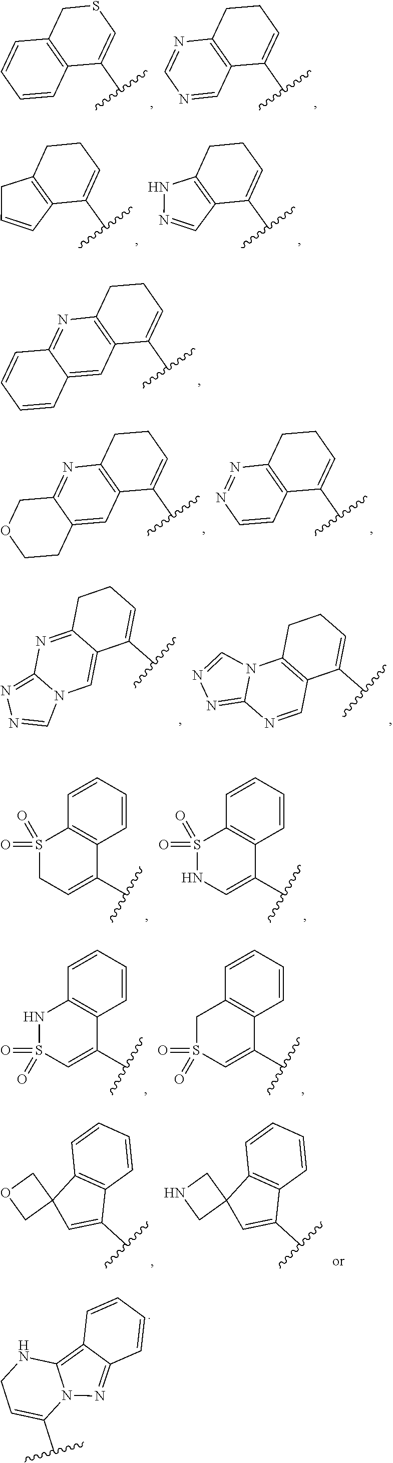

In some embodiments of Formula VI, wherein ring B is 6- to 10-membered aryl.

In some embodiments of Formula VI, wherein ring B is

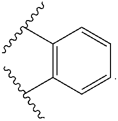 [SMILES: *c1ccccc1*]

In some embodiments of Formula VI, wherein ring C is 9- to 10-membered bicyclic heteroaryl or 9- to 14-membered partially unsaturated bicyclic heterocyclic ring.

In some embodiments of Formula VI, wherein ring C is

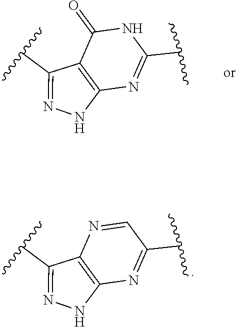 [SMILES: *c1nc2[nH]nc(*)c2c(=O)[nH]1 ; *c1cnc2c(*)n[nH]c2n1]

In some embodiments of Formula VI, wherein ring C is 12- to 14-membered tricyclic heteroaryl or 12- to 14-membered partially unsaturated tricyclic heterocyclic ring.

In some embodiments of Formula VI, wherein ring C is

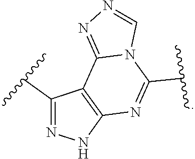 [SMILES: *c1n[nH]c2nc(*)n3cnnc3c12]

In some embodiments of Formula VI, wherein M is CH₂.

In some embodiments of Formula VI, wherein W is absent.

In some embodiments of Formula VI, wherein each R^{E} is independently selected from hydrogen, halogen, —CN, —NO₂, ═O, C₁₋₆ alkyl, 6- to 10-membered aryl, 5- to 10-membered heteroaryl, —C(═O)R⁵, —OR⁶, —NR⁷R⁸, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴ and —NR²⁵C(═O)R²⁶; wherein C₁₋₆ alkyl, 6- to 10-membered aryl and 5- to 10-membered heteroaryl are optionally substituted with one to four substituents independently selected from R³⁰.

In some embodiments of Formula VI, wherein each R^{E} is independently selected from H, CH₃, F, Cl, Br, CF₃, NH₂, CN, COCH₂CH₃, CH₂CF₃, CH₂CH₂CH₂ CH₂CH₃, NHCH₃ and

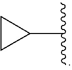 [SMILES: *C1CC1]

In some embodiments of Formula VI, wherein each R^{B} and R^{C} is independently selected from hydrogen, halogen, —CN, —NO₂, ═O and C₁₋₆ alkyl.

In some embodiments of Formula VI, wherein each R^{B} and R^{C} is independently selected from hydrogen, F, Cl, Br, ═O, methyl and ethyl.

In some embodiments of Formula VI, wherein Y is absent.

In some embodiments of Formula VI, wherein Y is O, NR^{Y}, C(═O), C(═O)O, C(═O)NR^{Y}, S, S(═O), S(═O)₂, S(═O)O, S(═O)NR^{Y}, S(═O)₂O or S(═O)₂NR^{Y}.

In some embodiments of Formula VI, wherein R^{I}, R^{II}, R^{III}, R^{IV} and R^{V} are independently selected from hydrogen, halogen, —CN, —NO₂, ═O, C₁₋₆ alkyl and C₃₋₈ cycloalkyl.

In some embodiments of Formula VI, wherein R^{I}, R^{II}, R^{III}, R^{IV} and R^{V} are independently selected from hydrogen, F, Cl, Br, methyl and ethyl.

In some embodiments of Formula VI, wherein R^{I} and R^{II} together with the atoms to which they are attached to form

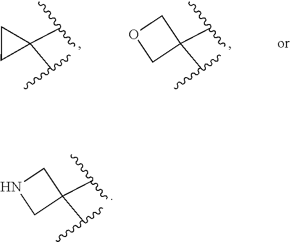 [SMILES: *C1(*)CC1 ; *C1(*)COC1 ; *C1(*)CNC1]

In some embodiments of Formula VI, wherein each R³⁰ is independently selected from hydrogen, halogen, —CN, —NO₂, ═O and C₁₋₆ alkyl.

In some embodiments of Formula VI, wherein each R³⁰ is independently selected from hydrogen, F, Cl, Br, ═O, methyl and ethyl.

In some embodiments of Formula VI, wherein n is selected from 0, 1 and 2.

In some embodiments of Formula VI, wherein p is selected from 1 and 2.

In some embodiments of Formula I, wherein the compound is:
(S)-6-(1-amino-1,3-dihydrospiro[indene-2,4′-piperidin]-1′-yl)-5-methyl-3-(1-phenylcyclopropyl)-1,5-dihydro-4H-pyrazolo[3,4-d]pyrimidin-4-one;
(S)-1′-(3-(1-phenylcyclopropyl)-1H-pyrazolo[3,4-b]pyrazin-6-yl)-1,3-dihydrospiro[indene-2,4′-piperidin]-1-amine;
(S)-1′-(9-(1-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
(S)-6-(1-amino-1,3-dihydrospiro[indene-2,4′-piperidin]-1′-yl)-3-(1-(3-chlorophenyl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(2,2-dimethyl-1-phenylcyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-fluorophenyl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(2,2-dichloro-1-phenylcyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(trifluoromethyl)phenyl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(2,2-difluoro-1-phenylcyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(m-tolyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-fluorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-chlorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2,2-difluorobenzo[d][1,3]dioxol-5-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(quinolin-6-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(trifluoromethyl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(trifluoromethoxy)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-methoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-methoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-methoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3,4-difluorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3,4-dichlorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-aminophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyridin-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(thiophen-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-chloropyridin-4-yl)cyclopropyl)-1,5-dihydro-4H-pyrazolo[3,4-d]pyrimidin-4-one;
(S)-4-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)picolinonitrile;
(S)-6-(1-amino-1,3-dihydrospiro[indene-2,4′-piperidin]-1′-yl)-3-(1-(2-(trifluoromethyl)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-methoxypyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(cyclopropylamino)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-cyclopropoxypyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-morpholinopyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(quinoxalin-6-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-phenylcyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(benzo[d][1,3]dioxol-5-yl)cyclopropyl)-1,5-dihydro-4H-pyrazolo[3,4-d]pyrimidin-4-one;
(S)-3-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)benzonitrile;
(S)-6-(1-amino-1,3-dihydrospiro[indene-2,4′-piperidin]-1′-yl)-3-(1-(3-(1-methyl-1H-pyrazol-4-yl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(2-methyl-2H-1,2,3-triazol-4-yl)phenyl)cyclopropyl)-1,5-dihydro-4H-pyrazolo[3,4-d]pyrimidin-4-one;
(S)-3-(1-(3-(1H-pyrazol-1-yl)phenyl)cyclopropyl)-6-(1-amino-1,3-dihydrospiro[indene-2,4′-piperidin]-1′-yl)-1,5-dihydro-4H-pyrazolo[3,4-d]pyrimidin-4-one;
6-((S)-1-amino-1,3-dihydrospiro[indene-2,4′-piperidin]-1′-yl)-3-(1-(3-(tetrahydrofuran-3-yl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(tetrahydro-2H-pyran-4-yl)phenyl)cyclopropyl)-1,5-dihydro-4H-pyrazolo[3,4-d]pyrimidin-4-one;
ethyl (S)-(3-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)phenyl)carbamate;
(S)-6-(1-amino-1,3-dihydrospiro[indene-2,4′-piperidin]-1′-yl)-3-(1-(3-hydroxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-aminophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(2-methoxyethoxy)phenyl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(1-(3-((tetrahydrofuran-3-yl)oxy)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-amino-3-chloropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-fluoropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2,3-dichloropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2,3-dichlorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-chloro-3-hydroxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyrazin-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(difluoromethoxy)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(difluoromethoxy)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(dimethylamino)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(pyrrolidin-1-ylmethyl)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(1-phenyl-TH-pyrazol-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(1-benzyl-1H-pyrazol-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-fluoropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-amino-3-fluoropyridin-4-yl)cyclopropyl)-1,5-dihydro-4H-pyrazolo[3,4-d]pyrimidin-4-one;
(S)-3-(1-(1-acetyl-3,3-difluoroindolin-4-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-(dimethylamino)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-methoxypyridin-4-yl)cyclopropyl)-1,5-dihydro-4H-pyrazolo[3,4-d]pyrimidin-4-one;
(S)-3-(1-([1,1′-biphenyl]-3-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-morpholinopyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(azetidin-1-yl)-3-chloropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-(cyclobutylamino)pyridin-4-yl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(bicyclo[4.2.0]octa-1(6),2,4-trien-7-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(6-chloropyridazin-3-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(6-chloropyridazin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyridazin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-(cyclopropylamino)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-1-methyl-2-oxo-1,2-dihydropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(naphthalen-1-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(quinolin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(5,6,7,8-tetrahydro-1,8-naphthyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(benzofuran-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(1-methyl-1H-indol-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-oxoindolin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(1,3-dihydroisobenzofuran-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(thiazol-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(oxazol-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-phenylpyridin-4-yl)cyclopropyl)-1,5-dihydro-4H-pyrazolo[3,4-d]pyrimidin-4-one;
(S)-3-(1-([2,2′-bipyridin]-4-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(1-methyl-1H-pyrazol-3-yl)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-methylpyridin-3-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-phenylcyclobutyl)-1,5-dihydro-4H-pyrazolo[3,4-d]pyrimidin-4-one;
(S)-6-(1-amino-1,3-dihydrospiro[indene-2,4′-piperidin]-1′-yl)-5-methyl-3-(1-phenylcyclobutyl)-1,5-dihydro-4H-pyrazolo[3,4-d]pyrimidin-4-one;
(S)-1′-(9-(1-phenylcyclobutyl)-7H-pyrazolo[4,3-e][1,2,4]triazolo[4,3-c]pyrimidin-5-yl)-1,3-dihydrospiro[indene-2,4′-piperidin]-1-amine;
(S)-6-(1-amino-1,3-dihydrospiro[indene-2,4′-piperidin]-1′-yl)-3-(1-(pyridin-2-yl)cyclobutyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(thiophen-2-yl)cyclobutyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-methoxyphenyl)cyclobutyl)-1,5-dihydro-4H-pyrazolo[3,4-d]pyrimidin-4-one;
(S)-6-(1-amino-1,3-dihydrospiro[indene-2,4′-piperidin]-1′-yl)-3-(3-phenyloxetan-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-phenylcyclopentyl)-1,5-dihydro-4H-pyrazolo[3,4-d]pyrimidin-4-one;
6-((S)-1-amino-1,3-dihydrospiro[indene-2,4′-piperidin]-1′-yl)-3-(3-phenyltetrahydrofuran-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-phenylcyclohexyl)-1,5-dihydro-4H-pyrazolo[3,4-d]pyrimidin-4-one;
(S)-6-(1-amino-1,3-dihydrospiro[indene-2,4′-piperidin]-1′-yl)-3-(4-phenyltetrahydro-2H-pyran-4-yl)-1,5-dihydro-4H-pyrazolo[3,4-d]pyrimidin-4-one;
6-((S)-1-amino-1,3-dihydrospiro[indene-2,4′-piperidin]-1′-yl)-3-(1-(2-methoxyphenyl)spiro[2.4]heptan-1-yl)-1,5-dihydro-4H-pyrazolo[3,4-d]pyrimidin-4-one;
6-((S)-1-amino-1,3-dihydrospiro[indene-2,4′-piperidin]-1′-yl)-3-((1S,2R)-2-phenylcyclopropyl)-1,5-dihydro-4H-pyrazolo[3,4-d]pyrimidin-4-one;
(S)-1′-(3-((1S,2R)-2-phenylcyclopropyl)-1H-pyrazolo[3,4-b]pyrazin-6-yl)-1,3-dihydrospiro[indene-2,4′-piperidin]-1-amine;
(S)-1′-(9-((1S,2R)-2-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
6-((S)-1-amino-1,3-dihydrospiro[indene-2,4′-piperidin]-1′-yl)-3-(5,6,7,8-tetrahydroquinolin-5-yl)-1,5-dihydro-4H-pyrazolo[3,4-d]pyrimidin-4-one;
6-((S)-1-amino-1,3-dihydrospiro[indene-2,4′-piperidin]-1′-yl)-3-(4-fluoro-3,3-dimethyl-2,3-dihydro-1H-inden-1-yl)-1,5-dihydro-4H-pyrazolo[3,4-d]pyrimidin-4-one;
6-((S)-1-amino-1,3-dihydrospiro[indene-2,4′-piperidin]-1′-yl)-3-(6,7,8,9-tetrahydro-5H-benzo[7]annulen-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(3,4-dihydronaphthalen-1-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(9-methyl-2,9-dihydro-1H-carbazol-4-yl)-1,5-dihydro-4H-pyrazolo[3,4-d]pyrimidin-4-one;
ethyl (S)-5-(6-(1-amino-1,3-dihydrospiro[indene-2,4′-piperidin]-1′-yl)-4-oxo-4,5-dihydro-1H-pyrazolo[3,4-d]pyrimidin-3-yl)-7,8-dihydroquinoline-3-carboxylate;
(S)-6-(1-amino-1,3-dihydrospiro[indene-2,4′-piperidin]-1′-yl)-3-(7-fluoro-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2H-pyrano[2,3-b]pyridin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6,7-dihydrobenzo[b]thiophen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-pentyl-6,7-dihydrobenzo[b]thiophen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6,7-dihydrobenzofuran-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-methyl-6,7-dihydro-1H-indol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-methyl-6,7-dihydro-2H-indazol-4-yl)-1,5-dihydro-4H-pyrazolo[3,4-d]pyrimidin-4-one;
(S)-5-(6-(1-amino-1,3-dihydrospiro[indene-2,4′-piperidin]-1′-yl)-4-oxo-4,5-dihydro-1H-pyrazolo[3,4-d]pyrimidin-3-yl)-7,8-dihydronaphthalene-2-carbonitrile;
(S)-6-(1-amino-1,3-dihydrospiro[indene-2,4′-piperidin]-1′-yl)-3-(4,4-difluoro-3,4-dihydronaphthalen-1-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8-fluoro-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6,7-dihydro-5H-benzo[7]annulen-9-yl)-1,5-dihydro-4H-pyrazolo[3,4-d]pyrimidin-4-one;
8-(6-(1-amino-1,3-dihydrospiro[indene-2,4′-piperidin]-1′-yl)-4-oxo-4,5-dihydro-1H-pyrazolo[3,4-d]pyrimidin-3-yl)-5,5-difluoro-5,6-dihydronaphthalene-2-carbonitrile;
6-(1-amino-1,3-dihydrospiro[indene-2,4′-piperidin]-1′-yl)-3-(8,8-dimethyl-7,8-dihydroquinolin-5-yl)-1,5-dihydro-4H-pyrazolo[3,4-d]pyrimidin-4-one;
6-(1-amino-1,3-dihydrospiro[indene-2,4′-piperidin]-1′-yl)-3-(5,6-dihydroimidazo[1,2-a]pyridin-8-yl)-1,5-dihydro-4H-pyrazolo[3,4-d]pyrimidin-4-one;
6-(1-amino-1,3-dihydrospiro[indene-2,4′-piperidin]-1′-yl)-3-(2,5,5-trimethyl-4,5-dihydrobenzo[d]thiazol-7-yl)-1,5-dihydro-4H-pyrazolo[3,4-d]pyrimidin-4-one;
6-(1-amino-1,3-dihydrospiro[indene-2,4′-piperidin]-1′-yl)-3-(2-amino-5,5-dimethyl-4,5-dihydrobenzo[d]thiazol-7-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-methyl-6,7-dihydro-TH-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2′H-spiro[cyclopropane-1,1′-naphthalen]-4′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7′H-spiro[cyclopropane-1,8′-quinolin]-5′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1H-isothiochromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6-chloro-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2,4-bis(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(3-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-chloro-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-methyl-4-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-cyclopropyl-4-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(4-(difluoromethyl)-2-methyl-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7,7-dimethyl-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7,7-dimethyl-2-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8,8-dimethyl-2-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7,7-dimethyl-2-(trifluoromethyl)-7,8-dihydroquinazolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2,7,7-trimethyl-7,8-dihydroquinazolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-cyclopropyl-7,7-dimethyl-7,8-dihydroquinazolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7,7-dimethyl-2-(methylamino)-7,8-dihydroquinazolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,6,6-trimethyl-6,7-dihydro-1H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6,6-dimethyl-1-(2,2,2-trifluoroethyl)-6,7-dihydro-1H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-cyclopropyl-6,6-dimethyl-6,7-dihydro-1H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2,6,6-trimethyl-6,7-dihydro-2H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6,6-dimethyl-2-(2,2,2-trifluoroethyl)-6,7-dihydro-2H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-cyclopropyl-6,6-dimethyl-6,7-dihydro-2H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(3-cyclopropyl-6,6-dimethyl-1-(2,2,2-trifluoroethyl)-6,7-dihydro-1H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(3,3-dimethyl-3,4-dihydroacridin-1-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8,8-dimethyl-3,4,8,9-tetrahydro-1H-pyrano[3,4-b]quinolin-6-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-amino-5,5-dimethyl-4,5-dihydrobenzo[d]thiazol-7-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(3-bromo-7,7-dimethyl-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-chloro-7,7-dimethyl-7,8-dihydroquinolin-5-yl)-1,5-dihydro-4H-pyrazolo[3,4-d]pyrimidin-4-one;
(S)-5-(6-(1-amino-1,3-dihydrospiro[indene-2,4′-piperidin]-1′-yl)-4-oxo-4,5-dihydro-H-pyrazolo[3,4-d]pyrimidin-3-yl)-7,7-dimethyl-7,8-dihydroquinoline-2-carbonitrile;
(S)-5-(6-(1-amino-1,3-dihydrospiro[indene-2,4′-piperidin]-1′-yl)-4-oxo-4,5-dihydro-1H-pyrazolo[3,4-d]pyrimidin-3-yl)-7,7-dimethyl-7,8-dihydroquinoline-3-carbonitrile;
(S)-6-(1-amino-1,3-dihydrospiro[indene-2,4′-piperidin]-1′-yl)-3-(3,7,7-trimethyl-7,8-dihydrocinnolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8,8-dimethyl-8,9-dihydro-[1,2,4]triazolo[4,3-a]quinazolin-6-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8,8-dimethyl-8,9-dihydro-[1,2,4]triazolo[3,4-b]quinazolin-6-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7-chloro-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7-(trifluoromethyl)-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8,8-difluoro-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(spiro[indene-1,3′-oxetan]-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-methylspiro[azetidine-3,1′-inden]-3′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1H-inden-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-oxo-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,1-difluoro-1H-inden-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,1-dimethyl-1H-inden-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,2-dihydroquinolin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-methyl-1,2-dihydroquinolin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,1-dimethyl-1,2-dihydroisoquinolin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,1-dioxido-2H-thiochromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,1-dioxido-2H-benzo[e][1,2]thiazin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2,2-dioxido-TH-isothiochromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2,2-dioxido-TH-benzo[c][1,2]thiazin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-methyl-2,2-dioxido-1H-benzo[c][1,2]thiazin-4-yl)-1,5-dihydro-4H-pyrazolo[3,4-d]pyrimidin-4-one;
6-((S)-1-amino-1,3-dihydrospiro[indene-2,4′-piperidin]-1′-yl)-3-((1S,2S)-2-phenylcyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(6-fluoro-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6-fluoro-2-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-chloro-6-fluoro-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6-fluoro-7,7-dimethyl-2-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6-fluoro-7,7-dimethyl-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7-methoxybenzofuran-3-yl)-1,5-dihydro-4H-pyrazolo[3,4-d]pyrimidin-4-one;
(S)-6-(1-amino-6-methoxy-1,3-dihydrospiro[indene-2,4′-piperidin]-1′-yl)-3-(1-phenylcyclopropyl)-1,5-dihydro-4H-pyrazolo[3,4-d]pyrimidin-4-one;
(S)-6-(4-amino-2-chloro-4,6-dihydrospiro[cyclopenta[d]thiazole-5,4′-piperidin]-1′-yl)-3-(1-phenylcyclopropyl)-1,5-dihydro-4H-pyrazolo[3,4-d]pyrimidin-4-one;
(S)-6-(1-amino-6-chloro-1,3-dihydrospiro[indene-2,4′-piperidin]-1′-yl)-3-(1-phenylcyclopropyl)-1,5-dihydro-4H-pyrazolo[3,4-d]pyrimidin-4-one;
(S)-6-(1-amino-6-fluoro-1,3-dihydrospiro[indene-2,4′-piperidin]-1′-yl)-3-(1-phenylcyclopropyl)-1,5-dihydro-4H-pyrazolo[3,4-d]pyrimidin-4-one;
(S)-6-(1-amino-6-(methylthio)-1,3-dihydrospiro[indene-2,4′-piperidin]-1′-yl)-3-(1-phenylcyclopropyl)-1,5-dihydro-4H-pyrazolo[3,4-d]pyrimidin-4-one;
(S)-1-amino-1′-(4-oxo-3-(1-phenylcyclopropyl)-4,5-dihydro-1H-pyrazolo[3,4-d]pyrimidin-6-yl)-1,3-dihydrospiro[indene-2,4′-piperidine]-6-carbonitrile; (R)-6-(2-amino-2,3-dihydrospiro[indene-1,4′-piperidin]-1′-yl)-3-(1-phenylcyclopropyl)-1,5-dihydro-4H-pyrazolo[3,4-d]pyrimidin-4-one;
(S)-6-(5′-amino-5′,6′-dihydrospiro[piperidine-4,4′-pyrrolo[1,2-b]pyrazol]-1-yl)-3-(1-phenylcyclopropyl)-1,5-dihydro-4H-pyrazolo[3,4-d]pyrimidin-4-one;
(S)-6-(5-amino-5,7-dihydrospiro[cyclopenta[b]pyridine-6,4′-piperidin]-1′-yl)-3-(1-phenylcyclopropyl)-1,5-dihydro-4H-pyrazolo[3,4-d]pyrimidin-4-one;
(S)-1′-(3-(1-phenylcyclopropyl)-1H-pyrazolo[3,4-b]pyrazin-6-yl)-5,7-dihydrospiro[cyclopenta[b]pyridine-6,4′-piperidin]-5-amine;
(S)-6-chloro-1′-(3-(1-phenylcyclopropyl)-1H-pyrazolo[3,4-b]pyrazin-6-yl)-1,3-dihydrospiro[indene-2,4′-piperidin]-1-amine;
(S)-6-fluoro-1′-(3-(1-phenylcyclopropyl)-1H-pyrazolo[3,4-b]pyrazin-6-yl)-1,3-dihydrospiro[indene-2,4′-piperidin]-1-amine;
(S)-6-(methylthio)-1′-(3-(1-phenylcyclopropyl)-1H-pyrazolo[3,4-b]pyrazin-6-yl)-1,3-dihydrospiro[indene-2,4′-piperidin]-1-amine;
(S)-2-chloro-1′-(3-(1-phenylcyclopropyl)-1H-pyrazolo[3,4-b]pyrazin-6-yl)-4,6-dihydrospiro[cyclopenta[d]thiazole-5,4′-piperidin]-4-amine;
(S)-1-(3-(1-phenylcyclopropyl)-1H-pyrazolo[3,4-b]pyrazin-6-yl)-5′,6′-dihydrospiro[piperidine-4,4′-pyrrolo[1,2-b]pyrazol]-5′-amine;
(S)-1-(9-(1-phenylcyclopropyl)-7H-pyrazolo[4,3-e][1,2,4]triazolo[4,3-c]pyrimidin-5-yl)-5′,6′-dihydrospiro[piperidine-4,4′-pyrrolo[1,2-b]pyrazol]-5′-amine;
(S)-6-methoxy-1′-(9-(1-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
(S)-1′-(9-(1-phenylcyclopropyl)-7H-pyrazolo[4,3-e][1,2,4]triazolo[4,3-c]pyrimidin-5-yl)-5,7-dihydrospiro[cyclopenta[b]pyridine-6,4′-piperidin]-5-amine;
(S)-6-chloro-1′-(9-(1-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
(S)-6-fluoro-1′-(9-(1-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
(S)-6-(methylthio)-1′-(9-(1-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
(S)-2-chloro-1′-(9-(1-phenylcyclopropyl)-7H-pyrazolo[4,3-e][1,2,4]triazolo[4,3-c]pyrimidin-5-yl)-4,6-dihydrospiro[cyclopenta[d]thiazole-5,4′-piperidin]-4-amine;
(S)-6-(1-amino-1,3-dihydrospiro[indene-2,4′-piperidin]-1′-yl)-3-(2-phenylpropan-2-yl)-1,5-dihydro-4H-pyrazolo[3,4-d]pyrimidin-4-one;
6-((S)-1-amino-1,3-dihydrospiro[indene-2,4′-piperidin]-1′-yl)-3-(1-phenylpropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-cyclopropylphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-fluorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3,4-dimethoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-ethynylphenyl)cyclopropyl)-1,5-dihydro-4H-pyrazolo[3,4-d]pyrimidin-4-one;
(S)-3-(1-(3-acetylphenyl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(dimethylphosphoryl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(methylthio)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(hydroxymethyl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-cyclopropoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-(trifluoromethyl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-aminophenyl)cyclopropyl)-1,5-dihydro-4H-pyrazolo[3,4-d]pyrimidin-4-one;
ethyl (S)-(4-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)phenyl)carbamate;
(S)-6-(1-amino-1,3-dihydrospiro[indene-2,4′-piperidin]-1′-yl)-3-(1-(2,4-dimethoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-(trifluoromethoxy)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-hydroxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2,4-dichlorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(thiophen-3-yl)cyclopropyl)-1,5-dihydro-4H-pyrazolo[3,4-d]pyrimidin-4-one;
(S)-4-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)benzonitrile;
(S)-5-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)-1,3,4-thiadiazole-2-carbonitrile;
(S)-3-(1-(1,3,4-thiadiazol-2-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-3-(1-(1,2,3-thiadiazol-4-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(5-methyl-1,2,3-thiadiazol-4-yl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(1-(1-oxidothiophen-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(oxazol-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(5-methyloxazol-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyrimidin-2-yl)cyclopropyl)-1,5-dihydro-4H-pyrazolo[3,4-d]pyrimidin-4-one;
(S)-3-(1-(2H-tetrazol-5-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyridin-3-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(6-methylpyridin-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(5-cyclopropyl-1,3,4-thiadiazol-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(benzofuran-3-yl)cyclopropyl)-1,5-dihydro-4H-pyrazolo[3,4-d]pyrimidin-4-one;
(S)-3-(1-(1H-benzo[d]imidazol-2-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(benzo[d]oxazol-2-yl)cyclopropyl)-1,5-dihydro-4H-pyrazolo[3,4-d]pyrimidin-4-one;
(S)-3-(1-(1H-1,2,3-triazol-4-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-3-(1-(1H-pyrrol-1-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-3-(1-(1H-pyrazol-1-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(furan-2-yl)cyclopropyl)-1,5-dihydro-4H-pyrazolo[3,4-d]pyrimidin-4-one; (R)-6-(1-amino-1,3-dihydrospiro[indene-2,4′-piperidin]-1′-yl)-3-(1-phenylcyclopropyl)-1,5-dihydro-4H-pyrazolo[3,4-d]pyrimidin-4-one;
(3S,4S)-3-methyl-8-(5-(1-phenylcyclopropyl)pyrazin-2-yl)-2-oxa-8-azaspiro[4.5]decan-4-amine;
3-((3S,4S)-4-amino-3-methyl-2-oxa-8-azaspiro[4.5]decan-8-yl)-6-(1-phenylcyclopropyl)pyrazine-2-carboxamide;
(3-((3S,4S)-4-amino-3-methyl-2-oxa-8-azaspiro[4.5]decan-8-yl)-6-(1-phenylcyclopropyl)pyrazin-2-yl)methanol;
(3-((3S,4S)-4-amino-3-methyl-2-oxa-8-azaspiro[4.5]decan-8-yl)-5-methyl-6-(1-phenylcyclopropyl)pyrazin-2-yl)methanol;
2-((3S,4S)-4-amino-3-methyl-2-oxa-8-azaspiro[4.5]decan-8-yl)-5-(1-phenylcyclopropyl)pyrimidin-4(3H)-one;
2-((3S,4S)-4-amino-3-methyl-2-oxa-8-azaspiro[4.5]decan-8-yl)-3-methyl-5-(1-phenylcyclopropyl)pyrimidin-4(3H)-one;
6-amino-2-((3S,4S)-4-amino-3-methyl-2-oxa-8-azaspiro[4.5]decan-8-yl)-3-methyl-5-(1-phenylcyclopropyl)pyrimidin-4(3H)-one;
(3S,4S)-8-(8-amino-9-(1-phenylcyclopropyl)-3,4-dihydro-2H-pyrimido[1,6-a]pyrimidin-6-yl)-3-methyl-2-oxa-8-azaspiro[4.5]decan-4-amine;
(3S,4S)-8-(5-amino-6-(1-phenylcyclopropyl)-2,3-dihydroimidazo[1,2-a]pyrimidin-7-yl)-3-methyl-2-oxa-8-azaspiro[4.5]decan-4-amine;
(3S,4S)-3-methyl-8-(3-(1-phenylcyclopropyl)-1H-pyrazolo[3,4-d]pyrimidin-6-yl)-2-oxa-8-azaspiro[4.5]decan-4-amine;
(3S,4S)-3-methyl-8-(3-(1-(thiophen-3-yl)cyclopropyl)-1H-pyrazolo[3,4-b]pyrazin-6-yl)-2-oxa-8-azaspiro[4.5]decan-4-amine;
(3S,4S)-3-methyl-8-(7-(1-phenylcyclopropyl)-5H-pyrrolo[2,3-b]pyrazin-3-yl)-2-oxa-8-azaspiro[4.5]decan-4-amine;
6-((3S,4S)-4-amino-3-methyl-2-oxa-8-azaspiro[4.5]decan-8-yl)-3-(1-phenylcyclopropyl)-1,5-dihydro-4H-pyrazolo[3,4-d]pyrimidin-4-one;
(S)-6-(4-amino-2-oxa-8-azaspiro[4.5]decan-8-yl)-3-(1-phenylcyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-5-methyl-3-(1-phenylcyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(pyrimidin-4-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(pyridin-4-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(pyridin-3-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(pyridin-2-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(thiazol-2-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(thiophen-2-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(oxazol-2-yl)cyclopropyl)-1,5-dihydro-4H-pyrazolo[3,4-d]pyrimidin-4-one;
3-(1-(1,3,4-thiadiazol-2-yl)cyclopropyl)-6-((3S,4S)-4-amino-3-methyl-2-oxa-8-azaspiro[4.5]decan-8-yl)-1,5-dihydro-4H-pyrazolo[3,4-d]pyrimidin-4-one;
(S)-6-(4-amino-2-oxa-8-azaspiro[4.5]decan-8-yl)-3-(1-(benzo[d]oxazol-2-yl)cyclopropyl)-1,5-dihydro-4H-pyrazolo[3,4-d]pyrimidin-4-one;
(S)-3-(1-(1H-benzo[d]imidazol-2-yl)cyclopropyl)-6-(4-amino-2-oxa-8-azaspiro[4.5]decan-8-yl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(benzo[d]oxazol-2-yl)cyclopropyl)-1,5-dihydro-4H-pyrazolo[3,4-d]pyrimidin-4-one;
3-(1-(1H-indol-2-yl)cyclopropyl)-6-((3S,4S)-4-amino-3-methyl-2-oxa-8-azaspiro[4.5]decan-8-yl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(benzo[d][1,3]dioxol-5-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(benzo[d]oxazol-5-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-fluoro-5-methoxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-fluoro-3-methoxyphenyl)cyclopropyl)-1,5-dihydro-4H-pyrazolo[3,4-d]pyrimidin-4-one;
3-(1-(6-((3S,4S)-4-amino-3-methyl-2-oxa-8-azaspiro[4.5]decan-8-yl)-4-oxo-4,5-dihydro-1H-pyrazolo[3,4-d]pyrimidin-3-yl)cyclopropyl)benzonitrile;
3-(1-(2-amino-3-chloropyridin-4-yl)cyclopropyl)-6-((3S,4S)-4-amino-3-methyl-2-oxa-8-azaspiro[4.5]decan-8-yl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-fluorophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-fluorophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2-fluorophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3,4-difluorophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-(trifluoromethyl)phenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-(trifluoromethyl)phenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2-(trifluoromethyl)phenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-aminophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(p-tolyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(m-tolyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(o-tolyl)cyclopropyl)-1,5-dihydro-4H-pyrazolo[3,4-d]pyrimidin-4-one;
ethyl (4-(1-(6-((3S,4S)-4-amino-3-methyl-2-oxa-8-azaspiro[4.5]decan-8-yl)-4-oxo-4,5-dihydro-1H-pyrazolo[3,4-d]pyrimidin-3-yl)cyclopropyl)phenyl)carbamate;
6-((3S,4S)-4-amino-3-methyl-2-oxa-8-azaspiro[4.5]decan-8-yl)-3-(1-(4-methoxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-methoxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2-methoxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-(trifluoromethoxy)phenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2,2-difluorobenzo[d][1,3]dioxol-5-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2,3-dichlorophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-hydroxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-hydroxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2,4-dichlorophenyl)cyclopropyl)-1,5-dihydro-4H-pyrazolo[3,4-d]pyrimidin-4-one;
3-(1-(3-(1H-pyrazol-1-yl)phenyl)cyclopropyl)-6-((3S,4S)-4-amino-3-methyl-2-oxa-8-azaspiro[4.5]decan-8-yl)-1,5-dihydro-4H-pyrazolo[3,4-d]pyrimidin-4-one;
4-(1-(6-((3S,4S)-4-amino-3-methyl-2-oxa-8-azaspiro[4.5]decan-8-yl)-4-oxo-4,5-dihydro-1H-pyrazolo[3,4-d]pyrimidin-3-yl)cyclopropyl)benzonitrile;
3-(1-(3-acetylphenyl)cyclopropyl)-6-((3R,4R)-4-amino-3-methyl-2-oxa-8-azaspiro[4.5]decan-8-yl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-bromophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-methylthiazol-2-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(6-methylpyridin-2-yl)cyclopropyl)-1,5-dihydro-4H-pyrazolo[3,4-d]pyrimidin-4-one;
6-((1R,2R)-1-amino-2-methyl-8-azaspiro[4.5]decan-8-yl)-3-(1-phenylcyclopropyl)-1,5-dihydro-4H-pyrazolo[3,4-d]pyrimidin-4-one;
(R)-6-(1-amino-8-azaspiro[4.5]decan-8-yl)-3-(1-phenylcyclopropyl)-1,5-dihydro-4H-pyrazolo[3,4-d]pyrimidin-4-one;
(R)-6-(3-amino-3H-spiro[benzofuran-2,4′-piperidin]-1′-yl)-3-(1-phenylcyclopropyl)-1,5-dihydro-4H-pyrazolo[3,4-d]pyrimidin-4-one;
(R)-1′-(3-(1-phenylcyclopropyl)-1H-pyrazolo[3,4-b]pyrazin-6-yl)-3H-spiro[benzofuran-2,4′-piperidin]-3-amine; or
(R)-1′-(9-(1-phenylcyclopropyl)-7H-pyrazolo[4,3-e][1,2,4]triazolo[4,3-c]pyrimidin-5-yl)-3H-spiro[benzofuran-2,4′-piperidin]-3-amine.

The present invention also provides a pharmaceutical composition comprising a compound of any one of the present invention, a pharmaceutically acceptable salt, isomeride, stereoisomer, prodrug, chelate, non-covalent complex, or solvate, and at least one pharmaceutically acceptable carrier or excipient.

The present invention also provides a use of the present compound or its pharmaceutical composition for the preparation of a medicament.

In some embodiments, wherein the medicament is used for treating, preventing, delaying or preventing cancer, metastasis of cancer, cardiovascular disease, immune disease, fibrosis or ocular disease.

In some embodiments, wherein the medicament is used for treating a disease mediated by SHP2.

In some embodiments, wherein the disease is cancer.

In some embodiments, wherein the cancer is Noonan syndrome, leopard spot syndrome, juvenile myelomonocytic leukemia, neuroblastoma, melanoma, head and neck squamous cell carcinoma, acute myeloid leukemia, breast cancer, esophageal cancer, lung cancer, colon cancer, head cancer, gastric cancer, lymphoma, glioblastoma, and/or pancreatic cancer.

The present invention also provides a use of the present compound or its pharmaceutical composition for the preparation of SHP2 inhibitors.

The present invention also provides a method for treating and/or preventing a disease mediated by SHP2, said method administering to the patient in need a compound of any one of the present invention, or pharmaceutical composition.

In some embodiments, wherein the disease is cancer.

The present invention also provides a method for treating a cancer, said method administering to the patient in need a compound of any one of the present invention, or pharmaceutical composition.

In some embodiments, wherein the cancer is Noonan syndrome, leopard spot syndrome, juvenile myelomonocytic leukemia, neuroblastoma, melanoma, head and neck squamous cell carcinoma, acute myeloid leukemia, breast cancer, esophageal cancer, lung cancer, colon cancer, head cancer, gastric cancer, lymphoma, glioblastoma, and/or pancreatic cancer.

The general chemical terms used in the formula above have their usual meanings. For example, the term “halogen”, as used herein, unless otherwise indicated, means fluoro, chloro, bromo or iodo. The preferred halogen groups include F, Cl and Br.

As used herein, unless otherwise indicated, alkyl includes saturated monovalent hydrocarbon radicals having straight, or branched moieties. For example, alkyl radicals include methyl, ethyl, propyl, isopropyl, n-butyl, isobutyl, sec-butyl, t-butyl, n-pentyl, 3-(2-methyl) butyl, 2-pentyl, 2-methylbutyl, neopentyl, n-hexyl, 2-hexyl, and 2-methylpentyl. Similary, C₁₋₈, as in C₁₋₈ alkyl is defined to identify the group as having 1, 2, 3, 4, 5, 6, 7 or 8 carbon atoms in a linear or branched arrangement.

Alkenyl and alkynyl groups include straight, branched chain or cyclic alkenes and alkynes. Likewise, “C₂₋₈ alkenyl” and “C₂₋₈ alkynyl” means an alkenyl or alkynyl radicals having 2, 3, 4, 5, 6, 7 or 8 carbon atoms in a linear or brached arrangement. For example, alkenyl radicals include ethenyl, propenyl, etc. For example, alkynyl radicals include ethynyl, propynyl, etc.

Alkoxy radicals are oxygen ethers formed from the previously described straight, branched chain or cyclic alkyl groups. For example, alkoxy radicals include methoxyl, ethoxyl, propoxyl, isopropoxyl, cyclcopropoxyl, n-butoxyl, isobutoxyl, sec-butoxyl, t-butoxyl, cyclcobutoxyl, n-pentoxyl, 3-(2-methyl) butoxyl, 2-pentoxyl, 2-methylbutoxyl, neopentoxyl, cyclcopentoxyl, n-hexoxyl, 2-hexoxyl, 2-methylpentoxyl and cyclohexoxyl.

The term “aryl”, as used herein, unless otherwise indicated, refers to an unsubstituted or substituted mono- or polycyclic ring system containing carbon ring atoms. The preferred aryls are mono cyclic or bicyclic 6-10 membered aromatic ring systems. Phenyl and naphthyl are preferred aryls. The most preferred aryl is phenyl.

The term “heterocyclyl”, as used herein, unless otherwise indicated, represents an unsubstituted or substituted stable three to ten membered ring system which consists of carbon atoms and one to three heteroatoms selected from N, O and S, and wherein the nitrogen or sulfur heteroatoms may optionally be oxidized, and the nitrogen heteroatom may optionally be quaternized. The heterocyclyl group may be attached at any heteroatom or carbon atom which results in the creation of a stable structure. The heterocyclyl group is formed by single bonds, or by single and double bonds. The term “heterocyclyl” represents an unsubstituted or substituted stable three or seven membered monocyclic ring system or an unsubstituted or substituted six or ten membered bicyclic ring system. Examples of such heterocyclyl groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl, 1,2,3,4-tetrahydroisoquinolinyl, thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “heteroaryl”, as used herein, unless otherwise indicated, represents an unsubstituted or substituted stable five or six membered monocyclic aromatic ring system or an unsubstituted or substituted nine or ten membered benzo-fused heteroaromatic ring system or bicyclic heteroaromatic ring system which consists of carbon atoms and from one to four heteroatoms selected from N, O and S, and wherein the nitrogen or sulfur heteroatoms may optionally be oxidized, and the nitrogen heteroatom may optionally be quaternized. The heteroaryl group may be attached at any heteroatom or carbon atom which results in the creation of a stable structure. Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “alkenyloxy” refers to the group —O-alkenyl, where alkenyl is defined as above.

The term “alknyloxy” refers to the group —O-alknyl, where alknyl is defined as above.

The term “cycloalkyl” to a cyclic saturated alkyl chain having from 3 to 12 carbon atoms, for example, cyclopropyl, cyclobutyl, cyclopentyl, or cyclohexyl.

The term “substituted” refers to a group in which one or more hydrogen atoms are each independently replaced with the same or different substituent(s). Typical substituents include, but are not limited to, halogen (F, Cl, Br or I), C₁₋₈ alkyl, C₃₋₁₂ cycloalkyl, —OR¹, SR¹, ═O, ═S, —C(O)R¹, —C(S)R¹, ═NR¹, —C(O)OR¹, —C(S)OR¹, —NR¹R², —C(O)NR¹R², cyano, nitro, —S(O)₂R¹, —OS(O₂)OR¹, —OS(O)₂R¹, —OP(O)(OR¹)(OR²); wherein R¹ and R² is independently selected from —H, lower alkyl and lower haloalkyl. In some embodiments, the substituent(s) is independently selected from the group consisting of —F, —Cl, —Br, —I, —OH, trifluromethoxy, ethoxy, propyloxy, iso-propyloxy, n-butyloxy, isobutyloxy, t-butyloxy, —SCH₃, —SC₂H₅, formaldehyde group, —C(OCH₃), cyano, nitro, CF₃, —OCF₃, amino, dimethylamino, methyl thio, sulfonyl and acetyl.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

Examples of substituted alkyl group include, but not limited to, 2-aminoethyl, 2-hydroxyethyl, pentachloroethyl, trifluoromethyl, methoxymethyl, pentafluoroethyl and piperazinylmethyl.

Examples of substituted alkoxy groups include, but not limited to, aminomethoxy, thrifluoromethoxy, 2-diethylaminoethoxy, 2-ethoxycarbonylethoxy, 3-hydroxypropoxy.

The compounds of the present invention may also be present in the form of pharmaceutically acceptable salts. For use in medicine, the salts of the compounds of this invention refer to non-toxic “pharmaceutically acceptable salts”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula I are shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula I exists, the present invention includes any possible tautomers and pharmaceutically acceptable salts thereof, and mixtures thereof, except where specifically stated otherwise.

When the compound of Formula I and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, formic, hydrochloric, maleic, phosphoric, sulfuric and tartaric acids, particularly preferred are formic and hydrochloric acid. Since the compounds of Formula I are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound represented by Formula I (or a pharmaceutically acceptable salt thereof) as an active ingredient, a pharmaceutically acceptable carrier and optionally other therapeutic ingredients or adjuvants.

The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or a prodrug, or a metabolite, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula I, or a pharmaceutically acceptable salt thereof, may also be administered by controlled release means and/or delivery devices. The compositions may be prepared by any of the methods of pharmacy.

In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound, or a pharmaceutically acceptable salt, of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include such as lactose, terra alba, sucrose, talc, gelatin, agar, pectin, acacia, magnesium stearate, and stearic acid. Examples of liquid carriers include such as sugar syrup, peanut oil, olive oil, and water. Examples of gaseous carriers include such as carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, colon cancer, rectal cancer, mantle cell lymphoma, multiple myeloma, breast cancer, prostate cancer, glioblastoma, squamous cell esophageal cancer, liposarcoma, T-cell lymphoma melanoma, pancreatic cancer, or lung cancer, may be effectively treated by the administration of from about 0.01 to 50 mg of the compound per kilogram of body weight per day, or alternatively about 0.5 mg to about 3.5 g per patient per day.

It is understood, however, that lower or higher doses than those recited above may be required. Specific dose level and treatment regimens for any particular subject will depend upon a variety of factors, including the activity of the specific compound employed, the age, body weight, general health, sex, diet, time of administration, route of administration, rate of excretion, drug combination, the severity and course of the particular disease undergoing therapy, the subject disposition to the disease, and the judgment of the treating physician.

These and other aspects will become apparent from the following written description of the invention.

The following Examples are provided to better illustrate the present invention. All parts and percentages are by weight and all temperatures are degrees Celsius, unless explicitly stated otherwise.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit SHP2 according to at least one assay described herein.

### EXAMPLES

Experimental procedures for compounds of the invention are provided below. And the starting materials are either commercially available or made by known procedures in the reported literature or as illustrated.

The following abbreviations have been used in the examples:
AcOH: Acetic acid;
B₂Pin₂: Octamethyl-2,2′-bi-1,3,2-dioxaborolane;
BOP: (benzotriazol-1-yloxytris(dimethylamino)phosphonium hexafluorophosphate);
CatacXium A Pd G₃: Mesylate[(di(1-adamantyl)-n-butylphosphine)-2-(2′-amino-1,1′-biphenyl)]palladium(II);
Cu(acac)₂: Copper(II) acetylacetonate;
DBU: 1,8-Diazabicyclo(5.4.0)undec-7-ene;
DIBALH or DIBAL-H: Diisobutylaluminium hydride;
DCM: Dichloromethane;
DIC: N, N-diisopropylcarbodiimide;
DIEA: N,N-Diisopropylethylamine;
DiFMUP: (6,8-Difluoro-4-Methylumbelliferyl Phosphate);
DMF: Dimethylformamide;
DMAP: 4-Dimethylaminopyridine;
DMSO: Dimethyl sulfoxide;
EA: Ethyl acetate;
EDTA: Ethylenediaminetetraacetic acid;
HATU: Hexafluorophosphate Azabenzotriazole Tetramethyl Uronium;
HEPES: 4-(2-hydroxyethyl)-1-piperazineethanesulfonic acid;
LCMS: Liquid chromatography-mass spectrometry;
LiTMP: Lithium 2,2,6,6-tetramethylpiperidide;
h or hrs: hour or hours;
PE: Petroleum ether;
PdCl₂(PPh₃)₂: Palladium(II)bis(triphenylphosphine) dichloride;
PdCl₂(dppf)CH₂Cl₂: 1,1′-Bis(diphenylphosphino)ferrocene-palladium(II)dichloride dichloromethane complex;
PhNTf2: N-Phenyl-bis(trifluoromethanesulfonimide);
PPA: polyphosphoric acid;
PPh₃: Triphenylphosphine;
MeOH: Methanol;
min: minute;
NaHMDS: sodium bis(trimethylsilyl)amide;
NCS: N-Chlorosuccinimide;
rt or R.T: room temperature;
TEA: Triethylamine;
TFA: Trifluoroacetic acid;
THF: Tetrahydrofuran;
TLC: Preparative thin layer chromatography;
1N:1 mol·L⁻¹, (2N:2 mol·L⁻¹, etc.).

### Preparation of Intermediate M1

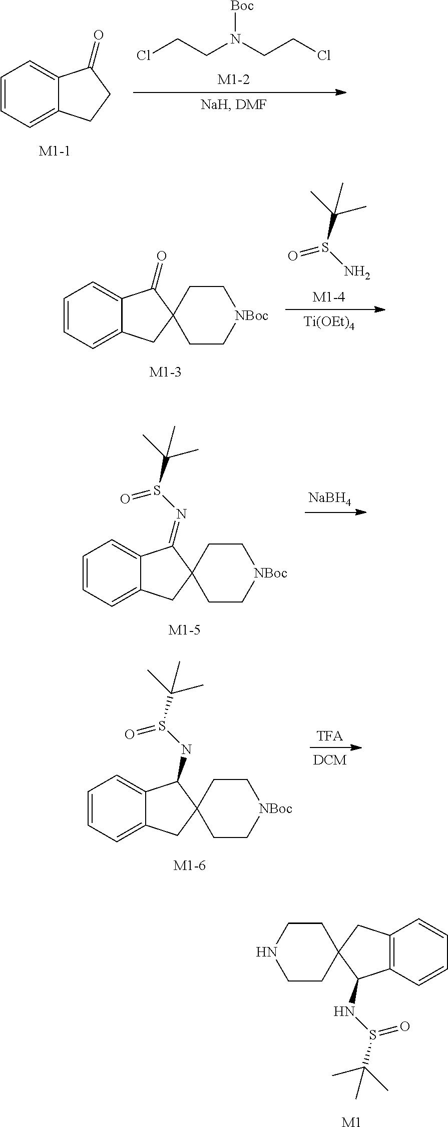

### Step 1: Preparation of Compound M1-3

To a solution of M1-1 (25.00 g) in 200 mL of DMF under nitrogen atmosphere was added NaH (22.7 g) batchwise at 0° C., the resulting mixture was stirred for 1.0 hour at 0° C. Then M1-2 (54.96 g) was added slowly. The resulting mixture was stirred at 0° C. for 1.0 hour and stirred at 60° C. for another 1.0 hour. When the reaction mixture was cooled to 0° C., the reaction mixture was quenched by the addition of 500 mL of ice-water. The mixture was extracted with EtOAc (500 mL*3) combined organic phase, and washed with saturated brine (200 mL*3), dried over anhydrous Na₂SO₄, filtered and concentrated under reduce pressure. The residue was purified by silica gel chromatography to give compound M1-3 (29.0 g) as brown oil. [M+H]+=302.

### Step 2: Preparation of Compound M1-5

To a solution of M1-3 (29.00 g) in 50 mL of Ti(OEt)₄ was added M1-4 (34.99 g) batchwise, the resulting mixture was stirred for 12.0 hours at 90° C. When the reaction mixture was cooled to room temperature, the reaction mixture was quenched by the addition of 500 mL of ice-water. The mixture was extracted with EtOAc (300 mL*3) and combined organic phase, and washed with saturated brine (200 mL*3), dried over anhydrous Na₂SO₄, filtered and concentrated under reduce pressure to give crude compound M1-5 (39.0 g) as brown oil. [M+H]+=405.

### Step 3: Preparation of Compound M1-6

To a solution of M1-5 (48.00 g) in 500 mL of THF under nitrogen atmosphere was added NaBH₄ (6.37 g) batchwise at −20° C. The resulting mixture was warmed to room temperature and stirred for 2.5 hours. The reaction mixture was quenched by the addition of 300 mL of ice-water. The mixture was extracted with EtOAc (300 mL*3) and combined organic layers. The organic layers were washed with saturated brine (200 mL*3), dried over anhydrous Na₂SO₄, filtered and concentrated under reduce pressure. The residue was purified by silica gel chromatography to give compound M1-6 (25.4 g) as brown oil. [M+H]+=407.

### Step 4: Preparation of Compound M1

To a solution of M1-6 (10.0 g) in 100 mL of DCM was added TFA (28.04 g). The resulting mixture was stirred at room temperature for 1.5 hours. The reaction mixture was quenched by the addition of 100 mL of saturated solution of NaHCO₃. The mixture was extracted with EtOAc (100 mL*3) and combined organic layers. The organic layers were washed with saturated brine (100 mL*3), dried over anhydrous Na₂SO₄, filtered and concentrated under reduce pressure. The residue was purified by silica gel chromatography to give compound M1 (7.64 g) as yellow solid. [M+H]+=307.

Compound M1: ¹H NMR (500 MHz, DMSO-d₆): δ 7.26-7.16 (m, 4H), 5.50 (d, J=10.0 Hz, 1H), 4.30 (d, J=10.0 Hz, 1H), 3.04 (d, J=16.0 Hz, 1H), 2.87-2.80 (m, 2H), 2.67-2.58 (m, 3H), 1.88-1.82 (m, 1H), 1.59-1.53 (m, 1H), 1.37-1.34 (m, 1H), 1.21 (s, 9H), 1.12-1.09 (m, 1H).

### Preparation of Intermediate Compound M2

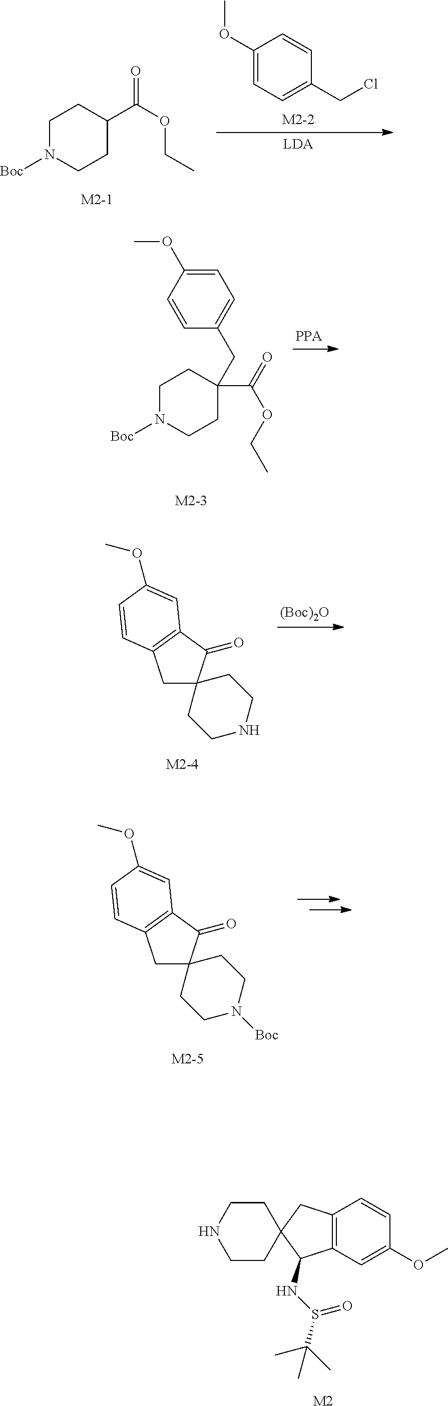

### Step 1: Preparation of Compound M2-3

To a −78° C. solution of M2-2 (2.83 g) in 50 mL of THF under nitrogen atmosphere was added the solution of LDA (2M, 6 mL) in THF/Hex dropwise. The resulting mixture was stirred for 1.0 hour at −78° C. Then the solution of M2-1 (1.56 g) in 3 mL of THF was added slowly. The resulting mixture was stirred at −78° C. for 1.0 hour. The reaction mixture was quenched by the addition of 50 mL of saturated brine. The mixture was extracted with EtOAc (30 mL*3) combined organic layers, and washed with saturated brine (50 mL*3), dried over anhydrous Na₂SO₄, filtered and concentrated under reduce pressure. The residue was purified by silica gel chromatography to give compound M2-3 (1.44 g) as light-yellow oil. [M+H]⁺=378.

### Step 2: Preparation of Compound M2-4

To 50 g of PPA was added M2-3 (1.9 g). The resulting mixture was stirred at 130° C. for 2 hours. When cooled to room temperature, the reaction mixture was quenched by the addition of 50 mL of ice-water. The mixture was adjusted with 4M solution of NaOH to pH=8 and extracted with EtOAc (150 mL*2) combined organic layers, and washed with saturated brine (50 mL*3), dried over anhydrous Na₂SO₄, filtered and concentrated under reduce pressure. The residue was purified by silica gel chromatography to give compound M2-4 (1.04 g) as light-yellow solid. [M+H]⁺=232.

### Step 3: Preparation of Compound M2-5

To a solution of M2-4 (10 g) in 50 mL of EtOH was added TEA (20 mL) and Boc₂O (2.1 g). The resulting mixture was stirred at room temperature for 3 hours. The reaction mixture was quenched by the addition of 50 mL of saturated brine, and extracted with EtOAc (150 mL*2) and combined organic layers, and washed with saturated brine (50 mL*3), dried over anhydrous Na₂SO₄, filtered and concentrated under reduce pressure. The residue was purified by silica gel chromatography to give compound M2-5 (1.2 g) as light-yellow solid. [M+H]⁺=332.

### Step 4: Preparation of Compound M2

The synthesis steps of compound M2 from intermediate M2-5 are the same as the steps of M1-3 to M1.

### Preparation of Intermediate Compound M3

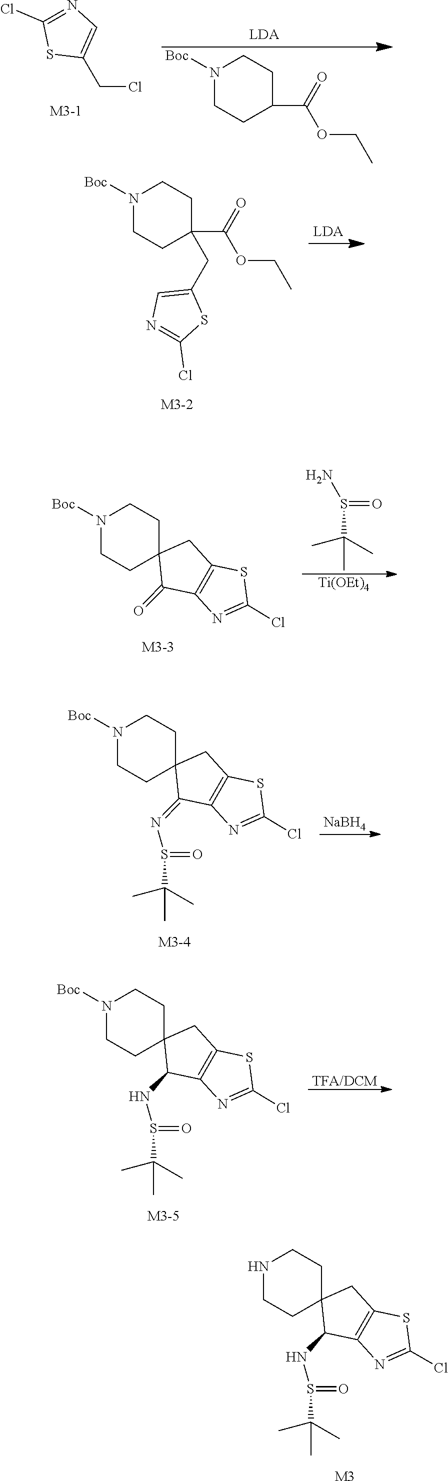

### Preparation of Compound M3-2

To a solution of 1-(tert-butyl) 4-ethyl piperidine-1,4-dicarboxylate (2.0 g) in THF (40 mL) was added LDA (2M, 5 mL) dropwise under N2 atmosphere at −78° C., The resulting mixture was stirred for 30 mins at this temperature. Then 2-chloro-5-(chloromethyl)thiazole (1.2 g) in 5 mL THF was added, stirred for 1 h. The mixture was quenched with saturated brine (50 mL), extracted with EA (30 mL*2), combined organic layers were dried over anhydrous Na₂SO₄, filtered and concentrated. The residue was purified by flash chromatography (EA/hexane=1:15) to afford compound M3-2 (10 g). [M+H]⁺=389.

### Preparation of Compound M3-3

To a solution of M3-2 (300 mg) in 10 mL THF was added LDA (2M, 1 mL) dropwise under N2 atmosphere at −78° C. The mixture was stirred for 30 mins at this temperature, then quenched with saturated brine (10 mL), extracted with EA (10 mL*2), the organic layers was concentrated to afford M3-3 (100 mg). [M+H]⁺=343.

M3 was synthesized in the manner similar to intermediate M1, except compound M1-3 was replaced with compound M3-3.

### Preparation of Intermediate Compounds of M4, M5, M6 and M7

The following compounds (such as M4, M5, M6 and M7) were synthesized using the above procedure (such as M2) or modified procedure with the corresponding starting materials.

M9 was synthesized by following procedures of synthesis of (5s)-5,6-dihydrospiro[piperidine]-4,4-pyrrolo[1,2-b]pyrazol]-5-amine dihydrochloride described in WO2020061101.

M8, M11 was synthesized by following procedures of WO2020063760.

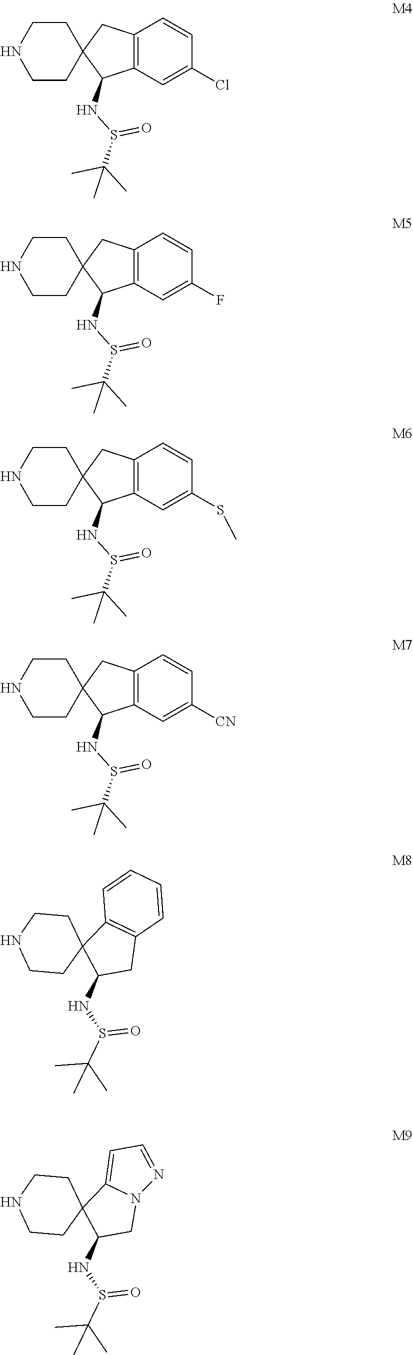

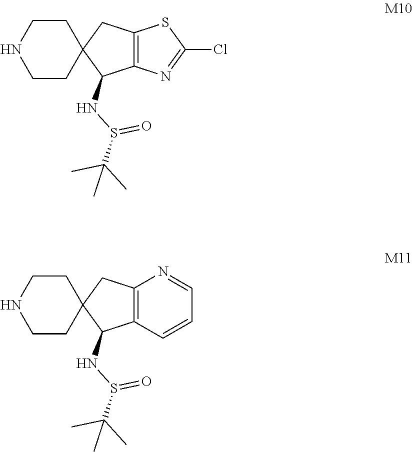 [SMILES: CC(C)(C)[S@@](=O)N[C@@H]1c2nc(Cl)sc2CC12CCNCC2 ; CC(C)(C)[S@@](=O)N[C@@H]1c2cccnc2CC12CCNCC2]

### Preparation of Intermediate M11-A

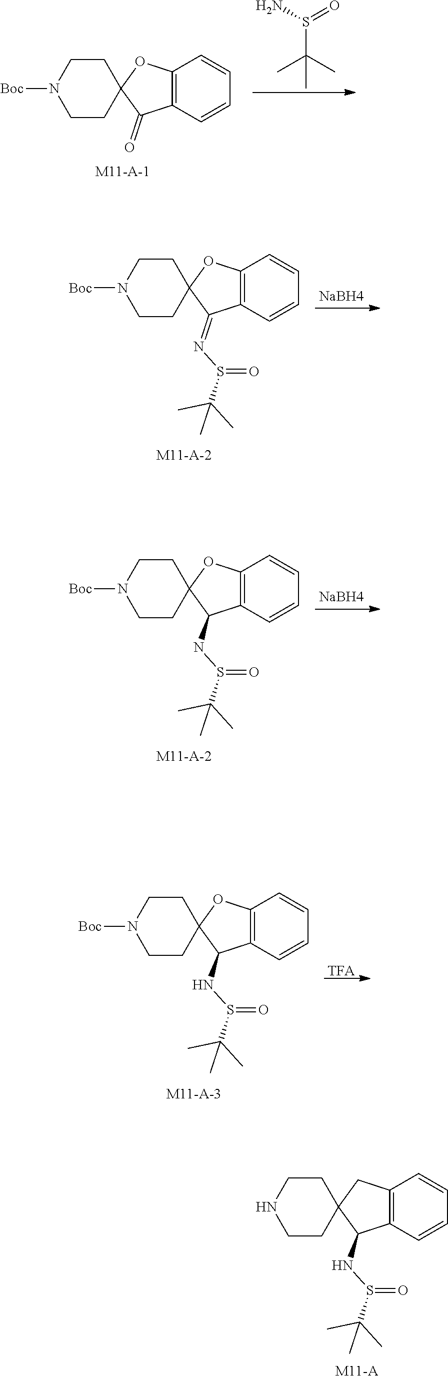

Following procedures of Y. Uto et al./Bioorg. Med. Chem. Lett. 20(2010)746-754, intermediate M11-A-1 was prepared. M11-A was synthesized in the manner similar to intermediate M1, except compound M1-3 was replaced with compound M11-A-1.

### Preparation of Intermediate Compounds of M12

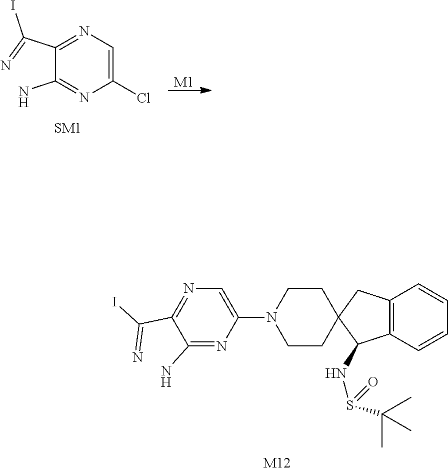

To a solution of SM1 (560 mg) in 20 mL of DMF was added M1 (670 mg) and DIPEA (860 mg). The resulting mixture was stirred at 80° C. for 3 hours. When cooled to room temperature, the reaction mixture was quenched by the addition of 20 mL of saturated brine, and extracted with EtOAc (100 mL*3) and combined organic layers, and washed with saturated brine (50 mL*3), dried over anhydrous Na₂SO₄, filtered and concentrated under reduce pressure. The residue was purified by silica gel chromatography to give compound M12 (990 mg) as light-yellow solid. [M+H]⁺=551.

### Preparation of Intermediate Compounds of M13, M14, M15, M16, M17 and M18

The following compounds (such as M13, M14, M15, M16, M17 and M18) were synthesized using the above procedure (such as M12) or modified procedure with the corresponding starting materials.

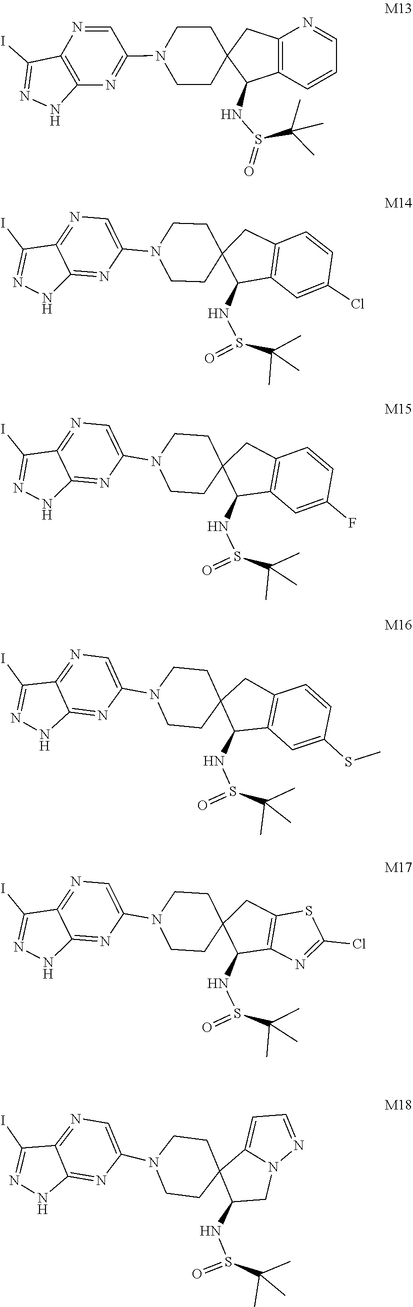 [SMILES: CC(C)(C)[S@@](=O)N[C@@H]1c2cccnc2CC12CCN(c1cnc3c(I)n[nH]c3n1)CC2 ; CC(C)(C)[S@@](=O)N[C@@H]1c2cc(Cl)ccc2CC12CCN(c1cnc3c(I)n[nH]c3n1)CC2 ; CC(C)(C)[S@@](=O)N[C@@H]1c2cc(F)ccc2CC12CCN(c1cnc3c(I)n[nH]c3n1)CC2 ; CSc1ccc2c(c1)[C@@H](N[S@](=O)C(C)(C)C)C1(CCN(c3cnc4c(I)n[nH]c4n3)CC1)C2 ; CC(C)(C)[S@@](=O)N[C@@H]1c2nc(Cl)sc2CC12CCN(c1cnc3c(I)n[nH]c3n1)CC2 ; CC(C)(C)[S@@](=O)N[C@@H]1Cn2nccc2C12CCN(c1cnc3c(I)n[nH]c3n1)CC2]

### Preparation of Intermediate Compound M19

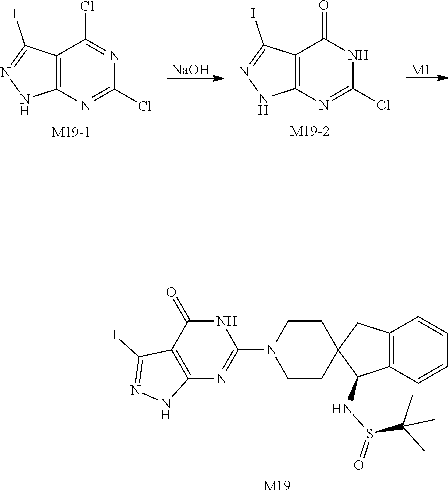

### Step 1: Preparation of Compound M19-2

To a solution of M19-1 (3.15 g) in 30 mL of dioxane was added 20 mL of NaOH (4M) solution. The resulting mixture was stirred for 24 hours at room temperature. The mixture was neutralized with a solution of HCl (1N) to pH=7. The solid was filtered and washed with water and dried under vacuum to give compound M19-2 (2.1 g) as light-yellow solid. [M+H]⁺=297.

### Step 2: Preparation of Compound M19

To a solution of M19-2 (572 mg) in 20 mL of DMF was added M1 (670 mg) and DIPEA (860 mg). The resulting mixture was stirred for 3 hours at 100° C. When cooled to room temperature, the mixture was quenched with a 20 mL of water. The mixture was extracted with EtOAc (100 mL*3), combined organic phase, and washed with saturated brine (50 mL*3), dried over anhydrous Na₂SO₄, filtered and concentrated under reduce pressure. The residue was purified by silica gel chromatography to give compound M19 (860 mg). [M+H]⁺=567.

### Preparation of Intermediate Compounds of M20, M21, M22, M23, M24 and M25

The following compounds (such as M20, M21, M22, M23, M24 and M25) were synthesized using the above procedure (such as M19) or modified procedure with the corresponding starting materials.

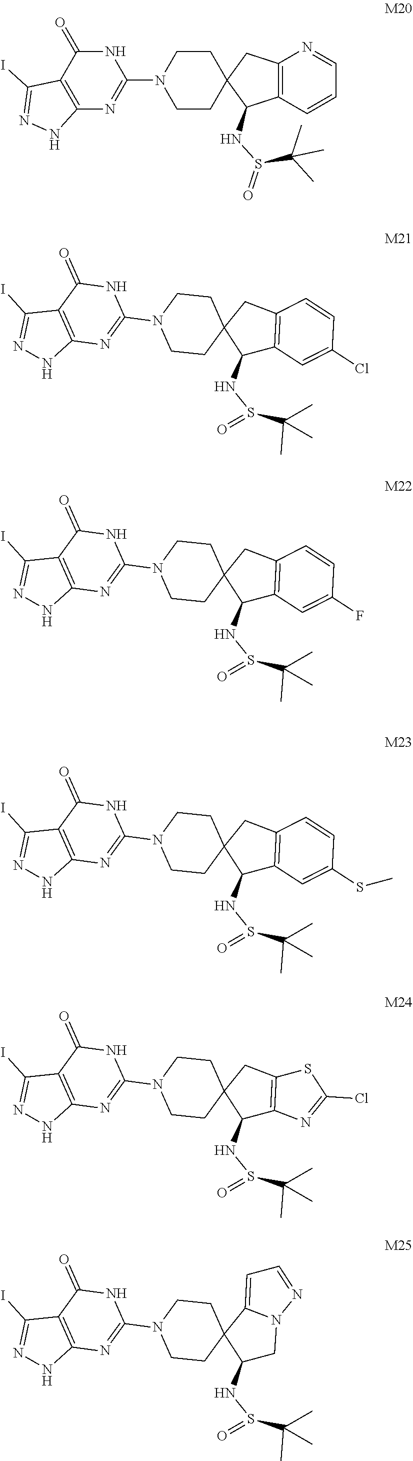 [SMILES: CC(C)(C)[S@@](=O)N[C@@H]1c2cccnc2CC12CCN(c1nc3[nH]nc(I)c3c(=O)[nH]1)CC2 ; CC(C)(C)[S@@](=O)N[C@@H]1c2cc(Cl)ccc2CC12CCN(c1nc3[nH]nc(I)c3c(=O)[nH]1)CC2 ; CC(C)(C)[S@@](=O)N[C@@H]1c2cc(F)ccc2CC12CCN(c1nc3[nH]nc(I)c3c(=O)[nH]1)CC2 ; CSc1ccc2c(c1)[C@@H](N[S@](=O)C(C)(C)C)C1(CCN(c3nc4[nH]nc(I)c4c(=O)[nH]3)CC1)C2 ; CC(C)(C)[S@@](=O)N[C@@H]1c2nc(Cl)sc2CC12CCN(c1nc3[nH]nc(I)c3c(=O)[nH]1)CC2 ; CC(C)(C)[S@@](=O)N[C@@H]1Cn2nccc2C12CCN(c1nc3[nH]nc(I)c3c(=O)[nH]1)CC2]

### Preparation of Intermediate Compound M26

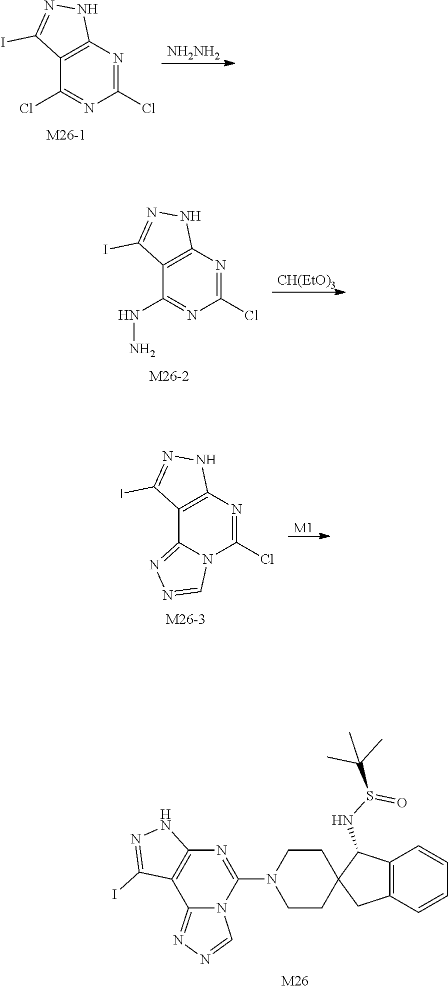

### Step 1: Preparation of Compound M26-2

To a solution of M26-1 (3.15 g, 10 mmol) in 30 mL of EtOH was added hydrazine hydrate (80%) (3.0 mL). The resulting mixture was stirred for 16 hours at room temperature. The solid was filtered, washed with EtOH, then the solid wa dried under vacuum to give compound M26-2 (2.0 g) as light-yellow solid. [M+H]⁺=311.

### Step 2: Preparation of Compound M26-3

To a solution of M26-2 (1.55 g, 5.0 mmol) in 20 mL of dioxane was added triethoxymethane (2.0 mL). The resulting mixture was stirred for 5 hours at 60° C. When cooled to room temperature, the reaction mixture was quenched by the addition of 100 mL of water and extracted with DCM (100 mL*2), combined organic layers, and washed with saturated brine (50 mL*3), dried over anhydrous Na₂SO₄, filtered and concentrated under reduce pressure. The residue was purified by silica gel chromatography to give compound M26-3 (1.11 g) as light-yellow solid. [M+H]⁺=321.

### Step 3: Preparation of Compound M26

To a solution of M26-3 (500 mg) in 20 mL of DMF was added M1 (650 mg) and DIPEA (850 mg). The resulting mixture was stirred at 80° C. for 3 hours. When cooled to room temperature, the reaction mixture was quenched by the addition of 20 mL of saturated brine, and extracted with EtOAc (80 mL*3) and combined organic layers, and washed with saturated brine (50 mL*3), dried over anhydrous Na₂SO₄, filtered and concentrated under reduce pressure. The residue was purified by silica gel chromatography to give compound M26 (600 mg). [M+H]⁺+=591.

### Preparation of Intermediate Compounds of M27, M28, M29, M30, M31 and M32

The following compounds (such as M27, M28, M29, M30, M31 and M32) were synthesized using the above procedure (such as M26) or modified procedure with the corresponding starting materials.

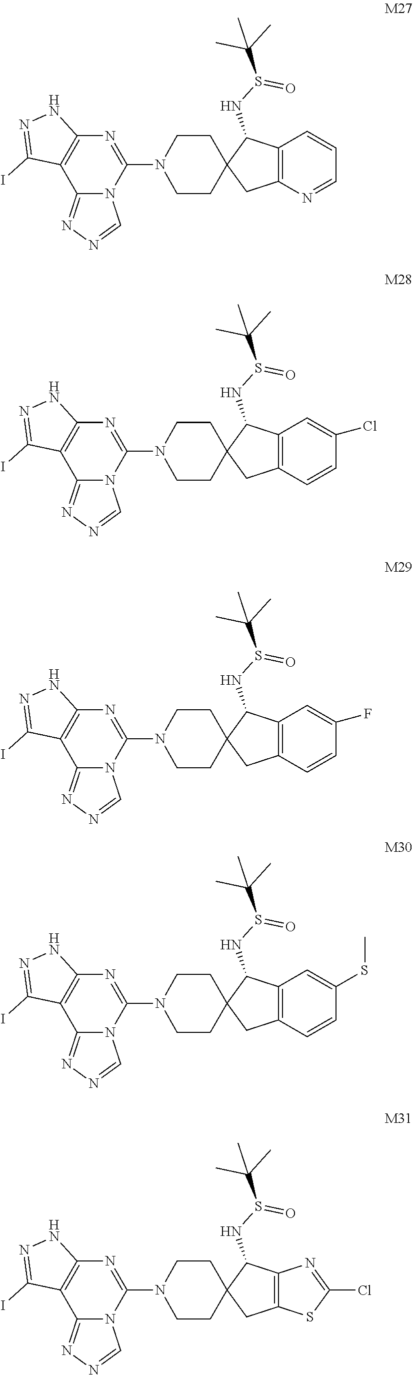 [SMILES: CC(C)(C)[S@@](=O)N[C@@H]1c2cccnc2CC12CCN(c1nc3[nH]nc(I)c3c3nncn13)CC2 ; CC(C)(C)[S@@](=O)N[C@@H]1c2cc(Cl)ccc2CC12CCN(c1nc3[nH]nc(I)c3c3nncn13)CC2 ; CC(C)(C)[S@@](=O)N[C@@H]1c2cc(F)ccc2CC12CCN(c1nc3[nH]nc(I)c3c3nncn13)CC2 ; CSc1ccc2c(c1)[C@@H](N[S@](=O)C(C)(C)C)C1(CCN(c3nc4[nH]nc(I)c4c4nncn34)CC1)C2 ; CC(C)(C)[S@@](=O)N[C@@H]1c2nc(Cl)sc2CC12CCN(c1nc3[nH]nc(I)c3c3nncn13)CC2]

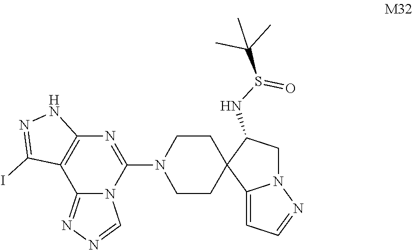 [SMILES: CC(C)(C)[S@@](=O)N[C@@H]1Cn2nccc2C12CCN(c1nc3[nH]nc(I)c3c3nncn13)CC2]

### Preparation of Intermediate Compound M33

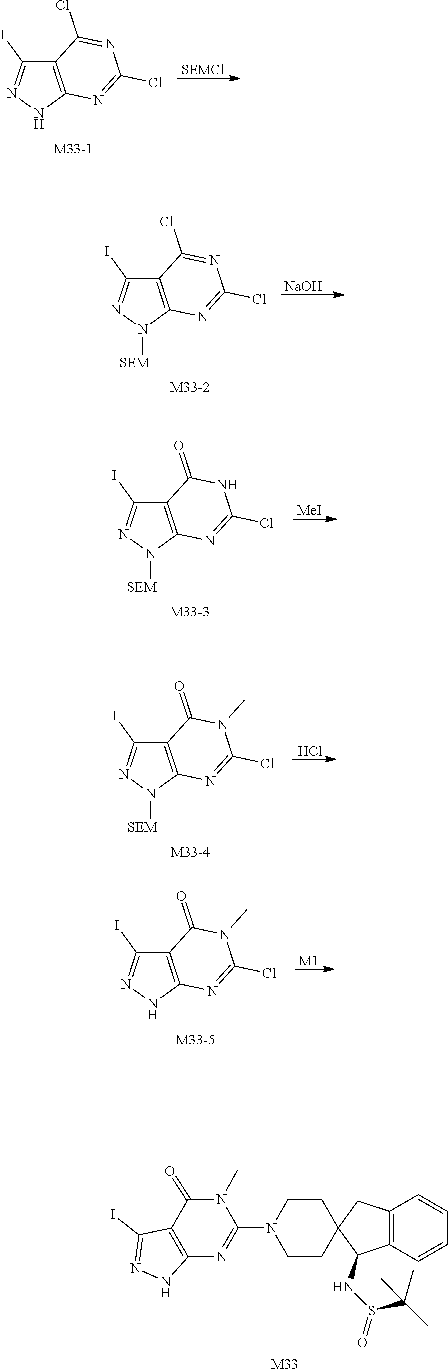

### Step 1: Preparation of Compound M33-2

To a solution of M33-1 (3.15 g) in 30 mL of DCM was added (2-(chloromethoxy)ethyl)trimethylsilane (2.0 g) and DIPEA (2.58 g). The resulting mixture was stirred for 1.5 hours at room temperature. The reaction mixture was quenched by the addition of 100 mL of water. The mixture was extracted with EtOAc (100 mL*3), combined organic layers, and washed with saturated brine (50 mL*3), dried over anhydrous Na₂SO₄, filtered and concentrated under reduce pressure. The residue was purified by silica gel chromatography to give compound M33-2 (3.61 g). [M+H]⁺=397.

### Step 2: Preparation of Compound M33-3

To a solution of M33-2 (2.22 g) in 20 mL of THF was added 4 mL of solution of NaOH (5M). The resulting mixture was stirred for 7.5 hours at room temperature. The reaction mixture was quenched by the addition of 100 mL of water. The mixture was extracted with EtOAc (100 mL*3), combined organic layers, and washed with saturated brine (100 mL*2), dried over anhydrous Na₂SO₄, filtered and concentrated under reduce pressure. The residue was purified by silica gel chromatography to give compound M33-3 (1.61 g). [M+H]⁺=379.

### Step 3: Preparation of Compound M33-4

To a solution of M33-3 (1.28 g) in 10 mL of DMF was added Mel (0.56 g) and K₂CO₃ (0.82 g). The resulting mixture was stirred for 1.5 hours at room temperature. The reaction mixture was quenched by 100 mL of water. The mixture was extracted with EtOAc (100 mL*3), combined organic layers, and washed with saturated brine (100 mL*2), dried over anhydrous Na₂SO₄, filtered and concentrated under reduce pressure. The residue was purified by silica gel chromatography to give compound M33-4 (0.81 g). [M+H]⁺=393.

### Step 4: Preparation of Compound M33-5

To a solution of M33-4 (441 mg) in 10 mL of dioxane was added 3 mL solution of HCl (4M) in dioxane. The resulting mixture was stirred for 8 hours at room temperature. The reaction mixture was quenched by 10 mL of water and neutralized with solution of NaOH (2N) to pH=8. The solid was filtered and washed with saturated brine (10 mL*2) and dried under vacuum to give compound M33-5 (210 mg). [M+H]⁺=263.

### Step 5: Preparation of Compound M33

To a solution of M33-5 (620 mg) in 20 mL of DMF was added M1 (670 mg) and DIPEA (860 mg). The resulting mixture was stirred for 3 hours at 90° C. When cooled to room temperature, the reaction mixture was quenched by 20 mL of water. The mixture was extracted with EtOAc (100 mL*3), combined organic layers, and washed with saturated brine (100 mL*2), dried over anhydrous Na₂SO₄, filtered and concentrated under reduce pressure. The residue was purified by silica gel chromatography to give compound M33 (810 mg). [M+H]⁺=533.

### Example 2 Preparation of Compound of A002

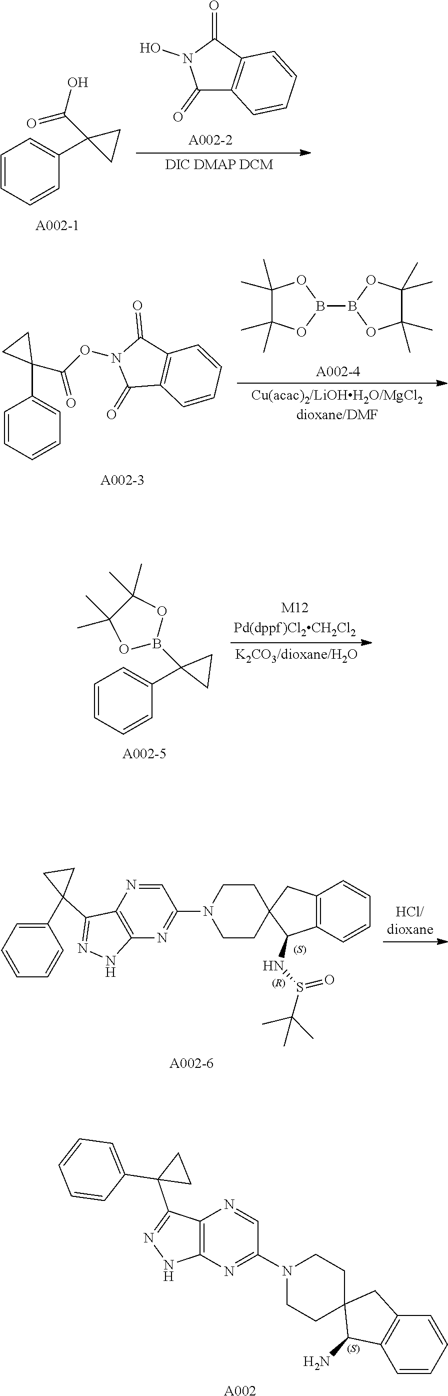

### Preparation of Compound A002-3

A round-bottom flask or culture tube equipped with a stir bar was charged with 1-phenylcyclopropanecarboxylic acid (500 mg), N-hydroxy-phthalimide (553.20 mg) and DMAP (37.66 mg). Dichloromethane (20 mL) was added followed by DIC (427.97 mg), and the mixture was allowed to stir vigorously for 2 hours. The mixture was filtered (over Celite, SiO₂, or through a fritted funnel) and rinsed with additional CH₂Cl₂/Et₂O. The solvent was removed under reduced pressure, and purified by column chromatography (DCM/MeOH=I/O) to afford the desired product A002-3 (767 mg). [M+H]⁺=308.

### Preparation of Compound A002-5

To a 15 mL culture tube equipped with a stir bar were added (1,3-dioxoisoindolin-2-yl) 1-phenylcyclopropanecarboxylate (307 mg), B₂Pin₂ (761.07 mg), LiOH·H₂O (628.79 mg), Cu(acac)₂ (78.45 mg) and MgCl₂ (142.68 mg). The tube was evacuated and backfilled with argon for 3 times. Degassed dioxane (6 mL) DMF (3 mL) was added and the resulting mixture was stirred under 1000 rpm at RT until dark brown color was observed (typical reaction time <10 min). The reaction mixture was diluted with EtOAc (20 mL) and saturated NH₄Cl (20 mL), and the resulting mixture was shaken vigorously until getting a clear biphasic solution. The organic phase was collected and dried over anhydrous Na₂SO₄, evaporated and purified by silica gel chromatography (Hexane/EA=100/0-100/3) to afford the desired product A002-5 (223 mg). [M+H]⁺=245.

### Preparation of Compound A002-6

To a 50 mL round bottom flask equipped with a stir bar were added A002-5 (67 mg), M12 (100 mg), Pd(dppf)Cl₂·CH₂Cl₂ (15 mg), K₂CO₃ (75 mg) and dioxane/H₂O (10 mL/1 mL). The flask was evacuated and backfilled with argon for 3 times, then stirred for 5 hours at 100° C., monitored by LCMS till M12 was consumed, cool down, evaporated and purified by silica gel chromatography (DCM/MeOH=100/0-100/6) to afford the desired product A002-6 (108 mg). [M+H]⁺=541.

### Preparation of Compound A002

To a 50 mL round bottom flask equipped with a stir bar were added A002-6 (108 mg), 4N dioxane/HCl (5 mL) stirred for 1 hour at r.t, then evaporated, the residue was washed by Et₂O (20 mL) to afford the desired product A002 (46 mg). [M+H]⁺=437.

¹H NMR (500 MHz, CD₃OD) δ 8.36 (s, 1H), 7.37-7.24 (m, 3H), 7.22-7.19 (m, 2H), 7.13 (t, J=7.2 Hz, 2H), 7.10 (t, J=7.5 Hz, 2H), 4.23-3.88 (m, 3H), 3.42-3.32 (m, 1H), 3.15 (q, J=16.4 Hz, 2H), 1.92-1.50 (m, 4H), 1.44-1.36 (m, 2H), 1.21 (s, 3H).

### Example 33 Preparation of Compound of A033

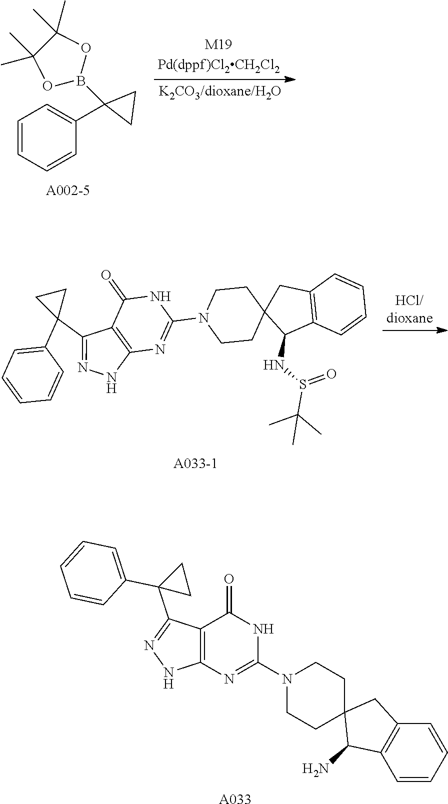 [SMILES: CC1(C)OB(C2(c3ccccc3)CC2)OC1(C)C ; CC(C)(C)[S@@](=O)N[C@@H]1c2ccccc2CC12CCN(c1nc3[nH]nc(C4(c5ccccc5)CC4)c3c(=O)[nH]1)CC2 ; N[C@@H]1c2ccccc2CC12CCN(c1nc3[nH]nc(C4(c5ccccc5)CC4)c3c(=O)[nH]1)CC2 ; "A033"]

### Preparation of Compound A033-1

To a 50 mL round bottom flask equipped with a stir bar were added A002-5 (73 mg), M19 (113 mg), Pd(dppf)Cl₂·CH₂Cl₂ (14 mg), K₂CO₃ (73 mg) and dioxane/H₂O (10 mL/1 mL). The flask was evacuated and backfilled with argon for 3 times, then stirred for 5 hours at 100° C., monitored by LCMS till M19 was consumed, cooled down, evaporated and purified by silica gel chromatography (DCM/MeOH=100/0-100/10) to afford the desired product A033-1 (100 mg). [M+H]=557.

### Preparation of Compound A033

To a 50 mL round bottom flask equipped with a stir bar were added A033-1 (100 mg), 4N dioxane/HCl (5 mL) stirred for 1 hour at r.t, then evaporated, the residue was washed by Et₂O (20 mL) to afford the desired product A033 (55 mg).

[M+H]⁺=453

¹H NMR (500 MHz, CD₃OD) δ 8.51 (sb, 1H), 7.51-7.44 (m, 1H), 7.42-7.35 (m, 2H), 7.33 (t, J=7.2 Hz, 3H), 7.20 (t, J=7.5 Hz, 2H), 7.11 (t, J=6.8 Hz, 1H), 4.50-3.98 (m, 3H), 3.42-3.32 (m, 1H), 3.15 (q, J=16.4 Hz, 2H), 1.92-1.50 (m, 4H), 1.49-1.39 (m, 2H), 1.30 (s, 3H).

### Example 3 Preparation of Compound of A003

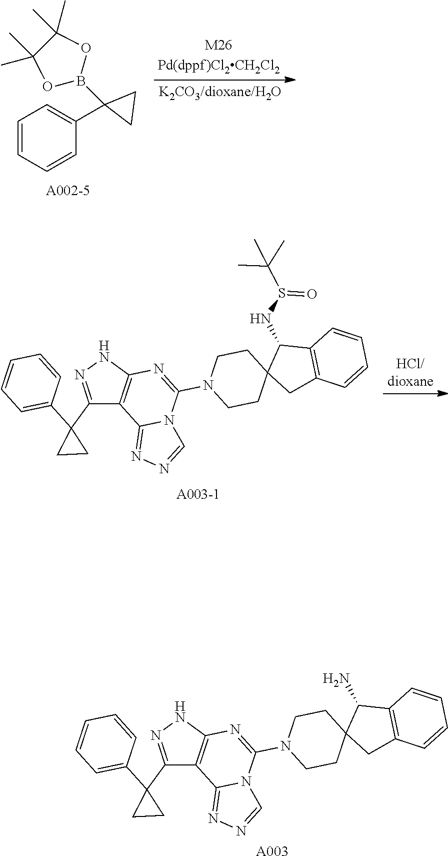

### Preparation of Compound A003-1

To a 50 mL round bottom flask equipped with a stir bar were added A002-5 (83 mg), M26 (100 mg), Pd(dppf)Cl₂·CH₂Cl₂ (14 mg), K₂CO₃ (70 mg) and dioxane/H₂O (10 mL/1 mL). The flask was evacuated and backfilled with argon for 3 times, then stirred for 5 hours at 100° C., monitored by LCMS till M26 was consumed, cool down, evaporated and purified by silica gel chromatography (DCM/MeOH=100/0-100/5) to afford the desired product A003-1 (90 mg). [M+H]⁺=581.

### Preparation of Compound A003

To a 50 mL round bottom flask equipped with a stir bar were added A003-1 (90 mg), 4N dioxane/HCl (5 mL) stirred for 1 hour at r.t, then evaporated, the residue was washed by Et₂O (30 mL) to afford the desired product A003 (35 mg). [M+H]⁺=477.

### Example 1 Preparation of Compound of A001

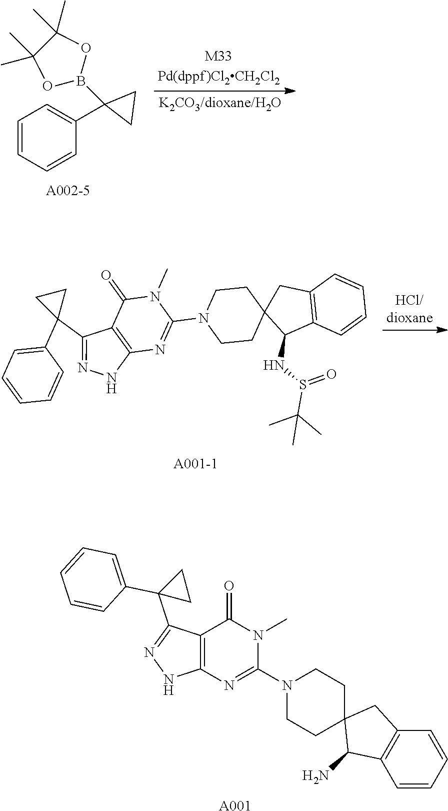 [SMILES: CC1(C)OB(C2(c3ccccc3)CC2)OC1(C)C ; Cn1c(N2CCC3(CC2)Cc2ccccc2[C@H]3N[S@](=O)C(C)(C)C)nc2[nH]nc(C3(c4ccccc4)CC3)c2c1=O ; Cn1c(N2CCC3(CC2)Cc2ccccc2[C@H]3N)nc2[nH]nc(C3(c4ccccc4)CC3)c2c1=O ; "A001"]

### Preparation of Compound A001-1

To a 50 mL round bottom flask equipped with a stir bar were added A002-5 (84 mg), M33 (100 mg), Pd(dppf)Cl₂·CH₂Cl₂ (14 mg), K₂CO₃ (73 mg) and dioxane/H₂O (10 mL/1 mL). The flask was evacuated and backfilled with argon for 3 times, then stirred for 8 hours at 120° C., monitored by LCMS till M33 was consumed, cool down, evaporated and purified by silica gel chromatography (DCM/MeOH=100/0-100/5) to afford the desired product A001-1 (91 mg, 0.16 mmol). [M+H]⁺=571.

### Preparation of Compound A001

To a 50 mL round bottom flask equipped with a stir bar were added A001-1 (91 mg), 4N dioxane/HCl (5 mL) stirred for 1 hour at r.t, then evaporated, the residue was washed by Et₂O (30 mL) to afford the desired product A001 (32 mg).

[M+H]⁺=467.

¹H NMR (500 MHz, CD₃OD) δ 8.51 (sb, 1H), 7.51-7.44 (m, 1H), 7.42-7.35 (m, 2H), 7.33 (t, J=7.2 Hz, 3H), 7.20 (t, J=7.5 Hz, 2H), 7.11 (t, J=6.8 Hz, 1H), 4.50-3.98 (m, 3H), 3.42-3.32 (m, 1H), 3.34 (s, 3H), 3.15 (q, J=16.4 Hz, 2H), 1.92-1.50 (m, 4H), 1.49-1.39 (m, 2H), 1.30 (s, 3H).

### Example 89 Preparation of Compound of A089

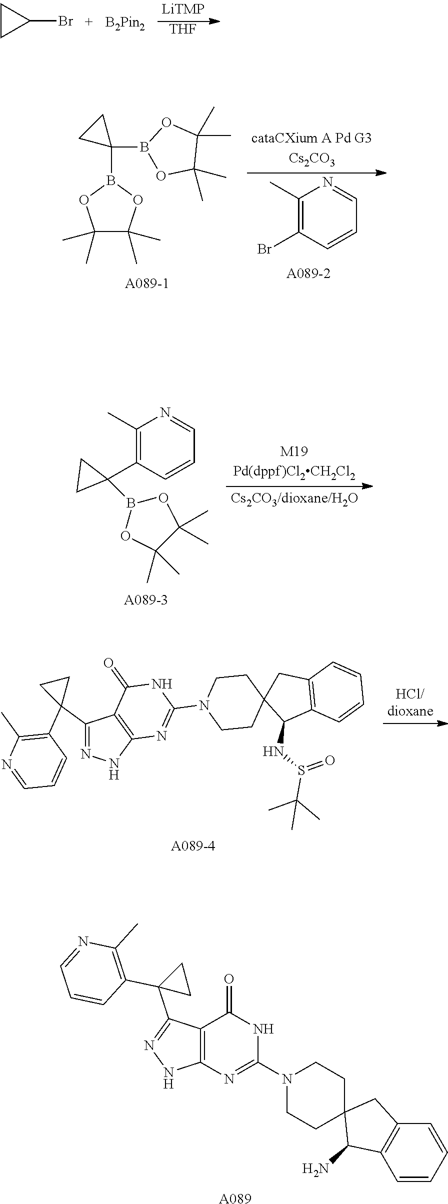

### Preparation of Compound A089-1

A solution of 2,2,6,6-tetramethylpiperidine (7.01 g) in THF (30 mL) was cooled to −78° C. To this solution was added n-BuLi (18 mL, 2.7 M in heptane) dropwise over 15 minutes. The reaction was stirred at −78° C. for 30 min, then allowed to warm to 0° C. Meanwhile, a solution of cyclopropyl bromide (5.00 g, 3.31 mL) and bis(pinacolato)diboron (10.1 g) was prepared in THF (100 mL) and cooled to −95° C. in an acetone/N2 bath. To this solution was added the freshly prepared LiTMP over 20 minutes. After stirring at −95° C. for 1 h, the reaction was complete by GC/MS monitoring. A saturated solution of NaHCO₃ was added to quench the reaction, and the mixture was allowed to warm to RT. Ether was added and the layers were separated. The aqueous phase was extracted with diethyl ether (3×100 mL) and the combined organics were washed with water (50 mL) and saturated brine (50 mL), dried over Na₂SO₄, filtered and concentrated to afford the crude product. Crude material was purified by flash column chromatography (0-10% EtOAc/Heptane) to afford the desired product A089-1 as a white solid (8.12 g, 27.6 mmol, 68%).

¹H NMR (CDCl₃, 500 MHz): 1.20 (s, 25H), 0.79 (s, 4H).

### Preparation of Compound A089-3

In a 50 mL round bottom flask equipped with a stir bar, reflux condenser and septum was added A089-1 (220 mg), CatacXium A Pd G₃ (34.8 mg), A089-2 (190 mg), cesium carbonate (731 mg), dioxane (20 mL) and water (2 mL). The resulting mixture was degassed by bubbling N2 through the solution for 15 min and was then heated to 100° C. for 24 h. The reaction was cooled to room temperature, and partitioned between EtOAc and water. The aqueous phase was extracted twice with EtOAc (2*20 mL), and the combined organics were washed with saturated brine (10 mL), dried over Na₂SO₄, filtered and concentrated to afford the crude product. Purified by flash column chromatography (0-30% EtOAc/heptanes) to afford the desired material A089-3 as a light brown solid (120 mg, 62%). [M+H]⁺=260.

### Preparation of Compound A089-4

In a 50 mL round bottom flask equipped with a stir bar, reflux condenser and septum was added A089-3 (55 mg), Pd(dppf)Cl₂·CH₂Cl₂ (14.4 mg), M19 (100 mg), cesium carbonate (173 mg), dioxane (10 mL) and water (1 mL). The resulting mixture was degassed by bubbling N2 through the solution for 15 min and was then heated to 100° C. for 24 h. The reaction was cooled to room temperature, and partitioned between EtOAc and water. The aqueous phase was extracted twice with EtOAc (2×10 mL), and the combined organics were washed with saturated brine (10 mL), dried over Na₂SO₄, filtered and concentrated to afford the crude product. Purified by flash column chromatography (0-30% EtOAc/n-hexanes) to afford the desired material A089-4 as a light brown solid (80 mg, 80%). [M+H]⁺=572.

### Preparation of Compound A089

To a 50 mL round bottom flask equipped with a stir bar were added A089-4 (80 mg) and 4N dioxane/HCl (5 mL), stirred for 1 hour at r.t, then evaporated, the residue was washed by Et₂O (30 mL) to afford the desired product A089 (15 mg, 21%). [M+H]⁺=468.

### Example 90 Preparation of Compound of A090

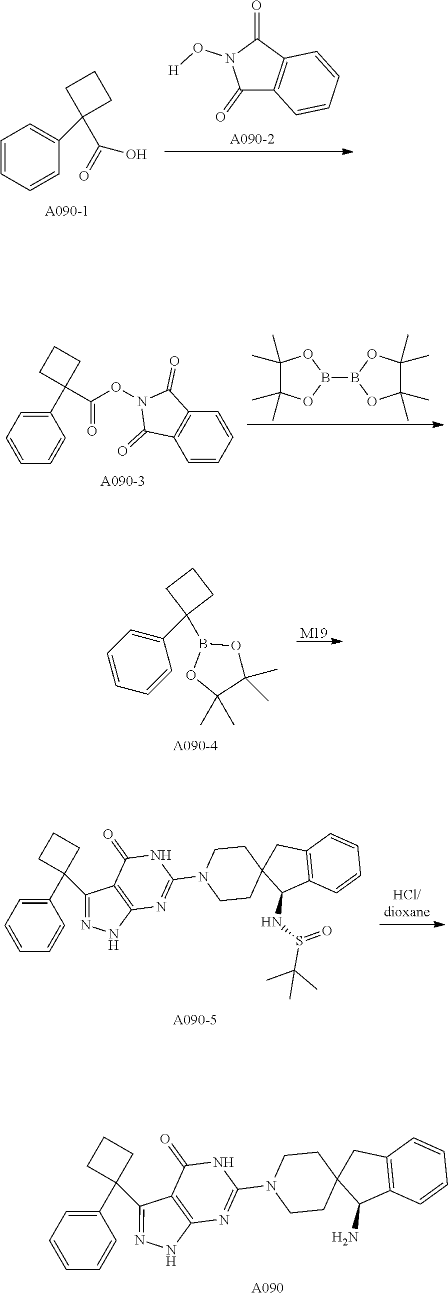

### Step 1 Preparation of Compound A090-3

The materials 1-phenylcyclobutanecarboxylic acid (1.76 g) and 2-hydroxyisoindoline-1,3-dione (1.79 g) was added into 100 mL of DCM, to which was added DIC (1.39 g). The mixture was stirred at room temperature for 5 hours. The mixture was washed with saturated solution of NaCl (200 mL*2) and the organic layer was concentrated. The residue was purified by silica gel column (PE/EA=5/95) to afford product A090-3 (3.08 g, yield: 91%) as white solid.

### Step 2 Preparation of Compound A090-4

A090-3 (1.6 g) was added into a mixed solvent of dioxane (40 mL) and DMF (10 mL), to which was added 4,4,4′,4′,5,5,5′,5′-octamethyl-2,2′-bi(1,3,2-dioxaborolane) (3.8 g), magnesium chloride (0.75 g), lithium hydroxide (3.15 g) and Cu(acac)₂ (0.35 g). The mixture was stirred under N2 at room temperature for 15 minutes. The mixture was diluted with EA (400 mL), and washed with saturated solution of NH₄Cl (400 mL) and NaCl (400 mL). The organic layer was concentrated and purified by silica gel column (PE/EA=5/95) to afford product A090-4 (0.13 g, yield: 11%) as white solid.

### Step 3 Preparation of Compound A090-5

A090-4 (0.13 g) was added into a mixed solvent of dioxane (4.0 mL) and water (10 mL), to which was added M19 (0.11 g), Pd(dppf)Cl₂·DCM (35 mg) and Cesium Carbonate (0.49 g). The mixture was stirred at 100° C. for 18 hours. The mixture was diluted with EA (50 mL) and washed with saturated solution of NaCl (50 mL). The organic layer was concentrated and purified by silica gel column (MeOH/DCM=5/95) to afford product 21 mg A090-5 as pale yellow solid.

### Step 4 Preparation of Compound A090

The mediate product of A090-5 (21 mg) was dissolved into DCM (8 mL), to which was added the solution of hydrocholoride (4M) in dioxane 0.2 mL. The mixture was stirred at room temperature for 3 hours. The mixture was concentrated and the solid was washed with diethyl ether (5 mL) and dried under vacuum to afford 8 mg A090 as pale yellow solid. [MS+H]=503.

### Example 185 Preparation of Compound of C004

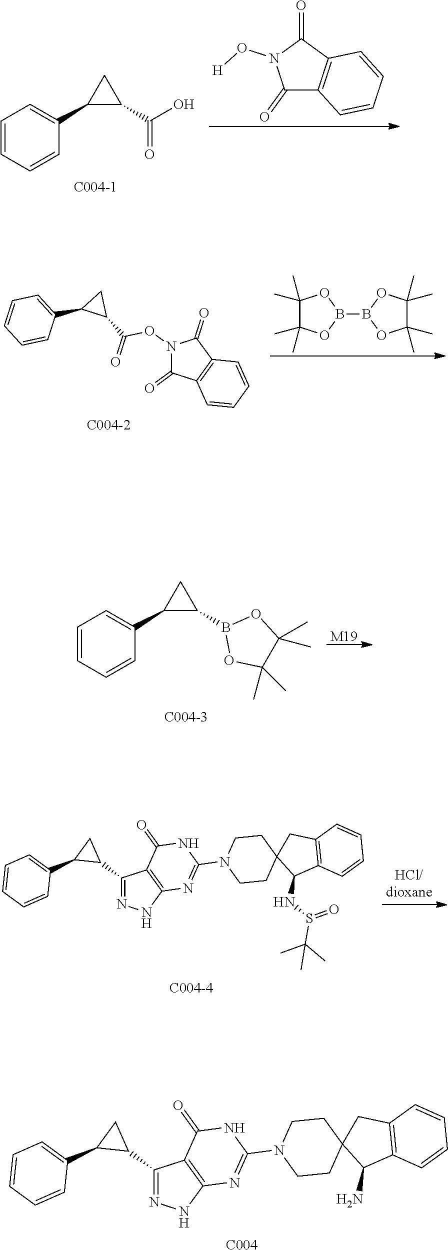

### Step 1 Preparation of Compound C004-2

The materials (1S,2S)-2-phenylcyclopropane-1-carboxylic acid (1.62 g) and 2-hydroxyisoindoline-1,3-dione (1.79 g) was added into DCM (30 mL), to which was added DIC (1.39 g). The mixture was stirred at room temperature for 5 hours. TLC show the reaction was completed. The mixture was added water (100 mL), separated; The organic layer was washed with saturated solution of NaCl (200 ml*2), dried over Na₂SO₄, concentrated. The residue was purified by silica gel column (PE/EA=5/95) to afford product compound C004-2 (2.76 g, yield: 90%) as off-white solid.

### Step 2 Preparation of Compound C004-3

Compound C004-2 (1.53 g) was added into a mixed solvent of dioxane (40 mL) and DMF (10 mL), to which was added 4,4,4′,4′,5,5,5′,5′-octamethyl-2,2′-bi(1,3,2-dioxaborolane) (3.8 g), magnesium chloride (0.75 g), lithium hydroxide (3.15 g) and Cu(acac)₂ (0.35 g). The mixture was stirred under N2 at room temperature for 15 minutes. The mixture was diluted with EA (400 mL), and washed with saturated solution of NH₄Cl (400 mL) and NaCl (400 mL). The organic layer was concentrated and purified by silica gel column (PE/EA=5/95) to afford product (0.37 g, yield: 30%) as colorless oil.

### Step 3 Preparation of Compound C004-4

Compound C004-3 (0.35 g) was added into a mixed of dioxane (4.0 mL) and water (1.0 mL), to which was added M19 (0.17 g), Pd(dppf)Cl₂·DCM (35 mg) and Cesium Carbonate (0.49 g). The mixture was stirred at 100° C. for 18 hours. The mixture was diluted with EA (50 mL) and washed with saturated solution of NaCl (50 mL). The organic layer was concentrated and purified by silica gel column (MeOH/DCM=5/95) to afford product compound C004-4 (84 mg, yield: 50%) as pale yellow solid.

### Step 4 Preparation of Compound C004

Compound C004-4 (84 mg) was dissolved into DCM (8 mL), to which was added the solution of hydrocholoride (4M) in dioxane 0.5 mL. The mixture was stirred at room temperature for 3 hours. The mixture was concentrated and the solid was washed with diethyl ether (5 mL) and dried under vacuum to afford product 40 mg compound C004 as pale yellow solid. [MS+H]=453.

### Example 200 Preparation of Compound 200

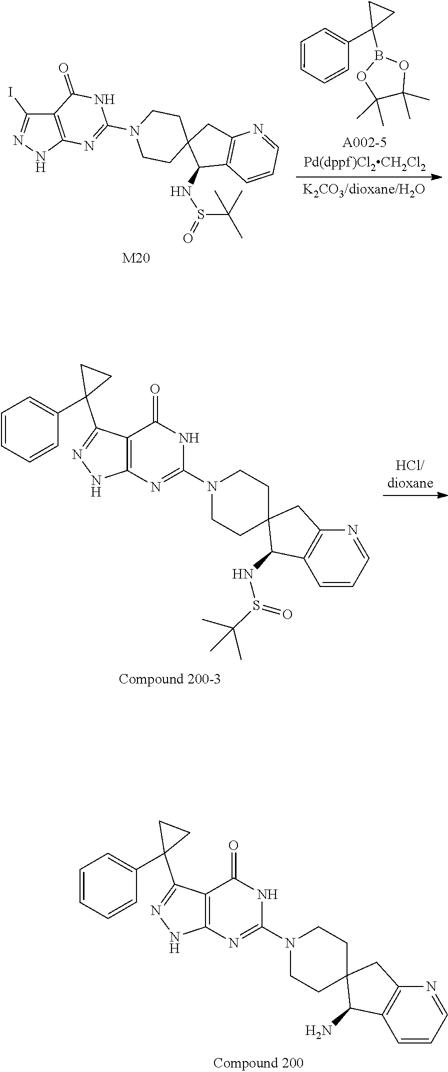

### Step 1 Preparation of Compound 200-3

To a 50 mL round bottom flask equipped with a stir bar were added M20 (567 mg), 4,4,5,5-tetramethyl-2-(1-phenylcyclopropyl)-1,3,2-dioxaborolane (292 mg), Pd(dppf)Cl₂·CH₂Cl₂ (75 mg), K₂CO₃ (276 mg) and dioxane/H₂O (20 mL/2 mL). The flask was evacuated and backfilled with argon for 3 times, then stirred for 5 hours at 100° C., monitored by LCMS till initial material was consumed, cooled down, evaporated and purified by silica gel chromatography (DCM/MeOH=100/0-100/6) to afford the desired product Compound 200-3 (488 mg). [M+H]+=558.26.

### Step 2 Preparation of Compound 200

To a 50 mL round bottom flask equipped with a stir bar were added Compound 200-3 (114 mg), 4N dioxane/HCl (5 mL) stirred for 1 hour at r.t, then evaporated, the residue was washed by Et₂O (20 mL) to afford the desired product Compound 200 (65 mg). [M+H]⁺=454.54.

The following compounds (such as A004-A032, A034-A067, A069-A088, A090-A101, C001-C003, Example 192-Example 257) showing in Table 1 were synthesized using the above procedures of A001, A002, A003, A033, A089, Example 200 or modified procedure with the corresponding starting materials.

TABLE 1

| | | | Physical |
|---|---|---|---|
| | | | Data |
| | | | (MS) |
| Example | Structure | Chemical Name | (M + H)⁺ |
| Example 4 (A004) | 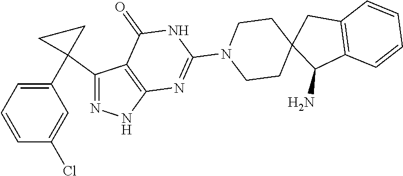 [SMILES: N[C@@H]1c2ccccc2CC12CCN(c1nc3[nH]nc(C4(c5cccc(Cl)c5)CC4)c3c(=O)[nH]1)CC2] | (S)-6-(1-amino-1,3- dihydrospiro[indene-2,4′- piperidin]-1′-yl)-3-(1-(3- chlorophenyl)cyclopropyl)-1,5- dihydro-4H-pyrazolo[3,4- d]pyrimidin-4-one | 487.19 |
| Example 5 (A005) | 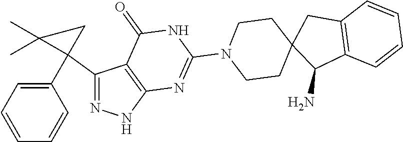 [SMILES: CC1(C)CC1(c1ccccc1)c1n[nH]c2nc(N3CCC4(CC3)Cc3ccccc3[C@H]4N)[nH]c(=O)c12] | 6-((S)-1-amino-1,3- dihydrospiro[indene-2,4′- piperidin]-1′-yl)-3-(2,2-dimethyl- 1-phenylcyclopropyl)-1,5- dihydro-4H-pyrazolo[3,4- d]pyrimidin-4-one | 481.26 |
| Example 6 (A006) | 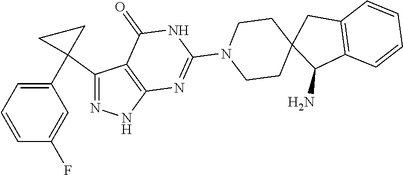 [SMILES: N[C@@H]1c2ccccc2CC12CCN(c1nc3[nH]nc(C4(c5cccc(F)c5)CC4)c3c(=O)[nH]1)CC2] | (S)-6-(1-amino-1,3- dihydrospiro[indene-2,4′- piperidin]-1′-yl)-3-(1-(3- fluorophenyl)cyclopropyl)-1,5- dihydro-4H-pyrazolo[3,4- d]pyrimidin-4-one | 471.22 |
| Example 7 (A007) | 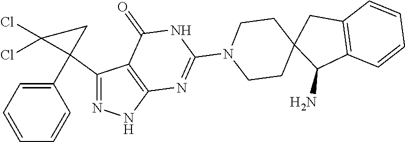 [SMILES: N[C@@H]1c2ccccc2CC12CCN(c1nc3[nH]nc(C4(c5ccccc5)CC4(Cl)Cl)c3c(=O)[nH]1)CC2] | 6-((S)-1-amino-1,3- dihydrospiro[indene-2,4′- piperidin]-1′-yl)-3-(2,2-dichloro- 1-phenylcyclopropyl)-1,5- dihydro-4H-pyrazolo[3,4- d]pyrimidin-4-one | 421.15 |
| Example 8 (A008) | 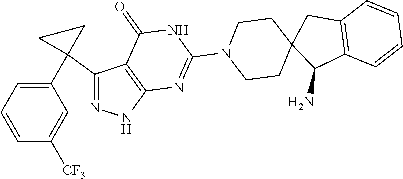 [SMILES: N[C@@H]1c2ccccc2CC12CCN(c1nc3[nH]nc(C4(c5cccc(C(F)(F)F)c5)CC4)c3c(=O)[nH]1)CC2] | (S)-6-(1-amino-1,3- dihydrospiro[indene-2,4′- piperidin]-1′-yl)-3-(1-(3- (trifluoromethyl)plienyl)cyclo- propyl)-1,5-dihydro-4H- pyrazolo[3,4-d]pyrimidin-4-one | 421.22 |
| Example 9 (A009) | 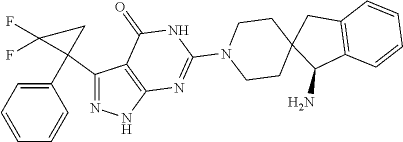 [SMILES: N[C@@H]1c2ccccc2CC12CCN(c1nc3[nH]nc(C4(c5ccccc5)CC4(F)F)c3c(=O)[nH]1)CC2] | 6-((S)-1-amino-1,3- dihydrospiro[indene-2,4′- piperidin]-1′-yl)-3-(2,2-difluoro- 1-phenylcyclopropyl)-1,5- dihydro-4H-pyrazolo[3,4- d]pyrimidin-4-one | 489.21 |
| Example 10 (A010) | 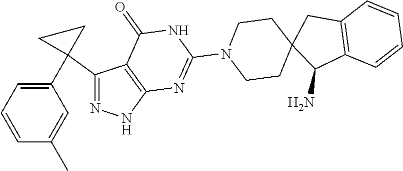 [SMILES: Cc1cccc(C2(c3n[nH]c4nc(N5CCC6(CC5)Cc5ccccc5[C@H]6N)[nH]c(=O)c34)CC2)c1] | (S)-6-(1-amino-1,3- dihydrospiro[indene-2,4′- piperidin]-1′-yl)-3-(1-(m- tolyl)cyclopropyl)-1,5-dihydro- 4H-pyrazolo[3,4-d]pyrimidin-4- one | 467.25 |
| Example 11 (A011) | 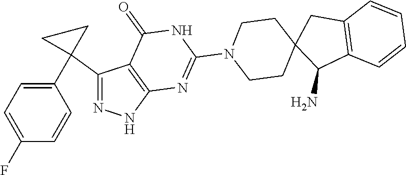 [SMILES: N[C@@H]1c2ccccc2CC12CCN(c1nc3[nH]nc(C4(c5ccc(F)cc5)CC4)c3c(=O)[nH]1)CC2] | (S)-6-(1-amino-1,3- dihydrospiro[indene-2,4′- piperidin]-1′-yl)-3-(1-(4- fluorophenyl)cyclopropyl)-1,5- dihydro-4H-pyrazolo[3,4- d]pyrimidin-4-one | 471.22 |
| Example 12 (A012) | 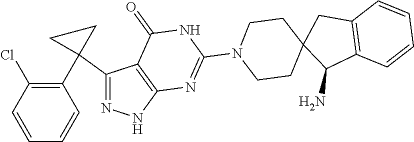 [SMILES: N[C@@H]1c2ccccc2CC12CCN(c1nc3[nH]nc(C4(c5ccccc5Cl)CC4)c3c(=O)[nH]1)CC2] | (S)-6-(1-amino-1,3- dihydrospiro[indene-2,4′- piperidin]-1′-yl)-3-(1-(2- chlorophenyl)cyclopropyl)-1,5- dihydro-4H-pyrazolo[3,4- d]pyrimidin-4-one | 487.19 |
| Example 13 (A013) | 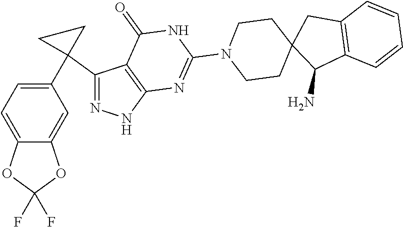 [SMILES: N[C@@H]1c2ccccc2CC12CCN(c1nc3[nH]nc(C4(c5ccc6c(c5)OC(F)(F)O6)CC4)c3c(=O)[nH]1)CC2] | (S)-6-(1-amino-1,3- dihydrospiro[indene-2,4′- piperidin]-1′-yl)-3-(1-(2,2- difluorobenzo[d][1,3]dioxol-5- yl)cyclopropyl)-1,5-dihydro-4H- pyrazolo[3,4-d]pyrimidin-4-one | 533.20 |
| Example 14 (A014) | 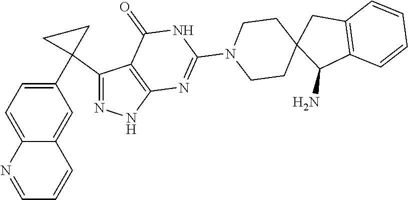 [SMILES: N[C@@H]1c2ccccc2CC12CCN(c1nc3[nH]nc(C4(c5ccc6ncccc6c5)CC4)c3c(=O)[nH]1)CC2] | (S)-6-(1-amino-1,3- dihydrospiro[indene-2,4′- piperidin]-1′-yl)-3-(1-(quinolin-6- yl)cyclopropyl)-1,5-dihydro-4H- pyrazolo[3,4-d]pyrimidin-4-one | 504.24 |
| Example 15 (A015) | 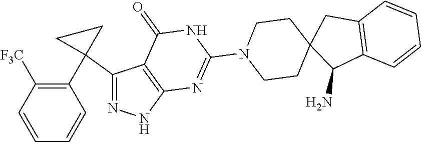 [SMILES: N[C@@H]1c2ccccc2CC12CCN(c1nc3[nH]nc(C4(c5ccccc5C(F)(F)F)CC4)c3c(=O)[nH]1)CC2] | (S)-6-(1-amino-1,3- dihydrospiro[indene-2,4′- piperidin]-1′-yl)-3-(1-(2- (trifluoromethyl)phenyl)cyclo- propyl)-1,5-dihydro-4H- pyrazolo[3,4-d]pyrimidin-4-one | 521.22 |
| Example 16 (A016) | 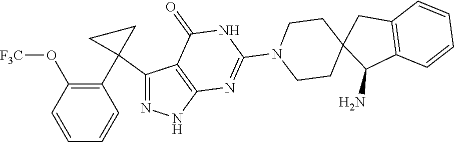 [SMILES: N[C@@H]1c2ccccc2CC12CCN(c1nc3[nH]nc(C4(c5ccccc5OC(F)(F)F)CC4)c3c(=O)[nH]1)CC2] | (S)-6-(1-amino-1,3- dihydrospiro[indene-2,4′- piperidin]-1′-yl)-3-(1-(2- (trifluoromethoxy)phenyl)cyclo- propyl)-1,5-dihydro-4H- pyrazolo[3,4-d]pyrimidin-4-one | 537.21 |
| Example 17 (A017) | 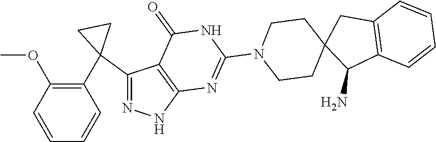 [SMILES: COc1ccccc1C1(c2n[nH]c3nc(N4CCC5(CC4)Cc4ccccc4[C@H]5N)[nH]c(=O)c23)CC1] | (S)-6-(1-amino-1,3- dihydrospiro[indene-2,4′- piperidin]-1′-yl)-3-(1-(2- methoxyphenyl)cyclopropyl)-1,5- dihydro-4H-pyrazolo[3,4- d]pyrimidin-4-one | 583.24 |
| Example 18 (A018) | 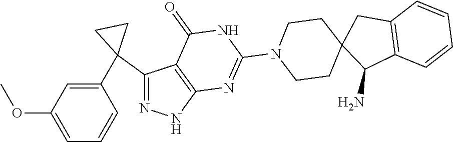 [SMILES: COc1cccc(C2(c3n[nH]c4nc(N5CCC6(CC5)Cc5ccccc5[C@H]6N)[nH]c(=O)c34)CC2)c1] | (S)-6-(1-amino-1,3- dihydrospiro[indene-2,4′- piperidin]-1′-yl)-3-(1-(3- methoxyphenyl)cyclopropyl)-1,5- dihydro-4H-pyrazolo[3,4- d]pyrimidin-4-one | 483.24 |
| Example 19 (A019) | 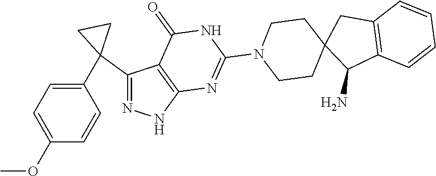 [SMILES: COc1ccc(C2(c3n[nH]c4nc(N5CCC6(CC5)Cc5ccccc5[C@H]6N)[nH]c(=O)c34)CC2)cc1] | (S)-6-(1-amino-1,3- dihydrospiro[indene-2,4′- piperidin]-1′-yl)-3-(1-(4- methoxyphenyl)cyclopropyl)-1,5- dihydro-4H-pyrazolo[3,4- d]pyrimidin-4-one | 483.24 |
| Example 20 (A020) | 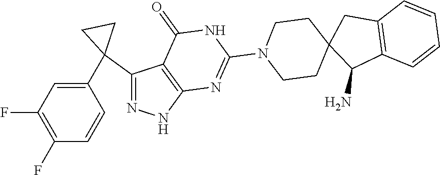 [SMILES: N[C@@H]1c2ccccc2CC12CCN(c1nc3[nH]nc(C4(c5ccc(F)c(F)c5)CC4)c3c(=O)[nH]1)CC2] | (S)-6-(1-amino-1,3- dihydrospiro[indene-2,4′- piperidin]-1′-yl)-3-(1-(3,4- difluorophenyl)cyclopropyl)-1,5- dihydro-4H-pyrazolo[3,4- d]pyrimidin-4-one | 489.21 |
| Example 21 (A021) | 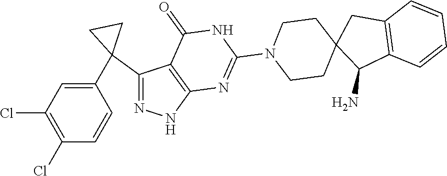 [SMILES: N[C@@H]1c2ccccc2CC12CCN(c1nc3[nH]nc(C4(c5ccc(Cl)c(Cl)c5)CC4)c3c(=O)[nH]1)CC2] | (S)-6-(1-amino-1,3- dihydrospiro[indene-2,4′- piperidin]-1′-yl)-3-(1-(3,4- dichlorophenyl)cyclopropyl)-1,5- dihydro-4H-pyrazolo[3,4- d]pyrimidin-4-one | 521.15 |
| Example 22 (A022) | 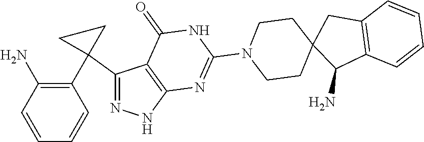 [SMILES: Nc1ccccc1C1(c2n[nH]c3nc(N4CCC5(CC4)Cc4ccccc4[C@H]5N)[nH]c(=O)c23)CC1] | (S)-6-(1-amino-1,3- dihydrospiro[indene-2,4′- piperidin]-1′-yl)-3-(1-(2- aminophenyl)cyclopropyl)-1,5- dihydro-4H-pyrazolo[3,4- d]pyrimidin-4-one | 468.24 |
| Example 23 (A023) | 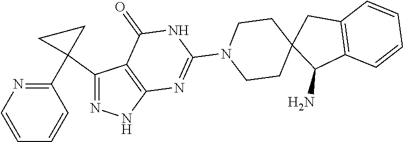 [SMILES: N[C@@H]1c2ccccc2CC12CCN(c1nc3[nH]nc(C4(c5ccccn5)CC4)c3c(=O)[nH]1)CC2] | (S)-6-(1-amino-1,3- dihydrospiro[indene-2,4′- piperidin]-1′-yl)-3-(1-(pyridin-2- yl)cyclopropyl)-1,5-dihydro-4H- pyrazolo[3,4-d]pyrimidin-4-one | 454.23 |
| Example 24 (A024) | 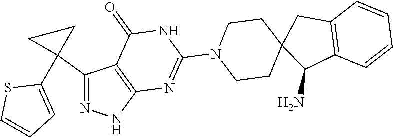 [SMILES: N[C@@H]1c2ccccc2CC12CCN(c1nc3[nH]nc(C4(c5cccs5)CC4)c3c(=O)[nH]1)CC2] | (S)-6-(1-amino-1,3- dihydrospiro[indene-2,4′- piperidin]-1′-yl)-3-(1-(thiophen-2- yl)cyclopropyl)-1,5-dihydro-4H- pyrazolo[3,4-d]pyrimidin-4-one | 459.19 |
| Example 25 (A025) | 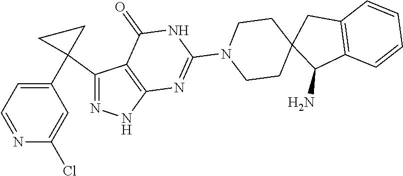 [SMILES: N[C@@H]1c2ccccc2CC12CCN(c1nc3[nH]nc(C4(c5ccnc(Cl)c5)CC4)c3c(=O)[nH]1)CC2] | (S)-6-(1-amino-1,3- dihydrospiro[indene-2,4′- piperidin]-1′-yl)-3-(1-(2- chloropyridin-4-yl)cyclopropyl)- 1,5-dihydro-4H-pyrazolo[3,4- d]pyrimidin-4-one | 488.19 |
| Example 26 (A026) | 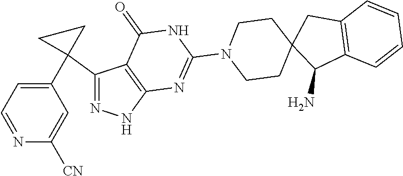 [SMILES: N#Cc1cc(C2(c3n[nH]c4nc(N5CCC6(CC5)Cc5ccccc5[C@H]6N)[nH]c(=O)c34)CC2)ccn1] | (S)-4-(1-(6-(1-amino-1,3- dihydrospiro[indene-2,4′- piperidin]-1′-yl)-4-oxo-4,5- dihydro-1H-pyrazolo[3,4- d]pyrimidin-3- yl)cyclopropyl)picolinonitrile | 479.22 |
| Example 27 (A027) | 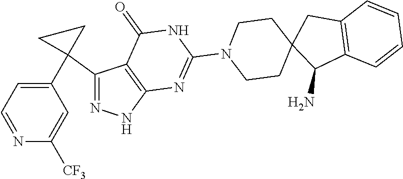 [SMILES: N[C@@H]1c2ccccc2CC12CCN(c1nc3[nH]nc(C4(c5ccnc(C(F)(F)F)c5)CC4)c3c(=O)[nH]1)CC2] | (S)-6-(1-amino-1,3- dihydrospiro[indene-2,4′- piperidin]-1′-yl)-3-(1-(2- (trifluoromethyl)pyridin-4- yl)cyclopropyl)-1,5-dihydro-4H- pyrazolo[3,4-d]pyrimidin-4-one | 522.22 |
| Example 28 (A028) | 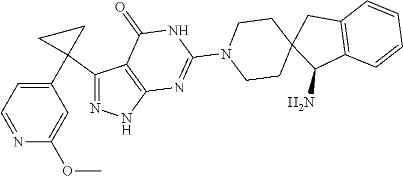 [SMILES: COc1cc(C2(c3n[nH]c4nc(N5CCC6(CC5)Cc5ccccc5[C@H]6N)[nH]c(=O)c34)CC2)ccn1] | (S)-6-(1-amino-1,3- dihydrospiro[indene-2,4′- piperidin]-1′-yl)-3-(1-(2- methoxypyridin-4- yl)cyclopropyl)-1,5-dihydro-4H- pyrazolo[3,4-d]pyrimidin-4-one | 484.24 |
| Example 29 (A029) | 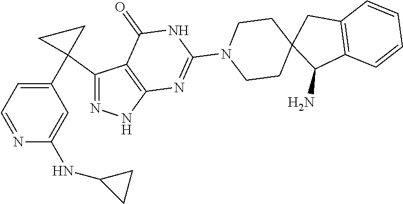 [SMILES: N[C@@H]1c2ccccc2CC12CCN(c1nc3[nH]nc(C4(c5ccnc(NC6CC6)c5)CC4)c3c(=O)[nH]1)CC2] | (S)-6-(1-amino-1,3- dihydrospiro[indene-2,4′- piperidin]-1′-yl)-3-(1-(2- (cyclopropylamino)pyridin-4- yl)cyclopropyl)-1,5-dihydro-4H- pyrazolo[3,4-d]pyrimidin-4-one | 509.27 |
| Example 30 (A030) | 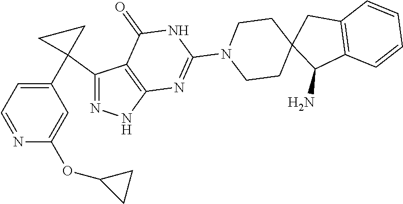 [SMILES: N[C@@H]1c2ccccc2CC12CCN(c1nc3[nH]nc(C4(c5ccnc(OC6CC6)c5)CC4)c3c(=O)[nH]1)CC2] | (S)-6-(1-amino-1,3- dihydrospiro[indene-2,4′- piperidin]-1′-yl)-3-(1-(2- cyclopropoxypyridin-4- yl)cyclopropyl)-1,5-dihydro-4H- pyrazolo[3,4-d]pyrimidin-4-one | 509.27 |
| Example 31 (A031) | 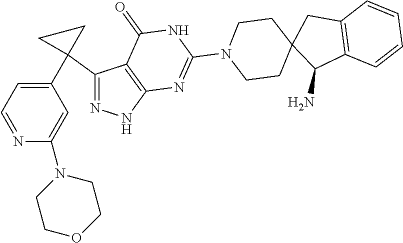 [SMILES: N[C@@H]1c2ccccc2CC12CCN(c1nc3[nH]nc(C4(c5ccnc(N6CCOCC6)c5)CC4)c3c(=O)[nH]1)CC2] | (S)-6-(1-amino-1,3- dihydrospiro[indene-2,4′- piperidin]-1′-yl)-3-(1-(2- morpholinopyridin-4- yl)cyclopropyl)-1,5-dihydro-4H- pyrazolo[3,4-d]pyrimidin-4-one | 539.28 |
| Example 32 (A032) | 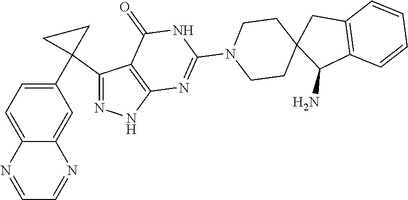 [SMILES: N[C@@H]1c2ccccc2CC12CCN(c1nc3[nH]nc(C4(c5ccc6nccnc6c5)CC4)c3c(=O)[nH]1)CC2] | (S)-6-(1-amino-1,3- dihydrospiro[indene-2,4′- piperidin]-1′-yl)-3-(1-(quinoxalin- 6-yl)cyclopropyl)-1,5-diliydro- 4H-pyrazolo[3,4-d]pyrimidin-4- one | 505.24 |
| Example 34 (A034) | 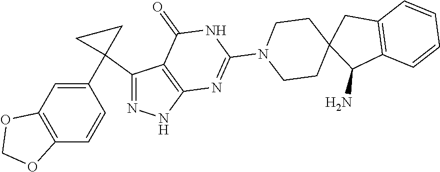 [SMILES: N[C@@H]1c2ccccc2CC12CCN(c1nc3[nH]nc(C4(c5ccc6c(c5)OCO6)CC4)c3c(=O)[nH]1)CC2] | (S)-6-(1-amino-1,3- dihydrospiro[indene-2,4′- piperidin]-1′-yl)-3-(1- (benzo[d][1,3]dioxol-5- yl)cyclopropyl)-1,5-dihydro-4H- pyrazolo[3,4-d]pyrimidin-4-one | 497.22 |
| Example 35 (A035) | 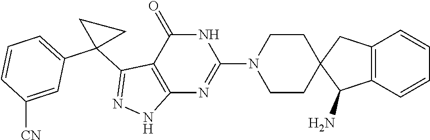 [SMILES: N#Cc1cccc(C2(c3n[nH]c4nc(N5CCC6(CC5)Cc5ccccc5[C@H]6N)[nH]c(=O)c34)CC2)c1] | (S)-3-(1-(6-(1-amino-1,3- dihydrospiro[indene-2,4′- piperidin]-1′-yl)-4-oxo-4,5- dihydro-1H-pyrazolo[3,4- d]pyrimidin-3- yl)cyclopropyl)benzonitrile | 478.23 |
| Example 36 (A036) | 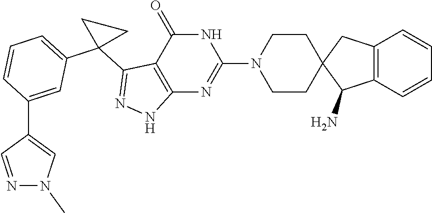 [SMILES: Cn1cc(-c2cccc(C3(c4n[nH]c5nc(N6CCC7(CC6)Cc6ccccc6[C@H]7N)[nH]c(=O)c45)CC3)c2)cn1] | (S)-6-(1-amino-1,3- dihydrospiro[indene-2,4′- piperidin]-1′-yl)-3-(1-(3-(1- methyl-1H-pyrazol-4- yl)phenyl)cyclopropyl)-1,5- dihydro-4H-pyrazolo[3,4- d]pyrimidin-4-one | 533.27 |
| Example 37 (A037) | 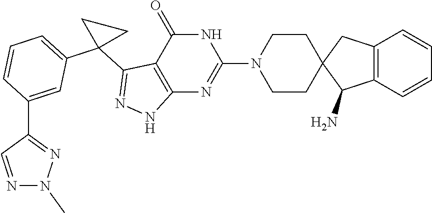 [SMILES: Cn1ncc(-c2cccc(C3(c4n[nH]c5nc(N6CCC7(CC6)Cc6ccccc6[C@H]7N)[nH]c(=O)c45)CC3)c2)n1] | (S)-6-(1-amino-1,3- dihydrospiro[indene-2,4′- piperidin]-1′-yl)-3-(1-(3-(2- methyl-2H-1,2,3-triazol-4- yl)phenyl)cyclopropyl)-1,5- dihydro-4H-pyrazolo[3,4- d]pyrimidin-4-one | 534.27 |
| Example 38 (A038) | 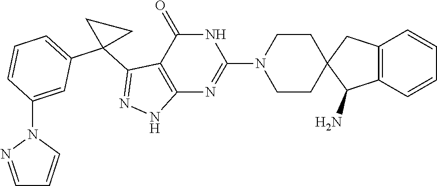 [SMILES: N[C@@H]1c2ccccc2CC12CCN(c1nc3[nH]nc(C4(c5cccc(-n6cccn6)c5)CC4)c3c(=O)[nH]1)CC2] | (S)-3-(1-(3-(1H-pyrazol-1- yl)phenyl)cyclopropyl)-6-(1- amino-1,3-dihydrospiro[indene- 2,4′-piperidin]-1′-yl)-1,5-dihydro- 4H-pyrazolo[3,4-d]pyrimidin-4- one | 519.25 |
| Example 39 (A039) | 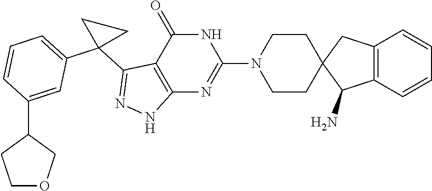 [SMILES: N[C@@H]1c2ccccc2CC12CCN(c1nc3[nH]nc(C4(c5cccc(C6CCOC6)c5)CC4)c3c(=O)[nH]1)CC2] | 6-((S)-1-amino-1,3- dihydrospiro[indene-2,4′- piperidin]-1′-yl)-3-(1-(3- (tetrahydrofuran-3- yl)phenyl)cyclopropyl)-1,5- dihydro-4H-pyrazolo[3,4- d]pyrimidin-4-one | 523.27 |
| Example 40 (A040) | 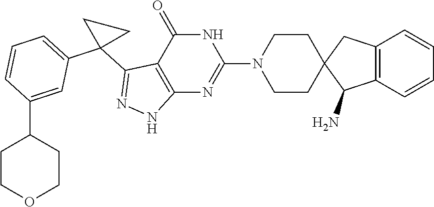 [SMILES: N[C@@H]1c2ccccc2CC12CCN(c1nc3[nH]nc(C4(c5cccc(C6CCOCC6)c5)CC4)c3c(=O)[nH]1)CC2] | (S)-6-(1-amino-1,3- dihydrospiro[indene-2,4′- piperidin]-1′-yl)-3-(1-(3- (tetrahydro-2H-pyran-4- yl)phenyl)cyclopropyl)-1,5- dihydro-4H-pyrazolo[3,4- d]pyrimidin-4-one | 537.29 |
| Example 41 (A041) | 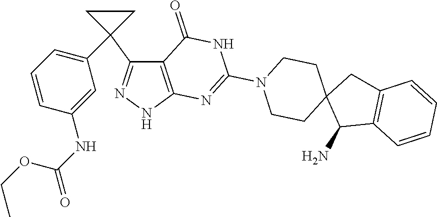 [SMILES: CCOC(=O)Nc1cccc(C2(c3n[nH]c4nc(N5CCC6(CC5)Cc5ccccc5[C@H]6N)[nH]c(=O)c34)CC2)c1] | ethyl (S)-(3-(1-(6-(1-amino-1,3- dihydrospiro[indene-2,4′- piperidin]-1′-yl)-4-oxo-4,5- dihydro-1H-pyrazolo[3,4- d]pyrimidin-3- yl)cyclopropyl)phenyl)carbamate | 540.26 |
| Example 42 (A042) | 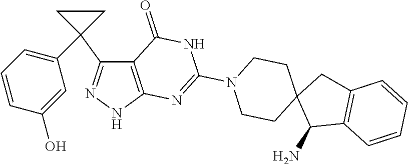 [SMILES: N[C@@H]1c2ccccc2CC12CCN(c1nc3[nH]nc(C4(c5cccc(O)c5)CC4)c3c(=O)[nH]1)CC2] | (S)-6-(1-amino-1,3- dihydrospiro[indene-2,4′- piperidin]-1′-yl)-3-(1-(3- hydroxyphenyl)cyclopropyl)-1,5- dihydro-4H-pyrazolo[3,4- d]pyrimidin-4-one | 569.23 |
| Example 43 (A043) | 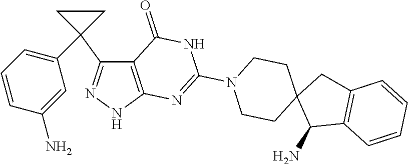 [SMILES: Nc1cccc(C2(c3n[nH]c4nc(N5CCC6(CC5)Cc5ccccc5[C@H]6N)[nH]c(=O)c34)CC2)c1] | (S)-6-(1-amino-1,3- dihydrospiro[indene-2,4′- piperidin]-1′-yl)-3-(1-(3- aminophenyl)cyclopropyl)-1,5- dihydro-4H-pyrazolo[3,4- d]pyrimidin-4-one | 468.24 |
| Example 44 (A044) | 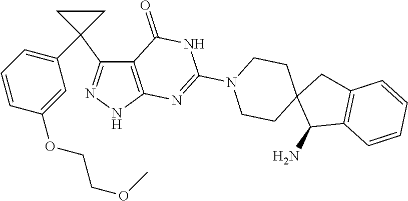 [SMILES: COCCOc1cccc(C2(c3n[nH]c4nc(N5CCC6(CC5)Cc5ccccc5[C@H]6N)[nH]c(=O)c34)CC2)c1] | (S)-6-(1-amino-1,3- dihydrospiro[indene-2,4′- piperidin]-1′-yl)-3-(1-(3-(2- methoxyethoxy)phenyl)cycloprop- yl)-1,5-dihydro-4H-pyrazolo[3,4- d]pyrimidin-4-one | 527.27 |
| Example 45 (A045) | 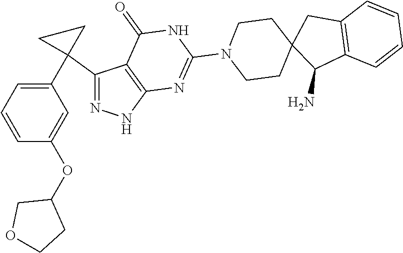 [SMILES: N[C@@H]1c2ccccc2CC12CCN(c1nc3[nH]nc(C4(c5cccc(OC6CCOC6)c5)CC4)c3c(=O)[nH]1)CC2] | 6-((S)-1-amino-1,3- dihydrospiro[indene-2,4′- piperidin]-1′-yl)-3-(1-(3- ((tetrahydrofuran-3- yl)oxy)phenyl)cyclopropyl)-1,5- dihydro-4H-pyrazolo[3,4- d]pyrimidin-4-one | 539.27 |
| Example 46 (A046) | 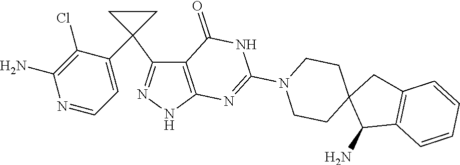 [SMILES: Nc1nccc(C2(c3n[nH]c4nc(N5CCC6(CC5)Cc5ccccc5[C@H]6N)[nH]c(=O)c34)CC2)c1Cl] | (S)-6-(1-amino-1,3- dihydrospiro[indene-2,4′- piperidin]-1′-yl)-3-(1-(2-amino-3- chloropyridin-4-yl)cyclopropyl)- 1,5-dihydro-4H-pyrazolo[3,4- d]pyrimidin-4-one | 503.20 |
| Example 47 (A047) | 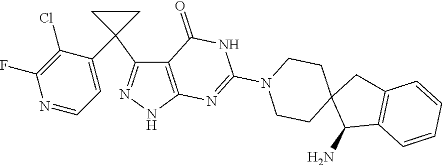 [SMILES: N[C@@H]1c2ccccc2CC12CCN(c1nc3[nH]nc(C4(c5ccnc(F)c5Cl)CC4)c3c(=O)[nH]1)CC2] | (S)-6-(1-amino-1,3- dihydrospiro[indene-2,4′- piperidin]-1′-yl)-3-(1-(3-chloro-2- fluoropyridin-4-yl)cyclopropyl)- 1,5-dihydro-4H-pyrazolo[3,4- d]pyrimidin-4-one | 506.18 |
| Example 48 (A048) | 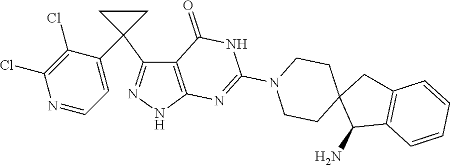 [SMILES: N[C@@H]1c2ccccc2CC12CCN(c1nc3[nH]nc(C4(c5ccnc(Cl)c5Cl)CC4)c3c(=O)[nH]1)CC2] | (S)-6-(1-amino-1,3- dihydrospiro[indene-2,4′- piperidin]-1′-yl)-3-(1-(2,3- dichloropyridin-4- yl)cyclopropyl)-1,5-dihydro-4H- pyrazolo[3,4-d]pyrimidin-4-one | 522.15 |
| Example 49 (A049) | 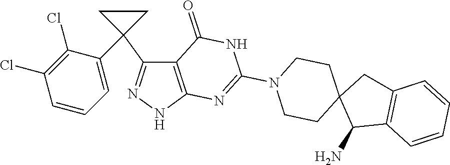 [SMILES: N[C@@H]1c2ccccc2CC12CCN(c1nc3[nH]nc(C4(c5cccc(Cl)c5Cl)CC4)c3c(=O)[nH]1)CC2] | (S)-6-(1-amino-1,3- dihydrospiro[indene-2,4′- piperidin]-1′-yl)-3-(1-(2,3- dichlorophenyl)cyclopropyl)-1,5- dihydro-4H-pyrazolo[3,4- d]pyrimidin-4-one | 521.15 |
| Example 50 (A050) | 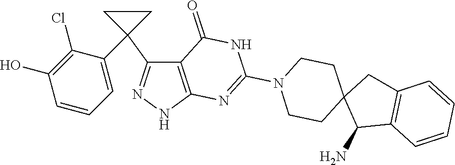 [SMILES: N[C@@H]1c2ccccc2CC12CCN(c1nc3[nH]nc(C4(c5cccc(O)c5Cl)CC4)c3c(=O)[nH]1)CC2] | (S)-6-(1-amino-1,3- dihydrospiro[indene-2,4′- piperidin]-1′-yl)-3-(1-(2-chloro-3- hydroxyphenyl)cyclopropyl)-1,5- dihydro-4H-pyrazolo[3,4- d]pyrimidin-4-one | 503.19 |
| Example 51 (A051) | 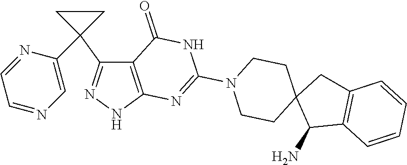 [SMILES: N[C@@H]1c2ccccc2CC12CCN(c1nc3[nH]nc(C4(c5cnccn5)CC4)c3c(=O)[nH]1)CC2] | (S)-6-(1-amino-1,3- dihydrospiro[indene-2,4′- piperidin]-1′-yl)-3-(1-(pyrazin-2- yl)cyclopropyl)-1,5-dihydro-4H- pyrazolo[3,4-d]pyrimidin-4-one | 455.22 |
| Example 52 (A052) | 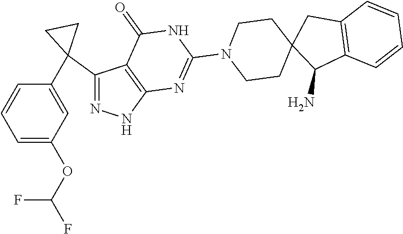 [SMILES: N[C@@H]1c2ccccc2CC12CCN(c1nc3[nH]nc(C4(c5cccc(OC(F)F)c5)CC4)c3c(=O)[nH]1)CC2] | (S)-6-(1-amino-1,3- dihydrospiro[indene-2,4′- piperidin]-1′-yl)-3-(1-(3- (difluoromethoxy)plienyl)cyclo- propyl)-1,5-dihydro-4H- pyrazolo[3,4-d]pyrimidin-4-one | 519.22 |
| Example 53 (A053) | 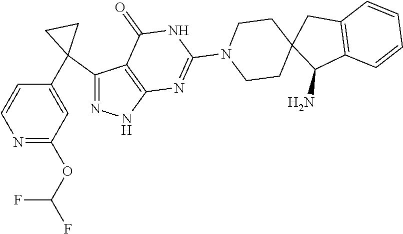 [SMILES: N[C@@H]1c2ccccc2CC12CCN(c1nc3[nH]nc(C4(c5ccnc(OC(F)F)c5)CC4)c3c(=O)[nH]1)CC2] | (S)-6-(1-amino-1,3- dihydrospiro[indene-2,4′- piperidin]-1′-yl)-3-(1-(2- (difluoromethoxy)pyridin-4- yl)cyclopropyl)-1,5-dihydro-4H- pyrazolo[3,4-d]pyrimidin-4-one | 520.22 |
| Example 54 (A054) | 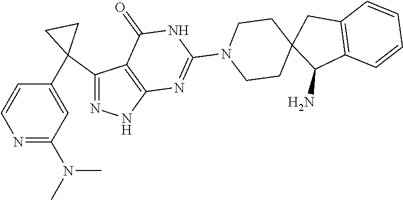 [SMILES: CN(C)c1cc(C2(c3n[nH]c4nc(N5CCC6(CC5)Cc5ccccc5[C@H]6N)[nH]c(=O)c34)CC2)ccn1] | (S)-6-(1-amino-1,3- dihydrospiro[indene-2,4′- piperidin]-1′-yl)-3-(1-(2- (dimethylamino)pyridin-4- yl)cyclopropyl)-1,5-dihydro-4H- pyrazolo[3,4-d]pyrimidin-4-one | 497.27 |
| Example 55 (A055) | 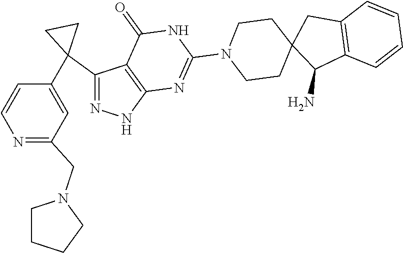 [SMILES: N[C@@H]1c2ccccc2CC12CCN(c1nc3[nH]nc(C4(c5ccnc(CN6CCCC6)c5)CC4)c3c(=O)[nH]1)CC2] | (S)-6-(1-amino-1,3- dihydrospiro[indene-2,4′- piperidin]-1′-yl)-3-(1-(2- (pyrrolidin-1-ylmethyl)pyridin-4- yl)cyclopropyl)-1,5-dihydro-4H- pyrazolo[3,4-d]pyrimidin-4-one | 537.30 |
| Example 56 (A056) | 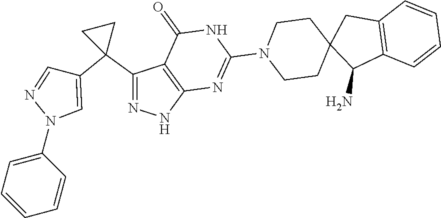 [SMILES: N[C@@H]1c2ccccc2CC12CCN(c1nc3[nH]nc(C4(c5cnn(-c6ccccc6)c5)CC4)c3c(=O)[nH]1)CC2] | (S)-6-(1-amino-1,3- dihydrospiro[indene-2,4′- piperidin]-1′-yl)-3-(1-(1-phenyl- 1H-pyrazol-4-yl)cyclopropyl)- 1,5-dihydro-4H-pyrazolo[3,4- d]pyrimidin-4-one | 519.25 |
| Example 57 (A057) | 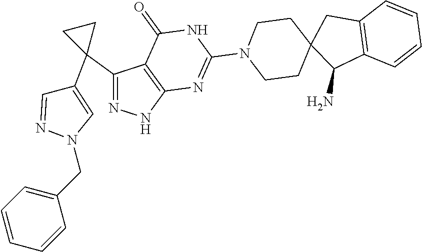 [SMILES: N[C@@H]1c2ccccc2CC12CCN(c1nc3[nH]nc(C4(c5cnn(Cc6ccccc6)c5)CC4)c3c(=O)[nH]1)CC2] | (S)-6-(1-amino-1,3- dihydrospiro[indene-2,4′- piperidin]-1′-yl)-3-(1-(1-benzyl- 1H-pyrazol-4-yl)cyclopropyl)- 1,5-dihydro-4H-pyrazolo[3,4- d]pyrimidin-4-one | 533.27 |
| Example 58 (A058) | 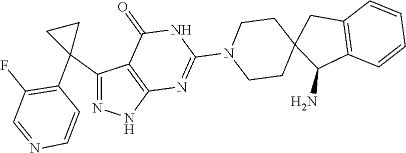 [SMILES: N[C@@H]1c2ccccc2CC12CCN(c1nc3[nH]nc(C4(c5ccncc5F)CC4)c3c(=O)[nH]1)CC2] | (S)-6-(1-amino-1,3- dihydrospiro[indene-2,4′- piperidin]-1′-yl)-3-(1-(3- fluoropyridin-4-yl)cyclopropyl)- 1,5-dihydro-4H-pyrazolo[3,4- d]pyrimidin-4-one | 472.22 |
| Example 59 (A059) | 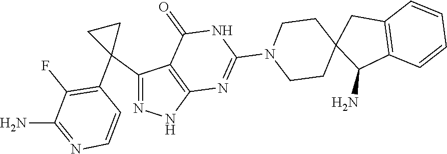 [SMILES: Nc1nccc(C2(c3n[nH]c4nc(N5CCC6(CC5)Cc5ccccc5[C@H]6N)[nH]c(=O)c34)CC2)c1F] | (S)-6-(1-amino-1,3- dihydrospiro[indene-2,4′- piperidin]-1′-yl)-3-(1-(2-amino-3- fluoropyridin-4-yl)cyclopropyl)- 1,5-dihydro-4H-pyrazolo[3,4- d]pyrimidin-4-one | 487.23 |
| Example 60 (A060) | 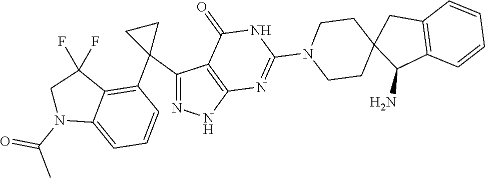 [SMILES: CC(=O)N1CC(F)(F)c2c1cccc2C1(c2n[nH]c3nc(N4CCC5(CC4)Cc4ccccc4[C@H]5N)[nH]c(=O)c23)CC1] | (S)-3-(1-(1-acetyl-3,3- difluoroindolin-4-yl)cyclopropyl)- 6-(1-amino-1,3- dihydrospiro[indene-2,4′- piperidin]-1′-yl)-1,5-dihydro-4H- pyrazolo[3,4-d]pyrimidin-4-one | 572.25 |
| Example 61 (A061) | 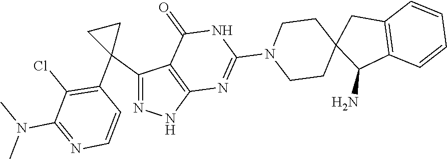 [SMILES: CN(C)c1nccc(C2(c3n[nH]c4nc(N5CCC6(CC5)Cc5ccccc5[C@H]6N)[nH]c(=O)c34)CC2)c1Cl] | (S)-6-(1-amino-1,3- dihydrospiro[indene-2,4′- piperidin]-1′-yl)-3-(1-(3-chloro-2- (dimethylamino)pyridin-4- yl)cyclopropyl)-1,5-dihydro-4H- pyrazolo[3,4-d]pyrimidin-4-one | 531.23 |
| Example 62 (A062) | 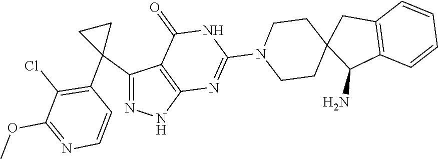 [SMILES: COc1nccc(C2(c3n[nH]c4nc(N5CCC6(CC5)Cc5ccccc5[C@H]6N)[nH]c(=O)c34)CC2)c1Cl] | (S)-6-(1-amino-1,3- dihydrospiro[indene-2,4′- piperidin]-1′-yl)-3-(1-(3-chloro-2- methoxypyridin-4- yl)cyclopropyl)-1,5-dihydro-4H- pyrazolo[3,4-d]pyrimidin-4-one | 518.20 |
| Example 63 (A063) | 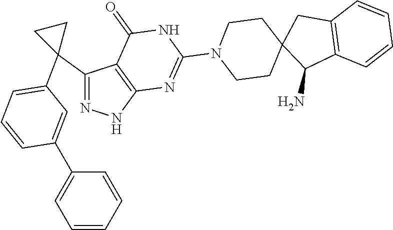 [SMILES: N[C@@H]1c2ccccc2CC12CCN(c1nc3[nH]nc(C4(c5cccc(-c6ccccc6)c5)CC4)c3c(=O)[nH]1)CC2] | (S)-3-(1-([1,1′-biphenyl]-3- yl)cyclopropyl)-6-(1-amino-1,3- dihydrospiro[indene-2,4′- piperidin]-1′-yl)-1,5-dihydro-4H- pyrazolo[3,4-d]pyrimidin-4-one | 529.26 |
| Example 65 (A065) | 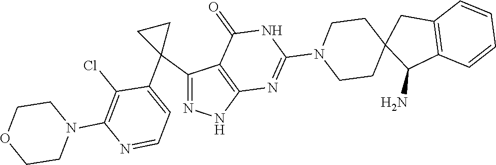 [SMILES: N[C@@H]1c2ccccc2CC12CCN(c1nc3[nH]nc(C4(c5ccnc(N6CCOCC6)c5Cl)CC4)c3c(=O)[nH]1)CC2] | (S)-6-(1-amino-1,3- dihydrospiro[indene-2,4′- piperidin]-1′-yl)-3-(1-(3-chloro-2- morpholinopyridin-4- yl)cyclopropyl)-1,5-dihydro-4H- pyrazolo[3,4-d]pyrimidin-4-one | 573.24 |
| Example 66 (A066) | 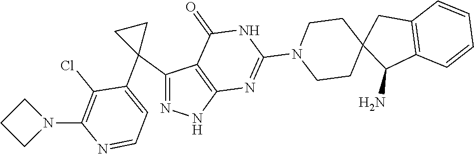 [SMILES: N[C@@H]1c2ccccc2CC12CCN(c1nc3[nH]nc(C4(c5ccnc(N6CCC6)c5Cl)CC4)c3c(=O)[nH]1)CC2] | (S)-6-(1-amino-1,3- dihydrospiro[indene-2,4′- piperidin]-1′-yl)-3-(1-(2-(azetidin- 1-yl)-3-chloropyridin-4- yl)cyclopropyl)-1,5-dihydro-4H- pyrazolo[3,4-d]pyrimidin-4-one | 543.23 |
| Example 67 (A067) | 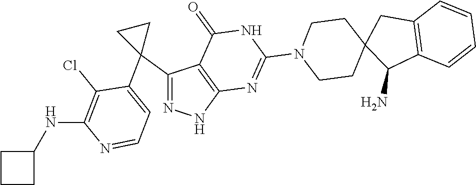 [SMILES: N[C@@H]1c2ccccc2CC12CCN(c1nc3[nH]nc(C4(c5ccnc(NC6CCC6)c5Cl)CC4)c3c(=O)[nH]1)CC2] | (S)-6-(1-amino-1,3- dihydrospiro[indene-2,4′- piperidin]-1′-yl)-3-(1-(3-chloro-2- (cyclobutylamino)pyridin-4- yl)cyclopropyl)-1,5-dihydro-4H- pyrazolo[3,4-d]pyrimidin-4-one | 557.25 |
| Example 69 (A069) | 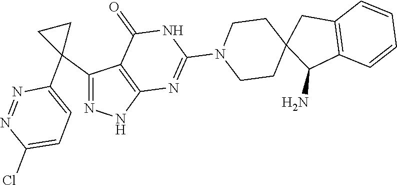 [SMILES: N[C@@H]1c2ccccc2CC12CCN(c1nc3[nH]nc(C4(c5ccc(Cl)nn5)CC4)c3c(=O)[nH]1)CC2] | (S)-6-(1-amino-1,3- dihydrospiro[indene-2,4′- piperidin]-1′-yl)-3-(1-(6- chloropyridazin-3- yl)cyclopropyl)-1,5-dihydro-4H- pyrazolo[3,4-d]pyrimidin-4-one | 489.18 |
| Example 70 (A070) | 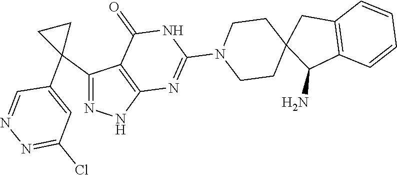 [SMILES: N[C@@H]1c2ccccc2CC12CCN(c1nc3[nH]nc(C4(c5cnnc(Cl)c5)CC4)c3c(=O)[nH]1)CC2] | (S)-6-(1-amino-1,3- dihydrospiro[indene-2,4′- piperidin]-1′-yl)-3-(1-(6- chloropyridazin-4- yl)cyclopropyl)-1,5-dihydro-4H- pyrazolo[3,4-d]pyrimidin-4-one | 489.18 |
| Example 71 (A071) | 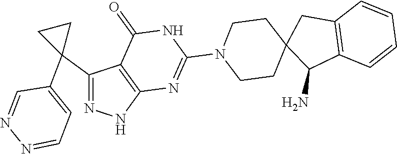 [SMILES: N[C@@H]1c2ccccc2CC12CCN(c1nc3[nH]nc(C4(c5ccnnc5)CC4)c3c(=O)[nH]1)CC2] | (S)-6-(1-amino-1,3- dihydrospiro[indene-2,4′- piperidin]-1′-yl)-3-(1-(pyridazin- 4-yl)cyclopropyl)-1,5-dihydro- 4H-pyrazolo[3,4-d]pyrimidin-4- one | 455.22 |
| Example 72 (A072) | 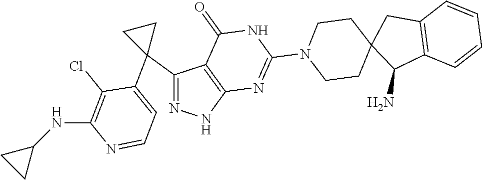 [SMILES: N[C@@H]1c2ccccc2CC12CCN(c1nc3[nH]nc(C4(c5ccnc(NC6CC6)c5Cl)CC4)c3c(=O)[nH]1)CC2] | (S)-6-(1-amino-1,3- dihydrospiro[indene-2,4′- piperidin]-1′-yl)-3-(1-(3-chloro-2- (cyclopropylamino)pyridin-4- yl)cyclopropyl)-1,5-dihydro-4H- pyrazolo[3,4-d]pyrimidin-4-one | 543.23 |
| Example 73 (A073) | 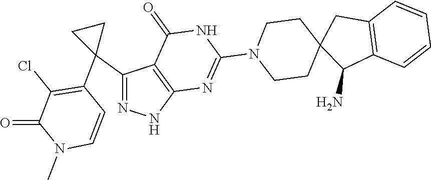 [SMILES: Cn1ccc(C2(c3n[nH]c4nc(N5CCC6(CC5)Cc5ccccc5[C@H]6N)[nH]c(=O)c34)CC2)c(Cl)c1=O] | (S)-6-(1-amino-1,3- dihydrospiro[indene-2,4′- piperidin]-1′-yl)-3-(1-(3-chloro-1- methyl-2-oxo-1,2-dihydropyridin- 4-yl)cyclopropyl)-1,5-dihydro- 4H-pyrazolo[3,4-d]pyrimidin-4- one | 518.20 |
| Example 77 (A077) | 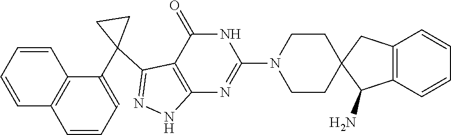 [SMILES: N[C@@H]1c2ccccc2CC12CCN(c1nc3[nH]nc(C4(c5cccc6ccccc56)CC4)c3c(=O)[nH]1)CC2] | (S)-6-(1-amino-1,3- dihydrospiro[indene-2,4′- piperidin]-1′-yl)-3-(1- (naphthalen-1-yl)cyclopropyl)- 1,5-dihydro-4H-pyrazolo[3,4- d]pyrimidin-4-one | 503.25 |
| Example 78 (A078) | 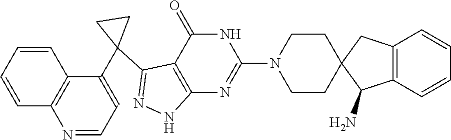 [SMILES: N[C@@H]1c2ccccc2CC12CCN(c1nc3[nH]nc(C4(c5ccnc6ccccc56)CC4)c3c(=O)[nH]1)CC2] | (S)-6-(1-amino-1,3- dihydrospiro[indene-2,4′- piperidin]-1′-yl)-3-(1-(quinolin-4- yl)cyclopropyl)-1,5-dihydro-4H- pyrazolo[3,4-d]pyrimidin-4-one | 504.24 |
| Example 79 (A079) | 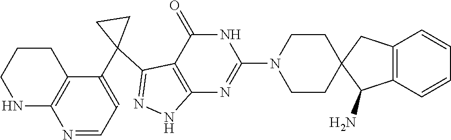 [SMILES: N[C@@H]1c2ccccc2CC12CCN(c1nc3[nH]nc(C4(c5ccnc6c5CCCN6)CC4)c3c(=O)[nH]1)CC2] | (S)-6-(1-amino-1,3- dihydrospiro[indene-2,4′- piperidin]-1′-yl)-3-(1-(5,6,7,8- tetrahydro-1,8-naplitliyridin-4- yl)cyclopropyl)-1,5-dihydro-4H- pyrazolo[3,4-d]pyrimidin-4-one | 509.27 |
| Example 80 (A080) | 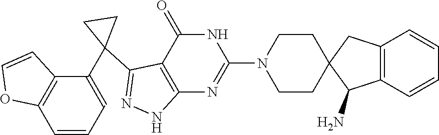 [SMILES: N[C@@H]1c2ccccc2CC12CCN(c1nc3[nH]nc(C4(c5cccc6occc56)CC4)c3c(=O)[nH]1)CC2] | (S)-6-(1-amino-1,3- dihydrospiro[indene-2,4′- piperidin]-1′-yl)-3-(1- (benzofuran-4-yl)cyclopropyl)- 1,5-dihydro-4H-pyrazolo[3,4- d]pyrimidin-4-one | 493.23 |
| Example 81 (A081) | 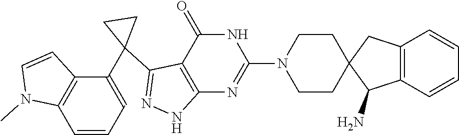 [SMILES: Cn1ccc2c(C3(c4n[nH]c5nc(N6CCC7(CC6)Cc6ccccc6[C@H]7N)[nH]c(=O)c45)CC3)cccc21] | (S)-6-(1-amino-1,3- dihydrospiro[indene-2,4′- piperidin]-1′-yl)-3-(1-(1-methyl- 1H-indol-4-yl)cyclopropyl)-1,5- dihydro-4H-pyrazolo[3,4- d]pyrimidin-4-one | 506.26 |
| Example 82 (A082) | 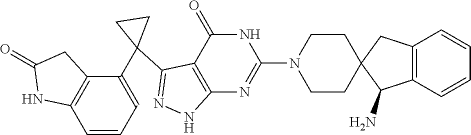 [SMILES: N[C@@H]1c2ccccc2CC12CCN(c1nc3[nH]nc(C4(c5cccc6c5CC(=O)N6)CC4)c3c(=O)[nH]1)CC2] | (S)-6-(1-amino-1,3- dihydrospiro[indene-2,4′- piperidin]-1′-yl)-3-(1-(2- oxoindolin-4-yl)cyclopropyl)-1,5- dihydro-4H-pyrazolo[3,4- d]pyrimidin-4-one | 508.24 |
| Example 83 (A083) | 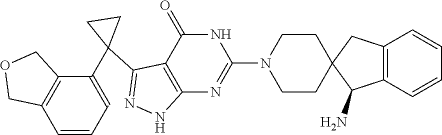 [SMILES: N[C@@H]1c2ccccc2CC12CCN(c1nc3[nH]nc(C4(c5cccc6c5COC6)CC4)c3c(=O)[nH]1)CC2] | (S)-6-(1-amino-1,3- dihydrospiro[indene-2,4′- piperidin]-1′-yl)-3-(1-(1,3- dihydroisobenzofuran-4- yl)cyclopropyl)-1,5-dihydro-4H- pyrazolo[3,4-d]pyrimidin-4-one | 495.24 |
| Example 84 (A084) | 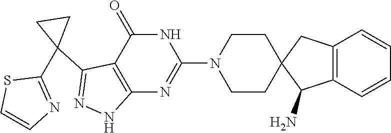 [SMILES: N[C@@H]1c2ccccc2CC12CCN(c1nc3[nH]nc(C4(c5nccs5)CC4)c3c(=O)[nH]1)CC2] | (S)-6-(1-amino-1,3- dihydrospiro[indene-2,4′- piperidin]-1′-yl)-3-(1-(thiazol-2- yl)cyclopropyl)-1,5-dihydro-4H- pyrazolo[3,4-d]pyrimidin-4-one | 460.18 |
| Example 85 (A085) | 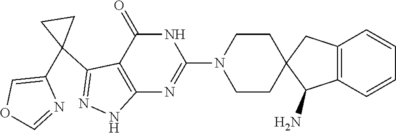 [SMILES: N[C@@H]1c2ccccc2CC12CCN(c1nc3[nH]nc(C4(c5cocn5)CC4)c3c(=O)[nH]1)CC2] | (S)-6-(1-amino-1,3- dihydrospiro[indene-2,4′- piperidin]-1′-yl)-3-(1-(oxazol-4- yl)cyclopropyl)-1,5-dihydro-4H- pyrazolo[3,4-d]pyrimidin-4-one | 444.21 |
| Example 86 (A086) | 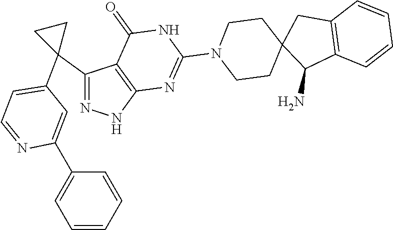 [SMILES: N[C@@H]1c2ccccc2CC12CCN(c1nc3[nH]nc(C4(c5ccnc(-c6ccccc6)c5)CC4)c3c(=O)[nH]1)CC2] | (S)-6-(1-amino-1,3- dihydrospiro[indene-2,4′- piperidin]-1′-yl)-3-(1-(2- phenylpyridin-4-yl)cyclopropyl)- 1,5-dihydro-4H-pyrazolo[3,4- d]pyrimidin-4-one | 530.26 |
| Example 87 (A087) | 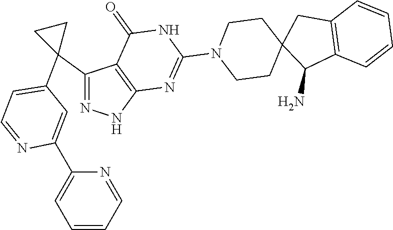 [SMILES: N[C@@H]1c2ccccc2CC12CCN(c1nc3[nH]nc(C4(c5ccnc(-c6ccccn6)c5)CC4)c3c(=O)[nH]1)CC2] | (S)-3-(1-([2,2′-bipyridin]-4- yl)cyclopropyl)-6-(1-amino-1,3- dihydrospiro[indene-2,4′- piperidin]-1′-yl)-1,5-dihydro-4H- pyrazolo[3,4-d]pyrimidin-4-one | 531.25 |
| Example 88 (A088) | 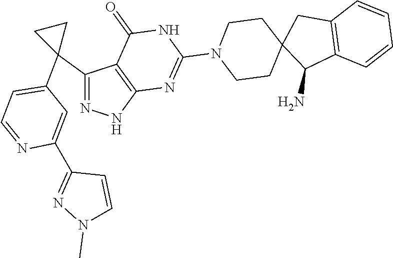 [SMILES: Cn1ccc(-c2cc(C3(c4n[nH]c5nc(N6CCC7(CC6)Cc6ccccc6[C@H]7N)[nH]c(=O)c45)CC3)ccn2)n1] | (S)-6-(1-amino-1,3- dihydrospiro[indene-2,4′- piperidin]-1′-yl)-3-(1-(2-(1- methyl-1H-pyrazol-3-yl)pyridin- 4-yl)cyclopropyl)-1,5-dihydro- 4H-pyrazolo[3,4-d]pyrimidin-4- one | 534.27 |
| Example 91 (A091) | 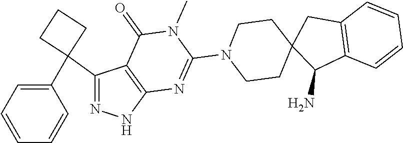 [SMILES: Cn1c(N2CCC3(CC2)Cc2ccccc2[C@H]3N)nc2[nH]nc(C3(c4ccccc4)CCC3)c2c1=O] | (S)-6-(1-amino-1,3- dihydrospiro[indene-2,4′- piperidin]-1′-yl)-5-methyl-3-(1- phenylcyclobutyl)-1,5-dihydro- 4H-pyrazolo[3,4-d]pyrimidin-4- one | 481.26 |
| Example 92 (A092) | 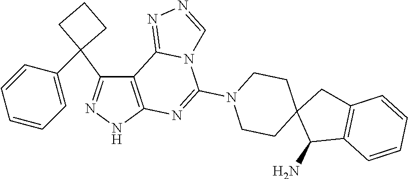 [SMILES: N[C@@H]1c2ccccc2CC12CCN(c1nc3[nH]nc(C4(c5ccccc5)CCC4)c3c3nncn13)CC2] | (S)-1′-(9-(1-phenylcyclobutyl)- 7H-pyrazolo[4,3- e][1,2,4]triazolo[4,3-c]pyrimidin- 5-yl)-1,3-dihydrospiro[indene- 2,4′-piperidin]-1-amine | 491.26 |
| Example 93 (A093) | 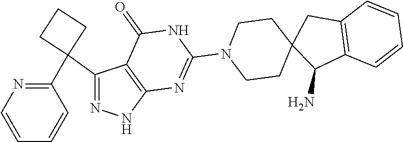 [SMILES: N[C@@H]1c2ccccc2CC12CCN(c1nc3[nH]nc(C4(c5ccccn5)CCC4)c3c(=O)[nH]1)CC2] | (S)-6-(1-amino-1,3- dihydrospiro[indene-2,4′- piperidin]-1′-yl)-3-(1-(pyridin-2- yl)cyclobutyl)-1,5-dihydro-4H- pyrazolo[3,4-d]pyrimidin-4-one | 468.24 |
| Example 94 (A094) | 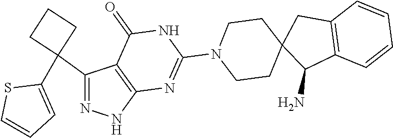 [SMILES: N[C@@H]1c2ccccc2CC12CCN(c1nc3[nH]nc(C4(c5cccs5)CCC4)c3c(=O)[nH]1)CC2] | (S)-6-(1-amino-1,3- dihydrospiro[indene-2,4′- piperidin]-1′-yl)-3-(1-(thiophen-2- yl)cyclobutyl)-1,5-dihydro-4H- pyrazolo[3,4-d]pyrimidin-4-one | 473.20 |
| Example 95 (A095) | 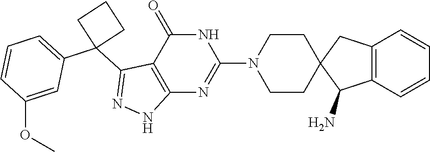 [SMILES: COc1cccc(C2(c3n[nH]c4nc(N5CCC6(CC5)Cc5ccccc5[C@H]6N)[nH]c(=O)c34)CCC2)c1] | (S)-6-(1-amino-1,3- dihydrospiro[indene-2,4′- piperidin]-1′-yl)-3-(1-(3- methoxyphenyl)cyclobutyl)-1,5- dihydro-4H-pyrazolo[3,4- d]pyrimidin-4-one | 497.26 |
| Example 96 (A096) | 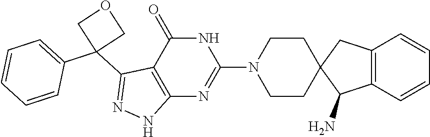 [SMILES: N[C@@H]1c2ccccc2CC12CCN(c1nc3[nH]nc(C4(c5ccccc5)COC4)c3c(=O)[nH]1)CC2] | (S)-6-(1-amino-1,3- dihydrospiro[indene-2,4′- piperidin]-1′-yl)-3-(3- phenyloxetan-3-yl)-1,5-dihydro- 4H-pyrazolo[3,4-d]pyrimidin-4- one | 469.23 |
| Example 97 (A097) | 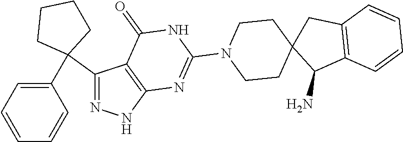 [SMILES: N[C@@H]1c2ccccc2CC12CCN(c1nc3[nH]nc(C4(c5ccccc5)CCCC4)c3c(=O)[nH]1)CC2] | (S)-6-(1-amino-1,3- dihydrospiro[indene-2,4′- piperidin]-1′-yl)-3-(1- phenylcyclopentyl)-1,5-dihydro- 4H-pyrazolo[3,4-d]pyrimidin-4- one | 481.26 |
| Example 98 (A098) | 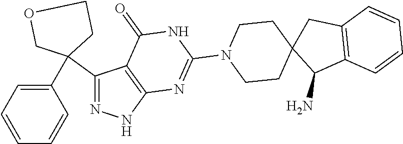 [SMILES: N[C@@H]1c2ccccc2CC12CCN(c1nc3[nH]nc(C4(c5ccccc5)CCOC4)c3c(=O)[nH]1)CC2] | 6-((S)-1-amino-1,3- dihydrospiro[indene-2,4′- piperidin]-1′-yl)-3-(3- phenyltetrahydrofuran-3-yl)-1,5- dihydro-4H-pyrazolo[3,4- d]pyrimidin-4-one | 483.24 |
| Example 99 (A099) | 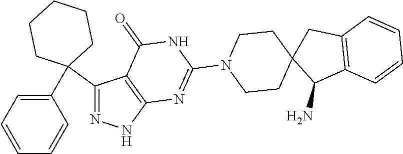 [SMILES: N[C@@H]1c2ccccc2CC12CCN(c1nc3[nH]nc(C4(c5ccccc5)CCCCC4)c3c(=O)[nH]1)CC2] | (S)-6-(1-amino-1,3- dihydrospiro[indene-2,4′- piperidin]-1′-yl)-3-(1- phenylcyclohexyl)-1,5-dihydro- 4H-pyrazolo[3,4-d]pyrimidin-4- one | 495.28 |
| Example 100 (A100) | 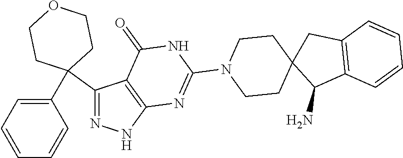 [SMILES: N[C@@H]1c2ccccc2CC12CCN(c1nc3[nH]nc(C4(c5ccccc5)CCOCC4)c3c(=O)[nH]1)CC2] | (S)-6-(1-amino-1,3- dihydrospiro[indene-2,4′- piperidin]-1′-yl)-3-(4- phenyltetrahydro-2H-pyran-4-yl)- 1,5-dihydro-4H-pyrazolo[3,4- d]pyrimidin-4-one | 497.26 |
| Example 101 (A101) | 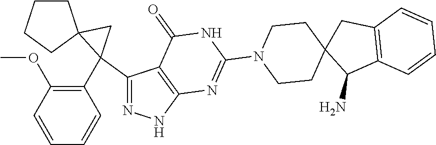 [SMILES: COc1ccccc1C1(c2n[nH]c3nc(N4CCC5(CC4)Cc4ccccc4[C@H]5N)[nH]c(=O)c23)CC12CCCC2] | 6-((S)-1-amino-1,3- dihydrospiro[indene-2,4′- piperidin]-1′-yl)-3-(1-(2- methoxyphenyl)spiro[2.4]heptan- 1-yl)-1,5-dihydro-4H- pyrazolo[3,4-d]pyrimidin-4-one | 537.29 |
| Example 105 (C001) | 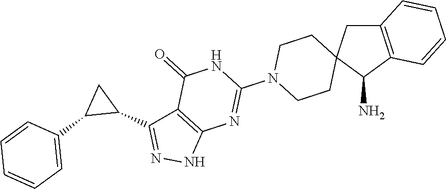 [SMILES: N[C@@H]1c2ccccc2CC12CCN(c1nc3[nH]nc([C@H]4C[C@H]4c4ccccc4)c3c(=O)[nH]1)CC2] | 6-((S)-1-amino-1,3- dihydrospiro[indene-2,4′- piperidin]-1′-yl)-3-((1S,2R)-2- phenylcyclopropyl)-1,5-dihydro- 4H-pyrazolo[3,4-d]pyrimidin-4- one | 453.23 |
| Example 106 (C002) | 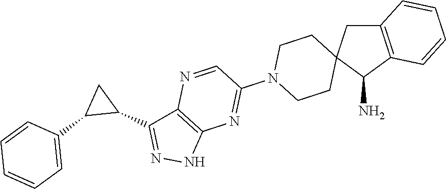 [SMILES: N[C@@H]1c2ccccc2CC12CCN(c1cnc3c([C@H]4C[C@H]4c4ccccc4)n[nH]c3n1)CC2] | (S)-1′-(3-((1S,2R)-2- phenylcyclopropyl)-1H- pyrazolo[3,4-b]pyrazin-6-yl)-1,3- dihydrospiro[indene-2,4′- piperidin]-1-amine | 437.24 |
| Example 107 (C003) | 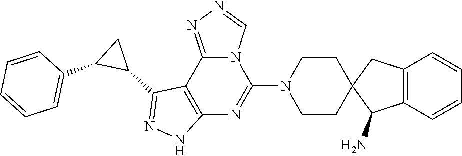 [SMILES: N[C@@H]1c2ccccc2CC12CCN(c1nc3[nH]nc([C@H]4C[C@H]4c4ccccc4)c3c3nncn13)CC2] | (S)-1′-(9-((1S,2R)-2- phenylcyclopropyl)-7H- pyrazolo[4,3- e][1,2,4]triazolo[4,3-c]pyrimidin- 5-yl)-1,3-dihydrospiro[indene- 2,4′-piperidin]-1-amine | 477.24 |
| Example 192 | 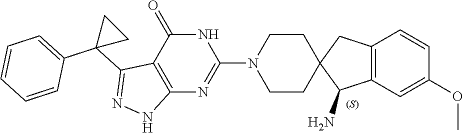 | (S)-6-(1-amino-6-methoxy-1,3- dihydrospiro[indene-2,4′- piperidin]-1′-yl)-3-(1- phenylcyclopropyl)-1,5-dihydro- 4H-pyrazolo[3,4-d]pyrimidin-4- one | 483.58 |
| Example 193 | 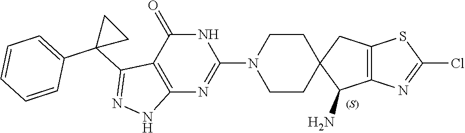 | (S)-6-(4-amino-2-chloro-4,6- dihydrospiro[cyclopenta[d]thiazole- 5,4′-piperidin]-1′-yl)-3-(1- phenylcyclopropyl)-1,5-dihydro- 4H-pyrazolo[3,4-d]pyrimidin-4- one | 495.01 |
| Example 194 | 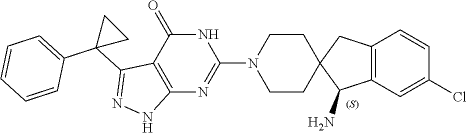 | (S)-6-(1-amino-6-chloro-1,3- dihydrospiro[indene-2,4′- piperidin]-1′-yl)-3-(1- phenylcyclopropyl)-1,5-dihydro- 4H-pyrazolo[3,4-d]pyrimidin-4- one | 488.01 |
| Example 195 | 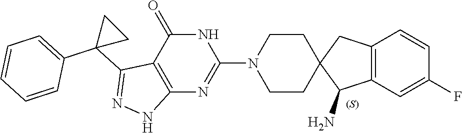 | (S)-6-(1-amino-6-fluoro-1,3- dihydrospiro[indene-2,4′- piperidin]-1′-yl)-3-(1- phenylcyclopropyl)-1,5-dihydro- 4H-pyrazolo[3,4-d]pyrimidin-4- one | 471.54 |
| Example 196 | 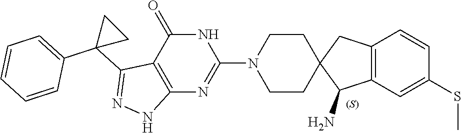 | (S)-6-(1-amino-6-(methylthio)- 1,3-dihydrospiro[indene-2,4′- piperidin]-1′-yl)-3-(1- phenylcyclopropyl)-1,5-dihydro- 4H-pyrazolo[3,4-d]pyrimidin-4- one | 499.64 |
| Example 197 | 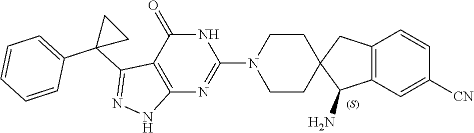 | (S)-1-amino-1′-(4-oxo-3-(1- phenylcyclopropyl)-4,5-dihydro- 1H-pyrazolo[3,4-d]pyrimidin-6- yl)-1,3-dihydrospiro[indene-2,4′- piperidine]-6-carbonitrile | 478.56 |
| Example 198 | 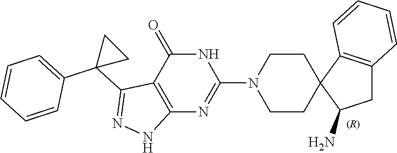 | (R)-6-(2-amino-2,3- dihydrospiro[indene-1,4′- piperidin]-1′-yl)-3-(1- phenylcyclopropyl)-1,5-dihydro- 4H-pyrazolo[3,4-d]pyrimidin-4- one | 453.55 |
| Example 199 | 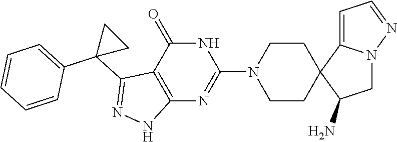 [SMILES: N[C@@H]1Cn2nccc2C12CCN(c1nc3[nH]nc(C4(c5ccccc5)CC4)c3c(=O)[nH]1)CC2] | (S)-6-(5′-amino-5′,6′- dihydrospiro[piperidine-4,4′- pyrrolo[1,2-b]pyrazol]-1-yl)-3-(1- phenylcyclopropyl)-1,5-dihydro- 4H-pyrazolo[3,4-d]pyrimidin-4- one | 453.52 |
| Example 200 | 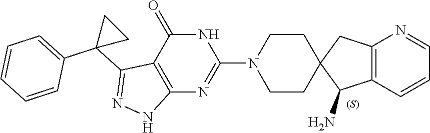 | (S)-6-(5-amino-5,7- dihydrospiro[cyclopenta[b]pyridine- 6,4′-piperidin]-1-yl)-3-(1- phenylcyclopropyl)-1,5-dihydro- 4H-pyrazolo[3,4-d]pyrimidin-4- one | 454.54 |
| Example 202 | 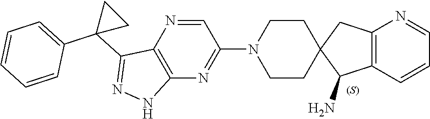 | (S)-1′-(3-(1-phenylcyclopropyl)- 1H-pyrazolo[3,4-b]pyrazin-6-yl)- 5,7- dihydrospiro[cyclopenta[b]pyridine- 6,4′-piperidin]-5-amine | 438.54 |
| Example 203 | 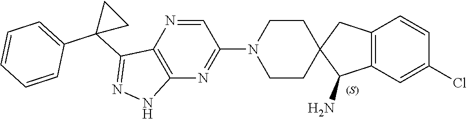 | (S)-6-chloro-1′-(3-(1- phenylcyclopropyl)-1H- pyrazolo[3,4-b]pyrazin-6-yl)-1,3- dihydrospiro[indene-2,4′- piperidin]-1-amine | 472.01 |
| Example 204 | 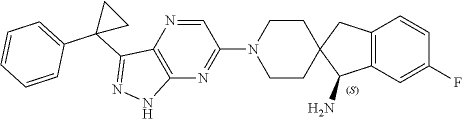 | (S)-6-fluoro-1′-(3-(1- phenylcyclopropyl)-1H- pyrazolo[3,4-b]pyrazin-6-yl)-1,3- dihydrospiro[indene-2,4′- piperidin]-1-amine | 455.54 |
| Example 205 | 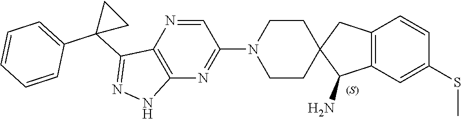 | (S)-6-(methylthio)-1′-(3-(1- phenylcyclopropyl)-1H- pyrazolo[3,4-b]pyrazin-6-yl)-1,3- dihydrospiro[indene-2,4′- piperidin]-1-amine | 483.64 |
| Example 206 | 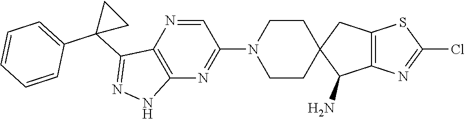 [SMILES: N[C@@H]1c2nc(Cl)sc2CC12CCN(c1cnc3c(C4(c5ccccc5)CC4)n[nH]c3n1)CC2] | (S)-2-chloro-1′-(3-(1- phenylcyclopropyl)-1H- pyrazolo[3,4-b]pyrazin-6-yl)-4,6- dihydrospiro[cyclopenta[d]thiazole- 5,4′-piperidin]-4-amine | 479.01 |
| Example 207 | 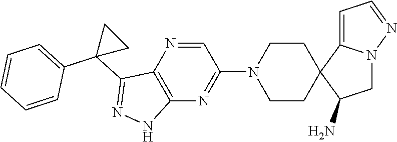 [SMILES: N[C@@H]1Cn2nccc2C12CCN(c1cnc3c(C4(c5ccccc5)CC4)n[nH]c3n1)CC2] | (S)-1-(3-(1-phenylcyclopropyl)- 1H-pyrazolo[3,4-b]pyrazin-6-yl)- 5′,6′-dihydrospiro[piperidine-4,4′- pyrrolo[1,2-b]pyrazol]-5′-amine | 427.52 |
| Example 209 | 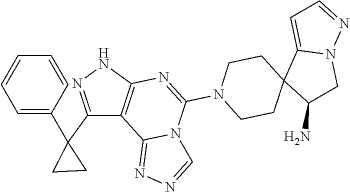 [SMILES: N[C@@H]1Cn2nccc2C12CCN(c1nc3[nH]nc(C4(c5ccccc5)CC4)c3c3nncn13)CC2] | (S)-1-(9-(1-phenylcyclopropyl)- 7H-pyrazolo[4,3- e][1,2,4]triazolo[4,3-c]pyrimidin- 5-yl)-5,6′- dihydrospiro[piperidine-4,4′- pyrrolo[1,2-b]pyrazol]-5′-amine | 467.54 |
| Example 210 | 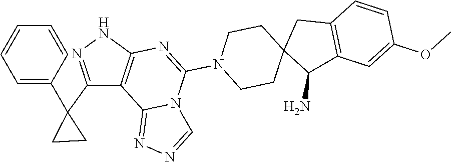 [SMILES: COc1ccc2c(c1)[C@@H](N)C1(CCN(c3nc4[nH]nc(C5(c6ccccc6)CC5)c4c4nncn34)CC1)C2] | (S)-6-methoxy-1′-(9-(1- phenylcyclopropyl)-7H- pyrazolo[4,3- e][1,2,4]triazolo[4,3-c]pyrimidin- 5-yl)-1,3-dihydrospiro[indene- 2,4′-piperidin]-1-amine | 507.60 |
| Example 211 | 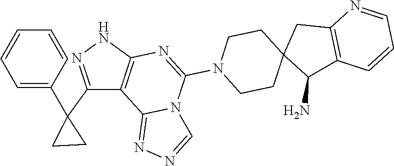 [SMILES: N[C@@H]1c2cccnc2CC12CCN(c1nc3[nH]nc(C4(c5ccccc5)CC4)c3c3nncn13)CC2] | (S)-1′-(9-(1-phenylcyclopropyl)- 7H-pyrazolo[4,3- e][1,2,4]triazolo[4,3-c]pyrimidin- 5-yl)-5,7- dihydrospiro[cyclopenta[b]pyridine- 6,4′-piperidin]-5-amine | 478.56 |
| Example 212 | 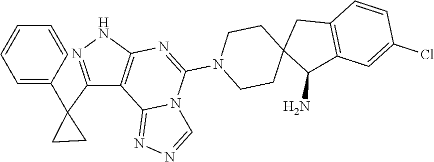 [SMILES: N[C@@H]1c2cc(Cl)ccc2CC12CCN(c1nc3[nH]nc(C4(c5ccccc5)CC4)c3c3nncn13)CC2] | (S)-6-chloro-1′-(9-(1- phenylcyclopropyl)-7H- pyrazolo[4,3- e][1,2,4]triazolo[4,3-c]pyrimidin- 5-yl)-1,3-dihydrospiro[indene- 2,4′-piperidin]-1-amine | 512.02 |
| Example 213 | 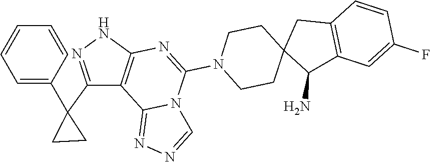 [SMILES: N[C@@H]1c2cc(F)ccc2CC12CCN(c1nc3[nH]nc(C4(c5ccccc5)CC4)c3c3nncn13)CC2] | (S)-6-fluoro-1′-(9-(1- phenylcyclopropyl)-7H- pyrazolo[4,3- e][1,2,4]triazolo[4,3-c]pyrimidin- 5-yl)-1,3-dihydrospiro[indene- 2,4′-piperidin]-1-amine | 495.57 |
| Example 214 | 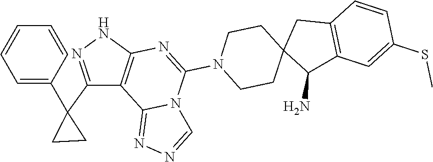 [SMILES: CSc1ccc2c(c1)[C@@H](N)C1(CCN(c3nc4[nH]nc(C5(c6ccccc6)CC5)c4c4nncn34)CC1)C2] | (S)-6-(methylthio)-1′-(9-(1- phenylcyclopropyl)-7H- pyrazolo[4,3- e][1,2,4]triazolo[4,3-c]pyrimidin- 5-yl)-1,3-dihydrospiro[indene- 2,4′-piperidin]-1-amine | 523.67 |
| Example 215 | 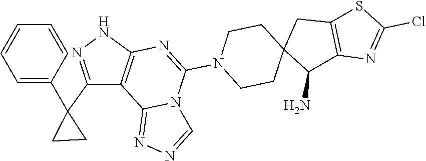 [SMILES: N[C@@H]1c2nc(Cl)sc2CC12CCN(c1nc3[nH]nc(C4(c5ccccc5)CC4)c3c3nncn13)CC2] | (S)-2-chloro-1′-(9-(1- phenylcyclopropyl)-7H- pyrazolo[4,3- e][1,2,4]triazolo[4,3-c]pyrimidin- 5-yl)-4,6- dihydrospiro[cyclopenta[d]thiazole- 5,4′-piperidin]-4-amine | 519.04 |
| Example 216 | 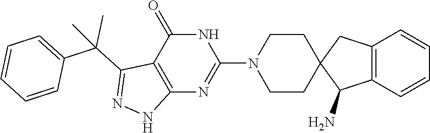 [SMILES: CC(C)(c1ccccc1)c1n[nH]c2nc(N3CCC4(CC3)Cc3ccccc3[C@H]4N)[nH]c(=O)c12] | (S)-6-(1-amino-1,3- dihydrospiro[indene-2,4′- piperidin]-1′-yl)-3-(2- phenylpropan-2-yl)-1,5-dihydro- 4H-pyrazolo[3,4-d]pyrimidin-4- one | 455.57 |
| Example 217 | 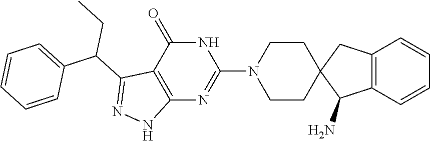 [SMILES: CCC(c1ccccc1)c1n[nH]c2nc(N3CCC4(CC3)Cc3ccccc3[C@H]4N)[nH]c(=O)c12] | 6-((S)-1-amino-1,3- dihydrospiro[indene-2,4′- piperidin]-1′-yl)-3-(1- phenylpropyl)-1,5-dihydro-4H- pyrazolo[3,4-d]pyrimidin-4-one | 455.57 |
| Example 218 | 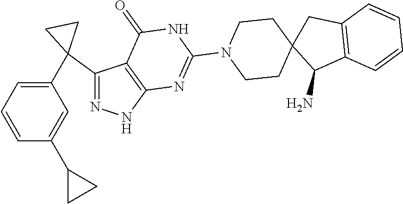 [SMILES: N[C@@H]1c2ccccc2CC12CCN(c1nc3[nH]nc(C4(c5cccc(C6CC6)c5)CC4)c3c(=O)[nH]1)CC2] | (S)-6-(1-amino-1,3- dihydrospiro[indene-2,4′- piperidin]-1′-yl)-3-(1-(3- cyclopropylphenyl)cyclopropyl)- 1,5-dihydro-4H-pyrazolo[3,4- d]pyrimidin-4-one | 493.61 |
| Example 219 | 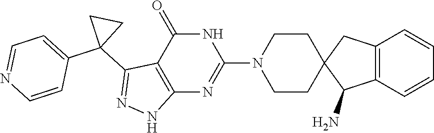 [SMILES: N[C@@H]1c2ccccc2CC12CCN(c1nc3[nH]nc(C4(c5ccncc5)CC4)c3c(=O)[nH]1)CC2] | (S)-6-(1-amino-1,3- dihydrospiro[indene-2,4′- piperidin]-1′-yl)-3-(1-(pyridin-4- yl)cyclopropyl)-1,5-dihydro-4H- pyrazolo[3,4-d]pyrimidin-4-one | 454.54 |
| Example 220 | 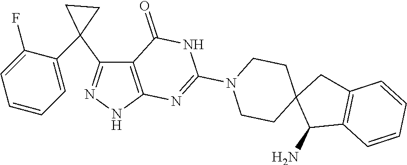 [SMILES: N[C@@H]1c2ccccc2CC12CCN(c1nc3[nH]nc(C4(c5ccccc5F)CC4)c3c(=O)[nH]1)CC2] | (S)-6-(1-amino-1,3- dihydrospiro[indene-2,4′- piperidin]-1′-yl)-3-(1-(2- fluorophenyl)cyclopropyl)-1,5- dihydro-4H-pyrazolo[3,4- d]pyrimidin-4-one | 471.54 |
| Example 221 | 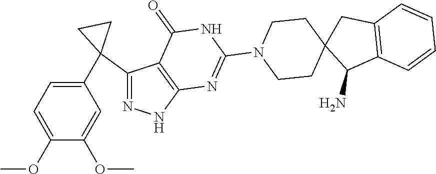 [SMILES: COc1ccc(C2(c3n[nH]c4nc(N5CCC6(CC5)Cc5ccccc5[C@H]6N)[nH]c(=O)c34)CC2)cc1OC] | (S)-6-(1-amino-1,3- dihydrospiro[indene-2,4′- piperidin]-1′-yl)-3-(1-(3,4- dimethoxyphenyl)cyclopropyl)- 1,5-dihydro-4H-pyrazolo[3,4- d]pyrimidin-4-one | 513.60 |
| Example 222 | 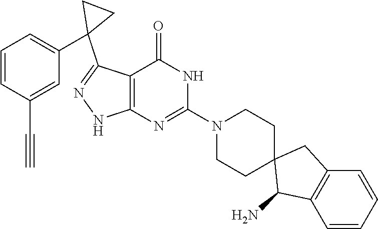 [SMILES: C#Cc1cccc(C2(c3n[nH]c4nc(N5CCC6(CC5)Cc5ccccc5[C@H]6N)[nH]c(=O)c34)CC2)c1] | (S)-6-(1-amino-1,3- dihydrospiro[indene-2,4′- piperidin]-1′-yl)-3-(1-(3- ethynylphenyl)cyclopropyl)-1,5- dihydro-4H-pyrazolo[3,4- d]pyrimidin-4-one | 477.57 |
| Example 223 | 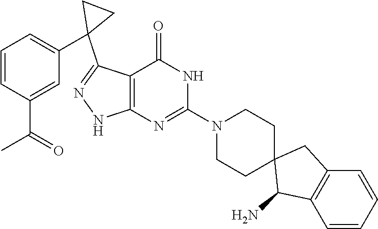 [SMILES: CC(=O)c1cccc(C2(c3n[nH]c4nc(N5CCC6(CC5)Cc5ccccc5[C@H]6N)[nH]c(=O)c34)CC2)c1] | (S)-3-(1-(3- acetylphenyl)cyclopropyl)-6-(1- amino-1,3-dihydrospiro[indene- 2,4′-piperidin]-1′-yl)-1,5-dihydro- 4H-pyrazolo[3,4-d]pyrimidin-4- one | 495.59 |
| Example 224 | 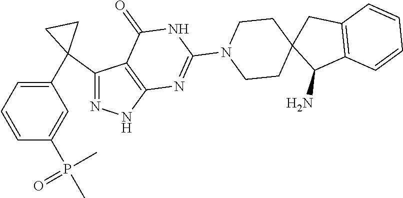 [SMILES: CP(C)(=O)c1cccc(C2(c3n[nH]c4nc(N5CCC6(CC5)Cc5ccccc5[C@H]6N)[nH]c(=O)c34)CC2)c1] | (S)-6-(1-amino-1,3- dihydrospiro[indene-2,4′- piperidin]-1′-yl)-3-(1-(3- (dimethylphosphoryl)phenyl)cyclo- propyl)-1,5-dihydro-4H- pyrazolo[3,4-d]pyrimidin-4-one | 529.59 |
| Example 225 | 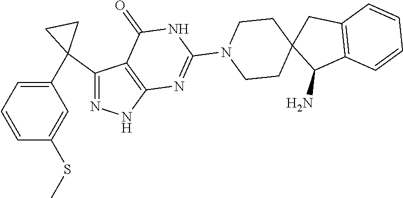 [SMILES: CSc1cccc(C2(c3n[nH]c4nc(N5CCC6(CC5)Cc5ccccc5[C@H]6N)[nH]c(=O)c34)CC2)c1] | (S)-6-(1-amino-1,3- dihydrospiro[indene-2,4′- piperidin]-1′-yl)-3-(1-(3- (methylthio)phenyl)cyclopropyl)- 1,5-dihydro-4H-pyrazolo[3,4- d]pyrimidin-4-one | 499.64 |
| Example 226 | 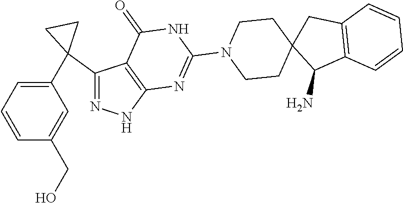 [SMILES: N[C@@H]1c2ccccc2CC12CCN(c1nc3[nH]nc(C4(c5cccc(CO)c5)CC4)c3c(=O)[nH]1)CC2] | (S)-6-(1-amino-1,3- dihydrospiro[indene-2,4′- piperidin]-1′-yl)-3-(1-(3- (hydroxymethyl)phenyl)cyclo- propyl)-1,5-dihydro-4H- pyrazolo[3,4-d]pyrimidin-4-one | 483.58 |
| Example 227 | 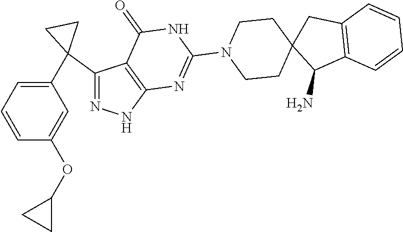 [SMILES: N[C@@H]1c2ccccc2CC12CCN(c1nc3[nH]nc(C4(c5cccc(OC6CC6)c5)CC4)c3c(=O)[nH]1)CC2] | (S)-6-(1-amino-1,3- dihydrospiro[indene-2,4′- piperidin]-1′-yl)-3-(1-(3- cyclopropoxyphenyl)cyclopropyl)- 1,5-dihydro-4H-pyrazolo[3,4- d]pyrimidin-4-one | 509.61 |
| Example 228 | 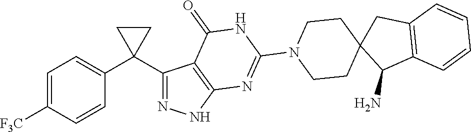 [SMILES: N[C@@H]1c2ccccc2CC12CCN(c1nc3[nH]nc(C4(c5ccc(C(F)(F)F)cc5)CC4)c3c(=O)[nH]1)CC2] | (S)-6-(1-amino-1,3- dihydrospiro[indene-2,4′- piperidin]-1′-yl)-3-(1-(4- (trifluoromethyl)phenyl)cyclo- propyl)-1,5-dihydro-4H- pyrazolo[3,4-d]pyrimidin-4-one | 521.55 |
| Example 230 | 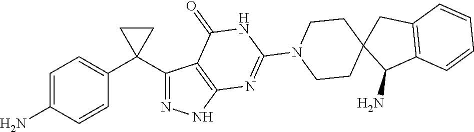 [SMILES: Nc1ccc(C2(c3n[nH]c4nc(N5CCC6(CC5)Cc5ccccc5[C@H]6N)[nH]c(=O)c34)CC2)cc1] | (S)-6-(1-amino-1,3- dihydrospiro[indene-2,4′- piperidin]-1′-yl)-3-(1-(4- aminophenyl)cyclopropyl)-1,5- dihydro-4H-pyrazolo[3,4- d]pyrimidin-4-one | 468.57 |
| Example 231 | 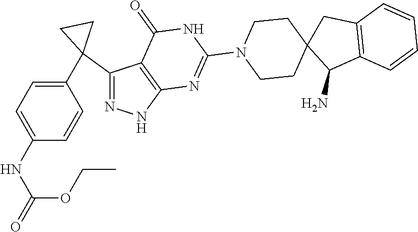 [SMILES: CCOC(=O)Nc1ccc(C2(c3n[nH]c4nc(N5CCC6(CC5)Cc5ccccc5[C@H]6N)[nH]c(=O)c34)CC2)cc1] | ethyl (S)-(4-(1-(6-(1-amino-1,3- dihydrospiro[indene-2,4′- piperidin]-1′-yl)-4-oxo-4,5- dihydro-1H-pyrazolo[3,4- d]pyrimidin-3- yl)cyclopropyl)phenyl)carbamate | 540.63 |
| Example 232 | 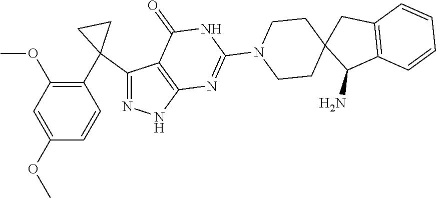 [SMILES: COc1ccc(C2(c3n[nH]c4nc(N5CCC6(CC5)Cc5ccccc5[C@H]6N)[nH]c(=O)c34)CC2)c(OC)c1] | (S)-6-(1-amino-1,3- dihydrospiro[indene-2,4′- piperidin]-1′-yl)-3-(1-(2,4- dimethoxyphenyl)cyclopropyl)- 1,5-dihydro-4H-pyrazolo[3,4- d]pyrimidin-4-one | 513.60 |
| Example 233 | 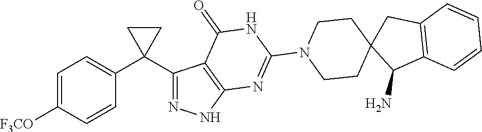 [SMILES: N[C@@H]1c2ccccc2CC12CCN(c1nc3[nH]nc(C4(c5ccc(OC(F)(F)F)cc5)CC4)c3c(=O)[nH]1)CC2] | (S)-6-(1-amino-1,3- dihydrospiro[indene-2,4′- piperidin]-1′-yl)-3-(1-(4- (trifluoromethoxy)phenyl)cyclo- propyl)-1,5-dihydro-4H- pyrazolo[3,4-d]pyrimidin-4-one | 537.55 |
| Example 234 | 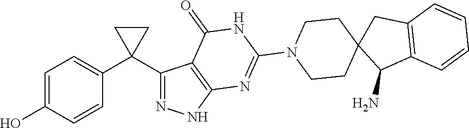 [SMILES: N[C@@H]1c2ccccc2CC12CCN(c1nc3[nH]nc(C4(c5ccc(O)cc5)CC4)c3c(=O)[nH]1)CC2] | (S)-6-(1-amino-1,3- dihydrospiro[indene-2,4′- piperidin]-1′-yl)-3-(1-(4- hydroxyphenyl)cyclopropyl)-1,5- dihydro-4H-pyrazolo[3,4- d]pyrimidin-4-one | 469.55 |
| Example 235 | 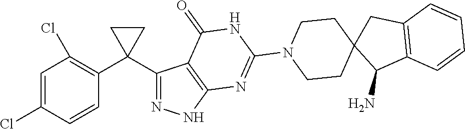 [SMILES: N[C@@H]1c2ccccc2CC12CCN(c1nc3[nH]nc(C4(c5ccc(Cl)cc5Cl)CC4)c3c(=O)[nH]1)CC2] | (S)-6-(1-amino-1,3- dihydrospiro[indene-2,4′- piperidin]-1′-yl)-3-(1-(2,4- dichlorophenyl)cyclopropyl)-1,5- dihydro-4H-pyrazolo[3,4- d]pyrimidin-4-one | 522.44 |
| Example 236 | 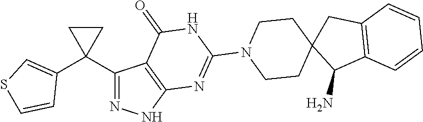 [SMILES: N[C@@H]1c2ccccc2CC12CCN(c1nc3[nH]nc(C4(c5ccsc5)CC4)c3c(=O)[nH]1)CC2] | (S)-6-(1-amino-1,3- dihydrospiro[indene-2,4′- piperidin]-1′-yl)-3-(1-(thiophen-3- yl)cy clopropyl)- l,5-dihydro-4H- pyrazolo[3,4-d]pyrimidin-4-one | 459.58 |
| Example 237 | 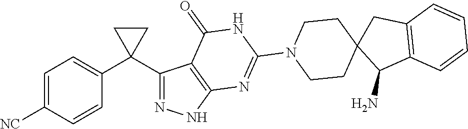 [SMILES: N#Cc1ccc(C2(c3n[nH]c4nc(N5CCC6(CC5)Cc5ccccc5[C@H]6N)[nH]c(=O)c34)CC2)cc1] | (S)-4-(1-(6-(1-amino-1,3- dihydrospiro[indene-2,4′- piperidin]-1′-yl)-4-oxo-4,5- dihydro-1H-pyrazolo[3,4- d]pyrimidin-3- yl)cyclopropyl)benzonitrile | 478.56 |
| Example 238 | 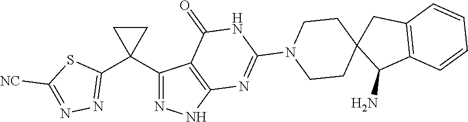 [SMILES: N#Cc1nnc(C2(c3n[nH]c4nc(N5CCC6(CC5)Cc5ccccc5[C@H]6N)[nH]c(=O)c34)CC2)s1] | (S)-5-(1-(6-(1-amino-1,3- dihydrospiro[indene-2,4′- piperidin]-1′-yl)-4-oxo-4,5- dihydro-1H-pyrazolo[3,4- d]pyrimidin-3-yl)cyclopropyl)- 1,3,4-thiadiazole-2-carbonitrile | 486.56 |
| Example 239 | 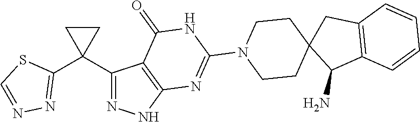 [SMILES: N[C@@H]1c2ccccc2CC12CCN(c1nc3[nH]nc(C4(c5nncs5)CC4)c3c(=O)[nH]1)CC2] | (S)-3-(1-(1,3,4-thiadiazol-2- yl)cyclopropyl)-6-(1-amino-1,3- dihydrospiro[indene-2,4′- piperidin]-1′-yl)-1,5-dihydro-4H- pyrazolo[3,4-d]pyrimidin-4-one | 461.55 |
| Example 240 | 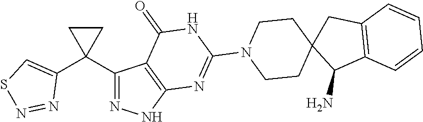 [SMILES: N[C@@H]1c2ccccc2CC12CCN(c1nc3[nH]nc(C4(c5csnn5)CC4)c3c(=O)[nH]1)CC2] | (S)-3-(1-(1,2,3-thiadiazol-4- yl)cyclopropyl)-6-(1-amino-1,3- dihydrospiro[indene-2,4′- piperidin]-1′-yl)-1,5-dihydro-4H- pyrazolo[3,4-d]pyrimidin-4-one | 461.55 |
| Example 241 | 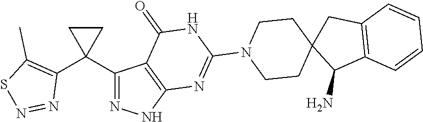 [SMILES: Cc1snnc1C1(c2n[nH]c3nc(N4CCC5(CC4)Cc4ccccc4[C@H]5N)[nH]c(=O)c23)CC1] | (S)-6-(1-amino-1,3- dihydrospiro[indene-2,4′- piperidin]-1′-yl)-3-(1-(5-methyl- 1,2,3-thiadiazol-4- yl)cyclopropyl)-1,5-dihydro-4H- pyrazolo[3,4-d]pyrimidin-4-one | 475.58 |
| Example 242 | 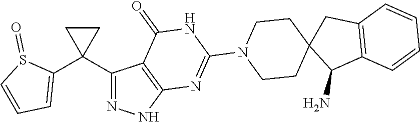 [SMILES: N[C@@H]1c2ccccc2CC12CCN(c1nc3[nH]nc(C4(C5=CC=CS5=O)CC4)c3c(=O)[nH]1)CC2] | 6-((S)-1-amino-1,3- dihydrospiro[indene-2,4′- piperidin]-1′-yl)-3-(1-(1- oxidothiophen-2-yl)cyclopropyl)- 1,5-dihydro-4H-pyrazolo[3,4- d]pyrimidin-4-one | 475.58 |
| Example 243 | 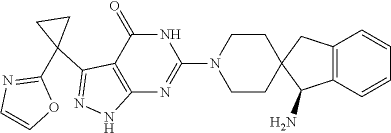 [SMILES: N[C@@H]1c2ccccc2CC12CCN(c1nc3[nH]nc(C4(c5ncco5)CC4)c3c(=O)[nH]1)CC2] | (S)-6-(1-amino-1,3- dihydrospiro[indene-2,4′- piperidin]-1′-yl)-3-(1-(oxazol-2- yl)cyclopropyl)-1,5-dihydro-4H- pyrazolo[3,4-d]pyrimidin-4-one | 444.50 |
| Example 244 | 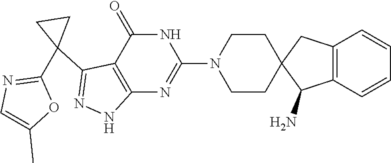 [SMILES: Cc1cnc(C2(c3n[nH]c4nc(N5CCC6(CC5)Cc5ccccc5[C@H]6N)[nH]c(=O)c34)CC2)o1] | (S)-6-(1-amino-1,3- dihydrospiro[indene-2,4′- piperidin]-1′-yl)-3-(1-(5- methyloxazol-2-yl)cyclopropyl)- 1,5-dihydro-4H-pyrazolo[3,4- d]pyrimidin-4-one | 458.53 |
| Example 245 | 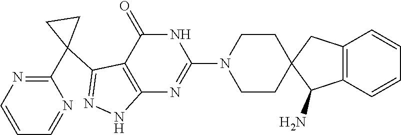 [SMILES: N[C@@H]1c2ccccc2CC12CCN(c1nc3[nH]nc(C4(c5ncccn5)CC4)c3c(=O)[nH]1)CC2] | (S)-6-(1-amino-1,3- dihydrospiro[indene-2,4′- piperidin]-1′-yl)-3-(1-(pyrimidin- 2-yl)cyclopropyl)-1,5-dihydro- 4H-pyrazolo[3,4-d]pyrimidin-4- one | 455.53 |
| Example 246 | 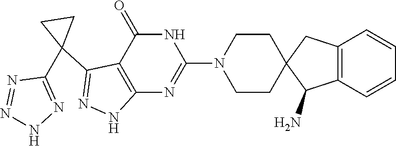 [SMILES: N[C@@H]1c2ccccc2CC12CCN(c1nc3[nH]nc(C4(c5nn[nH]n5)CC4)c3c(=O)[nH]1)CC2] | (S)-3-(1-(2H-tetrazol-5- yl)cyclopropyl)-6-(1-amino-1,3- dihydrospiro[indene-2,4′- piperidin]-1′-yl)-1,5-dihydro-4H- pyrazolo[3,4-d]pyrimidin-4-one | 445.49 |
| Example 247 | 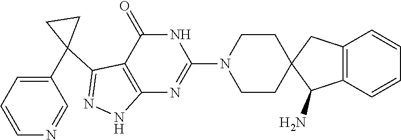 [SMILES: N[C@@H]1c2ccccc2CC12CCN(c1nc3[nH]nc(C4(c5cccnc5)CC4)c3c(=O)[nH]1)CC2] | (S)-6-(1-amino-1,3- dihydrospiro[indene-2,4′- piperidin]-1′-yl)-3-(1-(pyridin-3- yl)cyclopropyl)-1,5-dihydro-4H- pyrazolo[3,4-d]pyrimidin-4-one | 454.54 |
| Example 248 | 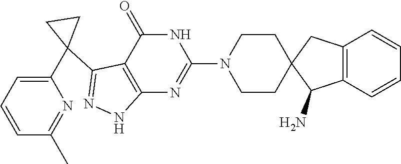 [SMILES: Cc1cccc(C2(c3n[nH]c4nc(N5CCC6(CC5)Cc5ccccc5[C@H]6N)[nH]c(=O)c34)CC2)n1] | (S)-6-(1-amino-1,3- dihydrospiro[indene-2,4′- piperidin]-1′-yl)-3-(1-(6- methylpyridin-2-yl)cyclopropyl)- 1,5-dihydro-4H-pyrazolo[3,4- d]pyrimidin-4-one | 468.57 |
| Example 249 | 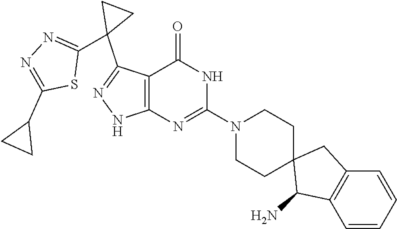 [SMILES: N[C@@H]1c2ccccc2CC12CCN(c1nc3[nH]nc(C4(c5nnc(C6CC6)s5)CC4)c3c(=O)[nH]1)CC2] | (S)-6-(1-amino-1,3- dihydrospiro[indene-2,4′- piperidin]-1′-yl)-3-(1-(5- cyclopropyl-1,3,4-thiadiazol-2- yl)cyclopropyl)-1,5-dihydro-4H- pyrazolo[3,4-d]pyrimidin-4-one | 501.62 |
| Example 250 | 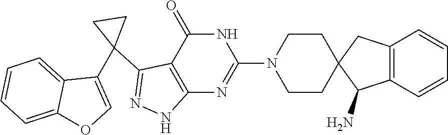 [SMILES: N[C@@H]1c2ccccc2CC12CCN(c1nc3[nH]nc(C4(c5coc6ccccc56)CC4)c3c(=O)[nH]1)CC2] | (S)-6-(1-amino-1,3- dihydrospiro[indene-2,4′- piperidin]-1′-yl)-3-(1- (benzofuran-3-yl)cyclopropyl)- 1,5-dihydro-4H-pyrazolo[3,4- d]pyrimidin-4-one | 493.57 |
| Example 251 | 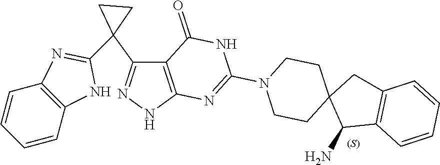 | (S)-3-(1-(1H-benzo[d]imidazol-2- yl)cyclopropyl)-6-(1-amino-1,3- dihydrospiro[indene-2,4′- piperidin]-1′-yl)-1,5-dihydro-4H- pyrazolo[3,4-d]pyrimidin-4-one | 493.57 |
| Example 252 | 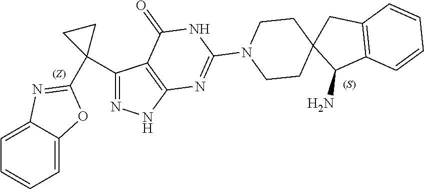 | (S)-6-(1-amino-1,3- dihydrospiro[indene-2,4′- piperidin]-1′-yl)-3-(1- (benzo[d]oxazol-2- yl)cyclopropyl)-1,5-dihydro-4H- pyrazolo[3,4-d]pyrimidin-4-one | 494.56 |
| Example 253 | 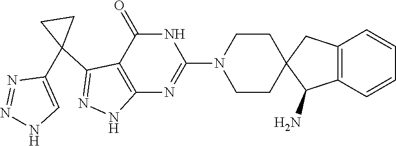 [SMILES: N[C@@H]1c2ccccc2CC12CCN(c1nc3[nH]nc(C4(c5c[nH]nn5)CC4)c3c(=O)[nH]1)CC2] | (S)-3-(1-(1H-1,2,3-triazol-4- yl)cyclopropyl)-6-(1-amino-1,3- dihydrospiro[indene-2,4′- piperidin]-1′-yl)-1,5-dihydro-4H- pyrazolo[3,4-d]pyrimidin-4-one | 444.50 |
| Example 254 | 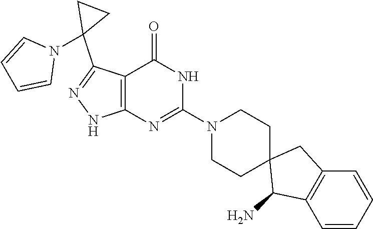 [SMILES: N[C@@H]1c2ccccc2CC12CCN(c1nc3[nH]nc(C4(n5cccc5)CC4)c3c(=O)[nH]1)CC2] | (S)-3-(1-(1H-pyrrol-1- yl)cyclopropyl)-6-(1-amino-1,3- dihydrospiro[indene-2,4′- piperidin]-1′-yl)-1,5-dihydro-4H- pyrazolo[3,4-d]pyrimidin-4-one | 442.53 |
| Example 255 | 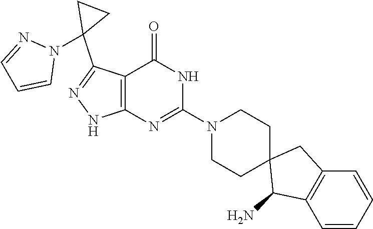 [SMILES: N[C@@H]1c2ccccc2CC12CCN(c1nc3[nH]nc(C4(n5cccn5)CC4)c3c(=O)[nH]1)CC2] | (S)-3-(1-(1H-pyrazol-1- yl)cyclopropyl)-6-(1-amino-1,3- dihydrospiro[indene-2,4′- piperidin]-1′-yl)-1,5-dihydro-4H- pyrazolo[3,4-d]pyrimidin-4-one | 443.52 |
| Example 256 | 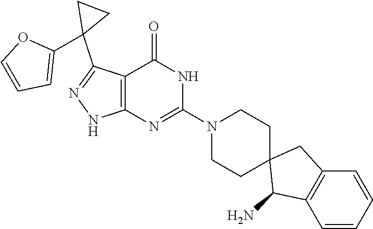 [SMILES: N[C@@H]1c2ccccc2CC12CCN(c1nc3[nH]nc(C4(c5ccco5)CC4)c3c(=O)[nH]1)CC2] | (S)-6-(1-amino-1,3- dihydrospiro[indene-2,4′- piperidin]-1′-yl)-3-(1-(furan-2- yl)cyclopropyl)-1,5-dihydro-4H- pyrazolo[3,4-d]pyrimidin-4-one | 443.51 |
| Example 257 | 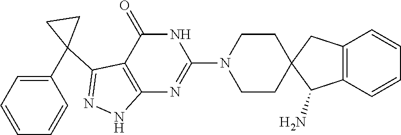 [SMILES: N[C@H]1c2ccccc2CC12CCN(c1nc3[nH]nc(C4(c5ccccc5)CC4)c3c(=O)[nH]1)CC2] | (R)-6-(1-amino-1,3- dihydrospiro[indene-2,4′- piperidin]-1′-yl)-3-(1- phenylcyclopropyl)-1,5-dihydro- 4H-pyrazolo[3,4-d]pyrimidin-4- one | 453.55 |
| Example 322 | 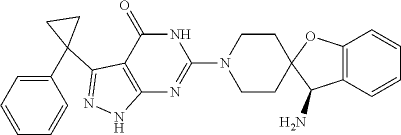 [SMILES: N[C@@H]1c2ccccc2OC12CCN(c1nc3[nH]nc(C4(c5ccccc5)CC4)c3c(=O)[nH]1)CC2] | (R)-6-(3-amino-3H- spiro[benzofuran-2,4′-piperidin]- 1′-yl)-3-(1-phenylcyclopropyl)- 1,5-dihydro-4H-pyrazolo[3,4- d]pyrimidin-4-one | 455.21 |
| Example 323 | 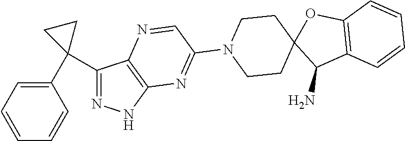 [SMILES: N[C@@H]1c2ccccc2OC12CCN(c1cnc3c(C4(c5ccccc5)CC4)n[nH]c3n1)CC2] | (R)-1′-(3-(1-phenylcyclopropyl)- 1H-pyrazolo[3,4-b]pyrazin-6-yl)- 3H-spiro[benzofuran-2,4′- piperidin]-3-amine | 439.22 |
| Example 324 | 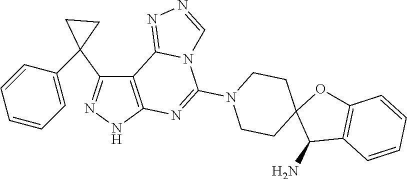 [SMILES: N[C@@H]1c2ccccc2OC12CCN(c1nc3[nH]nc(C4(c5ccccc5)CC4)c3c3nncn13)CC2] | (R)-1′-(9-(1-phenylcyclopropyl)- 7H-pyrazolo[4,3- e][1,2,4]triazolo[4,3-c]pyrimidin- 5-yl)-3H-spiro[benzofuran-2,4′- piperidin]-3-amine | 479.22 |

The NMR data of the compound (A004, A024, A048, A084, Example 192) are as follows:

A004:

¹H NMR (500 MHz, CD₃OD) δ 8.57 (sb, 1H), 7.51-7.44 (m, 1H), 7.42-7.35 (m, 2H), 7.33 (t, J=7.2 Hz, 2H), 7.22 (t, J=7.5 Hz, 2H), 7.11 (t, J=6.8 Hz, 1H), 4.50-3.98 (m, 3H), 3.42-3.34 (m, 1H), 3.15 (q, J=16.4 Hz, 2H), 1.92-1.50 (m, 4H), 1.47-1.36 (m, 2H), 1.31 (s, 3H).

A024:

¹H NMR (500 MHz, CD₃OD) δ 8.01 (sb, 1H), 7.44 (s, 1H), 7.22-7.19 (m, 4H), 6.95-6.88 (m, 2H), 4.40-3.88 (m, 3H), 3.42-3.32 (m, 1H), 3.15 (q, J=16.4 Hz, 2H), 1.92-1.50 (m, 4H), 1.49-1.39 (m, 2H), 1.30 (s, 3H).

A048:

¹H NMR (500 MHz, CD₃OD) δ 8.71 (d, J=6.2 Hz, 1H), 7.93 (d, J=6.2 Hz, 1H), 7.33 (t, J=7.2 Hz, 2H), 7.20 (t, J=7.5 Hz, 2H), 4.50-3.98 (m, 3H), 3.42-3.32 (m, 1H), 3.15 (q, J=16.4 Hz, 2H), 1.92-1.50 (m, 4H), 1.49-1.39 (m, 2H), 1.30 (s, 3H).

A084:

¹H NMR (500 MHz, CD₃OD) 7.33 (d, J=6.2 Hz, 1H), 7.31 (t, J=6.8 Hz, 2H), 7.24 (t, J=7.5 Hz, 2H), 7.21 (t, J=7.5 Hz, 2H), 4.46-3.98 (m, 3H), 3.42-3.33 (m, 1H), 3.16 (q, J=16.4 Hz, 2H), 1.92-1.58 (m, 4H), 1.49-1.37 (m, 2H), 1.32 (s, 3H).

Example 192:

¹H NMR (500 MHz, CD₃OD) δ 8.51 (sb, 1H), 7.51-7.44 (m, 1H), 7.42-7.35 (m, 2H), 7.33 (t, J=7.2 Hz, 3H), 7.10 (t, J=7.5 Hz, 2H), 6.91 (t, J=6.8 Hz, 1H), 4.50-3.98 (m, 3H), 3.83 (s, 3H), 3.42-3.32 (m, 1H), 3.15 (q, J=16.4 Hz, 2H), 1.92-1.50 (m, 4H), 1.49-1.39 (m, 2H), 1.30 (s, 3H).

### Example 74 Preparation of Compound 74

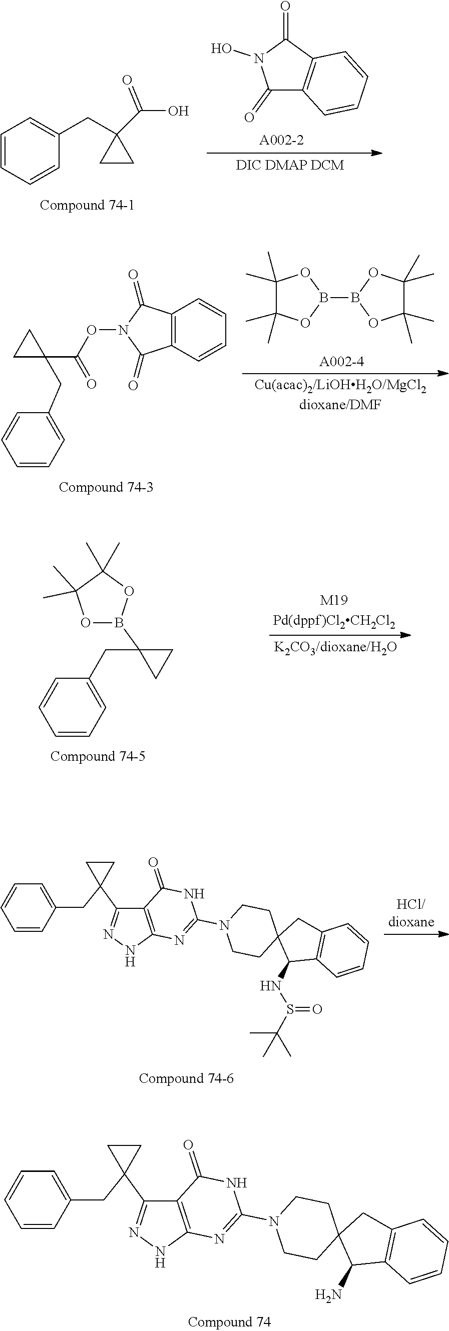

### Step 1: Preparation of Compound 74-3

A round-bottom flask or culture tube equipped with a stir bar was charged with 1-benzylcyclopropanecarboxylic acid (528 mg), N-hydroxy-phthalimide (553.20 mg) and DMAP (37.66 mg). Dichloromethane (20 mL) was added followed by DIC (427.97 mg), and the mixture was allowed to stir vigorously for 2 hours. The mixture was filtered (over Celite, SiO₂, or through a fritted funnel) and rinsed with additional CH₂Cl₂/Et₂₀. The solvent was removed under reduced pressure, and purified by column chromatography (DCM/MeOH=I/O) to afford Compound 74-3 (781 mg). [M+H]⁺=322.

### Step 2: Preparation of Compound 74-5

To a 15 mL culture tube equipped with a stir bar were added compound 74-3 (321 mg), B₂Pin₂ (761.07 mg), LiOH·H₂O (628.79 mg), Cu(acac)₂ (78.45 mg) and MgCl₂ (142.68 mg). The tube was evacuated and backfilled with argon for 3 times. Degassed dioxane (6 mL) DMF (3 mL) was added and the resulting mixture was stirred under 1000 rpm at RT until dark brown color was observed (typical reaction time <10 min). The reaction mixture was diluted with EtOAc (20 mL) and saturated NH₄Cl (20 mL), and the resulting mixture was shaken vigorously until getting a clear biphasic solution. The organic phase was collected and dried over anhydrous Na₂SO₄, evaporated and purified by silica gel chromatography (Hexane/EA=100/0-100/3) to afford the desired product Compound 74-5 (230 mg).

### Step 3: Preparation of Compound 74-6

To a 50 mL round bottom flask equipped with a stir bar were added Compound 74-5 (70 mg), M19 (102 mg), Pd(dppf)Cl₂·CH₂Cl₂ (15 mg), K₂CO₃ (75 mg) and dioxane/H₂O (10 mL/1 mL). The flask was evacuated and backfilled with argon for 3 times, then stirred for 5 hours at 100° C., monitored by LCMS till M19 was consumed, cool down, evaporated and purified by silica gel chromatography (DCM/MeOH=100/0-100/6) to afford the desired product Compound 74-6 (98 mg). [M+H]⁺=571.

### Step 4: Preparation of Compound 74

To a 50 mL round bottom flask equipped with a stir bar were added Compound 74-6 (98 mg) and 4N dioxane/HCl (5 mL), stirred for 1 hour at r.t, then evaporated, the residue was washed by Et₂O (20 mL) to afford the desired product Compound 74 (51 mg). [M+H]⁺=467.

### Example 6 Preparation of Compound of D001

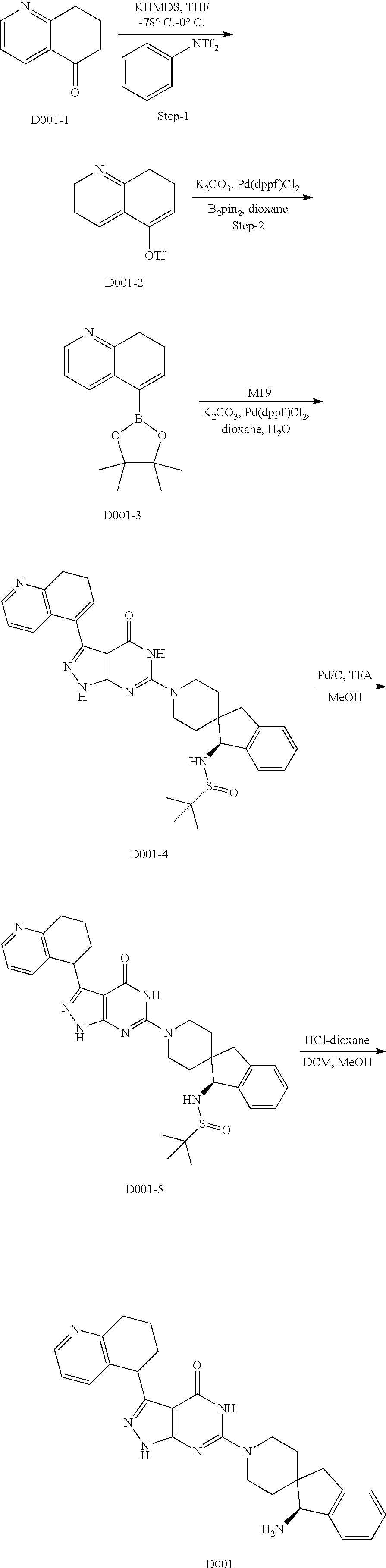

### Step 1 Preparation of Compound D001-2

D001-1 (1.47 g) was added to a round bottom flask and dissolved with THF (15 mL), with N2 replacement for three times, cooled down to −78° C., KHMDS (15 mL, 1M) was dropped in, and warmed to 0° C., 1,1,1-trifluoro-n-phenyl-n-((trifluoromethyl) sulfonyl) methanesulfonamide (5.36 g) was dissolved in THF (10 mL) and then added into the flask. The reaction was monitored by TLC/LCMS after stirring at room temperature for 15 hours. The reaction was quenched with saturated ammonium chloride solution at 0° C. and was extracted with ethyl acetate, filtrate was concentrated and purified by silica gel column (PE:EA=9:1), to obtain light yellow oily liquid compound D001-2 (2.3 g, 95%).

### Step 2 Preparation of Compound D001-3

Compound D001-2 (2.23 g) was added to dioxane (25 mL), followed by K₂CO₃ (3.31 g), Pd(dppf)Cl₂—CH₂Cl₂ (293 mg) and B₂pin₂ (3.05 g). N₂ protection was conducted, reaction was at 80° C. for 2 hours. TLC/LCMS monitored the reaction, and the sample passes through the silica gel column to obtain light yellow oily liquid compound D001-3 (1.65 g, 90%).

### Step 3 Preparation of Compound D001-4

M21 (1.95 g) was added to dioxane (20 mL), followed by K₂CO₃ (1.25 g), Pd(dppf)Cl₂—CH₂Cl₂ (183 mg), D001-3 (3.05 g), and H₂O (3 mL). N₂ protection was conducted, reaction was at 80° C. for 2 hours, and was monitored by TLC/LCMS, cooled the reaction, poured into H₂O (20 mL) and extracted with ethyl acetate (100 mL). The organic phase was dried on silica gel column to obtain product as Light yellow solid (1.3 g).

### Step 4 Preparation of Compound D001-5

D001-4 (325 mg) was added to MeOH (9 mL), and followed by three drops of TFA, Pd/C (100 mg), H₂ was added. Reaction was at 70° C. for 24 hours and was monitored by TLC/LCMS, and the reaction liquid was filtered, and filtrate was concentration under reduced pressure to get crude product compound D001-5 (289 mg).

### Step 5 Preparation of Compound D001

D001-5 (280 mg, 0.43 mmol) was added to DCM/MeOH (9:1, 10 mL), dropped by HCl/dioxane (0.5 mL, 4M). Reaction was at room temperature for 2 hours, and was monitored by LCMS, and the reaction liquid was purified by silica gel chromatography and concentration under reduced pressure to obtain compound D001 as a white solid (59 mg). [M+H]⁺=504.

The following compounds showing in Table 2 were synthesized using the above procedure of D001 or modified procedure with the corresponding starting materials.

TABLE 2

| | | | Physical |
|---|---|---|---|
| | | | Data |
| | | | (MS) |
| Example | Structure | Chemical Name | (M + H)⁺ |
| Example 108 (D001) | 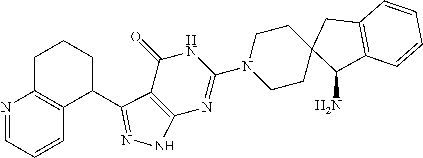 [SMILES: N[C@@H]1c2ccccc2CC12CCN(c1nc3[nH]nc(C4CCCc5ncccc54)c3c(=O)[nH]1)CC2] | 6-((S)-1-amino-1,3- dihydrospiro[indene-2,4′-piperidin]- 1′-yl)-3-(5,6,7,8-tetrahydroquinolin- 5-yl)-1,5-dihydro-4H-pyrazolo[3,4- d]pyrimidin-4-one | 468.24 |
| Example 109 (D002) | 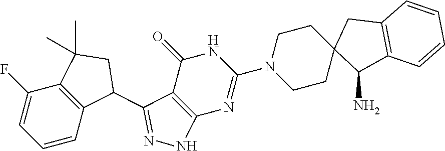 [SMILES: CC1(C)CC(c2n[nH]c3nc(N4CCC5(CC4)Cc4ccccc4[C@H]5N)[nH]c(=O)c23)c2cccc(F)c21] | 6-((S)-1-amino-1,3- dihydrospiro[indene-2,4′-piperidin]- 1′-yl)-3-(4-fluoro-3,3-dimethyl-2,3- dihydro-1H-inden-1-yl)-1,5-dihydro- 4H-pyrazolo[3,4-d]pyrimidin-4-one | 499.25 |
| Example 110 (D003) | 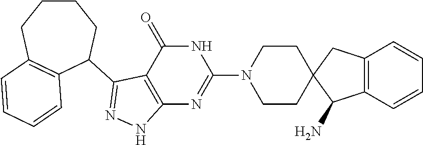 [SMILES: N[C@@H]1c2ccccc2CC12CCN(c1nc3[nH]nc(C4CCCCc5ccccc54)c3c(=O)[nH]1)CC2] | 6-((S)-1-amino-1,3- dihydrospiro[indene-2,4′-piperidin]- 1′-yl)-3-(6,7,8,9-tetrahydro-5H- benzo[7]annulen-5-yl)-1,5-dihydro- 4H-pyrazolo[3,4-d]pyrimidin-4-one | 481.26 |
| Example 68 | 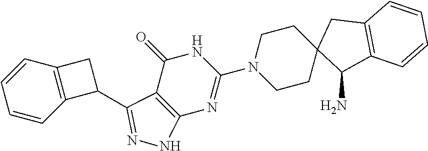 [SMILES: N[C@@H]1c2ccccc2CC12CCN(c1nc3[nH]nc(C4Cc5ccccc54)c3c(=O)[nH]1)CC2] | 6-((S)-1-amino-1,3- dihydrospiro[indene-2,4′-piperidin]- 1′-yl)-3-(bicyclo[4.2.0]octa-1(6),2,4- trien-7-yl)-1,5-dihydro-4H- pyrazolo[3,4-d\|pyrimidin-4-one | 439.22 |

### Example 176: Preparation of D069

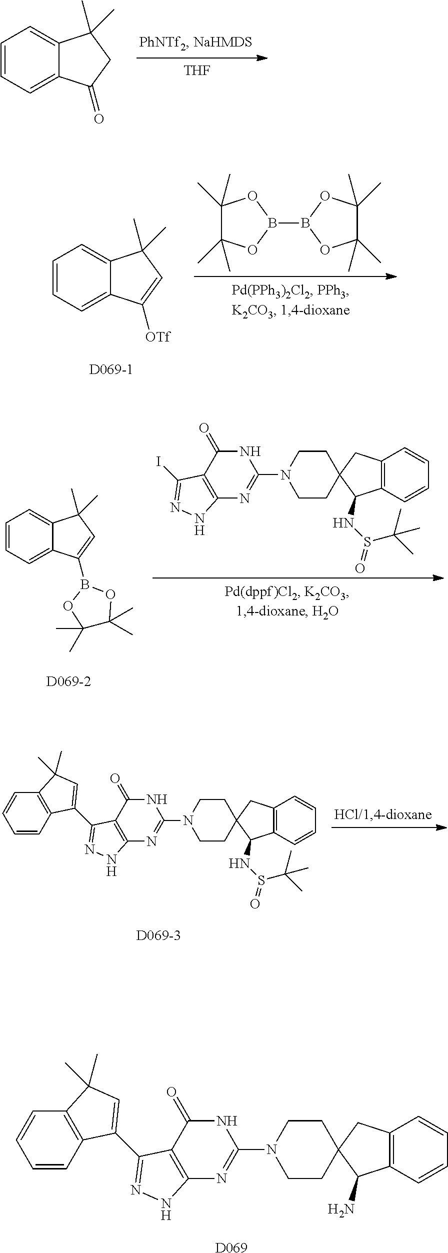

### Step 1: Preparation of D69-1

NaHMDS (4.7 mL, 2M in THF) was added dropwise into the mixture of 3,3-dimethyl-2,3-dihydro-1H-inden-1-one (1.00 g) and THF (20 mL) at −78° C. under nitrogen atmosphere. The reaction mixture was stirred for 30 min at −78° C. A mixture of 1,1,1-trifluoro-N-phenyl-N-((trifluoromethyl)sulfonyl)methanesulfonamide (3.34 g) in THF (5 mL) was added into the reaction mixture, and then stirred for 12 h while the temperature was allowed to warm up to room temperature naturally. The mixture was quenched by aqueous solution of NH₄Cl, extracted with ethyl acetate, washed with water, dried over anhydrous sodium sulfate. The mixture was filtered and the filtrate was concentration under reduced pressure. The crude product was purified by silica gel chromatography eluting with Hexane:EA=0%-50% afforded the target product (1.46 g) as off-white solid.

### Step 2: Preparation of D69-2

A mixture of D69-1 (200 mg), 4,4,4′,4′,5,5,5′,5′-octamethyl-2,2′-bi(1,3,2-dioxaborolane) (209 mg), K₂CO₃ (141 mg), Pd(PPh₃)₂Cl₂ (48 mg), PPh₃ (36 mg), and 1,4-dioxane (5 mL) was stirred for 1.5 h at 80° C. under nitrogen atmosphere. The mixture was concentrated under vacuum. The residue was dissolved in ethyl acetate, washed with water, dried over anhydrous sodium sulfate, and then filtered. The filtrate was concentration under reduced pressure to afford the target product (350 mg) as light brown solid, which was directly used to the next step without any purification.

### Step 3: Preparation of D69-3

A mixture of D69-2 (56 mg), M19 (60 mg), K₂CO₃ (44 mg), Pd(dppf)Cl₂ (8 mg), 1,4-dioxane (5 mL), and water (0.5 mL) was stirred for 4 h at 100° C. under nitrogen atmosphere. The mixture was concentrated under vacuum. The crude was purified by silica gel chromatography eluting with Hexane:EA=0%-50% afforded the target product (25 mg) as light yellow solid. LCMS [M+H]⁺=583.28.

### Step 4: Preparation of Compound D69

A mixture of D69-3 (25 mg) and HCl in 1,4-dioxane (5 mL) was stirred for overnight at room temperature. The reaction mixture was concentrated under vacuum. The residue was dissolved in water, and the pH value was adjusted to 8-9 with saturated solution of sodium bicarbonate. The mixture was extracted with ethyl acetate, washed with saturated brine, dried over anhydrous sodium sulfate, and then filtered. The filtrate was concentration under reduced pressure. The crude was purified by silica gel chromatography eluting with DCM:MeOH=0%-5% to afford the target product (14 mg) as off white solid. LCMS [M+H]⁺=479.25.

### Example 161: Preparation of D054

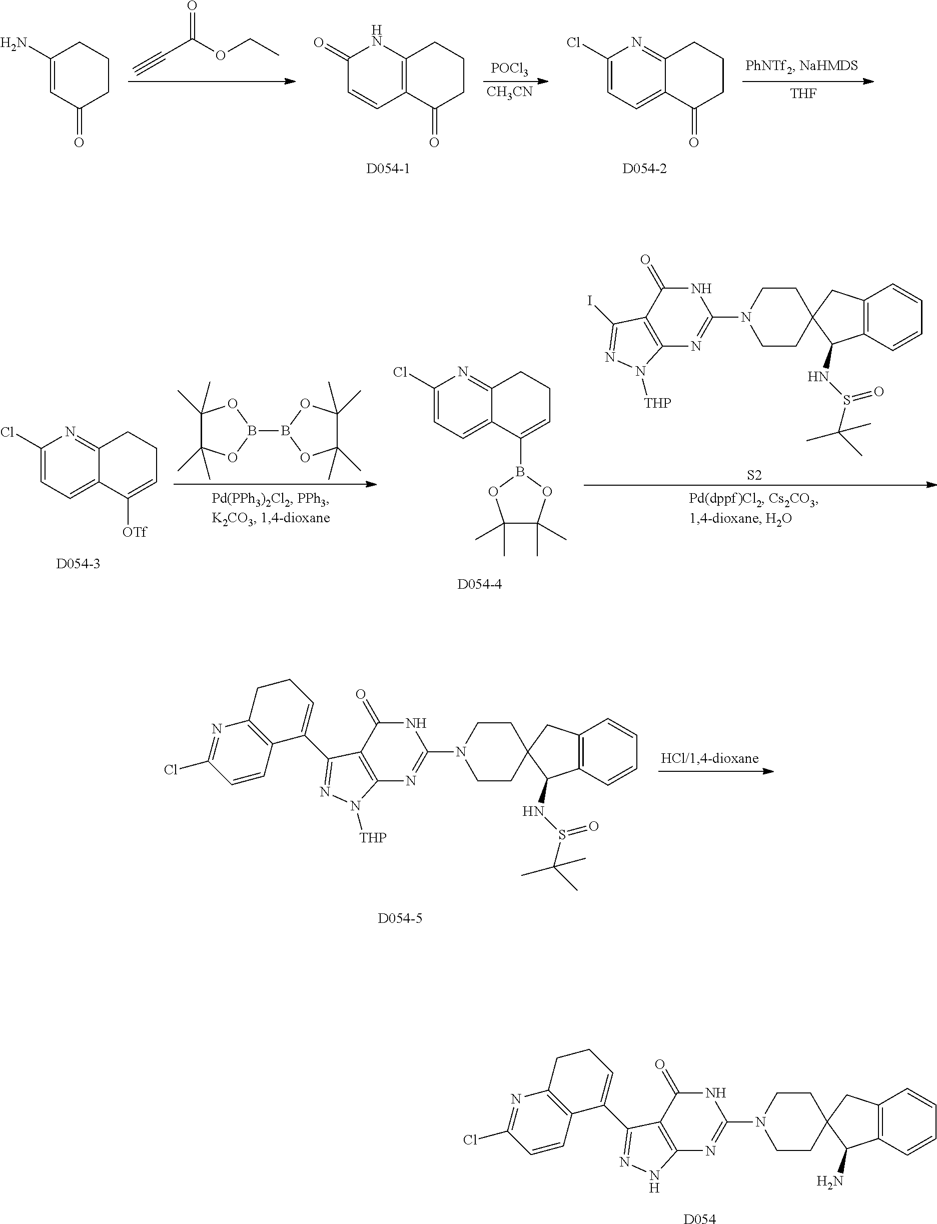

### Step 1: Preparation of D054-1

A mixture of 3-aminocyclohex-2-en-1-one (2.05 g) and ethyl propiolate (2.71 g) was stirred for overnight at 105° C. The reaction mixture was concentrated under vacuum. The residue was triturated with dichloromethane for 1 h. The mixture was filtered and washed with dichloromethane. The filter cake was dried under vacuum to afford the title compound (0.90 g) as yellow solid. LCMS [M+H]⁺=164.06.

### Step 2: Preparation of D054-2

A mixture of 7,8-dihydroquinoline-2,5(1H,6H)-dione (0.90 g), Phosphorus oxychloride (4.23 g), and acetonitrile (20 mL) was refluxed for 3 h. The reaction mixture was concentrated under vacuum. The residue was diluted with dichloromethane, washed with saturated solution of sodium bicarbonate and saturated brine respectively. The organic phase was dried over anhydrous sodium sulfate, filtered and then the filtrate was concentration under reduced pressure. The crude was purified by silica gel chromatography eluting with Hexane:EA=0%-50% to afford the tittle compound (810 mg) as off-white solid. LCMS [M+H]⁺=182.03.

### Step 3: Preparation of D054-3

KHMDS (6.7 mL, 1M in THF) was added dropwise into a mixture of D054-2 (810 mg) and THF (30 mL) at −78° C. under nitrogen atmosphere. The reaction was stirred for 30 min at −78° C. A solution of PhNTf₂ (1.92 g) in THF was added into the reaction mixture at 0° C. The reaction mixture was stirred for 30 min at 0° C. and then was allowed to warm up to room temperature. The reaction was quenched by saturated solutions of ammonium chloride, extracted with ethyl acetate, washed with saturated brine, dried over anhydrous sodium sulfate, and filtered. The filtrate was concentration under reduced pressure. The crude was purified by silica gel chromatography eluting with Hexane:EA=0%-50% to afford the tittle compound (1.32 g) as yellow solid. LCMS [M+H]⁺=313.98.

### Step 4: Preparation of D054-4

A mixture of D054-3 (157 mg), 4,4,4′,4′,5,5,5′,5′-octamethyl-2,2′-bi(1,3,2-dioxaborolane) (152 mg), PdCl₂(PPh₃)₂ (35 mg), PPh₃ (26 mg), K₂CO₃ (99 mg), and toluene (8 mL) was stirred for 1.5 h at 50° C. under nitrogen atmosphere. The reaction mixture was concentrated under vacuum. The residue was dissolved in dichloromethane, washed with saturated brine, dried over anhydrous sodium sulfate, and filtered. The filtrate was concentrated under reduced pressure to afford the title compound (150 mg) as brown solid, which was used for the next step without any purification. LCMS [M+H]⁺=292.12.

### Step 5: Preparation of D054-5

A mixture of M19 (100 mg), D054-4 (150 mg), PdCl₂(dppf)CH₂Cl₂ (30 mg), Cs₂CO₃ (260 mg), 1,4-dioxane (6 mL), and water (1 mL) was stirred for 2 h at 50° C. under nitrogen atmosphere. The reaction mixture was concentrated under vacuum. The crude was purified by silica gel chromatography eluting with DCM:MeOH=20:1 to afford the tittle compound (45 mg) as off-white solid. LCMS [M+H]⁺=688.28.

### Step 6: Preparation of D054

A mixture of D054-5 (45 mg), dichloromethane (8 mL), and HCl in 1,4-dioxane (0.8 mL, 4M) was stirred for 20 min at room temperature. The reaction mixture was concentrated under vacuum. The residue was dissolved in water, and the pH value was adjusted to 8-9 with saturated solution of sodium bicarbonate. The mixture was extracted with ethyl acetate, washed with saturated brine, dried over anhydrous sodium sulfate, and then filtered. The filtrate was concentrated under reduced pressure. The crude was purified by silica gel chromatography eluting with DCM:MeOH=0%-5%, afforded the target product compound D054 (24 mg) as light yellow solid. LCMS [M+H]⁺=500.19.

### Example 144: Preparation of D037

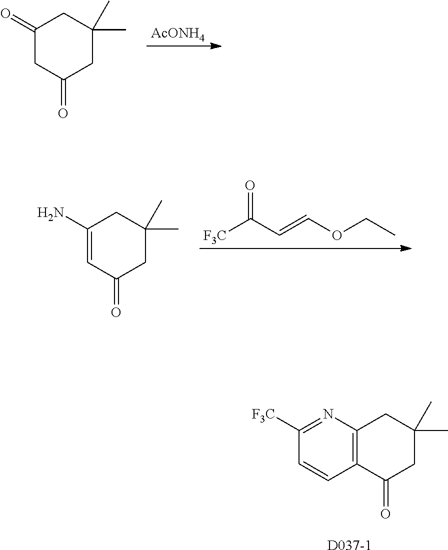

### Step 1: Preparation of 3-amino-5,5-dimethylcyclohex-2-en-1-one

A mixture of 5,5-dimethylcyclohexane-1,3-dione (5.00 g) and ammonium acetate (13.75 g) was stirred for 10 min at 120° C.-130° C., The reaction mixture was cooled down to room temperature, suspended in water, and then extracted with ethyl acetate. The organic layers were combined and concentrated under vacuum to afford the title compound (4.38 g) as light yellow solid. LCMS [M+H]⁺=140.10.

### Step 2: Preparation of Compound D037-1

A mixture of 3-amino-5,5-dimethylcyclohex-2-en-1-one (1.40 g), 4-ethoxy-1,1,1-trifluorobut-3-en-2-one (2.00 g), and acetic acid (20 mL) was stirred for 30 min at 100° C. and then for 3 h at reflux temperature. The reaction mixture was concentrated under vacuum. The residue was dissolved in ethyl acetate, washed with saturated sodium bicarbonate aqueous and then saturated brine. The organic phase was dried over anhydrous sodium sulfate, and then filtered. The filtrate was concentrated under reduced pressure. The crude product was purified by silica gel chromatography eluting with Hexane:EA=0%-50%, afforded the target product compound D037-1 (1.25 g) as light yellow solid. LCMS [M+H]⁺=244.09.

The method for preparing compound D037 from intermediate D037-1 is the same as the method for preparing compound D054 from D054-2.

### Example 147: Preparation of D040

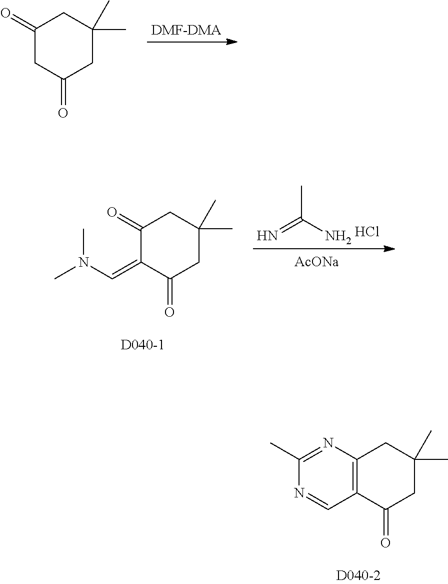

### Step 1: Preparation of D040-1

N,N dimethylformamide dimethyl-acetal (5 mL) was added slowly into a solution of 5,5-dimethylcyclohexane-1,3-dione (5 g) in CHCl₃ (50 mL) at 0° C. The reaction mixture was heated to reflux for 1 h. The mixture was concentrated under vacuum to afford the title compound (7.10 g) as yellow solid, which was used for the next step without any purification. LCMS [M+H]⁺=196.13.

### Step 2: Preparation of D040-2

A mixture of D040-1 (7.10 g), sodium acetate (5.97 g), acetamidine hydrochloride (4.13 g), and ethanol (50 mL) was refluxed for 12 h. The reaction mixture was concentrated under vacuum. The residue was diluted with ethyl acetate, washed with water, dried over anhydrous sodium sulfate, and filtered. The filtrate was concentrated under reduced pressure. The crude was purified by silica gel chromatography eluting with Hexane:EA=0%-50%, afforded the target product D040-2 (4.50 g) as light yellow solid. LCMS [M+H]⁺=191.11.

The method for preparing compound D040 from intermediate D040-2 is the same as the method for preparing compound D054 from D054-2.

### Example 186: Preparation of D078

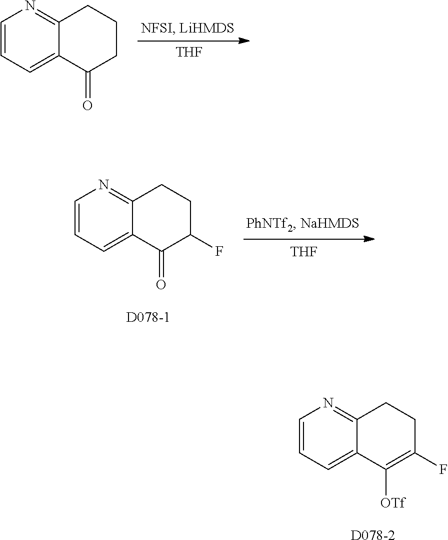

### Step 1: Preparation of D078-1

LiHMDS (10.3 mL, 2M in THF) was added dropwise into a mixture of 7,8-dihydroquinolin-5(6H)-one (2.02 g) and THF (30 mL) at −78° C. under nitrogen atmosphere. The reaction mixture was stirred for 30 min at −78° C. A solution of N-Fluorobenzenesulfonimide (5.19 g) in THF was added drop wise into the reaction. The mixture was allowed to warm up to room temperature and stirred for 12 h. The reaction was quenched with saturated solution of ammonium chloride, extracted with ethyl acetate, washed with saturated brine, dried over anhydrous sodium sulfate, and filtered. The filtrate was concentrated under reduced pressure. The crude was purified by silica gel chromatography eluting with Hexane:EA=0%-50%, afforded the target product (1.93 g) as light yellow solid. LCMS [M+H]⁺=166.06.

### Step 2: Preparation of D078-2

NaHMDS (9 mL, 2M in THF) was added dropwise into a mixture of D078-1 (1.93 g) and THF (20 mL) at −78° C. under nitrogen atmosphere. The reaction mixture was stirred for 30 min at −78° C. A mixture of 1,1,1-trifluoro-N-phenyl-N-((trifluoromethyl)sulfonyl)methanesulfonamide (6.43 g) in THF (10 mL) was added into the reaction mixture, and then stirred for 12 h while the temperature was allowed to warm up to room temperature naturally. The mixture was quenched by aqueous solution of NH₄Cl, extracted with ethyl acetate, washed with water, dried over anhydrous sodium sulfate. The mixture was filtered and the filtrate was concentrated under reduced pressure. The crude product was purified by silica gel chromatography eluting with Hexane:EA=0%-50% afforded the target product compound D078-2 (1.84 g) as light yellow solid.

The method for preparing compound D078 from intermediate D078-2 is the same as the method for preparing compound D054 from D054-3.

The following compounds (such as D004-D053, D055-D068, D070-D083) showing in Table 3 were synthesized using the above procedures of D054, D069 or modified procedure with the corresponding starting materials.

TABLE 3

| | | | Physical |
|---|---|---|---|
| | | | Data |
| | | | (MS) |
| Example | Structure | Chemical Name | (M + H)⁺ |
| Example 111 (D004) | 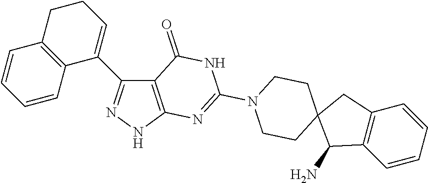 [SMILES: N[C@@H]1c2ccccc2CC12CCN(c1nc3[nH]nc(C4=CCCc5ccccc54)c3c(=O)[nH]1)CC2] | (S)-6-(1-amino-1,3- dihydrospiro[indene-2,4′-piperidin]- 1′-yl)-3-(3,4-dihydronaphthalen-1- yl)-1,5-dihydro-4H-pyrazolo[3,4- d]pyrimidin-4-one | 465.23 |
| Example 112 (D005) | 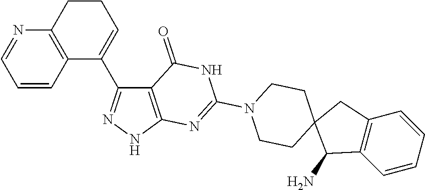 [SMILES: N[C@@H]1c2ccccc2CC12CCN(c1nc3[nH]nc(C4=CCCc5ncccc54)c3c(=O)[nH]1)CC2] | (S)-6-(1-amino-1,3- dihydrospiro[indene-2,4′-piperidin]- 1′-yl)-3-(7,8-dihydroquinolin-5-yl)- 1,5-dihydro-4H-pyrazolo[3,4- d]pyrimidin-4-one | 466.23 |
| Example 113 (D006) | 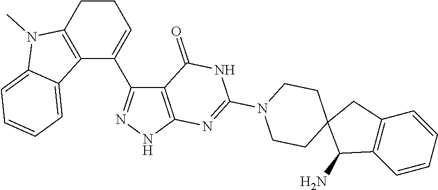 [SMILES: Cn1c2c(c3ccccc31)C(c1n[nH]c3nc(N4CCC5(CC4)Cc4ccccc4[C@H]5N)[nH]c(=O)c13)=CCC2] | (S)-6-(1-amino-1,3- dihydrospiro[indene-2,4′-piperidin]- 1′-yl)-3-(9-methyl-2,9-dihydro-1H- carbazol-4-yl)-1,5-dihydro-4H- pyrazolo[3,4-d]pyrimidin-4-one | 518.26 |
| Example 114 (D007) | 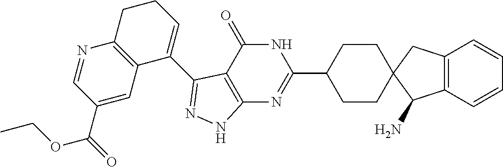 [SMILES: CCOC(=O)c1cnc2c(c1)C(c1n[nH]c3nc(C4CCC5(CC4)Cc4ccccc4[C@H]5N)[nH]c(=O)c13)=CCC2] | ethyl(S)-5-(6-(1-amino-1,3- dihydrospiro[indene-2,4′-piperidin]- 1′-yl)-4-oxo-4,5-dihydro-1H- pyrazolo[3,4-d]pyrimidin-3-yl)-7,8- dihydroquinoline-3-carboxylate | 438.25 |
| Example 115 (D008) | 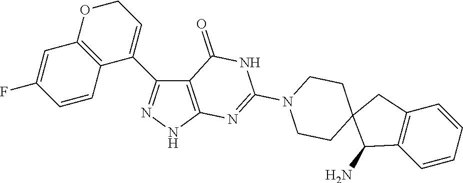 [SMILES: N[C@@H]1c2ccccc2CC12CCN(c1nc3[nH]nc(C4=CCOc5cc(F)ccc54)c3c(=O)[nH]1)CC2] | (S)-6-(1-amino-1,3- dihydrospiro[indene-2,4′-piperidin]- 1′-yl)-3-(7-fluoro-2H-chromen-4-yl)- 1,5-dihydro-4H-pyrazolo[3,4- d]pyrimidin-4-one | 485.20 |
| Example 116 (D009) | 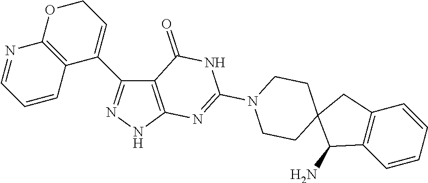 [SMILES: N[C@@H]1c2ccccc2CC12CCN(c1nc3[nH]nc(C4=CCOc5ncccc54)c3c(=O)[nH]1)CC2] | (S)-6-(1-amino-1,3- dihydrospiro[indene-2,4′-piperidin]- 1′-yl)-3-(2H-pyrano[2,3-b]pyridin-4- yl)-1,5-dihydro-4H-pyrazolo[3,4- d]pyrimidin-4-one | 468.21 |
| Example 117 (D010) | 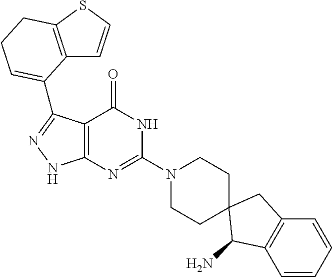 [SMILES: N[C@@H]1c2ccccc2CC12CCN(c1nc3[nH]nc(C4=CCCc5sccc54)c3c(=O)[nH]1)CC2] | (S)-6-(1-amino-1,3- dihydrospiro[indene-2,4′-piperidin]- 1′-yl)-3-(6,7- dihydrobenzo[b]thiophen-4-yl)-1,5- dihydro-4H-pyrazolo[3,4- d]pyrimidin-4-one | 471.19 |
| Example 118 (D011) | 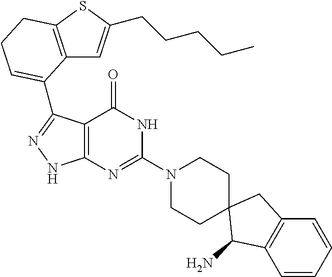 [SMILES: CCCCCc1cc2c(s1)CCC=C2c1n[nH]c2nc(N3CCC4(CC3)Cc3ccccc3[C@H]4N)[nH]c(=O)c12] | (S)-6-(1-amino-1,3- dihydrospiro[indene-2,4′-piperidin]- 1′-yl)-3-(2-pentyl-6,7- dihydrobenzo[b]thiophen-4-yl)-1,5- dihydro-4H-pyrazolo[3,4- d]pyrimidin-4-one | 541.27 |
| Example 119 (D012) | 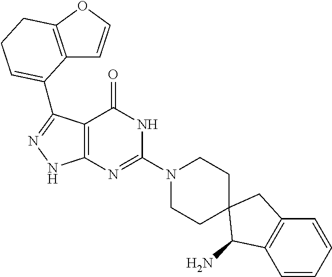 [SMILES: N[C@@H]1c2ccccc2CC12CCN(c1nc3[nH]nc(C4=CCCc5occc54)c3c(=O)[nH]1)CC2] | (S)-6-(1-amino-1,3- dihydrospiro[indene-2,4′-piperidin]- 1′-yl)-3-(6,7-dihydrobenzofuran-4- yl)-1,5-dihydro-4H-pyrazolo[3,4- d]pyrimidin-4-one | 455.21 |
| Example 120 (D013) | 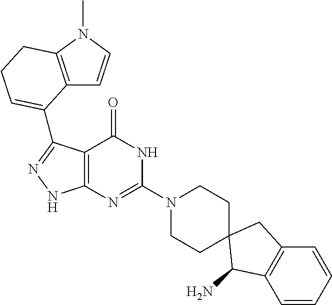 [SMILES: Cn1ccc2c1CCC=C2c1n[nH]c2nc(N3CCC4(CC3)Cc3ccccc3[C@H]4N)[nH]c(=O)c12] | (S)-6-(1-amino-1,3- dihydrospiro[indene-2,4′-piperidin]- 1′-yl)-3-(1-methyl-6,7-dihydro-1H- indol-4-yl)-1,5-dihydro-4H- pyrazolo[3,4-d]pyrimidin-4-one | 468.24 |
| Example 121 (D014) | 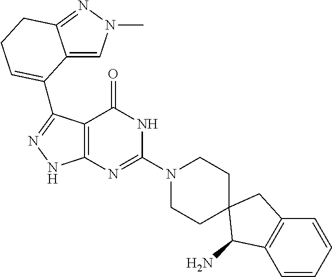 [SMILES: Cn1cc2c(n1)CCC=C2c1n[nH]c2nc(N3CCC4(CC3)Cc3ccccc3[C@H]4N)[nH]c(=O)c12] | (S)-6-(1-amino-1,3- dihydrospiro[indene-2,4′-piperidin]- 1′-yl)-3-(2-methyl-6,7-dihydro-2H- indazol-4-yl)-1,5-dihydro-4H- pyrazolo[3,4-d]pyrimidin-4-one | 469.24 |
| Example 122 (D015) | 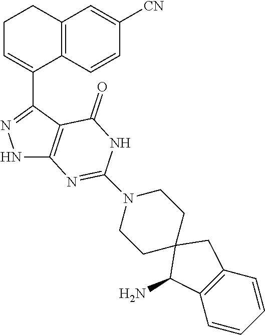 [SMILES: N#Cc1ccc2c(c1)CCC=C2c1n[nH]c2nc(N3CCC4(CC3)Cc3ccccc3[C@H]4N)[nH]c(=O)c12] | (S)-5-(6-(1-amino-1,3- dihydrospiro[indene-2,4′-piperidin]- 1′-yl)-4-oxo-4,5-dihydro-1H- pyrazolo[3,4-d]pyrimidin-3-yl)-7,8- dihydronaphthalene-2-carbonitrile | 490.23 |
| Example 123 (D016) | 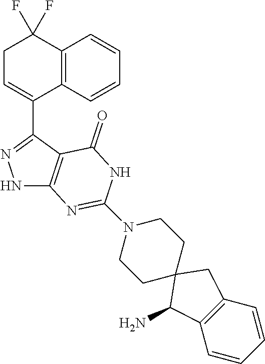 [SMILES: N[C@@H]1c2ccccc2CC12CCN(c1nc3[nH]nc(C4=CCC(F)(F)c5ccccc54)c3c(=O)[nH]1)CC2] | (S)-6-(1-amino-1,3- dihydrospiro[indene-2,4′-piperidin]- 1′-yl)-3-(4,4-difluoro-3,4- dihydronaphtlialen-1-yl)-1,5-dihydro- 4H-pyrazolo[3,4-d]pyrimidin-4-one | 501.21 |
| Example 124 (D017) | 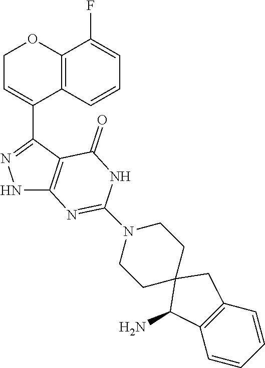 [SMILES: N[C@@H]1c2ccccc2CC12CCN(c1nc3[nH]nc(C4=CCOc5c(F)cccc54)c3c(=O)[nH]1)CC2] | 1-yl)-3-(8-fluoro-2H-chromen-4-yl)- 1,5-dihydro-4H-pyrazolo[3,4- d]pyrimidin-4-one | 485.20 |
| Example 125 (D018) | 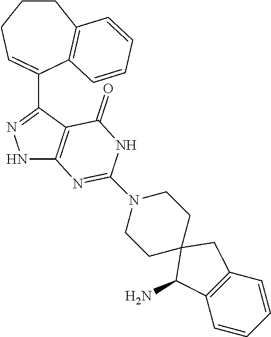 [SMILES: N[C@@H]1c2ccccc2CC12CCN(c1nc3[nH]nc(C4=CCCCc5ccccc54)c3c(=O)[nH]1)CC2] | dihydrospiro[indene-2,4′-piperidin]- 1′-yl)-3-(6,7-dihydro-5H- benzo[7]annulen-9-yl)-1,5-dihydro- 4H-pyrazolo[3,4-d]pyrimidin-4-one | 479.25 |
| Example 126 (D019) | 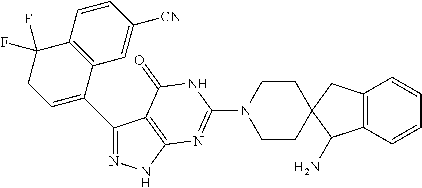 [SMILES: N#Cc1ccc2c(c1)C(c1n[nH]c3nc(N4CCC5(CC4)Cc4ccccc4C5N)[nH]c(=O)c13)=CCC2(F)F] | dihydrospiro[indene-2,4′-piperidin]- 1′-yl)-4-oxo-4,5-dihydro-1H- pyrazolo[3,4-d]pyrimidin-3-yl)-5,5- difluoro-5,6-dihydronaphthalene-2- carbonitrile | 526.21 |
| Example 127 (D020) | 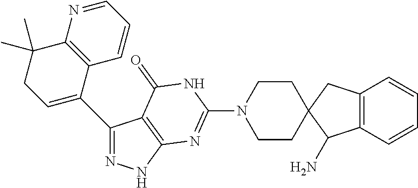 [SMILES: CC1(C)CC=C(c2n[nH]c3nc(N4CCC5(CC4)Cc4ccccc4C5N)[nH]c(=O)c23)c2cccnc21] | 6-(1-amino-1,3-dihydrospiro[indene- 2,4′-piperidin]-1′-yl)-3-(8,8-dimethyl- 7,8-dihydroquinolin-5-yl)-1,5- dihydro-4H-pyrazolo[3,4- d]pyrimidin-4-one | 494.26 |
| Example 128 (D021) | 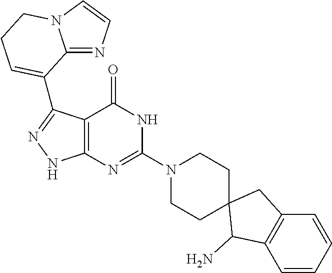 [SMILES: NC1c2ccccc2CC12CCN(c1nc3[nH]nc(C4=CCCn5ccnc54)c3c(=O)[nH]1)CC2] | 2,4′-piperidin]-1′-yl)-3-(5,6- dihydroimidazo[1,2-a]pyridin-8-yl)- 1,5-dihydro-4H-pyrazolo[3,4- d]pyrimidin-4-one | 455.22 |
| Example 129 (D022) | 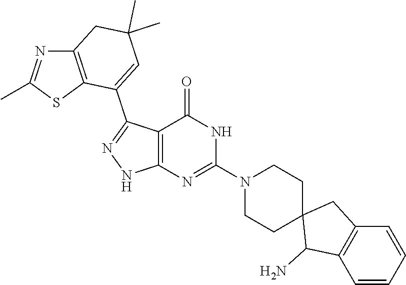 [SMILES: Cc1nc2c(s1)C(c1n[nH]c3nc(N4CCC5(CC4)Cc4ccccc4C5N)[nH]c(=O)c13)=CC(C)(C)C2] | 2,4′-piperidin]-1′-yl)-3-(2,5,5- trimethyl-4,5- dihydrobenzo[d]thiazol-7-yl)-1,5- dihydro-4H-pyrazolo[3,4- d]pyrimidin-4-one | 514.23 |
| Example 130 (D023) | 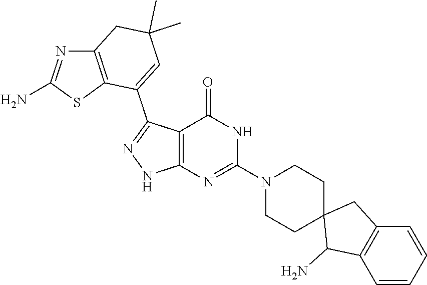 [SMILES: CC1(C)C=C(c2n[nH]c3nc(N4CCC5(CC4)Cc4ccccc4C5N)[nH]c(=O)c23)c2sc(N)nc2C1] | 6-(1-amino-1,3-dihydrospiro[indene- 2,4′-piperidin]-1′-yl)-3-(2-amino-5,5- dimethyl-4,5- dihydrobenzo[d]thiazol-7-yl)-1,5- dihydro-4H-pyrazolo[3,4- d]pyrimidin-4-one | 515.23 |
| Example 131 (D024) | 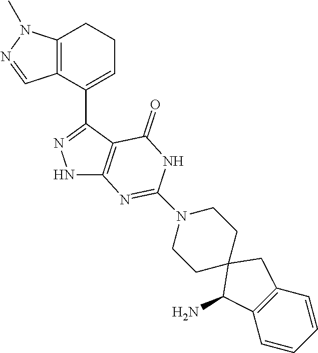 [SMILES: Cn1ncc2c1CCC=C2c1n[nH]c2nc(N3CCC4(CC3)Cc3ccccc3[C@H]4N)[nH]c(=O)c12] | (S)-6-(1-amino-1,3- dihydrospiro[indene-2,4′-piperidin]- 1′-yl)-3-(1-methyl-6,7-dihydro-1H- indazol-4-yl)-1,5-dihydro-4H- pyrazolo[3,4-d]pyrimidin-4-one | 469.24 |
| Example 132 (D025) | 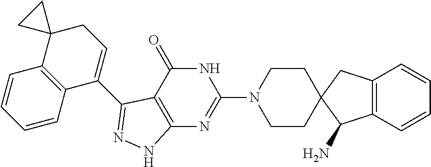 [SMILES: N[C@@H]1c2ccccc2CC12CCN(c1nc3[nH]nc(C4=CCC5(CC5)c5ccccc54)c3c(=O)[nH]1)CC2] | (S)-6-(1-amino-1,3- dihydrospiro[indene-2,4′-piperidin]- 1′-yl)-3-(2′H-spiro[cyclopropane- 1,1′-naphthalen]-4′-yl)-1,5-dihydro- 4H-pyrazolo[3,4-d]pyrimidin-4-one | 491.25 |
| Example 133 (D026) | 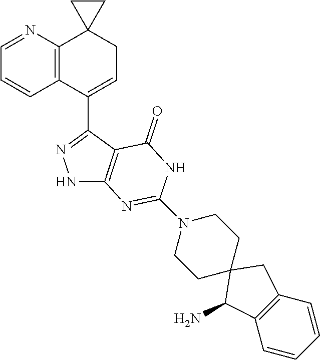 [SMILES: N[C@@H]1c2ccccc2CC12CCN(c1nc3[nH]nc(C4=CCC5(CC5)c5ncccc54)c3c(=O)[nH]1)CC2] | (S)-6-(1-amino-1,3- dihydrospiro[indene-2,4′-piperidin]- 1′-yl)-3-(7′H-spiro[cyclopropane- 1,8′-quinolin]-5′-yl)-1,5-dihydro-4H- pyrazolo[3,4-d]pyrimidin-4-one | 492.24 |
| Example 134 (D027) | 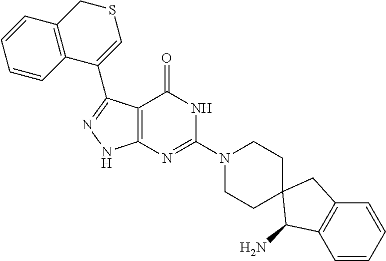 [SMILES: N[C@@H]1c2ccccc2CC12CCN(c1nc3[nH]nc(C4=CSCc5ccccc54)c3c(=O)[nH]1)CC2] | (S)-6-(1-amino-1,3- dihydrospiro[indene-2,4′-piperidin]- 1′-yl)-3-(1H-isothiochromen-4-yl)- 1,5-dihydro-4H-pyrazolo[3,4- d]pyrimidin-4-one | 483.19 |
| Example 135 (D028) | 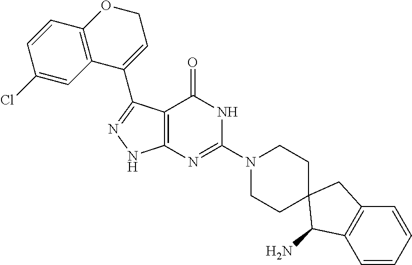 [SMILES: N[C@@H]1c2ccccc2CC12CCN(c1nc3[nH]nc(C4=CCOc5ccc(Cl)cc54)c3c(=O)[nH]1)CC2] | (S)-6-(1-amino-1,3- dihydrospiro[indene-2,4′-piperidin]- 1′-yl)-3-(6-chloro-2H-chromen-4-yl)- 1,5-dihydro-4H-pyrazolo[3,4- d]pyrimidin-4-one | 501.17 |
| Example 136 (D029) | 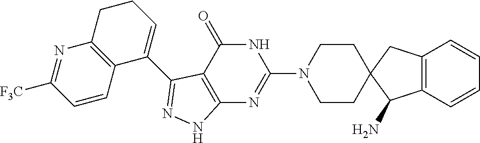 [SMILES: N[C@@H]1c2ccccc2CC12CCN(c1nc3[nH]nc(C4=CCCc5nc(C(F)(F)F)ccc54)c3c(=O)[nH]1)CC2] | (S)-6-(1-amino-1,3- dihydrospiro[indene-2,4′-piperidin]- 1′-yl)-3-(2-(trifluoromethyl)-7,8- dihydroquinolin-5-yl)-1,5-dihydro- 4H-pyrazolo[3,4-d]pyrimidin-4-one | 534.22 |
| Example 137 (D030) | 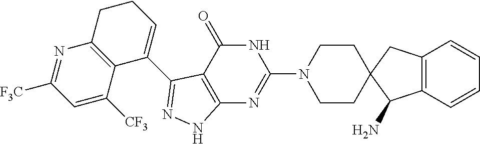 [SMILES: N[C@@H]1c2ccccc2CC12CCN(c1nc3[nH]nc(C4=CCCc5nc(C(F)(F)F)cc(C(F)(F)F)c54)c3c(=O)[nH]1)CC2] | (S)-6-(1-amino-1,3- dihydrospiro[indene-2,4′-piperidin]- 1′-yl)-3-(2,4-bis(trifluoromethyl)-7,8- dihydroquinolin-5-yl)-1,5-dihydro- 4H-pyrazolo[3,4-d]pyrimidin-4-one | 602.20 |
| Example 138 (D031) | 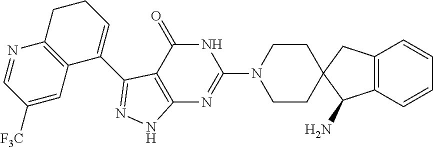 [SMILES: N[C@@H]1c2ccccc2CC12CCN(c1nc3[nH]nc(C4=CCCc5ncc(C(F)(F)F)cc54)c3c(=O)[nH]1)CC2] | (S)-6-(1-amino-1,3- dihydrospiro[indene-2,4′-piperidin]- 1′-yl)-3-(3-(trifluoromethyl)-7,8- dihydroquinolin-5-yl)-1,5-dihydro- 4H-pyrazolo[3,4-d]pyrimidin-4-one | 534.22 |
| Example 139 (D032) | 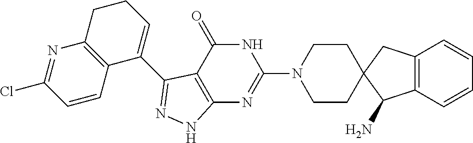 [SMILES: N[C@@H]1c2ccccc2CC12CCN(c1nc3[nH]nc(C4=CCCc5nc(Cl)ccc54)c3c(=O)[nH]1)CC2] | (S)-6-(1-amino-1,3- dihydrospiro[indene-2,4′-piperidin]- 1′-yl)-3-(2-chloro-7,8- dihydroquinolin-5-yl)-1,5-dihydro- 4H-pyrazolo[3,4-d]pyrimidin-4-one | 500.19 |
| Example 140 (D033) | 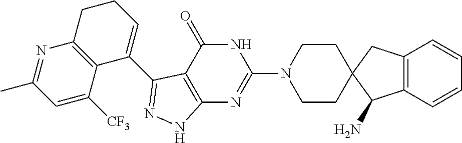 [SMILES: Cc1cc(C(F)(F)F)c2c(n1)CCC=C2c1n[nH]c2nc(N3CCC4(CC3)Cc3ccccc3[C@H]4N)[nH]c(=O)c12] | (S)-6-(1-amino-1,3- dihydrospiro[indene-2,4′-piperidin]- 1′-yl)-3-(2-methyl-4- (trifluoromethyl)-7,8- dihydroquinolin-5-yl)-1,5-dihydro- 4H-pyrazolo[3,4-d]pyrimidin-4-one | 548.23 |
| Example 141 (D034) | 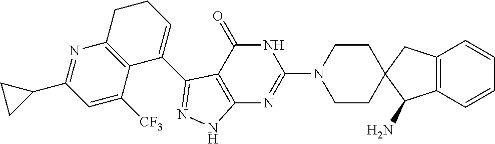 [SMILES: N[C@@H]1c2ccccc2CC12CCN(c1nc3[nH]nc(C4=CCCc5nc(C6CC6)cc(C(F)(F)F)c54)c3c(=O)[nH]1)CC2] | (S)-6-(1-amino-1,3- dihydrospiro[indene-2,4′-piperidin]- 1′-yl)-3-(2-cyclopropyl-4- (trifluoromethyl)-7,8- dihydroquinolin-5-yl)-1,5-dihydro- 4H-pyrazolo[3,4-d]pyrimidin-4-one | 574.25 |
| Example 142 (D035) | 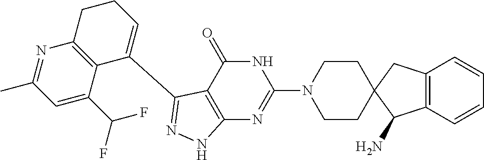 [SMILES: Cc1cc(C(F)F)c2c(n1)CCC=C2c1n[nH]c2nc(N3CCC4(CC3)Cc3ccccc3[C@H]4N)[nH]c(=O)c12] | (S)-6-(1-amino-1,3- dihydrospiro[indene-2,4′-piperidin]- 1′-yl)-3-(4-(difluoromethyl)-2- methyl-7,8-dihydroquinolin-5-yl)- 1,5-dihydro-4H-pyrazolo[3,4- d]pyrimidin-4-one | 530.24 |
| Example 143 (D036) | 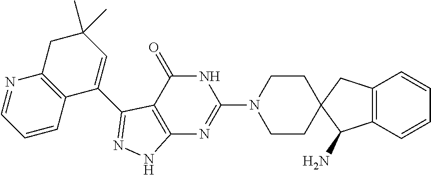 [SMILES: CC1(C)C=C(c2n[nH]c3nc(N4CCC5(CC4)Cc4ccccc4[C@H]5N)[nH]c(=O)c23)c2cccnc2C1] | (S)-6-(1-amino-1,3- dihydrospiro[indene-2,4′-piperidin]- 1′-yl)-3-(7,7-dimethyl-7,8- dihydroquinolin-5-yl)-1,5-dihydro- 4H-pyrazolo[3,4-d]pyrimidin-4-one | 494.26 |
| Example 144 (D037) | 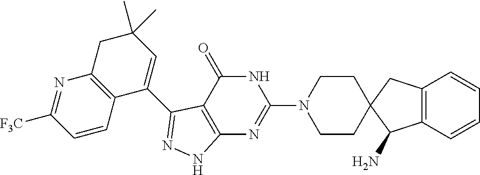 [SMILES: CC1(C)C=C(c2n[nH]c3nc(N4CCC5(CC4)Cc4ccccc4[C@H]5N)[nH]c(=O)c23)c2ccc(C(F)(F)F)nc2C1] | (S)-6-(1-amino-1,3- dihydrospiro[indene-2,4′-piperidin]- 1′-yl)-3-(7,7-dimethyl-2- (trifluoromethyl)-7,8- dihydroquinolin-5-yl)-1,5-dihydro- 4H-pyrazolo[3,4-d]pyrimidin-4-one | 562.25 |
| Example 145 (D038) | 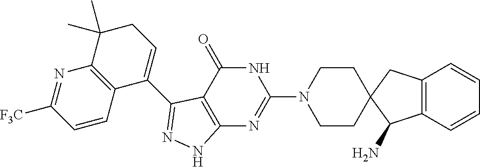 [SMILES: CC1(C)CC=C(c2n[nH]c3nc(N4CCC5(CC4)Cc4ccccc4[C@H]5N)[nH]c(=O)c23)c2ccc(C(F)(F)F)nc21] | (S)-6-(1-amino-1,3- dihydrospiro[indene-2,4′-piperidin]- 1′-yl)-3-(8,8-dimethyl-2- (trifluoromethyl)-7,8- dihydroquinolin-5-yl)-1,5-dihydro- 4H-pyrazolo[3,4-d]pyrimidin-4-one | 562.25 |
| Example 146 (D039) | 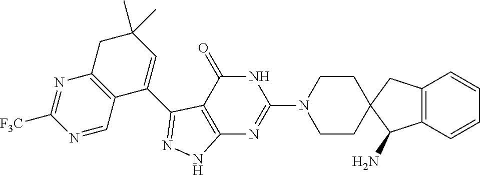 [SMILES: CC1(C)C=C(c2n[nH]c3nc(N4CCC5(CC4)Cc4ccccc4[C@H]5N)[nH]c(=O)c23)c2cnc(C(F)(F)F)nc2C1] | (S)-6-(l-amino-1,3- dihydrospiro[indene-2,4'-piperidin]- 1′-yl)-3-(7,7-dimethyl-2- (trifluoromethyl)-7,8- dihydroquinazolin-5-yl)-1,5-dihydro- 4H-pyrazolo[3,4-d]pyrimidin-4-one | 563.24 |
| Example 147 (D040) | 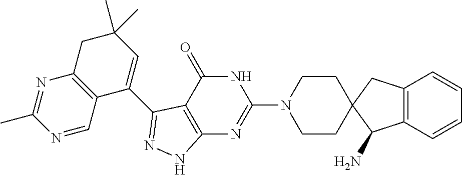 [SMILES: Cc1ncc2c(n1)CC(C)(C)C=C2c1n[nH]c2nc(N3CCC4(CC3)Cc3ccccc3[C@H]4N)[nH]c(=O)c12] | (S)-6-(l-amino-1,3- dihydrospiro[indene-2,4'-piperidin]- 1′-yl)-3-(2,7,7-trimethyl-7,8- dihydroquinazolin-5-yl)-1,5-dihydro- 4H-pyrazolo[3,4-d]pyrimidin-4-one | 509.27 |
| Example 148 (D041) | 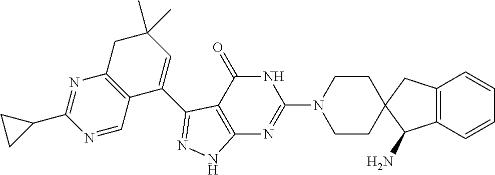 [SMILES: CC1(C)C=C(c2n[nH]c3nc(N4CCC5(CC4)Cc4ccccc4[C@H]5N)[nH]c(=O)c23)c2cnc(C3CC3)nc2C1] | (S)-6-(l-amino-1,3- dihydrospiro[indene-2,4'-piperidin]- 1′-yl)-3-(2-cyclopropyl-7,7-dimethyl- 7,8-dihydroquinazolin-5-yl)-1,5- dihydro-4H-pyrazolo[3,4- d]pyrimidin-4-one | 535.29 |
| Example 149 (D042) | 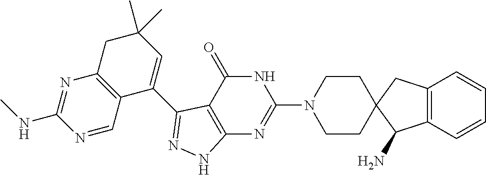 [SMILES: CNc1ncc2c(n1)CC(C)(C)C=C2c1n[nH]c2nc(N3CCC4(CC3)Cc3ccccc3[C@H]4N)[nH]c(=O)c12] | (S)-6-(1-amino-1,3- dihydrospiro[indene-2,4′-piperidin]- 1′-yl)-3-(7,7-dimethyl-2- (methylamino)-7,8- dihydroquinazolin-5-yl)-1,5-dihydro- 4H-pyrazolo[3,4-d]pyrimidin-4-one | 524.28 |
| Example 150 (D043) | 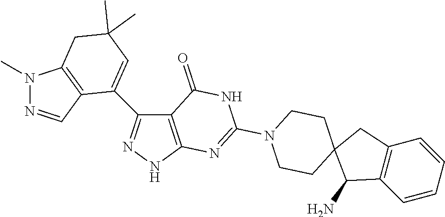 [SMILES: Cn1ncc2c1CC(C)(C)C=C2c1n[nH]c2nc(N3CCC4(CC3)Cc3ccccc3[C@H]4N)[nH]c(=O)c12] | (S)-6-(1-amino-1,3- dihydrospiro[indene-2,4′-piperidin]- 1′-yl)-3-(1,6,6-trimethyl-6,7-dihydro- 1H-indazol-4-yl)-1,5-dihydro-4H- pyrazolo[3,4-d]pyrimidin-4-one | 497.27 |
| Example 151 (D044) | 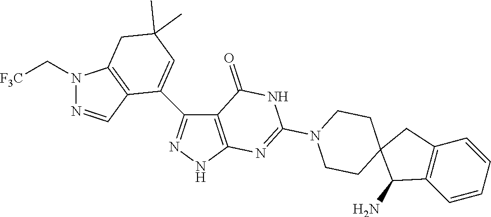 [SMILES: CC1(C)C=C(c2n[nH]c3nc(N4CCC5(CC4)Cc4ccccc4[C@H]5N)[nH]c(=O)c23)c2cnn(CC(F)(F)F)c2C1] | (S)-6-(1-amino-1,3- dihydrospiro[indene-2,4′-piperidin]- 1′-yl)-3-(6,6-dimethyl-l-(2,2,2- trifluoroethyl)-6,7-dihydro-1H- indazol-4-yl)-1,5-dihydro-4H- pyrazolo[3,4-d]pyrimidin-4-one | 565.25 |
| Example 152 (D045) | 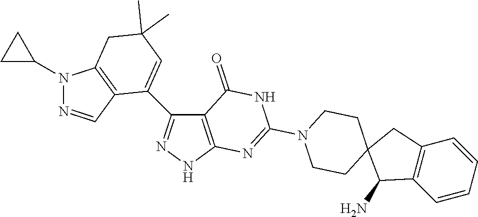 [SMILES: CC1(C)C=C(c2n[nH]c3nc(N4CCC5(CC4)Cc4ccccc4[C@H]5N)[nH]c(=O)c23)c2cnn(C3CC3)c2C1] | (S)-6-(1-amino-1,3- dihydrospiro[indene-2,4′-piperidin]- 1′-yl)-3-(1-cyclopropyl-6,6-dimethyl- 6,7-dihydro-1H-indazol-4-yl)-1,5- dihydro-4H-pyrazolo[3,4- d]pyrimidin-4-one | 523.29 |
| Example 153 (D046) | 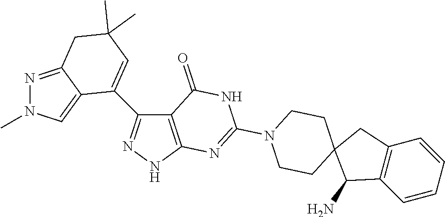 [SMILES: Cn1cc2c(n1)CC(C)(C)C=C2c1n[nH]c2nc(N3CCC4(CC3)Cc3ccccc3[C@H]4N)[nH]c(=O)c12] | (S)-6-(1-amino-1,3- dihydrospiro[indene-2,4′-piperidin]- 1′-yl)-3-(2,6,6-trimethyl-6,7-dihydro- 2H-indazol-4-yl)-1,5-dihydro-4H- pyrazolo[3,4-d]pyrimidin-4-one | 497.27 |
| Example 154 (D047) | 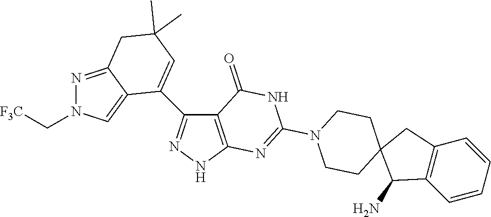 [SMILES: CC1(C)C=C(c2n[nH]c3nc(N4CCC5(CC4)Cc4ccccc4[C@H]5N)[nH]c(=O)c23)c2cn(CC(F)(F)F)nc2C1] | (S)-6-(1-amino-1,3- dihydrospiro[indene-2,4′-piperidin]- 1′-yl)-3-(6,6-dimethyl-2-(2,2,2- trifluoroethyl)-6,7-dihydro-2H- indazol-4-yl)-1,5-dihydro-4H- pyrazolo[3,4-d]pyrimidin-4-one | 565.26 |
| Example 155 (D048) | 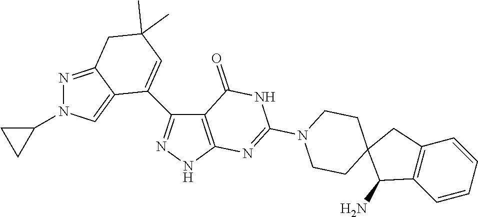 [SMILES: CC1(C)C=C(c2n[nH]c3nc(N4CCC5(CC4)Cc4ccccc4[C@H]5N)[nH]c(=O)c23)c2cn(C3CC3)nc2C1] | (S)-6-(1-amino-1,3- dihydrospiro[indene-2,4′-piperidin]- 1′-y1)-3-(2-cyclopropyl-6,6-dimethyl- 6,7-dihydro-2H-indazol-4-yl)-1,5- dihydro-4H-pyrazolo[3,4- d]pyrimidin-4-one | 523.29 |
| Example 156 (D049) | 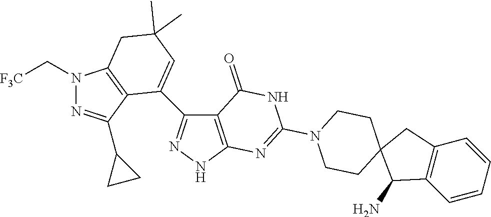 [SMILES: CC1(C)C=C(c2n[nH]c3nc(N4CCC5(CC4)Cc4ccccc4[C@H]5N)[nH]c(=O)c23)c2c(C3CC3)nn(CC(F)(F)F)c2C1] | (S)-6-(1-amino-1,3- dihydrospiro[indene-2,4′-piperidin]- 1′-y1)-3-(3-cyclopropyl-6,6-dimethyl- 1-(2,2,2-trifluoroethyl)-6,7-dihydro- 1H-indazol-4-yl)-1,5-dihydro-4H- pyrazolo[3,4-d]pyrimidin-4-one | 605.29 |
| Example 157 (D050) | 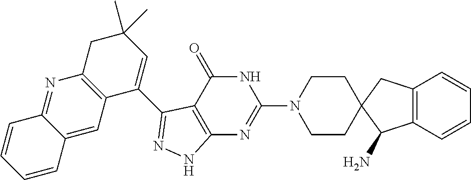 [SMILES: CC1(C)C=C(c2n[nH]c3nc(N4CCC5(CC4)Cc4ccccc4[C@H]5N)[nH]c(=O)c23)c2cc3ccccc3nc2C1] | (S)-6-(1-amino-1,3- dihydrospiro[indene-2,4′-piperidin]- 1′-yl)-3-(3,3-dimethyl-3,4- dihydroacridin-1-yl)-1,5-dihydro-4H- pyrazolo[3,4-d]pyrimidin-4-one | 544.27 |
| Example 158 (D051) | 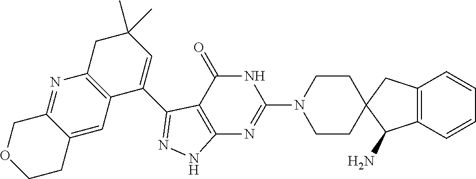 [SMILES: CC1(C)C=C(c2n[nH]c3nc(N4CCC5(CC4)Cc4ccccc4[C@H]5N)[nH]c(=O)c23)c2cc3c(nc2C1)COCC3] | (S)-6-(1-amino-1,3- dihydrospiro[indene-2,4′-piperidin]- 1′-yl)-3-(8,8-dimethyl-3,4,8,9- tetrahydro-1H-pyrano[3,4- b]quinolin-6-yl)-1,5-dihydro-4H- pyrazolo[3,4-d]pyrimidin-4-one | 550.29 |
| Example 159 (D052) | 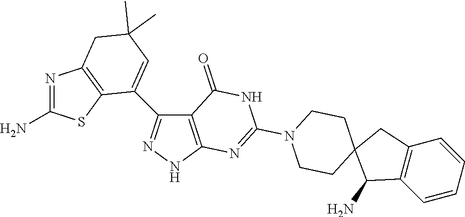 [SMILES: CC1(C)C=C(c2n[nH]c3nc(N4CCC5(CC4)Cc4ccccc4[C@H]5N)[nH]c(=O)c23)c2sc(N)nc2C1] | (S)-6-(1-amino-1,3- dihydrospiro[indene-2,4′-piperidin]- 1′-yl)-3-(2-amino-5,5-dimethyl-4,5- dihydrobenzo[d]thiazol-7-yl)-1,5- dihydro-4H-pyrazolo[3,4- d]pyrimidin-4-one | 515.23 |
| Example 160 (D053) | 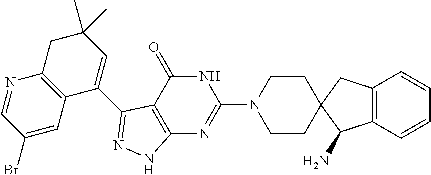 [SMILES: CC1(C)C=C(c2n[nH]c3nc(N4CCC5(CC4)Cc4ccccc4[C@H]5N)[nH]c(=O)c23)c2cc(Br)cnc2C1] | (S)-6-(1-amino-1,3- dihydrospiro[indene-2,4′-piperidin]- 1′-yl)-3-(3-bromo-7,7-dimethyl-7,8- dihydroquinolin-5-yl)-1,5-dihydro- 4H-pyrazolo[3,4-d]pyrimidin-4-one | 572.17 |
| Example 161 (D054) | 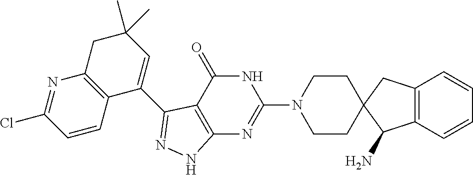 [SMILES: CC1(C)C=C(c2n[nH]c3nc(N4CCC5(CC4)Cc4ccccc4[C@H]5N)[nH]c(=O)c23)c2ccc(Cl)nc2C1] | (S)-6-(1-amino-1,3- dihydrospiro[indene-2,4′-piperidin]- 1′-yl)-3-(2-chloro-7,7-dimethyl-7,8- dihydroquinolin-5-yl)-1,5-dihydro- 4H-pyrazolo[3,4-d]pyrimidin-4-one | 528.22 |
| Example 162 (D055) | 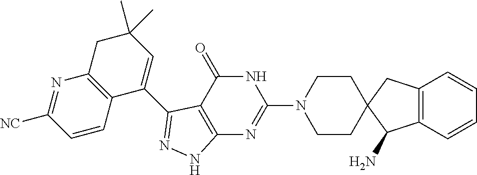 [SMILES: CC1(C)C=C(c2n[nH]c3nc(N4CCC5(CC4)Cc4ccccc4[C@H]5N)[nH]c(=O)c23)c2ccc(C#N)nc2C1] | (S)-5-(6-(1-amino-1,3- dihydrospiro[indene-2,4′-piperidin]- 1′-yl)-4-oxo-4,5-dihydro-1H- pyrazolo[3,4-d]pyrimidin-3-yl)-7,7- dimethyl-7,8-dihydroquinoline-2- carbonitrile | 519.25 |
| Example 163 (D056) | 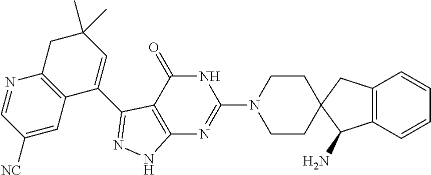 [SMILES: CC1(C)C=C(c2n[nH]c3nc(N4CCC5(CC4)Cc4ccccc4[C@H]5N)[nH]c(=O)c23)c2cc(C#N)cnc2C1] | (S)-5-(6-(1-amino-1,3- dihydrospiro[indene-2,4′-piperidin]- 1′-yl)-4-oxo-4,5-dihydro-1H- pyrazolo[3,4-d]pyrimidin-3-yl)-7,7- dimethyl-7,8-dihydroquinoline-3- carbonitrile | 519.25 |
| Example 164 (D057) | 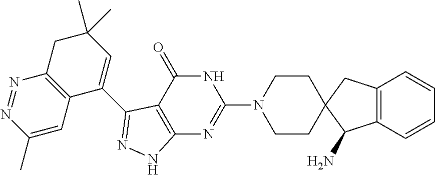 [SMILES: Cc1cc2c(nn1)CC(C)(C)C=C2c1n[nH]c2nc(N3CCC4(CC3)Cc3ccccc3[C@H]4N)[nH]c(=O)c12] | (S)-6-(1-amino-1,3- dihydrospiro[indene-2,4′-piperidin]- 1′-yl)-3-(3,7,7-trimethyl-7,8- dihydrocinnolin-5-yl)-1,5-dihydro- 4H-pyrazolo[3,4-d]pyrimidin-4-one | 509.27 |
| Example 165 (D058) | 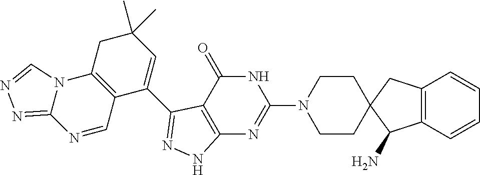 [SMILES: CC1(C)C=C(c2n[nH]c3nc(N4CCC5(CC4)Cc4ccccc4[C@H]5N)[nH]c(=O)c23)c2cnc3nncn3c2C1] | (S)-6-(1-amino-1,3- dihydrospiro[indene-2,4′-piperidin]- 1′-yl)-3-(8,8-dimethyl-8,9-dihydro- [1,2,4]triazolo[4,3-a]quinazolin-6- yl)-1,5-dihydro-4H-pyrazolo[3,4- d]pyrimidin-4-one | 535.26 |
| Example 166 (D059) | 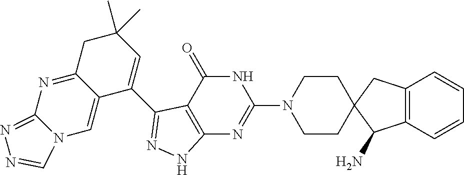 [SMILES: CC1(C)C=C(c2n[nH]c3nc(N4CCC5(CC4)Cc4ccccc4[C@H]5N)[nH]c(=O)c23)c2cn3cnnc3nc2C1] | (S)-6-(1-amino-1,3- dihydrospiro[indene-2,4′-piperidin]- 1′-yl)-3-(8,8-dimethyl-8,9-dihydro- [1,2,4]triazolo[3,4-b]quinazolin-6- yl)-1,5-dihydro-4H-pyrazolo[3,4- d\|pyrimidin-4-one | 535.26 |
| Example 167 (D060) | 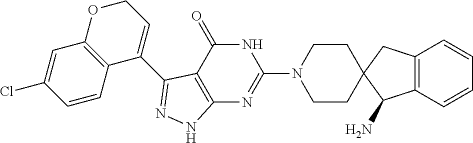 [SMILES: N[C@@H]1c2ccccc2CC12CCN(c1nc3[nH]nc(C4=CCOc5cc(Cl)ccc54)c3c(=O)[nH]1)CC2] | (S)-6-(1-amino-1,3- dihydrospiro[indene-2,4′-piperidin]- 1′-yl)-3-(7-chloro-2H-chromen-4-yl)- 1,5-dihydro-4H-pyrazolo[3,4- d]pyrimidin-4-one | 501.17 |
| Example 168 (D061) | 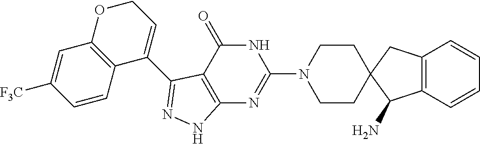 [SMILES: N[C@@H]1c2ccccc2CC12CCN(c1nc3[nH]nc(C4=CCOc5cc(C(F)(F)F)ccc54)c3c(=O)[nH]1)CC2] | (S)-6-(1-amino-1,3- dihydrospiro[indene-2,4′-piperidin]- 1′-yl)-3-(7-(trifluoromethyl)-2H- chromen-4-yl)-1,5-dihydro-4H- pyrazolo[3,4-d]pyrimidin-4-one | 535.20 |
| Example 169 (D062) | 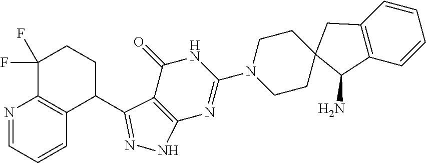 [SMILES: N[C@@H]1c2ccccc2CC12CCN(c1nc3[nH]nc(C4CCC(F)(F)c5ncccc54)c3c(=O)[nH]1)CC2] | (S)-6-(1-amino-1,3- dihydrospiro[indene-2,4′-piperidin]- 1′-yl)-3-(8,8-difluoro-7,8- dihydroquinolin-5-yl)-1,5-dihydro- 4H-pyrazolo[3,4-d]pyrimidin-4-one | 502.21 |
| Example 170) (D063) | 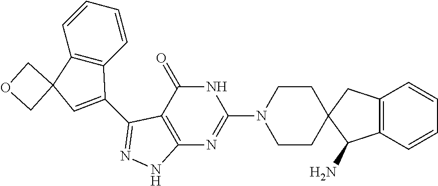 [SMILES: N[C@@H]1c2ccccc2CC12CCN(c1nc3[nH]nc(C4=CC5(COC5)c5ccccc54)c3c(=O)[nH]1)CC2] | (S)-6-(1-amino-1,3- dihydrospiro[indene-2,4′-piperidin]- 1′-yl)-3-(spiro[indene-1,3′-oxetan]-3- yl)-1,5-dihydro-4H-pyrazolo[3,4- d]pyrimidin-4-one | 493.23 |
| Example 171 (D064) | 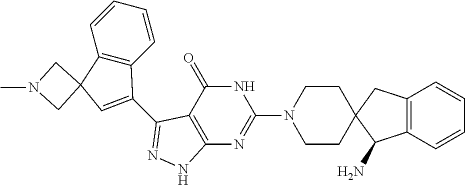 [SMILES: CN1CC2(C=C(c3n[nH]c4nc(N5CCC6(CC5)Cc5ccccc5[C@H]6N)[nH]c(=O)c34)c3ccccc32)C1] | (S)-6-(1-amino-1,3- dihydrospiro[indene-2,4′-piperidin]- 1′-yl)-3-(1-methylspiro[azetidine- 3,1′-inden]-3′-yl)-1,5-dihydro-4H- pyrazolo[3,4-d]pyrimidin-4-one | 506.26 |
| Example 172 (D065) | 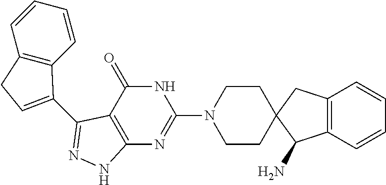 [SMILES: N[C@@H]1c2ccccc2CC12CCN(c1nc3[nH]nc(C4=CCc5ccccc54)c3c(=O)[nH]1)CC2] | (S)-6-(1-amino-1,3- dihydrospiro[indene-2,4′-piperidin]- 1′-yl)-3-(1H-inden-3-yl)-1,5-dihydro- 4H-pyrazolo[3,4-d]pyrimidin-4-one | 451.22 |
| Example 173 (D066) | 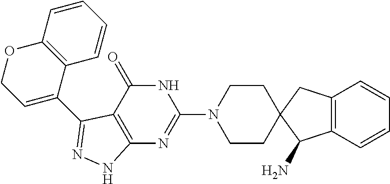 [SMILES: N[C@@H]1c2ccccc2CC12CCN(c1nc3[nH]nc(C4=CCOc5ccccc54)c3c(=O)[nH]1)CC2] | (S)-6-(1-amino-1,3- dihydrospiro[indene-2,4′-piperidin]- 1′-yl)-3-(2H-chromen-4-yl)-1,5- dihydro-4H-pyrazolo[3,4- d]pyrimidin-4-one | 467.21 |
| Example 174 (D067) | 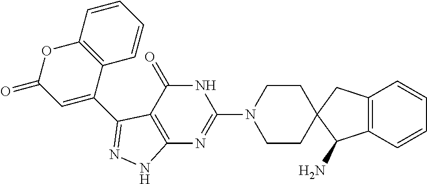 [SMILES: N[C@@H]1c2ccccc2CC12CCN(c1nc3[nH]nc(-c4cc(=O)oc5ccccc45)c3c(=O)[nH]1)CC2] | (S)-6-(1-amino-1,3- dihydrospiro[indene-2,4′-piperidin]- 1′-yl)-3-(2-oxo-2H-chromen-4-yl)- 1,5-dihydro-4H-pyrazolo[3,4- d]pyrimidin-4-one | 481.19 |
| Example 175 (D068) | 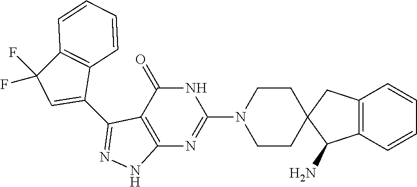 [SMILES: N[C@@H]1c2ccccc2CC12CCN(c1nc3[nH]nc(C4=CC(F)(F)c5ccccc54)c3c(=O)[nH]1)CC2] | (S)-6-(1-amino-1,3- dihydrospiro[indene-2,4′-piperidin]- 1′-yl)-3-(1,1-difluoro-1H-inden-3-yl)- 1,5-dihydro-4H-pyrazolo[3,4- d]pyrimidin-4-one | 487.20 |
| Example 176 (D069) | 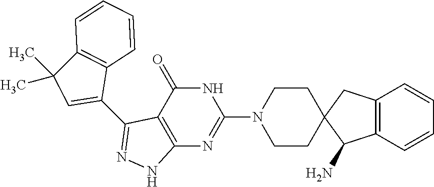 [SMILES: CC1(C)C=C(c2n[nH]c3nc(N4CCC5(CC4)Cc4ccccc4[C@H]5N)[nH]c(=O)c23)c2ccccc21] | (S)-6-(1-amino-1,3- dihydrospiro[indene-2,4′-piperidin]- 1′-yl)-3-(1,1-dimethyl-1H-inden-3- yl)-1,5-dihydro-4H-pyrazolo[3,4- d]pyrimidin-4-one | 479.25 |
| Example 177 (D070) | 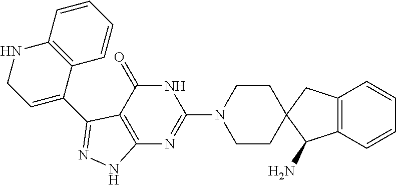 [SMILES: N[C@@H]1c2ccccc2CC12CCN(c1nc3[nH]nc(C4=CCNc5ccccc54)c3c(=O)[nH]1)CC2] | (S)-6-(1-amino-1,3- dihydrospiro[indene-2,4′-piperidin]- 1′-yl)-3-(1,2-dihydroquinolin-4-yl)- 1,5-dihydro-4H-pyrazolo[3,4- d]pyrimidin-4-one | 466.23 |
| Example 178 (D071) | 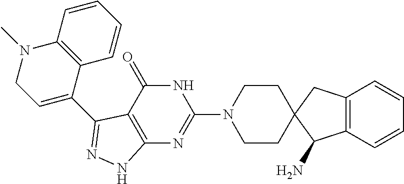 [SMILES: CN1CC=C(c2n[nH]c3nc(N4CCC5(CC4)Cc4ccccc4[C@H]5N)[nH]c(=O)c23)c2ccccc21] | (S)-6-(1-amino-1,3- dihydrospiro[indene-2,4′-piperidin]- 1′-yl)-3-(1-methyl-1,2- dihydroquinolin-4-yl)-1,5-dihydro- 4H-pyrazolo[3,4-d]pyrimidin-4-one | 480.24 |
| Example 179 (D072) | 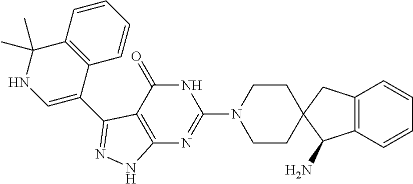 [SMILES: CC1(C)NC=C(c2n[nH]c3nc(N4CCC5(CC4)Cc4ccccc4[C@H]5N)[nH]c(=O)c23)c2ccccc21] | (S)-6-(1-amino-1,3- dihydrospiro[indene-2,4′-piperidin]- 1′-yl)-3-(1,1-dimethyl-1,2- dihydroisoquinolin-4-yl)-1,5- dihydro-4H-pyrazolo[3,4- d]pyrimidin-4-one | 494.26 |
| Example 180 (D073) | 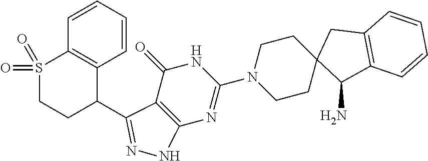 [SMILES: N[C@@H]1c2ccccc2CC12CCN(c1nc3[nH]nc(C4CCS(=O)(=O)c5ccccc54)c3c(=O)[nH]1)CC2] | (S)-6-(1-amino-1,3- dihydrospiro[indene-2,4′-piperidin]- 1′-yl)-3-(1,1-dioxido-2H- thiochromen-4-yl)-1,5-dihydro-4H- pyrazolo[3,4-d]pyrimidin-4-one | 515.18 |
| Example 181 (D074) | 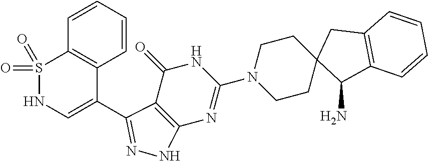 [SMILES: N[C@@H]1c2ccccc2CC12CCN(c1nc3[nH]nc(C4=CNS(=O)(=O)c5ccccc54)c3c(=O)[nH]1)CC2] | (S)-6-(1-amino-1,3- dihydrospiro[indene-2,4′-piperidin]- 1′-yl)-3-(1,1-dioxido-2H- benzo[e][1,2]thiazin-4-yl)-1,5- dihydro-4H-pyrazolo[3,4- d]pyrimidin-4-one | 516.17 |
| Example 182 (D075) | 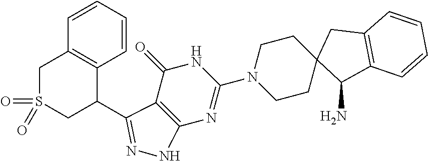 [SMILES: N[C@@H]1c2ccccc2CC12CCN(c1nc3[nH]nc(C4CS(=O)(=O)Cc5ccccc54)c3c(=O)[nH]1)CC2] | (S)-6-(1-amino-1,3- dihydrospiro[indene-2,4′-piperidin]- 1′-yl)-3-(2,2-dioxido-1H- isothiochromen-4-yl)-1,5-dihydro- 4H-pyrazolo[3,4-d]pyrimidin-4-one | 515.18 |
| Example 183 (D076) | 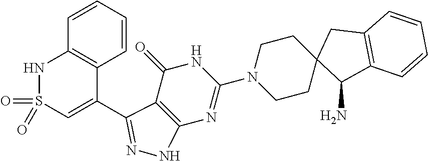 [SMILES: N[C@@H]1c2ccccc2CC12CCN(c1nc3[nH]nc(C4=CS(=O)(=O)Nc5ccccc54)c3c(=O)[nH]1)CC2] | (S)-6-(1-amino-1,3- dihydrospiro[indene-2,4′-piperidin]- 1′-yl)-3-(2,2-dioxido-1H- benzo[c][1,2]thiazin-4-yl)-1,5- dihydro-4H-pyrazolo[3,4- d]pyrimidin-4-one | 516.17 |
| Example 184 (D077) | 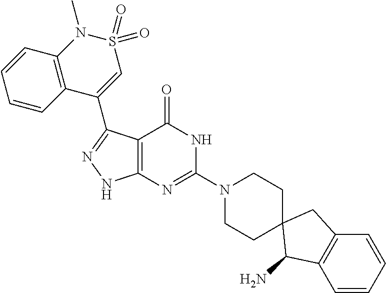 [SMILES: CN1c2ccccc2C(c2n[nH]c3nc(N4CCC5(CC4)Cc4ccccc4[C@H]5N)[nH]c(=O)c23)=CS1(=O)=O] | (S)-6-(1-amino-1,3- dihydrospiro[indene-2,4′-piperidin]- 1′-yl)-3-(1-methyl-2,2-dioxido-1H- benzo[c][1,2]thiazin-4-yl)-1,5- dihydro-4H-pyrazolo[3,4- d]pyrimidin-4-one | 530.19 |
| Example 186 (D078) | 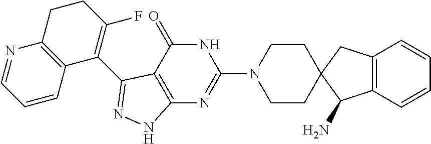 [SMILES: N[C@@H]1c2ccccc2CC12CCN(c1nc3[nH]nc(C4=C(F)CCc5ncccc54)c3c(=O)[nH]1)CC2] | (S)-6-(1-amino-1,3- dihydrospiro[indene-2,4′-piperidin]- 1′-yl)-3-(6-fluoro-7,8- dihydroquinolin-5-yl)-1,5-dihydro- 4H-pyrazolo[3,4-d]pyrimidin-4-one | 484.22 |
| Example 187 (D079) | 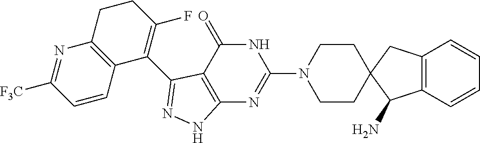 [SMILES: N[C@@H]1c2ccccc2CC12CCN(c1nc3[nH]nc(C4=C(F)CCc5nc(C(F)(F)F)ccc54)c3c(=O)[nH]1)CC2] | (S)-6-(1-amino-1,3- dihydrospiro[indene-2,4′-piperidin]- 1′-yl)-3-(6-fluoro-2- (trifluoromethyl)-7,8- dihydroquinolin-5-yl)-1,5-dihydro- 4H-pyrazolo[3,4-d]pyrimidin-4-one | 552.21 |
| Example 188 (D080) | 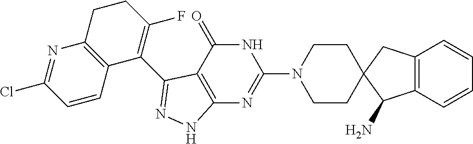 [SMILES: N[C@@H]1c2ccccc2CC12CCN(c1nc3[nH]nc(C4=C(F)CCc5nc(Cl)ccc54)c3c(=O)[nH]1)CC2] | (S)-6-(1-amino-1,3- dihydrospiro[indene-2,4′-piperidin]- 1′-yl)-3-(2-chloro-6-fluoro-7,8- dihydroquinolin-5-yl)-1,5-dihydro- 4H-pyrazolo[3,4-d]pyrimidin-4-one | 518.18 |
| Example 189 (D081) | 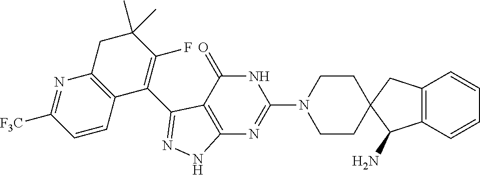 [SMILES: CC1(C)Cc2nc(C(F)(F)F)ccc2C(c2n[nH]c3nc(N4CCC5(CC4)Cc4ccccc4[C@H]5N)[nH]c(=O)c23)=C1F] | (S)-6-(1-amino-1,3- dihydrospiro[indene-2,4′-piperidin]- 1′-yl)-3-(6-fluoro-7,7-dimethyl-2- (trifluoromethyl)-7,8- dihydroquinolin-5-yl)-1,5-dihydro- 4H-pyrazolo[3,4-d]pyrimidin-4-one | 580.24 |
| Example 190 (D082) | 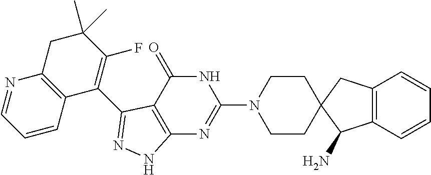 [SMILES: CC1(C)Cc2ncccc2C(c2n[nH]c3nc(N4CCC5(CC4)Cc4ccccc4[C@H]5N)[nH]c(=O)c23)=C1F] | (S)-6-(1-amino-1,3- dihydrospiro[indene-2,4′-piperidin]- 1′-yl)-3-(6-fluoro-7,7-dimethyl-7,8- dihydroquinolin-5-yl)-1,5-dihydro- 4H-pyrazolo[3,4-d]pyrimidin-4-one | 511.61 |
| Example 191 (D083) | 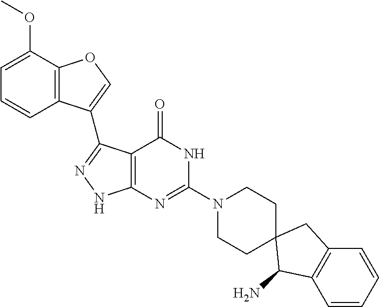 [SMILES: COc1cccc2c(-c3n[nH]c4nc(N5CCC6(CC5)Cc5ccccc5[C@H]6N)[nH]c(=O)c34)coc12] | (S)-6-(1-amino-1,3- dihydrospiro[indene-2,4′-piperidin]- 1′-yl)-3-(7-methoxybenzofuran-3-yl)- 1,5-dihydro-4H-pyrazolo[3,4- d]pyrimidin-4-one | 483.21 |

### Example 259 Preparation of Compound 259

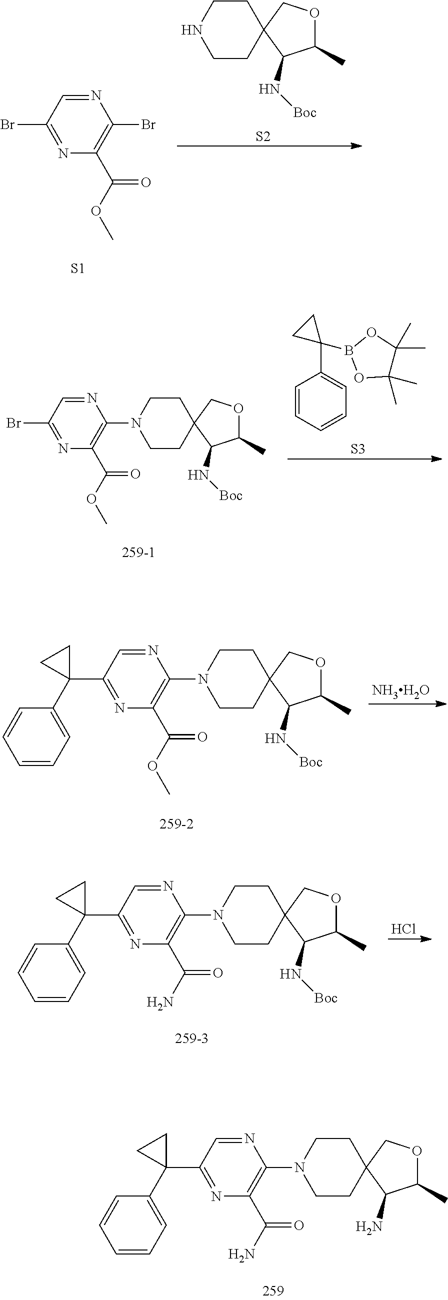

### Step 1: Preparation of Compound 259-1

To a solution of S1 (2.96 g) and S2 (3.24 g) in DMF (50 mL) was added TEA (2.02 g). The mixture was stirred at 80° C. for 5 hours. The mixture was cooled down to room temperature and poured into ice water (200 mL) with stirring, the solid was collected by filtration and washed with water (100 mL), which was dried to give crude product compound 259-1 (3.55 g) as yellow solid.

### Step 2: Preparation of Compound 259-2

To a 250 mL dried flask was added dioxane (100 mL) and water (30 mL), followed by adding Compound 259-1 (2.2 g), 4,4,5,5-tetramethyl-2-(1-phenylcyclopropyl)-1,3,2-dioxaborolane (2.44 g), PddppfCl₂ (70 mg) and K₂CO₃ (1.4 g). The mixture was stirred at 100° C. for 3 hours. When cooled to room temperature, the mixture was diluted with EA (300 mL) and washed with saturated brine (100 mL*2). The organic layer was concentrated and purified by Flash column (PE/EA=1/1) to afford product compound 259-2 (1.98 g, 76%) as light yellow solid.

### Step 3: Preparation of Compound 259-3

To a solution of Compound 259-2 (0.52 g) in methanol (10 mL) was added NH₃·H₂O (30 mL). The mixture was stirred at 50° C. for 4 hours. Removed the solvent and the residue was purified by Flash column (DCM/MeOH=10/1) to afford product Compound 259-3 (0.31 g, 62%) as light yellow solid.

### Step 4: Preparation of Compound 259

To a solution of Compound 259-3 (0.25 g) in DCM (5 mL) was added a solution of hydrochloric in dioxane (4M)(1 mL). The mixture was stirred at room temperature for 3 hours. To the mixture was added saturated solution of NaHCO₃ (50 mL) and textured with DCM (50 mL*3). The organic layers was concentrated to give product Compound 259 (0.18 g, 90%) as white solid.

### Example 260 Preparation of Compound 260

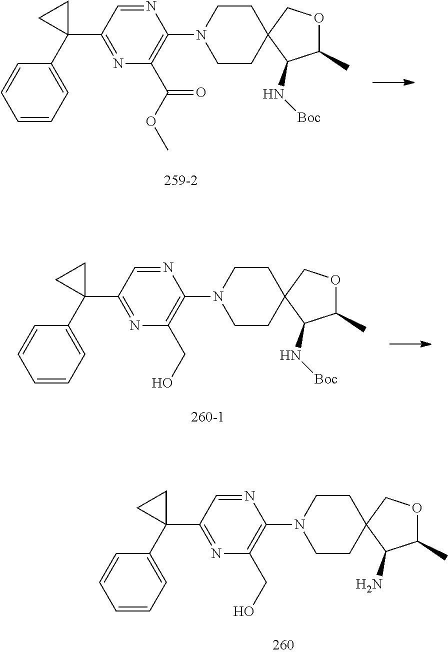 [SMILES: COC(=O)c1nc(C2(c3ccccc3)CC2)cnc1N1CCC2(CC1)CO[C@@H](C)[C@H]2NC(=O)OC(C)(C)C ; C[C@@H]1OCC2(CCN(c3ncc(C4(c5ccccc5)CC4)nc3CO)CC2)[C@@H]1NC(=O)OC(C)(C)C ; C[C@@H]1OCC2(CCN(c3ncc(C4(c5ccccc5)CC4)nc3CO)CC2)[C@@H]1N]

### Step 1: Preparation of Compound 260-1

To a solution of compound 259-2 (0.52 g) in THF (10 mL) was added DIBALH (2 mL) slowly at −20° C. The mixture was stirred at room temperature for 1.5 hours. The reaction mixture was quenched by dropping diluted hydrochloric acid (2M) and extracted with DCM (50 mL*3). The organic layer was concentrated and purified by Flash column (PE/EA=1/1) to afford product compound 260-1 (0.35 g, 71%) as colorless oil.

### Step 2: Preparation of Compound 260

To a solution of compound 260-1 (0.25 g) in DCM (5 mL) was added a solution of hydrochloric in dioxane (4M)(1 mL). The mixture was stirred at room temperature for 3 hours. To the mixture was added saturated solution of NaHCO₃ (50 mL) and extracted with DCM (50 mL*3). The organic layers was concentrated to give product compound 260 (0.16 g, 81%) as colorless oil.

### Example 264 Preparation of Compound 264

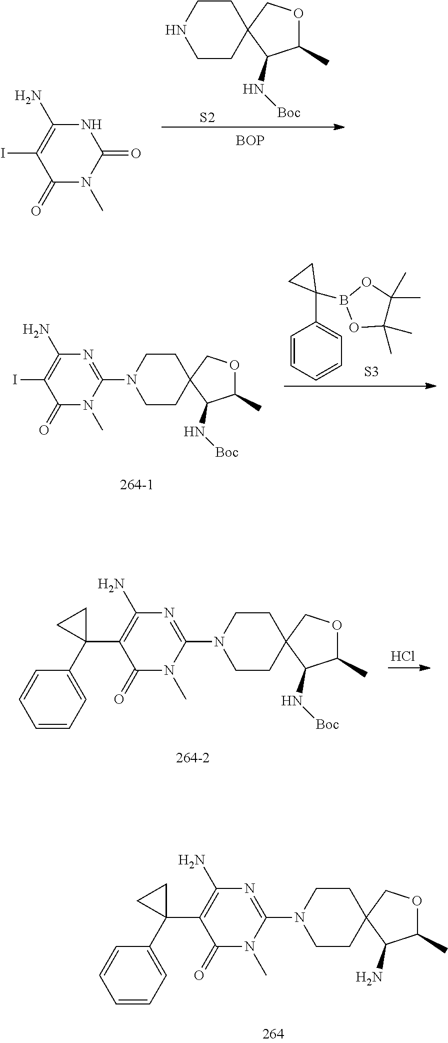

### Step 1: Preparation of Compound 264-1

A mixture of 6-amino-5-iodo-3-methylpyrimidine-2,4(1H,3H)-dione (2.67 g), tert-butyl ((3S,4S)-3-methyl-2-oxa-8-azaspiro[4.5]decan-4-yl)carbamate (3.25 g), BOP (8.9 g), and DBU (7.6 g) in DMF (30 mL) was stirred 2 hours at room temperature. The reaction mixture was poured into ice-water (100 mL) and extracted with DCM (100 mL*3), the organic phases were combined and concentrated, the residue was purified by flash column (DCM/MeOH=40/1) to give compound 264-1 (4.6 g, 88%) as light yellow solid.

### Step 2: Preparation of Compound 264-2

To a 250 mL dried flask was added dioxane (100 mL) and water (30 mL), followed by adding compound 264-1 (2.6 g), 4,4,5,5-tetramethyl-2-(1-phenylcyclopropyl)-1,3,2-dioxaborolane (2.44 g), PddppfCl₂ (70 mg) and K₂CO₃ (1.4 g). The mixture was stirred at 100° C. for 3 hours at N₂ atmosphere. When cooled to room temperature, the mixture was diluted with EA (300 mL) and washed with saturated brine (100 mL*2). The organic layer was concentrated and purified by Flash column (DCM/MeOH=40/1) to afford product compound 264-2 (2.0 g, 80%) as light yellow solid.

### Step 3: Preparation of Compound 264

To a solution of compound 264-2 (0.5 g) in DCM (5 mL) was added a solution of hydrochloric in dioxane (4M)(1 mL). The mixture was stirred at room temperature for 3 hours. To the mixture was added saturated solution of NaHCO₃ (50 mL) and extracted with DCM (50 mL*3). The organic layers was concentrated to give product compound 264 (0.32 g, 80%) as light yellow solid.

### Example 268 Preparation of Compound 268

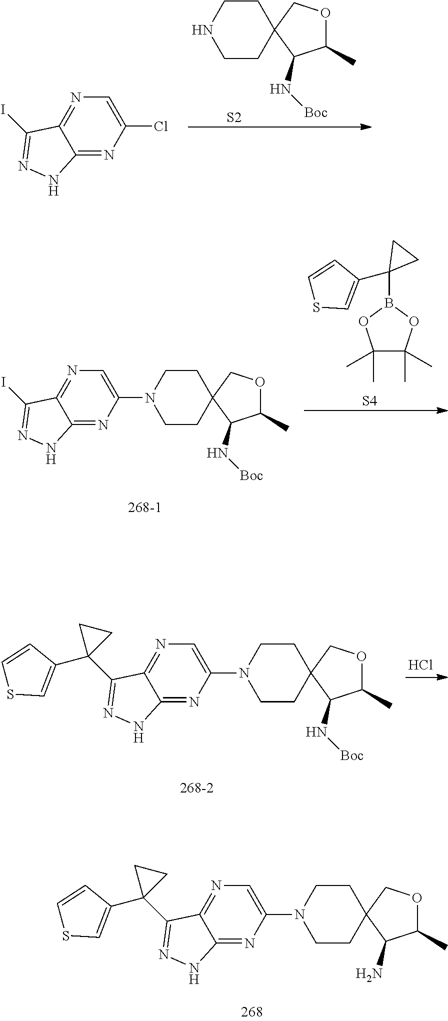

### Step 1: Preparation of Compound 268-1

To a solution of 6-chloro-3-iodo-1H-pyrazolo[3,4-b]pyrazine (2.8 g) and S2 (3.2 g) in DMF (50 mL) was added TEA (2.02 g). The mixture was stirred at 80° C. for 5 hours. The mixture was cooled down to room temperature and poured into ice water (200 mL) with stirring, the solid was collected by filtration and washed with water (100 mL), which was dried to give crude product compound 268-1 (4.5 g) as yellow solid.

### Step 2: Preparation of Compound 268-2

To a 250 mL dried flask was added dioxane (100 mL) and water (30 mL), followed by adding compound 268-1 (2.5 g), 4,4,5,5-tetramethyl-2-(1-(thiophen-3-yl)cyclopropyl)-1,3,2-dioxaborolane (2.5 g), PddppfCl₂ (70 mg) and K₂CO₃ (1.4 g). The mixture was stirred at 100° C. for 3 hours at N₂ atmosphere. When cooled to room temperature, the mixture was diluted with EA (300 mL) and washed with saturated brine (100 mL*2). The organic layer was concentrated and purified by Flash column (DCM/MeOH=40/1) to afford product compound 268-2 (1.8 g, 70%) as yellow solid.

### Step 3: Preparation of Compound 268

To a solution of compound 268-2 (0.5 g) in DCM (5 mL) was added a solution of hydrochloric in dioxane (4M)(1 mL). The mixture was stirred at room temperature for 3 hours. To the mixture was added saturated solution of NaHCO₃ (50 mL) and extracted with DCM (50 mL*3). The organic layers was concentrated to give product compound 268 (0.36 g, 90%) as yellow solid.

### Example 282 Preparation of Compound 282

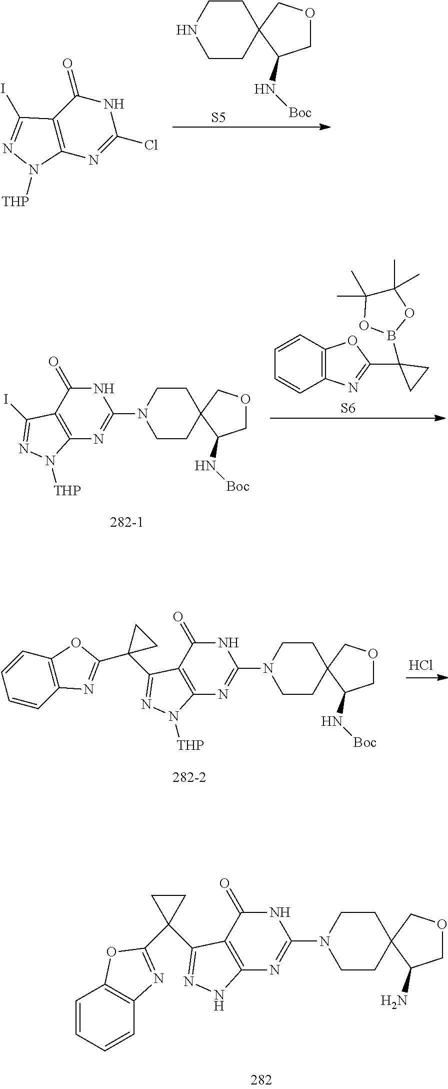

### Step 1: Preparation of Compound 282-1

To a solution of 6-chloro-3-iodo-1-(tetrahydro-2H-pyran-2-yl)-1H-pyrazolo[3,4-d]pyrimidin-4(5H)-one (3.8 g) and (S)-tert-butyl 2-oxa-8-azaspiro[4.5]decan-4-ylcarbamate S5 (3.1 g) in DMF (50 mL) was added TEA (2.02 g). The mixture was stirred at 80° C. for 5 hours. The mixture was cooled down to room temperature and poured into ice water (200 mL) with stirring, the solid was collected by filtration and washed with water (100 mL), which was dried to give crude product compound 282-1 (5.5 g) as yellow solid.

### Step 2: Preparation of Compound 282-2

To a 250 mL flask was added dioxane (100 mL) and water (30 mL), followed by adding compound 282-1 (3.0 g), compound S6 (2.85 g. 10 mmoL), PddppfCl₂ (70 mg, 0.1 mmoL) and K₂CO₃ (1.4 g). The mixture was stirred at 100° C. for 3 hours at N₂ atmosphere. When cooled to room temperature, the mixture was diluted with EA (300 mL) and washed with saturated brine (100 mL*2). The organic layer was concentrated and purified by Flash column (DCM/MeOH=40/1) to afford product compound 282-2 (2.3 g, 70%) as yellow solid.

### Step 3: Preparation of Compound 282

To a solution of compound 282-2 (0.63 g) in DCM (5 mL) was added a solution of hydrochloric in dioxane (4M)(1 mL). The mixture was stirred at room temperature for 3 hours. To the mixture was added saturated solution of NaHCO₃ (50 mL) and extracted with DCM (50 mL*3). The organic layers was concentrated to give product Compound 282 (0.33 g, 75%) as yellow solid.

The following compounds showing in Table 4 were synthesized using the above procedures of Compound 259, Compound 260, Compound 264, Compound 268, Compound 282, or modified procedure with the corresponding starting materials.

TABLE 4

| | | | Physical |
|---|---|---|---|
| | | | Data (MS) |
| Example | Structure | Chemical Name | (M + H)⁺ |
| Example 258 | 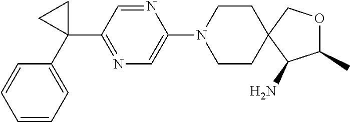 [SMILES: C[C@@H]1OCC2(CCN(c3cnc(C4(c5ccccc5)CC4)cn3)CC2)[C@@H]1N] | (3S,4S)-3-methyl-8-(5-(1- phenylcyclopropyl)pyrazin-2-yl)-2- oxa-8-azaspiro[4.5]decan-4-amine | 364.48 |
| Example 259 | 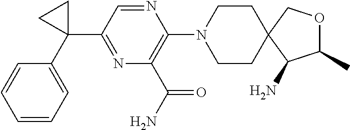 [SMILES: C[C@@H]1OCC2(CCN(c3ncc(C4(c5ccccc5)CC4)nc3C(N)=O)CC2)[C@@H]1N] | 3-((3S,4S)-4-amino-3-methyl-2-oxa- 8-azaspiro[4.5]decan-8-yl)-6-(1- phenylcyclopropyl)pyrazine-2- carboxamide | 407.51 |
| Example 260 | 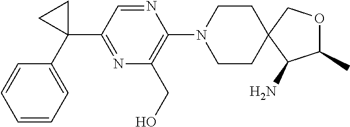 [SMILES: C[C@@H]1OCC2(CCN(c3ncc(C4(c5ccccc5)CC4)nc3CO)CC2)[C@@H]1N] | (3-((3S,4S)-4-amino-3-methyl-2-oxa- 8-azaspiro[4.5]decan-8-yl)-6-(1- phenylcyclopropyl)pyrazin-2- yl)methanol | 394.51 |
| Example 261 | 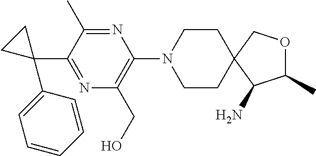 [SMILES: Cc1nc(N2CCC3(CC2)CO[C@@H](C)[C@H]3N)c(CO)nc1C1(c2ccccc2)CC1] | (3-((3S,4S)-4-amino-3-methyl-2-oxa- 8-azaspiro[4.5]decan-8-yl)-5-methyl- 6-(1-phenylcyclopropyl)pyrazin-2- yl)methanol | 408.55 |
| Example 262 | 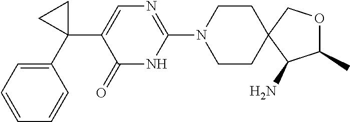 [SMILES: C[C@@H]1OCC2(CCN(c3ncc(C4(c5ccccc5)CC4)c(=O)[nH]3)CC2)[C@@H]1N] | 2-((3S,4S)-4-amino-3-methyl-2-oxa- 8-azaspiro[4.5]decan-8-yl)-5-(1- phenylcyclopropyl)pyrimidin-4(3H)- one | 380.48 |
| Example 263 | 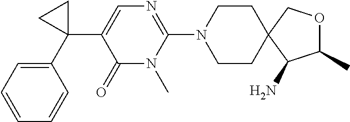 [SMILES: C[C@@H]1OCC2(CCN(c3ncc(C4(c5ccccc5)CC4)c(=O)n3C)CC2)[C@@H]1N] | 2-((3S,4S)-4-amino-3-methyl-2-oxa- 8-azaspiro[4.5]decan-8-yl)-3-methyl- 5-(1-phenylcyclopropyl)pyrimidin- 4(3H)-one | 394.51 |
| Example 264 | 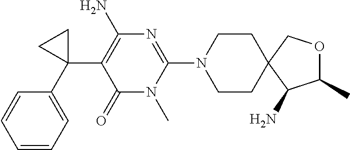 [SMILES: C[C@@H]1OCC2(CCN(c3nc(N)c(C4(c5ccccc5)CC4)c(=O)n3C)CC2)[C@@H]1N] | 6-amino-2-((3S,4S)-4-amino-3- methyl-2-oxa-8-azaspiro[4.5]decan- 8-yl)-3-methyl-5-(1- phenylcyclopropyl)pyrimidin-4(3H)- one | 409.52 |
| Example 265 | 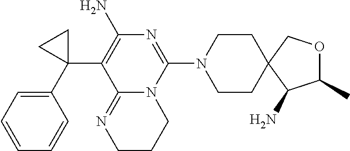 [SMILES: C[C@@H]1OCC2(CCN(C3=NC(N)=C(C4(c5ccccc5)CC4)C4=NCCCN43)CC2)[C@@H]1N] | (3S,4S)-8-(8-amino-9-(1- phenylcyclopropyl)-3,4-dihydro-2H- pyrimido[1,6-a]pyrimidin-6-yl)-3- methyl-2-oxa-8-azaspiro[4.5]decan- 4-amine | 434.58 |
| Example 266 | 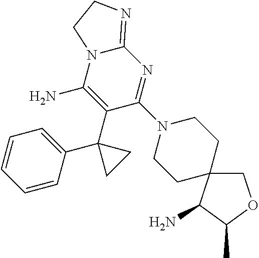 [SMILES: C[C@@H]1OCC2(CCN(C3=NC4=NCCN4C(N)=C3C3(c4ccccc4)CC3)CC2)[C@@H]1N] | (3S,4S)-8-(5-amino-6-(1- phenylcyclopropyl)-2,3- dihydroimidazo[1,2-a]pyrimidin-7- yl)-3-methyl-2-oxa-8- azaspiro[4.5]decan-4-amine | 420.55 |
| Example 267 | 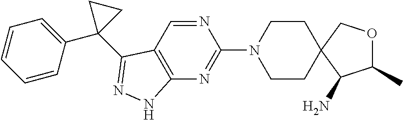 [SMILES: C[C@@H]1OCC2(CCN(c3ncc4c(C5(c6ccccc6)CC5)n[nH]c4n3)CC2)[C@@H]1N] | (3S,4S)-3-methyl-8-(3-(1- phenylcyclopropyl)-1H-pyrazolo[3,4- d]pyrimidin-6-yl)-2-oxa-8- azaspiro[4.5]decan-4-amine | 404.51 |
| Example 268 | 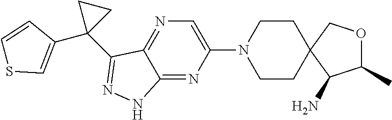 [SMILES: C[C@@H]1OCC2(CCN(c3cnc4c(C5(c6ccsc6)CC5)n[nH]c4n3)CC2)[C@@H]1N] | (3S,4S)-3-methyl-8-(3-(1-(thiophen- 3-yl)cyclopropyl)-1H-pyrazolo[3,4- b]pyrazin-6-yl)-2-oxa-8- azaspiro[4.5]decan-4-amine | 410.54 |
| Example 269 | 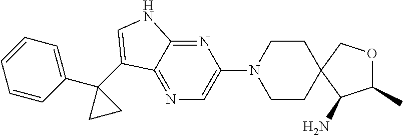 [SMILES: C[C@@H]1OCC2(CCN(c3cnc4c(C5(c6ccccc6)CC5)c[nH]c4n3)CC2)[C@@H]1N] | (3S,4S)-3-methyl-8-(7-(1- phenylcyclopropyl)-5H-pyrrolo[2,3- b]pyrazin-3-yl)-2-oxa-8- azaspiro[4.5]decan-4-amine | 403.52 |
| Example 270 | 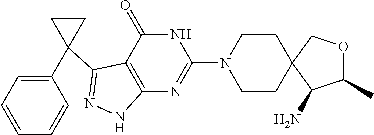 [SMILES: C[C@@H]1OCC2(CCN(c3nc4[nH]nc(C5(c6ccccc6)CC5)c4c(=O)[nH]3)CC2)[C@@H]1N] | 6-((3S,4S)-4-amino-3-methyl-2-oxa- 8-azaspiro[4.5]decan-8-yl)-3-(1- phenylcyclopropyl)-1,5-dihydro-4H- pyrazolo[3,4-d]pyrimidin-4-one | 420.51 |
| Example 271 | 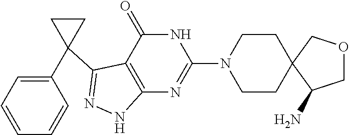 [SMILES: N[C@@H]1COCC12CCN(c1nc3[nH]nc(C4(c5ccccc5)CC4)c3c(=O)[nH]1)CC2] | (S)-6-(4-amino-2-oxa-8- azaspiro[4.5]decan-8-yl)-3-(1- phenylcyclopropyl)-1,5-dihydro-4H- pyrazolo[3,4-d]pyrimidin-4-one | 406.48 |
| Example 272 | 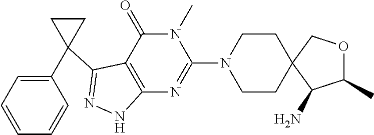 [SMILES: C[C@@H]1OCC2(CCN(c3nc4[nH]nc(C5(c6ccccc6)CC5)c4c(=O)n3C)CC2)[C@@H]1N] | 6-((3S,4S)-4-amino-3-methyl-2-oxa- 8-azaspiro[4.5]decan-8-yl)-5-methyl- 3-(1-phenylcyclopropyl)-1,5- dihydro-4H-pyrazolo[3,4- d]pyrimidin-4-one | 434.53 |
| Example 273 | 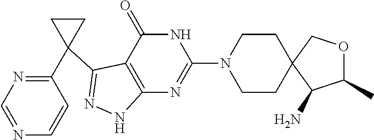 [SMILES: C[C@@H]1OCC2(CCN(c3nc4[nH]nc(C5(c6ccncn6)CC5)c4c(=O)[nH]3)CC2)[C@@H]1N] | 6-((3S,4S)-4-amino-3-methyl-2-oxa- 8-azaspiro[4.5]decan-8-yl)-3-(1- (pyrimidin-4-yl)cyclopropyl)-1,5- dihydro-4H-pyrazolo[3,4- d]pyrimidin-4-one | 422.48 |
| Example 274 | 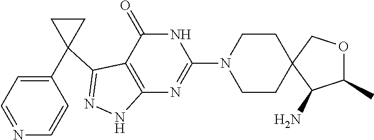 [SMILES: C[C@@H]1OCC2(CCN(c3nc4[nH]nc(C5(c6ccncc6)CC5)c4c(=O)[nH]3)CC2)[C@@H]1N] | 6-((3S,4S)-4-amino-3-methyl-2-oxa- 8-azaspiro[4.5]decan-8-yl)-3-(1- (pyridin-4-yl)cyclopropyl)-1,5- dihydro-4H-pyrazolo[3,4- d]pyrimidin-4-one | 421.50 |
| Example 275 | 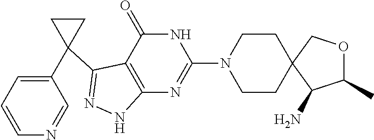 [SMILES: C[C@@H]1OCC2(CCN(c3nc4[nH]nc(C5(c6cccnc6)CC5)c4c(=O)[nH]3)CC2)[C@@H]1N] | 6-((3S,4S)-4-amino-3-methyl-2-oxa- 8-azaspiro[4.5]decan-8-yl)-3-(1- (pyridin-3-yl)cyclopropyl)-1,5- dihydro-4H-pyrazolo[3,4- d]pyrimidin-4-one | 421.50 |
| Example 276 | 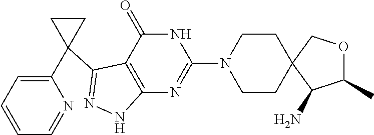 [SMILES: C[C@@H]1OCC2(CCN(c3nc4[nH]nc(C5(c6ccccn6)CC5)c4c(=O)[nH]3)CC2)[C@@H]1N] | 6-((3S,4S)-4-amino-3-methyl-2-oxa- 8-azaspiro[4.5]decan-8-yl)-3-(1- (pyridin-2-yl)cyclopropyl)-1,5- dihydro-4H-pyrazolo[3,4- d]pyrimidin-4-one | 421.50 |
| Example 277 | 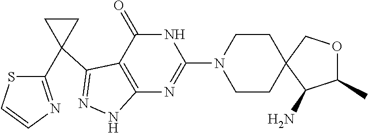 [SMILES: C[C@@H]1OCC2(CCN(c3nc4[nH]nc(C5(c6nccs6)CC5)c4c(=O)[nH]3)CC2)[C@@H]1N] | 6-((3S,4S)-4-amino-3-methyl-2-oxa- 8-azaspiro[4.5]decan-8-yl)-3-(1- (thiazol-2-yl)cyclopropyl)-1,5- dihydro-4H-pyrazolo[3,4- d]pyrimidin-4-one | 427.52 |
| Example 278 | 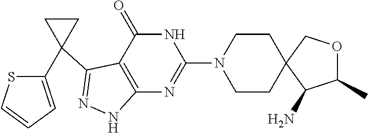 [SMILES: C[C@@H]1OCC2(CCN(c3nc4[nH]nc(C5(c6cccs6)CC5)c4c(=O)[nH]3)CC2)[C@@H]1N] | 6-((3S,4S)-4-amino-3-methyl-2-oxa- 8-azaspiro[4.5]decan-8-yl)-3-(1- (thiophen-2-yl)cyclopropyl)-1,5- dihydro-4H-pyrazolo[3,4- d]pyrimidin-4-one | 426.54 |
| Example 279 | 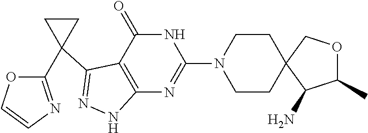 [SMILES: C[C@@H]1OCC2(CCN(c3nc4[nH]nc(C5(c6ncco6)CC5)c4c(=O)[nH]3)CC2)[C@@H]1N] | 6-((3S,4S)-4-amino-3-methyl-2-oxa- 8-azaspiro[4.5]decan-8-yl)-3-(1- (oxazol-2-yl)cyclopropyl)-1,5- dihydro-4H-pyrazolo[3,4- d]pyrimidin-4-one | 411.46 |
| Example 280 | 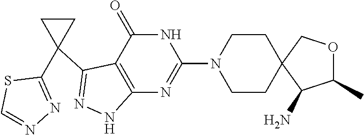 [SMILES: C[C@@H]1OCC2(CCN(c3nc4[nH]nc(C5(c6nncs6)CC5)c4c(=O)[nH]3)CC2)[C@@H]1N] | 3-(1-(1,3,4-thiadiazol-2- yl)cyclopropyl)-6-((3S,4S)-4-amino- 3-methyl-2-oxa-8- azaspiro[4.5]decan-8-yl)-1,5- dihydro-4H-pyrazolo[3,4- d]pyrimidin-4-one | 428.51 |
| Example 282 | 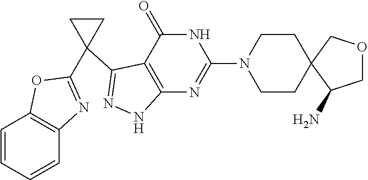 [SMILES: N[C@@H]1COCC12CCN(c1nc3[nH]nc(C4(c5nc6ccccc6o5)CC4)c3c(=O)[nH]1)CC2] | (S)-6-(4-amino-2-oxa-8- azaspiro[4.5]decan-8-yl)-3-(1- (benzo[d]oxazol-2-yl)cyclopropyl)- 1,5-dihydro-4H-pyrazolo[3,4- d]pyrimidin-4-one | 447.49 |
| Example 283 | 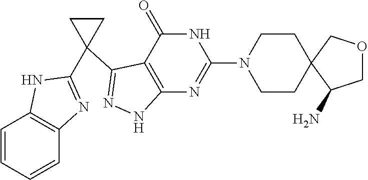 [SMILES: N[C@@H]1COCC12CCN(c1nc3[nH]nc(C4(c5nc6ccccc6[nH]5)CC4)c3c(=O)[nH]1)CC2] | (S)-3-(1-(1H-benzo[d]imidazol-2- yl)cyclopropyl)-6-(4-amino-2-oxa-8- azaspiro[4.5]decan-8-yl)-1,5- dihydro-4H-pyrazolo[3,4- d]pyrimidin-4-one | 446.50 |
| Example 284 | 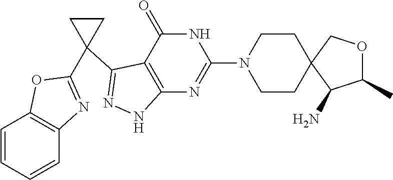 [SMILES: C[C@@H]1OCC2(CCN(c3nc4[nH]nc(C5(c6nc7ccccc7o6)CC5)c4c(=O)[nH]3)CC2)[C@@H]1N] | 6-((3S,4S)-4-amino-3-methyl-2-oxa- 8-azaspiro[4.5]decan-8-yl)-3-(1- (benzo[d]oxazol-2-yl)cyclopropyl)- 1,5-dihydro-4H-pyrazolo[3,4- d]pyrimidin-4-one | 461.52 |
| Example 285 | 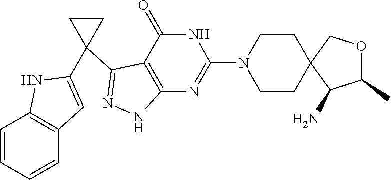 [SMILES: C[C@@H]1OCC2(CCN(c3nc4[nH]nc(C5(c6cc7ccccc7[nH]6)CC5)c4c(=O)[nH]3)CC2)[C@@H]1N] | 3-(1-(1H-indol-2-yl)cyclopropyl)-6- ((3S,4S)-4-amino-3-methyl-2-oxa-8- azaspiro[4.5]decan-8-yl)-1,5- dihydro-4H-pyrazolo[3,4- d]pyrimidin-4-one | 459.54 |
| Example 286 | 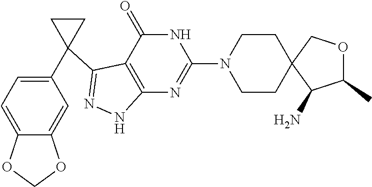 [SMILES: C[C@@H]1OCC2(CCN(c3nc4[nH]nc(C5(c6ccc7c(c6)OCO7)CC5)c4c(=O)[nH]3)CC2)[C@@H]1N] | 6-((3S,4S)-4-amino-3-methyl-2-oxa- 8-azaspiro[4.5]decan-8-yl)-3-(1- (benzo[d][1,3]dioxol-5- yl)cyclopropyl)-1,5-dihydro-4H- pyrazolo[3,4-d]pyrimidin-4-one | 464.52 |
| Example 287 | 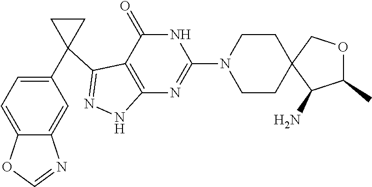 [SMILES: C[C@@H]1OCC2(CCN(c3nc4[nH]nc(C5(c6ccc7ocnc7c6)CC5)c4c(=O)[nH]3)CC2)[C@@H]1N] | 6-((3S,4S)-4-amino-3-methyl-2-oxa- 8-azaspiro[4.5]decan-8-yl)-3-(1- (benzo[d]oxazol-5-yl)cyclopropyl)- 1,5-dihydro-4H-pyrazolo[3,4- d]pyrimidin-4-one | 461.52 |
| Example 288 | 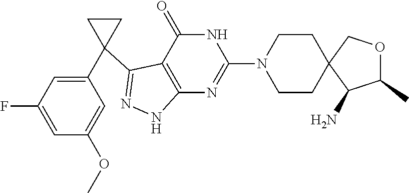 [SMILES: COc1cc(F)cc(C2(c3n[nH]c4nc(N5CCC6(CC5)CO[C@@H](C)[C@H]6N)[nH]c(=O)c34)CC2)c1] | 6-((3S,4S)-4-amino-3-methyl-2-oxa- 8-azaspiro[4.5]decan-8-yl)-3-(1-(3- fluoro-5- methoxyphenyl)cyclopropyl)-1,5- dihydro-4H-pyrazolo[3,4- d]pyrimidin-4-one | 468.52 |
| Example 289 | 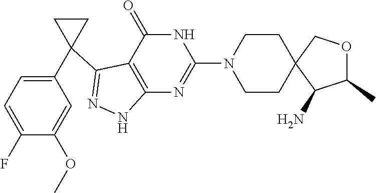 [SMILES: COc1cc(C2(c3n[nH]c4nc(N5CCC6(CC5)CO[C@@H](C)[C@H]6N)[nH]c(=O)c34)CC2)ccc1F] | 6-((3S,4S)-4-amino-3-methyl-2-oxa- 8-azaspiro[4.5]decan-8-yl)-3-(1-(4- fluoro-3- methoxyphenyl)cyclopropyl)-1,5- dihydro-4H-pyrazolo[3,4- d]pyrimidin-4-one | 468.52 |
| Example 290 | 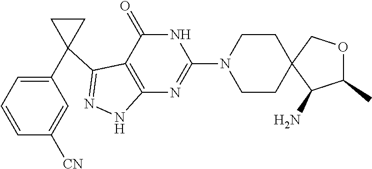 [SMILES: C[C@@H]1OCC2(CCN(c3nc4[nH]nc(C5(c6cccc(C#N)c6)CC5)c4c(=O)[nH]3)CC2)[C@@H]1N] | 3-(1-(6-((3S,4S)-4-amino-3-methyl- 2-oxa-8-azaspiro[4.5]decan-8-yl)-4- oxo-4,5-dihydro-1H-pyrazolo[3,4- d]pyrimidin-3- yl)cyclopropyl)benzonitrile | 445.52 |
| Example 291 | 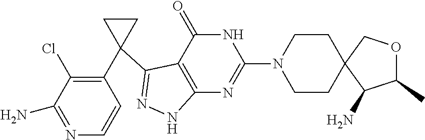 [SMILES: C[C@@H]1OCC2(CCN(c3nc4[nH]nc(C5(c6ccnc(N)c6Cl)CC5)c4c(=O)[nH]3)CC2)[C@@H]1N] | 3-(1-(2-amino-3-chloropyridin-4- yl)cyclopropyl)-6-((3S,4S)-4-amino- 3-methyl-2-oxa-8- azaspiro[4.5]decan-8-yl)-1,5- dihydro-4H-pyrazolo[3,4- d]pyrimidin-4-one | |
| Example 292 | 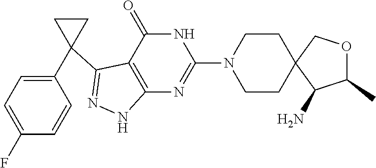 [SMILES: C[C@@H]1OCC2(CCN(c3nc4[nH]nc(C5(c6ccc(F)cc6)CC5)c4c(=O)[nH]3)CC2)[C@@H]1N] | 6-((3S,4S)-4-amino-3-methyl-2-oxa- 8-azaspiro[4.5]decan-8-yl)-3-(1-(4- fluorophenyl)cyclopropyl)-1,5- dihydro-4H-pyrazolo[3,4- d]pyrimidin-4-one | 438.50 |
| Example 293 | 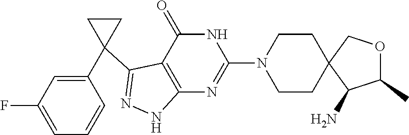 [SMILES: C[C@@H]1OCC2(CCN(c3nc4[nH]nc(C5(c6cccc(F)c6)CC5)c4c(=O)[nH]3)CC2)[C@@H]1N] | 6-((3S,4S)-4-amino-3-methyl-2-oxa- 8-azaspiro[4.5]decan-8-yl)-3-(1-(3- fluorophenyl)cyclopropyl)-1,5- dihydro-4H-pyrazolo[3,4- d]pyrimidin-4-one | 438.50 |
| Example 294 | 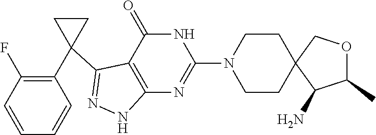 [SMILES: C[C@@H]1OCC2(CCN(c3nc4[nH]nc(C5(c6ccccc6F)CC5)c4c(=O)[nH]3)CC2)[C@@H]1N] | 6-((3S,4S)-4-amino-3-methyl-2-oxa- 8-azaspiro[4.5]decan-8-yl)-3-(1-(2- fluorophenyl)cyclopropyl)-1,5- dihydro-4H-pyrazolo[3,4- d]pyrimidin-4-one | 438.50 |
| Example 295 | 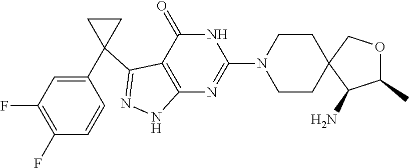 [SMILES: C[C@@H]1OCC2(CCN(c3nc4[nH]nc(C5(c6ccc(F)c(F)c6)CC5)c4c(=O)[nH]3)CC2)[C@@H]1N] | 6-((3S,4S)-4-amino-3-methyl-2-oxa- 8-azaspiro[4.5]decan-8-yl)-3-(1-(3,4- difluorophenyl)cyclopropyl)-1,5- dihydro-4H-pyrazolo[3,4- d]pyrimidin-4-one | 456.49 |
| Example 296 | 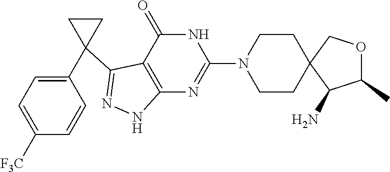 [SMILES: C[C@@H]1OCC2(CCN(c3nc4[nH]nc(C5(c6ccc(C(F)(F)F)cc6)CC5)c4c(=O)[nH]3)CC2)[C@@H]1N] | 6-((3S,4S)-4-amino-3-methyl-2-oxa- 8-azaspiro[4.5]decan-8-yl)-3-(1-(4- (trifluoromethyl)phenyl)cyclopropyl)- 1,5-dihydro-4H-pyrazolo[3,4- d]pyrimidin-4-one | 488.51 |
| Example 297 | 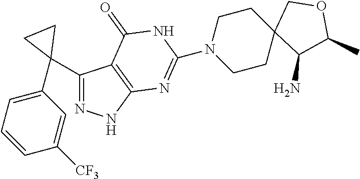 [SMILES: C[C@@H]1OCC2(CCN(c3nc4[nH]nc(C5(c6cccc(C(F)(F)F)c6)CC5)c4c(=O)[nH]3)CC2)[C@@H]1N] | 6-((3S,4S)-4-amino-3-methyl-2-oxa- 8-azaspiro[4.5]decan-8-yl)-3-(1-(3- (trifluoromethyl)phenyl)cyclopropyl)- 1,5-dihydro-4H-pyrazolo[3,4- d]pyrimidin-4-one | 488.51 |
| Example 298 | 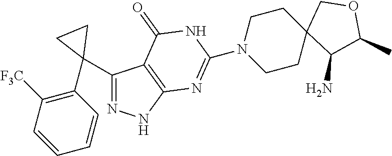 [SMILES: C[C@@H]1OCC2(CCN(c3nc4[nH]nc(C5(c6ccccc6C(F)(F)F)CC5)c4c(=O)[nH]3)CC2)[C@@H]1N] | 6-((3S,4S)-4-amino-3-methyl-2-oxa- 8-azaspiro[4.5]decan-8-yl)-3-(1-(2- (trifluoromethyl)phenyl)cyclopropyl)- 1,5-dihydro-4H-pyrazolo[3,4- d]pyrimidin-4-one | 488.51 |
| Example 299 | 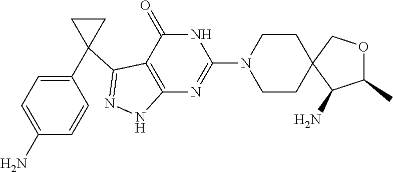 [SMILES: C[C@@H]1OCC2(CCN(c3nc4[nH]nc(C5(c6ccc(N)cc6)CC5)c4c(=O)[nH]3)CC2)[C@@H]1N] | 6-((3S,4S)-4-amino-3-methyl-2-oxa- 8-azaspiro[4.5]decan-8-yl)-3-(1-(4- aminophenyl)cyclopropyl)-1,5- dihydro-4H-pyrazolo[3,4- d]pyrimidin-4-one | 435.52 |
| Example 300 | 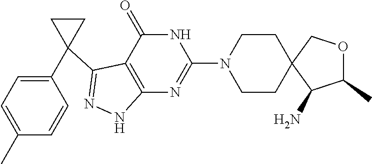 [SMILES: Cc1ccc(C2(c3n[nH]c4nc(N5CCC6(CC5)CO[C@@H](C)[C@H]6N)[nH]c(=O)c34)CC2)cc1] | 6-((3S,4S)-4-amino-3-methyl-2-oxa- 8-azaspiro[4.5]decan-8-yl)-3-(1-(p- tolyl)cyclopropyl)-1,5-dihydro-4H- pyrazolo[3,4-d]pyrimidin-4-one | 434.53 |
| Example 301 | 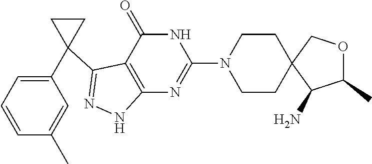 [SMILES: Cc1cccc(C2(c3n[nH]c4nc(N5CCC6(CC5)CO[C@@H](C)[C@H]6N)[nH]c(=O)c34)CC2)c1] | 6-((3S,4S)-4-amino-3-methyl-2-oxa- 8-azaspiro[4.5]decan-8-yl)-3-(1-(m- tolyl)cyclopropyl)-1,5-dihydro-4H- pyrazolo[3,4-d]pyrimidin-4-one | 434.53 |
| Example 302 | 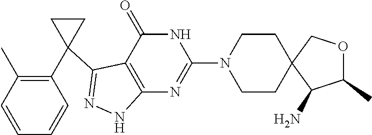 [SMILES: Cc1ccccc1C1(c2n[nH]c3nc(N4CCC5(CC4)CO[C@@H](C)[C@H]5N)[nH]c(=O)c23)CC1] | 6-((3S,4S)-4-amino-3-methyl-2-oxa- 8-azaspiro[4.5]decan-8-yl)-3-(1-(o- tolyl)cyclopropyl)-1,5-dihydro-4H- pyrazolo[3,4-d]pyrimidin-4-one | 434.53 |
| Example 303 | 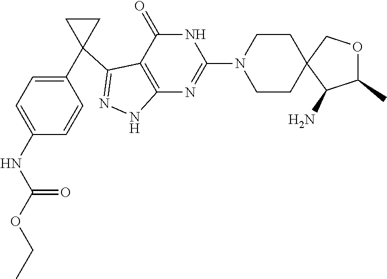 [SMILES: CCOC(=O)Nc1ccc(C2(c3n[nH]c4nc(N5CCC6(CC5)CO[C@@H](C)[C@H]6N)[nH]c(=O)c34)CC2)cc1] | ethyl(4-(1-(6-((3S,4S)-4-amino-3- methyl-2-oxa-8-azaspiro[4.5]decan- 8-yl)-4-oxo-4,5-dihydro-1H- pyrazolo[3,4-d]pyrimidin-3- yl)cyclopropyl)phenyl)carbamate | 507.58 |
| Example 304 | 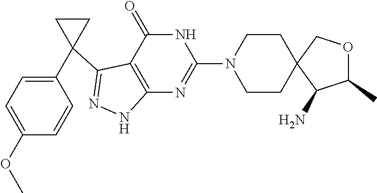 [SMILES: COc1ccc(C2(c3n[nH]c4nc(N5CCC6(CC5)CO[C@@H](C)[C@H]6N)[nH]c(=O)c34)CC2)cc1] | 6-((3S,4S)-4-amino-3-methyl-2-oxa- 8-azaspiro[4.5]decan-8-yl)-3-(1-(4- methoxyphenyl)cyclopropyl)-1,5- dihydro-4H-pyrazolo[3,4- d]pyrimidin-4-one | 450.53 |
| Example 305 | 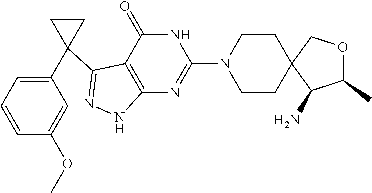 [SMILES: COc1cccc(C2(c3n[nH]c4nc(N5CCC6(CC5)CO[C@@H](C)[C@H]6N)[nH]c(=O)c34)CC2)c1] | 6-((3S,4S)-4-amino-3-methyl-2-oxa- 8-azaspiro[4.5]decan-8-yl)-3-(1-(3- methoxyphenyl)cyclopropyl)-1,5- dihydro-4H-pyrazolo[3,4- d]pyrimidin-4-one | 450.53 |
| Example 306 | 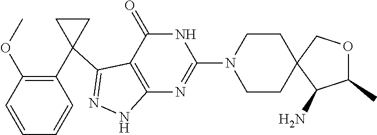 [SMILES: COc1ccccc1C1(c2n[nH]c3nc(N4CCC5(CC4)CO[C@@H](C)[C@H]5N)[nH]c(=O)c23)CC1] | 6-((3S,4S)-4-amino-3-methyl-2-oxa- 8-azaspiro[4.5]decan-8-yl)-3-(1-(2- methoxyphenyl)cyclopropyl)-1,5- dihydro-4H-pyrazolo[3,4- d]pyrimidin-4-one | 450.53 |
| Example 307 | 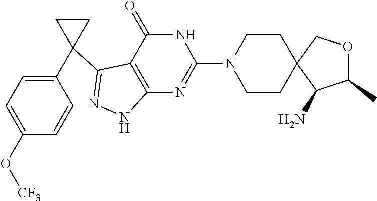 [SMILES: C[C@@H]1OCC2(CCN(c3nc4[nH]nc(C5(c6ccc(OC(F)(F)F)cc6)CC5)c4c(=O)[nH]3)CC2)[C@@H]1N] | 6-((3S,4S)-4-amino-3-methyl-2-oxa- 8-azaspiro[4.5]decan-8-yl)-3-(1-(4- (trifluoromethoxy)phenyl)cyclopropyl)- 1,5-dihydro-4H-pyrazolo[3,4- d]pyrimidin-4-one | 504.50 |
| Example 308 | 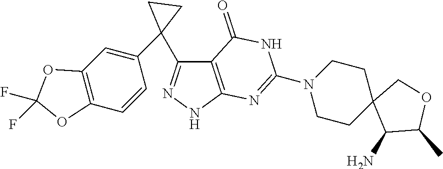 [SMILES: C[C@@H]1OCC2(CCN(c3nc4[nH]nc(C5(c6ccc7c(c6)OC(F)(F)O7)CC5)c4c(=O)[nH]3)CC2)[C@@H]1N] | 6-((3S,4S)-4-amino-3-methyl-2-oxa- 8-azaspiro[4.5]decan-8-yl)-3-(1-(2,2- difluorobenzo[d][1,3]dioxol-5- yl)cyclopropyl)-1,5-dihydro-4H- pyrazolo[3,4-d]pyrimidin-4-one | 500.50 |
| Example 309 | 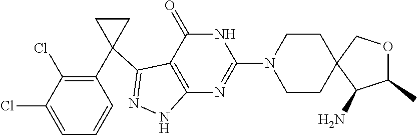 [SMILES: C[C@@H]1OCC2(CCN(c3nc4[nH]nc(C5(c6cccc(Cl)c6Cl)CC5)c4c(=O)[nH]3)CC2)[C@@H]1N] | 6-((3S,4S)-4-amino-3-methyl-2-oxa- 8-azaspiro[4.5]decan-8-yl)-3-(1-(2,3- dichlorophenyl)cyclopropyl)-1,5- dihydro-4H-pyrazolo[3,4- d]pyrimidin-4-one | 489.40 |
| Example 310 | 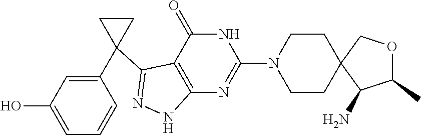 [SMILES: C[C@@H]1OCC2(CCN(c3nc4[nH]nc(C5(c6cccc(O)c6)CC5)c4c(=O)[nH]3)CC2)[C@@H]1N] | 6-((3S,4S)-4-amino-3-methyl-2-oxa- 8-azaspiro[4.5]decan-8-yl)-3-(1-(3- hydroxyphenyl)cyclopropyl)-1,5- dihydro-4H-pyrazolo[3,4- d]pyrimidin-4-one | 436.51 |
| Example 311 | 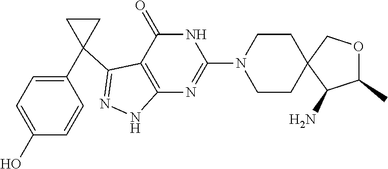 [SMILES: C[C@@H]1OCC2(CCN(c3nc4[nH]nc(C5(c6ccc(O)cc6)CC5)c4c(=O)[nH]3)CC2)[C@@H]1N] | 6-((3S,4S)-4-amino-3-methyl-2-oxa- 8-azaspiro[4.5]decan-8-yl)-3-(1-(4- hydroxyphenyl)cyclopropyl)-1,5- dihydro-4H-pyrazolo[3,4- d]pyrimidin-4-one | 436.51 |
| Example 313 | 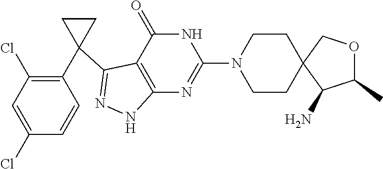 [SMILES: C[C@@H]1OCC2(CCN(c3nc4[nH]nc(C5(c6ccc(Cl)cc6Cl)CC5)c4c(=O)[nH]3)CC2)[C@@H]1N] | 6-((3S,4S)-4-amino-3-methyl-2-oxa- 8-azaspiro[4.5]decan-8-yl)-3-(1-(2,4- dichlorophenyl)cyclopropyl)-1,5- dihydro-4H-pyrazolo[3,4- d]pyrimidin-4-one | 489.40 |
| Example 314 | 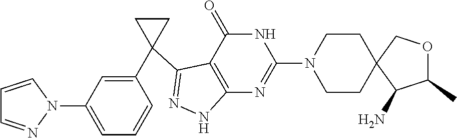 [SMILES: C[C@@H]1OCC2(CCN(c3nc4[nH]nc(C5(c6cccc(-n7cccn7)c6)CC5)c4c(=O)[nH]3)CC2)[C@@H]1N] | 3-(1-(3-(1H-pyrazol-1- yl)phenyl)cyclopropyl)-6-((3S,4S)-4- amino-3-methyl-2-oxa-8- azaspiro[4.5]decan-8-yl)-1,5- dihydro-4H-pyrazolo[3,4- d]pyrimidin-4-one | 486.57 |
| Example 315 | 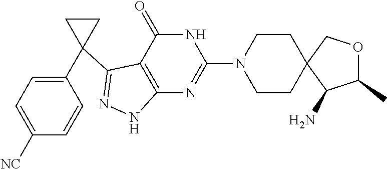 [SMILES: C[C@@H]1OCC2(CCN(c3nc4[nH]nc(C5(c6ccc(C#N)cc6)CC5)c4c(=O)[nH]3)CC2)[C@@H]1N] | 4-(1-(6-((3S,4S)-4-amino-3-methyl- 2-oxa-8-azaspiro[4.5]decan-8-yl)-4- oxo-4,5-dihydro-1H-pyrazolo[3,4- d]pyrimidin-3- yl)cyclopropyl)benzonitrile | 445.52 |
| Example 316 | 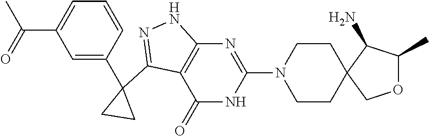 [SMILES: CC(=O)c1cccc(C2(c3n[nH]c4nc(N5CCC6(CC5)CO[C@H](C)[C@@H]6N)[nH]c(=O)c34)CC2)c1] | 3-(1-(3-acetylphenyl)cyclopropyl)-6- ((3R,4R)-4-amino-3-methyl-2-oxa-8- azaspiro[4.5]decan-8-yl)-1,5- dihydro-4H-pyrazolo[3,4- d]pyrimidin-4-one | 462.54 |
| Example 317 | 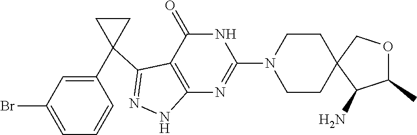 [SMILES: C[C@@H]1OCC2(CCN(c3nc4[nH]nc(C5(c6cccc(Br)c6)CC5)c4c(=O)[nH]3)CC2)[C@@H]1N] | 6-((3S,4S)-4-amino-3-methyl-2-oxa- 8-azaspiro[4.5]decan-8-yl)-3-(1-(3- bromophenyl)cyclopropyl)-1,5- dihydro-4H-pyrazolo[3,4- d]pyrimidin-4-one | 499.40 |
| Example 318 | 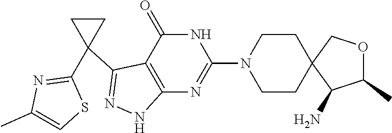 | 6-((3S,4S)-4-amino-3-methyl-2-oxa- 8-azaspiro[4.5]decan-8-yl)-3-(1-(4- methylthiazol-2-yl)cyclopropyl)-1,5- dihydro-4H-pyrazolo[3,4- d]pyrimidin-4-one | 442.56 |
| Example 319 | 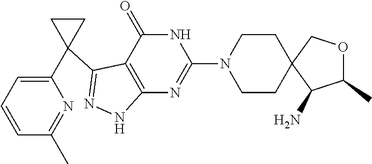 [SMILES: Cc1cccc(C2(c3n[nH]c4nc(N5CCC6(CC5)CO[C@@H](C)[C@H]6N)[nH]c(=O)c34)CC2)n1] | 6-((3S,4S)-4-amino-3-methyl-2-oxa- 8-azaspiro[4.5]decan-8-yl)-3-(1-(6- methylpyridin-2-yl)cyclopropyl)-1,5- dihydro-4H-pyrazolo[3,4- d]pyrimidin-4-one | 436.52 |
| Example 320 | 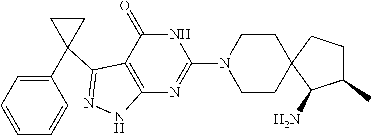 [SMILES: C[C@@H]1CCC2(CCN(c3nc4[nH]nc(C5(c6ccccc6)CC5)c4c(=O)[nH]3)CC2)[C@@H]1N] | 6-((1R,2R)-1-amino-2-methyl-8- azaspiro[4.5]decan-8-yl)-3-(1- phenylcyclopropyl)-1,5-dihydro-4H- pyrazolo[3,4-d]pyrimidin-4-one | 419.53 |
| Example 321 | 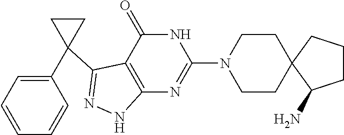 [SMILES: N[C@@H]1CCCC12CCN(c1nc3[nH]nc(C4(c5ccccc5)CC4)c3c(=O)[nH]1)CC2] | (R)-6-(1-amino-8- azaspiro[4.5]decan-8-yl)-3-(1- phenylcyclopropyl)-1,5-dihydro-4H- pyrazolo[3,4-d]pyrimidin-4-one | 405.51 |

Example 270:

¹H NMR (500 MHI-z, CD₃OD) δ 8.11 (s, 1H), 7.41-7.33 (m, 3H), 7.32-7.28 (m, 2H), 4.20-3.98 (m, 2H), 3.42-3.32 (m, 2H), 3.15 (q, J=16.4 Hz, 2H), 3.01 (m, 2H), 1.92-1.50 (m, 4H), 1.49-1.39 (m, 4H), 1.20 (s, 3H).

### Comparative Compound 1

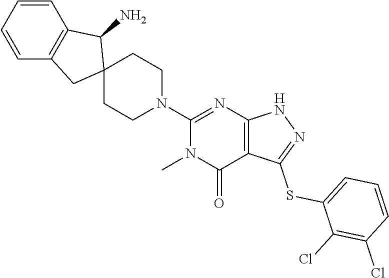 [SMILES: Cn1c(N2CCC3(CC2)Cc2ccccc2[C@H]3N)nc2[nH]nc(Sc3cccc(Cl)c3Cl)c2c1=O]

The above comparative compound 1 is Compound 178 in WO2019183367.

The synthesis method of Comparative compound 1 is as follows:

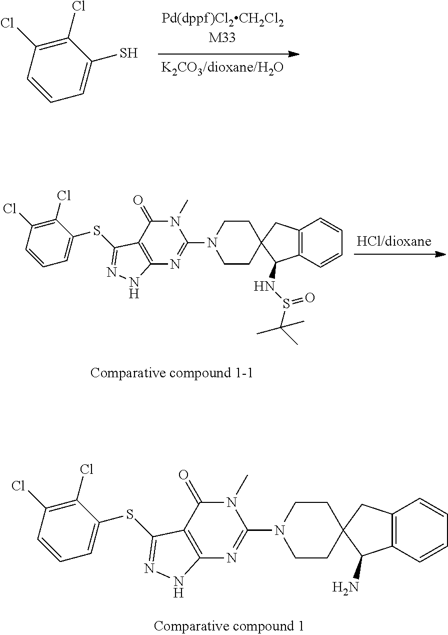 [SMILES: Sc1cccc(Cl)c1Cl ; Cn1c(N2CCC3(CC2)Cc2ccccc2[C@H]3NS(=O)C(C)(C)C)nc2[nH]nc(Sc3cccc(Cl)c3Cl)c2c1=O ; Cn1c(N2CCC3(CC2)Cc2ccccc2[C@H]3N)nc2[nH]nc(Sc3cccc(Cl)c3Cl)c2c1=O]

### Step 1: Preparation of Comparative Compound 1-1

To a 50 mL round bottom flask equipped with a stir bar were added 2,3-dichlorobenzenethiol (179 mg), M33 (580 mg), Pd(dppf)Cl₂·CH₂Cl₂ (15 mg), K₂CO₃ (276 mg) and dioxane/H₂O (10 mL/1 mL). The flask was evacuated and backfilled with argon for 3 times, then stirred for 16 hours at 100° C., cooled down, evaporated and purified by silica gel chromatography (DCM/MeOH=100/0-100/6) to afford the desired product Comparative compound 1-1 (408 mg). [M+H]⁺=631.

### Step 2: Preparation of Comparative Compound 1

To a 50 mL round bottom flask equipped with a stir bar were added Comparative compound 1-1 (126 mg), 4N dioxane/HCl (5 mL) stirred for 1 hour at r.t, then evaporated, the residue was washed by Et₂O (20 mL) to afford the desired product Comparative compound 1 (103 mg). [M+H]⁺=527.

### Comparative Compound 2

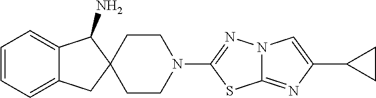 [SMILES: N[C@@H]1c2ccccc2CC12CCN(c1nn3cc(C4CC4)nc3s1)CC2]

The above comparative compound 2 is Compound 253 in WO2019183367.

### Pharmacological Test

### Example A SHP2 Allosteric Inhibitor Enzyme Activity Assay

SHP2 is activated by the binding of a bis-tyrosyl-phosphorylated peptide to its Src homologous 2 (SH2) domain. This subsequent activation step leads to the release of the automatic inhibition interface of SHP2, which in turn activates the SHP2 protein tyrosine phosphatase (PTP) and can be used for substrate recognition and reaction catalysis. The catalytic activity of SHP2 was monitored using the alternative DiFMUP in a rapid fluorescence assay format.

Assay procedures are as follows:

(1) Compound Formulation:

The compound of the present invention (10 mM stock solution) was diluted to a suitable multiple with 100% DMSO and assay buffer, and the final test concentration of the compound of the present invention was 1 μM, 0.333 μM, 0.111 μM, 0.0370 μM, 0.0124 μM, 0.00412 μM, 0.00137 μM, 0.00046 μM, 0.00015 μM, 0.00 μM;

(2) Preparation of Enzyme Reaction Working Fluid:

SHP2 enzyme activity was performed using a final reaction volume of 50 μL and the following assay buffer conditions in 96-well black polystyrene plates (flat bottom, low flange, non-bonding surface) (Perki Elmer, Cat #6005270) at room temperature: 60 mM HEPES, 75 mM NaCl, 75 mM KCl, 0.05% BRIJ-35, 1 mM EDTA, 5 mM DTT.

(3) Enzyme Catalytic Reaction and Data Monitoring:

Compounds of the present invention were added to the corresponding 96-well plates, and no compound and enzyme were provided as blank test wells. SHP2 Activating Peptide (IRS1_pY1172 (dPEG8)pY1222) was placed on ice for melting, and 0.5 μM was added per well, then 0.2 ng SHP2 protein samples were added to corresponding well plates, and incubated at room temperature for 1 hour. Substrate DiFMUP (Invitrogen, Cat #D6567) was added to the reaction at room temperature for 1 hour. Fluorescence signals were monitored using an enzyme reader (Envision, Perki Elmer) using excitation wavelengths and emission wavelengths of 340 nm and 450 nm, respectively.

(4) Data Analysis:

Inhibition %=[1−(Conversion_{_sample}−Conversion_{_min})/(Conversion_{_max}−Conversion_{_min})]*100% Calculation formula:

where: Conversion_{_sample} is the mean reading of the sample wells; Conversion_{_min} is the mean reading of blank control wells, representing the reading of the wells without enzyme; Conversion_{_max} is the mean of positive control wells, representing the reading of the wells without inhibitor.

The dose-response curve is fitted with GraphPad Prism software and IC₅₀ was calculated by the “log[inhibitor] vs. response-variable slope” program.

The result is expressed with IC₅₀, the inhibitory activity of compounds against SHP2, shown in Table 5, Compounds of the present disclosure, as exemplified in the Examples, showed IC₅₀ values in the following ranges: “A” stands for “IC₅₀≤20 nM”; “B” stands for “20 nM<IC₅₀≤60 nM”; “C” stands for “IC₅₀>60 nM”.

TABLE 5

| | SHP2 |
|---|---|
| | IC₅₀ |
| Example | (nM) |
| Example 1 | A |
| (A001) | |
| Example 2 | A |
| (A002) | |
| Example 3 | A |
| (A003) | |
| Example 4 | A |
| (A004) | |
| Example 5 | C |
| (A005) | |
| Example 6 | 0.62 |
| (A006) | |
| Example 7 | C |
| (A007) | |
| Example 8 | 2.2 |
| (A008) | |
| Example 9 | A |
| (A009) | |
| Example 10 | 0.9 |
| (A010) | |
| Example 11 | A |
| (A011) | |
| Example 12 | A |
| (A012) | |
| Example 13 | A |
| (A013) | |
| Example 14 | A |
| (A014) | |
| Example 15 | A |
| (A015) | |
| Example 16 | A |
| (A016) | |
| Example 17 | A |
| (A017) | |
| Example 18 | A |
| (A018) | |
| Example 20 | A |
| (A020) | |
| Example 21 | A |
| (A021) | |
| Example 22 | A |
| (A022) | |
| Example 23 | A |
| (A023) | |
| Example 24 | 1.6 |
| (A024) | |
| Example 25 | A |
| (A025) | |
| Example 26 | A |
| (A026) | |
| Example 27 | A |
| (A027) | |
| Example 28 | A |
| (A028) | |
| Example 29 | A |
| (A029) | |
| Example 30 | A |
| (A030) | |
| Example 31 | B |
| (A031) | |
| Example 32 | B |
| (A032) | |
| Example 33 | 0.9 |
| (A033) | |
| Example 34 | 2.7 |
| (A034) | |
| Example 35 | A |
| (A035) | |
| Example 36 | A |
| (A036) | |
| Example 37 | A |
| (A037) | |
| Example 38 | 3.1 |
| (A038) | |
| Example 39 | A |
| (A039) | |
| Example 40 | A |
| (A040) | |
| Example 41 | A |
| (A041) | |
| Example 42 | 1.9 |
| (A042) | |
| Example 43 | A |
| (A043) | |
| Example 44 | A |
| (A044) | |
| Example 46 | A |
| (A046) | |
| Example 48 | A |
| (A048) | |
| Example 49 | A |
| (A049) | |
| Example 50 | A |
| (A050) | |
| Example 51 | A |
| (A051) | |
| Example 52 | A |
| (A052) | |
| Example 53 | A |
| (A053) | |
| Example 54 | A |
| (A054) | |
| Example 55 | B |
| (A055) | |
| Example 56 | B |
| (A056) | |
| Example 57 | A |
| (A057) | |
| Example 58 | A |
| (A058) | |
| Example 59 | A |
| (A059) | |
| Example 60 | A |
| (A060) | |
| Example 61 | A |
| (A061) | |
| Example 62 | A |
| (A062) | |
| Example 63 | A |
| (A063) | |
| Example 65 | B |
| (A065) | |
| Example 66 | A |
| (A066) | |
| Example 67 | A |
| (A067) | |
| Example 69 | B |
| (A069) | |
| Example 70 | A |
| (A070) | |
| Example 71 | A |
| (A071) | |
| Example 72 | A |
| (A072) | |
| Example 73 | A |
| (A073) | |
| Example 77 | A |
| (A077) | |
| Example 78 | A |
| (A078) | |
| Example 79 | A |
| (A079) | |
| Example 80 | A |
| (A080) | |
| Example 81 | A |
| (A081) | |
| Example 82 | A |
| (A082) | |
| Example 83 | A |
| (A083) | |
| Example 84 | A |
| (A084) | |
| Example 85 | A |
| (A085) | |
| Example 86 | A |
| (A086) | |
| Example 87 | A |
| (A087) | |
| Example 88 | A |
| (A088) | |
| Example 89 | A |
| (A089) | |
| Example 90 | A |
| (A090) | |
| Example 91 | A |
| (A091) | |
| Example 92 | A |
| (A092) | |
| Example 93 | A |
| (A093) | |
| Example 94 | A |
| (A094) | |
| Example 95 | A |
| (A095) | |
| Example 96 | A |
| (A096) | |
| Example 97 | B |
| (A097) | |
| Example 98 | B |
| (A098) | |
| Example 99 | B |
| (A099) | |
| Example 100 | B |
| (A100) | |
| Example 101 | B |
| (A101) | |
| Example 105 | A |
| (C001) | |
| Example 106 | A |
| (C002) | |
| Example 107 | B |
| (C003) | |
| Example 185 | 0.48 |
| (C004) | |
| Example 108 | 1.9 |
| (D001) | |
| Example 109 | A |
| (D002) | |
| Example 110 | A |
| (D003) | |
| Example 111 | A |
| (D004) | |
| Example 112 | A |
| (D005) | |
| Example 113 | A |
| (D006) | |
| Example 114 | A |
| (D007) | |
| Example 115 | A |
| (D008) | |
| Example 116 | A |
| (D009) | |
| Example 117 | A |
| (D010) | |
| Example 118 | B |
| (D011) | |
| Example 119 | A |
| (D012) | |
| Example 120 | A |
| (D013) | |
| Example 121 | A |
| (0014) | |
| Example 122 | 1.1 |
| (D015) | |
| Example 123 | A |
| (D016) | |
| Example 124 | 0.48 |
| (0017) | |
| Example 125 | 1.6 |
| (D018) | |
| Example 126 | A |
| (D019) | |
| Example 127 | A |
| (D020) | |
| Example 128 | A |
| (D021) | |
| Example 129 | B |
| (D022) | |
| Example 130 | B |
| (D023) | |
| Example 131 | A |
| (D024) | |
| Example 132 | A |
| (D025) | |
| Example 133 | A |
| (D026) | |
| Example 134 | 1.7 |
| (D027) | |
| Example 135 | A |
| (D028) | |
| Example 137 | B |
| (D030) | |
| Example 138 | B |
| (D031) | |
| Example 139 | 1.6 |
| (D032) | |
| Example 140 | B |
| (D033) | |
| Example 141 | B |
| (D034) | |
| Example 142 | B |
| (D035) | |
| Example 143 | A |
| (D036) | |
| Example 144 | A |
| (D037) | |
| Example 145 | A |
| (D038) | |
| Example 146 | A |
| (D039) | |
| Example 147 | A |
| (D040) | |
| Example 148 | B |
| (D041) | |
| Example 149 | A |
| (D042) | |
| Example 150 | A |
| (D043) | |
| Example 151 | A |
| (D044) | |
| Example 152 | B |
| (D045) | |
| Example 153 | A |
| (D046) | |
| Example 154 | A |
| (D047) | |
| Example 155 | B |
| (D048) | |
| Example 159 | B |
| (D052) | |
| Example 160 | A |
| (D053) | |
| Example 161 | A |
| (D054) | |
| Example 162 | A |
| (D055) | |
| Example 163 | A |
| (D056) | |
| Example 164 | A |
| (D057) | |
| Example 167 | A |
| (D060) | |
| Example 168 | A |
| (D061) | |
| Example 169 | A |
| (D062) | |
| Example 170 | A |
| (DO63) | |
| Example 171 | A |
| (D064) | |
| Example 172 | A |
| (D065) | |
| Example 173 | A |
| (D066) | |
| Example 174 | A |
| (D067) | |
| Example 175 | A |
| (D068) | |
| Example 176 | A |
| (D069) | |
| Example 177 | A |
| (D070) | |
| Example 178 | A |
| (D071) | |
| Example 179 | A |
| (D072) | |
| Example 180 | A |
| (D073) | |
| Example 181 | A |
| (D074) | |
| Example 182 | A |
| (D075) | |
| Example 183 | A |
| (D076) | |
| Example 184 | A |
| (D077) | |
| Example 186 | A |
| (D078) | |
| Example 187 | A |
| (D079) | |
| Example 188 | A |
| (D080) | |
| Example 189 | A |
| (D081) | |
| Example 190 | A |
| (D082) | |
| Example 192 | A |
| Example 193 | A |
| Example 194 | A |
| Example 195 | A |
| Example 196 | A |
| Example 197 | A |
| Example 198 | B |
| Example 199 | B |
| Example 200 | A |
| Example 216 | 1.1 |
| Example 217 | A |
| Example 218 | A |
| Example 219 | A |
| Example 220 | A |
| Example 221 | A |
| Example 222 | A |
| Example 223 | A |
| Example 224 | A |
| Example 225 | A |
| Example 226 | A |
| Example 227 | A |
| Example 228 | A |
| Example 230 | A |
| Example 232 | A |
| Example 233 | A |
| Example 234 | A |
| Example 235 | A |
| Example 236 | A |
| Example 237 | A |
| Example 238 | A |
| Example 239 | A |
| Example 240 | A |
| Example 241 | A |
| Example 242 | A |
| Example 243 | A |
| Example 244 | A |
| Example 245 | A |
| Example 246 | A |
| Example 247 | A |
| Example 248 | A |
| Example 250 | A |
| Example 251 | B |
| Example 252 | A |
| Example 253 | A |
| Example 256 | A |
| Example 258 | C |
| Example 259 | C |
| Example 260 | C |
| Example 261 | C |
| Example 262 | C |
| Example 263 | C |
| Example 264 | C |
| Example 265 | C |
| Example 266 | C |
| Example 267 | B |
| Example 268 | B |
| Example 269 | C |
| Example 270 | B |
| Example 271 | B |
| Example 272 | B |
| Example 273 | B |
| Example 274 | B |
| Example 275 | B |
| Example 276 | B |
| Example 277 | B |
| Example 278 | B |
| Example 279 | B |
| Example 280 | C |
| Example 282 | B |
| Example 283 | C |
| Example 284 | B |
| Example 285 | C |
| Example 286 | B |
| Example 287 | B |
| Example 288 | C |
| Example 289 | C |
| Example 290 | B |
| Example 291 | B |
| Example 292 | B |
| Example 293 | B |
| Example 294 | B |
| Example 295 | B |
| Example 296 | C |
| Example 297 | B |
| Example 298 | C |
| Example 299 | B |
| Example 300 | B |
| Example 301 | B |
| Example 302 | B |
| Example 303 | B |
| Example 304 | B |
| Example 305 | B |
| Example 306 | B |
| Example 307 | B |
| Example 308 | C |
| Example 309 | B |
| Example 310 | B |
| Example 311 | B |
| Example 313 | C |
| Example 314 | C |
| Example 315 | B |
| Example 316 | B |
| Example 317 | B |
| Example 318 | B |
| Example 319 | C |
| Example 320 | B |
| Example 321 | B |

Unexpectedly, we found that the compounds of the invention have greatly improved the inhibition activity of SHP2 enzyme.

### Example B Cell Proliferation Assay

The effects of the compounds on the proliferation of leukemia MV-4-11 cell and lung cancer NCI-H358 cell were evaluated by in vitro cell test. The assay used in this study was the CELL TITER-GLO (CTG) luminescence assay, which can detect the number of living cells by quantitative determination of ATP. Because ATP is involved in a variety of enzymatic reactions in vivo and is an indicator of the metabolism of living cells, its content can directly reflects the number and state of cells. During the experiment, Celltiter-Glo™ reagent was added to the cell culture medium to measure the luminescence value. The luminescence value was proportional to the amount of ATP, which in turn was positively correlated with the number of living cells. Therefore, ATP content can be used to detect cell viability.

Test Procedure:

(1) Cell Plating:

A bottle of MV-4-11 cells in logarithmic growth phase was taken, the cells were collected, centrifuged, resuspend, counted, and then inoculated into 96-well Microplate (Corning #3917), with 4000 cells inoculated in each well. The plates were placed in an incubator at 37° C. and 5% CO₂ for 24 hrs culture, and the compounds of the invention were added for conducting.

A bottle of NCI-H358 cells in logarithmic growth phase was taken, the cells were digested and resuspend, counted, and then the cell density was adjusted. After that, the cells were inoculated into a 96-well Ultra-Low Attachment Microplate (Corning #3474), 2000 cells were inoculated in each well, and the well plate was placed in an incubator at 37° C. and 5% CO₂, and the compounds of the invention were added for conducting.

(2) Compound Conducting:

An appropriate amount of the compound of the invention was taken for cell treatment, and the final concentration of the compound from high to low was 1000 nM, 333.3 nM, 111.1 nM, 37.04 nM, 12.35 nM, 4.115 nM, 1.372 nm, 0.4572 nM, 0.1524 nM, 0 nM, respectively. The orifice plate was cultured in an incubator at 37° C. and 5% CO₂. Only adding medium without adding cell hole was set as blank group. Compound concentration of 0 nM group was zero control group.

(3) CTG Detection:

NCI-H358 cells were cultured for 96 hrs, then 50 μL CellTiter-Glo® Luminescent cell viability assay solution was added to each well, and the cells were gently shaken for 2 mins, and incubated at room temperature for 10 mins. The cell reaction system was transferred to 96-well Microplate (Corning #3917). The detection values of each well were read on the multi-functional microplate reader.

After cultured for 120 hrs, MV-4-11 cells were added with 50 μL CellTiter-Glo® Luminescent cell viability assay solution in each well, gently shaken for 2 mins, and incubated at room temperature for 10 mins. The detection values of each well were read on the multi-functional microplate reader.

(4) Data Analysis:

The inhibition rate is calculated according to the luminous value reading,

Inhibition rate %=(1−(administration group value−blank group value)/(control group value−blank group value)*100

The log (inhibitor) vs. response variable slope of GraphPad Prism was used to fit the dose-response curve and calculate the IC₅₀ of compounds inhibiting cell proliferation.

Compounds of the present disclosure, as exemplified in the Examples, showed IC₅₀ values in the following ranges: “A” stands for “IC₅₀≤20 nM”; “B” stands for “20 nM<IC₅₀≤60 nM”; “C” stands for “IC₅₀>60 nM”.

The experimental data are shown in Table 6.

TABLE 6

| | Compound | Compound |
|---|---|---|
| | on MV-4-11 | on NCI-H358 |
| | cell IC₅₀ | cell IC₅₀ |
| Example | (nM) | (nM) |
| Comparative | 454 | 1813 |
| compound 2 | | |
| Example 1 | A | |
| Example 4 | A | A |
| Example 6 | 6.5 | 8.6 |
| Example 8 | 6.1 | 1.7 |
| Example 9 | A | A |
| Example 10 | A | A |
| Example 11 | 1.2 | 2.1 |
| Example 13 | A | A |
| Example 17 | B | B |
| Example 18 | A | A |
| Example 19 | A | B |
| Example 21 | A | A |
| Example 24 | A | A |
| Example 33 | A | A |
| Example 34 | A | A |
| Example 96 | B | C |
| Example 115 | 1.4 | 1.2 |
| Example 122 | 2.1 | 1.1 |
| Example 124 | 1.7 | 1.6 |
| Example 125 | 1.9 | 1.1 |
| Example 139 | 2.8 | 2 |
| Example 144 | B | B |
| Example 222 | A | A |
| Example 223 | 2.8 | 11 |
| Example 228 | B | B |
| Example 231 | A | B |
| Example 237 | A | A |
| Example 240 | A | A |

Unexpectedly, we found that compared to Comparative compound 2, the activity of the compounds in the present invention on MV-4-11 and NCI-H358 cells was greatly improved.

### Example C Patch Clamp Assay to Test the Effect of Compound on hERG Channel

### Test Formulation:

10 mL extracellular solution was used to dilute the stock solution, making the final concentrations of test compound were 0.3 μM, 1 μM, 3 μM, 10 μM and 30 μM.

The solubility of compound was visually observed.

### Cell Culture and Plating:

The cell line was derived from HEK-293 cells, and grown in a humidified environment at 37° C. under 5% CO₂, using the media formulation below. The cell line should not be allowed to exceed 80% confluence within the culture vessel to prevent contact inhibition causing senescence and should thus be passaged every 3/4 days using a seeding density of 2*10⁶ cells per T175 flask. Cell lines were be pre-washed with phosphate buffered saline before harvesting with Trypsin/EDTA and seeded into new flasks.

### Manual Patch Clamp:

HEK 293 cells expressed with hERG were plated on cover slips overnight with the cell density less than 50% of confluence. Cells used for electrophysiological study were transferred to a small cell bath (1 mL) mounted on the stage of an inverted microscope (Diaphot, Nikon) and were perfused with external solution containing (in mM) 130 NaCl, 4 KCl, 1.8 CaCl₂), 1 MgCl₂, 10 glucose and 10 HEPES (pH 7.4 with NaOH), the perfusion rate was 4 mL/min. The internal pipette solution contained (in mM) 130 KCl, 1 MgCl₂, 5 ethylene glycol-bis(baminoethyl ether)-N,N,N8,N8-tetraacetic acid, 5 MgATP and 10 HEPES (pH 7.2 with KOH). An HEKA EPC-10 patch-clamp amplifier and PATCHMASTER acquisition program were used to record membrane currents (HEKA Instruments IncD-67466 Lambrecht/Pfalz Germany). All experiments were performed at room temperature (22-23° C.).

The Model P-97 micropipette puller (Sutter Instrument Company, One Digital Drive, Novato, Calif. 94949) was used to pull glass patch pipettes (BF150-86-10) in all experiments. The pipette had an inner diameter of 1-1.5 mm and when filled with internal pipette solution had a resistance of 2-4 MΩ.

### Experimental Protocol:

The experiments were initiated with a 2 min vehicle control period after forming a whole cell configuration with less 5% run-down in 5 min and the tail currents in the report were at least greater than 500 pA. After a 2 min vehicle control period, the perfusion was switched to the reservoir containing the compound at the first concentration. The same procedure was repeated 3-5 times so that each cell was exposed to 4-6 escalating concentrations of compound. The time courses for block and unblock of hERG during compounds exposure and washout were continuously recorded.

The peak tail of hERG was generated by applying 2-sec depolarizing every 12 sec steps from a holding potential of −80 mV, the peak of tail currents were measured at a 5-sec repolarizing pulse at −50 mV.

### Parameters Analyzed:

hERG peak tail currents were directly measured from a 5 sec repolarization pulse at −50 mV. The fraction of control current was plotted as a function of logarithm of compound concentration. To determine the concentration of compound for halfmaximum effect, the concentration-response curve was fitted by Hill equation as shown follow.

$$Y = Bottom + \frac{Top - Bottom}{1 + {( \frac{x}{E C 50} )}^{Hillcoefficient}}$$

where Y is the observed value, Bottom is the lowest observed value (0), Top is the highest observed value (1), and the Hillcoefficient gives the largest absolute value of the slope of the curve.

### Data Analysis and Statistics

Data were analyzed by a combination of PATCHMASTER ((HEKA Instruments IncD-67466 Lambrecht/Pfalz Germany) and Origin (OriginLab Corporation, Northampton, Mass.) software programs.

Data were expressed as mean±SEM. Changes in measured parameters were evaluated with T-Test to determine whether the change from the vehicle after equilibration in each drug concentration was significantly different (P<0.05) from that observed in the time-matched vehicle control group. The results are shown in Table 7.

TABLE 7

| | hERG |
|---|---|
| Example | (μM) |
| Comparative compound 1 | 0.09 |
| Example 1 (A001) | 1.76 |
| Example 9 (A009) | 10.4 |
| Example 19 (A019) | 11.7 |
| Example 24 (A024) | 14.8 |
| Example 33 (A033) | 9.5 |
| Example 34 (A034) | 6.9 |
| Example 38 (A038) | 14.2 |
| Example 42 (A042) | >30 |
| Example 74 | 1.84 |
| Example 90 (A090) | 4.95 |
| Example 96 (A096) | >30 |
| Example 185 (C004) | 0.59 |
| Example 217 | 0.54 |
| Example 221 | >30 |
| Example 230 | >30 |
| Example 231 | 7.5 |
| Example 234 | >30 |
| Example 237 | 7.9 |
| Example 240 | 17.1 |
| Example 252 | 20.9 |
| Example 255 | >30 |

Unexpectedly, it has been confirmed that the exemplified compounds of the present invention has a significant improvement effect on hERG compared to the comparative compound 1.

### Example D In Vitro Metabolic Stability in Human and Rat Liver Microsome

Buffers:

1900 mg MgCl₂ was dissolved into a final volume of 400 mL ultra-pure water.

17.42 g K₂HPO₄ and 13.65 g KH₂PO₄ were dissolved into a final volume of 1000 mL ultra-pure water, respectively. K₂HPO₄ and KH₂PO₄ were mixed to prepare 100 mM potassium phosphate (PBS) buffer. The pH value of the final solution was adjusted to pH 7.30±0.10.

Stop Solution:

Cold ACN (including 10 ng/mL Labetalol and 10 ng/mL Glibenclamid) was stored at 4° C.

Working Solution Preparation:

Verapamil (positive control) and test compound stock solution were diluted into a concentration of 50 μM and 200 μM respectively, using MeOH/ACN/H₂O solution (1:1:2, v/v/v).

Procedures

1) 40 μL MgCl₂ and 306 μL PBS were added to 96 plate wells (blank, compound wells, compounds without NADPH wells)

2) 4 μL compound working solution was added to above wells (blank: 4 μL PBS buffer) (Note: the volume of DMSO in final incubation system ≤0.5%)

3) 10 μL microsomes (20 mg/mL) was added in each well. The mixture was warmed up for 10 minutes at 37.0° C.

4) 40 μL 10 mM NADPH working solution was added to start reaction. The total volume was 400 μL.

5) Aliquots of 50 μL samples were taken from the reaction solution at 0, 5, 15, 45 min. The reaction solutions were stopped by the addition of 400 μL stop solution.

6) The sampling plates were shaked for approx. 5 min.

7) Samples were centrifuged at 3200 rcf for 10 min. then transferred 50 μL to a new plate dilute with 200 μL H₂O for LC/MS/MS analysis.

Data Analysis

Use equation of first order kinetics to calculate t_{1/2} and CL:

k=−slope

t_{1/2}=0.693/k

CLᵢₙₜ=k/Cₚᵣₒₜₑᵢₙ

Where k represents elimination constant, which is calculated from a log linear plot of % Remaining versus time. t_{1/2} represents the half-life. Cₚᵣₒₜₑᵢₙ is the concentration of liver microsomes. The results of metabolic stability in human and rat of liver microsomes are shown in Table 8.

TABLE 8

| | CLᵢₙₜ | |
|---|---|---|
| | (μL/min/mg proteins) | |
| Example | Human | Rat |
| Comparative Example 1 | 171.8 | 89.9 |
| Example 1 (A001) | 95.2 | 34.7 |
| Example 6 (A006) | 19.5 | 11.2 |
| Example 8 (A008) | 9.2 | 11.6 |
| Example 9 (A009) | 16.9 | 10.5 |
| Example 11 (A011) | 8.6 | 6.6 |
| Example 13 (A013) | 9.8 | 14.3 |
| Example 21 (A021) | 10.4 | 3.8 |
| Example 33 (A033) | 10.6 | 17.4 |
| Example 34 (A034) | 16.5 | 19.1 |
| Example 74 | 182.7 | 102.0 |
| Example 216 | 50.0 | 32.5 |
| Example 228 | 6.2 | 12.4 |
| Example 230 | 9.2 | 3.2 |
| Example 233 | 0.2 | 3.4 |
| Example 234 | 10.4 | 6.6 |
| Example 240 | 11.8 | 15.0 |

Unexpectedly, it has been confirmed that the exemplified compounds of the present invention have drastically improved metabolic stability in Human/Rat liver microsomes compared with the Comparative compound 1. This improved stability indicated superior pharmacokinetic properties and better clinical output in human.

### Example E In Vivo Efficacy of Subcutaneous Xenograft of MIA-PACA2 Cells in Tumor Model

BALB/c nude mice, female, 6-8 weeks old, weighing approximately 18-22 grams. Each mouse was subcutaneously inoculated with 0.2 mL (1*107) of MIA-PaCa2 cells (add matrigel, with the volume ratio being 1:1) on the right back. The administration was performed when the average tumor volume reached 100-150 mm³ cubic millimeters. The test compounds were orally administered daily, and the administration dose was 10 mpk QD. The tumor volume was measured twice a week, with the volume measured in cubic millimeters, and calculated by the following formula: V=0.5a*b², where a and b were the long and short diameters of the tumor, respectively.

The tumor suppressive effect of the compounds was evaluated by TGI (%). TGI (%) reflects the tumor growth inhibition rate. Calculation of TGI (%): TGI (%)=[(1−(average tumor volume at the end of administration in a treatment group−average tumor volume at the beginning of administration in the treatment group))/(average tumor volume at the end of treatment in the solvent control group−average tumor volume at the beginning of treatment in the solvent control group)]×100%.

In conclusion, most of the compounds listed in the present invention are highly effective, and demonstrate significant improvements in safety and pharmacokinetics, as well as excellent antitumor activity in in vivo models.

Although the present invention has been comprehensively described through its implementation, it is worth noting that various changes and modifications are obvious to those skilled in the art. Such changes and modifications shall be included in the scope of the claims attached to the invention.

1. A compound of Formula I, or a pharmaceutically acceptable salt, isomeride, stereoisomer, prodrug, chelate, non-covalent complex, or solvate thereof, 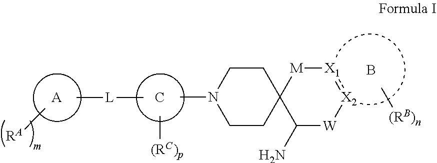
wherein, [symbol]
is a single bond or a double bond;
ring A is 6- to 14-membered aryl, 5- to 14-membered heteroaryl, 5- to 15-membered partially unsaturated heterocyclic ring, or 5- to 15-membered partially unsaturated carbocyclic ring; wherein the heteroaryl and the heterocyclic ring having 1-4 heteroatoms independently selected from N, O, and S;
ring B is absent, 6- to 10-membered aryl, 5- to 10-membered heteroaryl or 5- to 10-membered partially unsaturated heterocyclic ring;
provided that if ring B is 6- to 10-membered aryl, 5- to 10-membered heteroaryl or 5- to 10-membered partially unsaturated heterocyclic ring, then X₁ and X₂ are independently selected from C and N; or if ring B is absent, then X₁ and X₂ are independently selected from O, S, NR¹⁰⁰ and CR¹⁰⁰R¹⁰¹;
R¹⁰⁰ and R¹⁰¹ are independently selected from absent, hydrogen, halo, hydroxy, —C₁₋₆ alkyl and —C₁₋₆ alkoxy;
ring C is 5- to 14-membered heteroaryl or 5- to 14-membered partially unsaturated heterocyclic ring;
M is selected from absent, CH₂, O, NH and S;
W is absent or —CR³¹R³²—;
L is a single bond, —CR¹R²—, 3- to 6-membered monocyclic carbocyclic ring, 3- to 6-membered monocyclic heterocyclic ring, 7- to 12-membered bicyclic carbocyclic ring or 7- to 12-membered bicyclic heterocyclic ring; wherein, the 3- to 6-membered monocyclic carbocyclic ring, 3- to 6-membered monocyclic heterocyclic ring, 7- to 12-membered bicyclic carbocyclic ring and 7- to 12-membered bicyclic heterocyclic ring are optionally substituted with one to four substituents independently selected from R^{L};
each R^{A} is independently selected from hydrogen, halogen, —CN, —NO₂, ═O, C₁₋₆ alkyl, 3- to 14-membered saturated or partially unsaturated carbocyclic ring, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —P(═O)R¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or
two R^{A} together with the atoms to which they are attached form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰;
each R^{B}, R^{C} and R^{L} is independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;
R¹ and R² are independently selected from hydrogen, halogen, —CN, —NO₂, and C₁₋₆ alkyl; wherein the C₁₋₆ alkyl is optionally substituted with one or more substituents independently selected from halogen, —CN, —NO₂, —OR⁶, and —NR⁷R⁸; wherein, R¹ and R² are not simultaneously hydrogen; and provided that if R¹ is hydrogen, R² is not methyl;
each R³⁰ is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;
R³¹ and R³² are independently selected from hydrogen, halogen, —CN, —NO₂, ═O, C₁₋₆ alkyl, —OR⁶, —NR⁷R⁸ and —SR⁹; wherein the C₁₋₆ alkyl is optionally substituted with one or more substituents independently selected from halogen, —CN, —NO₂, —OR⁶, and —NR⁷R⁸;
R³, R⁴, R⁵, R¹³, R²⁶, R²⁷ and R²⁹ are independently selected from hydrogen, halogen, —CN, —NO₂, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OR⁶, —NR⁷R⁸, —SR⁹, —C(═O)OR¹⁰, —C(═O)NR¹¹R¹², —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;
R⁶, R⁷, R⁸, R⁹, R¹⁰, R¹¹, R¹², R¹⁴, R¹⁵, R¹⁶, R¹⁷, R¹⁸, R¹⁹, R²⁰, R²¹, R²², R²³, R²⁴, R²⁵ and R²⁸ are independently selected from hydrogen, hydroxyl, halogen, —CN, —NO₂, ═O, C₁₋₆ alkyl, C₁₋₆ alkoxy, C₁₋₆haloalkyl, C₃₋₈ cycloalkyl, 6- to 14-membered aryl, 5- to 14-membered heteroaryl, and 3- to 8-membered saturated or partially unsaturated heterocyclic ring;
m is selected from 0, 1, 2, 3, 4, 5 and 6;
n is selected from 0, 1, 2, 3 and 4;
p is selected from 0, 1, 2, 3 and 4;
r is selected from 1, 2, 3 and 4; and 5 is selected from 1, 2, 3 and 4.

2. (canceled)

3. (canceled)

4. (canceled)

5. (canceled)

6. The compound of claim 1, wherein the compound is of Formula II, or a pharmaceutically acceptable salt, isomeride, stereoisomer, prodrug, chelate, non-covalent complex, or solvate thereof, 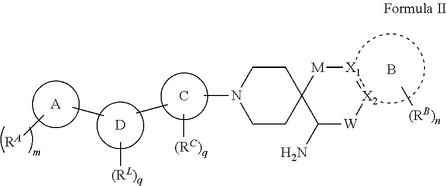
wherein, [symbol]
is a single bond or a double bond;
ring A is 6- to 14-membered aryl, 5- to 14-membered heteroaryl, 5- to 15-membered partially unsaturated heterocyclic ring, or 5- to 15-membered partially unsaturated carbocyclic ring; wherein the heteroaryl and the heterocyclic ring having 1-4 heteroatoms independently selected from N, O, and S;
ring B is absent, 6- to 10-membered aryl, 5- to 10-membered heteroaryl or 5- to 10-membered partially unsaturated heterocyclic ring;
provided that if ring B is 6- to 10-membered aryl, 5- to 10-membered heteroaryl or 5- to 10-membered partially unsaturated heterocyclic ring, then X₁ and X₂ are independently selected from C and N; or if ring B is absent, then X₁ and X₂ are independently selected from O, S, NR¹⁰⁰ and CR¹⁰⁰R¹⁰¹;
R¹⁰⁰ and R¹⁰¹ are independently selected from absent, hydrogen, halo, hydroxy, —C₁₋₆ alkyl and —C₁₋₆ alkoxy;
ring C is 5- to 14-membered heteroaryl or 5- to 14-membered partially unsaturated heterocyclic ring;
ring D is 3- to 6-membered monocyclic carbocyclic ring, 3- to 6-membered monocyclic heterocyclic ring, 7- to 12-membered bicyclic carbocyclic ring or 7- to 12-membered bicyclic heterocyclic ring;
M is selected from absent, CH₂, O, NH and S;
W is absent or —CR³¹R³²—;
each R^{A} is independently selected from hydrogen, halogen, —CN, —NO₂, ═O, C₁₋₆ alkyl, 3- to 14-membered saturated or partially unsaturated carbocyclic ring, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —P(═O)R¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or
two R^{A} together with the atoms to which they are attached to form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰;
each R^{B}, R^{C} and R^{L} are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;
each R³⁰ is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;
R³¹ and R³² are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;
R³, R⁴, R⁵, R¹³, R²⁶, R²⁷ and R²⁹ are independently selected from hydrogen, halogen, —CN, —NO₂, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OR⁶, —NR⁷R⁸, —SR⁹, —C(═O)OR¹⁰, —C(═O)NR¹¹R¹², —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;
R⁶, R⁷, R⁸, R⁹, R¹⁰, R¹¹, R¹², R¹⁴, R¹, R¹⁶, R¹⁷, R¹⁸, R¹⁹, R²⁰, R²¹, R²², R²³, R²⁴, R²⁵ and R²⁸ are independently selected from hydrogen, hydroxyl, halogen, —CN, —NO₂, ═O, C₁₋₆ alkyl, C₁₋₆ alkoxy, C₁₋₆haloalkyl, C₃₋₈ cycloalkyl, 6- to 14-membered aryl, 5- to 14-membered heteroaryl, and 3- to 8-membered saturated or partially unsaturated heterocyclic ring;
m is selected from 0, 1, 2, 3, 4, 5 and 6;
n is selected from 0, 1, 2, 3 and 4;
p is selected from 0, 1, 2, 3 and 4;
q is selected from 1, 2, 3 and 4;
r is selected from 1, 2, 3 and 4; and
s is selected from 1, 2, 3 and 4.

7. The compound of claim 1, wherein the compound is of Formula III, or a pharmaceutically acceptable salt, isomeride, stereoisomer, prodrug, chelate, non-covalent complex, or solvate thereof, 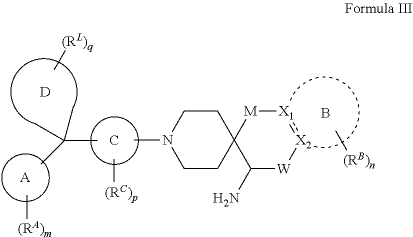
wherein, [symbol]
is a single bond or a double bond;
ring A is 6- to 14-membered aryl, 5- to 14-membered heteroaryl, 5- to 15-membered partially unsaturated heterocyclic ring, or 5- to 15-membered partially unsaturated carbocyclic ring; wherein the heteroaryl and the heterocyclic ring having 1-4 heteroatoms independently selected from N, O, and S;
ring B is absent, 6- to 10-membered aryl, 5- to 10-membered heteroaryl or 5- to 10-membered partially unsaturated heterocyclic ring;
provided that if ring B is 6- to 10-membered aryl, 5- to 10-membered heteroaryl or 5- to 10-membered partially unsaturated heterocyclic ring, then X₁ and X₂ are independently selected from C and N; or if ring B is absent, then X₁ and X₂ are independently selected from O, S, NR¹⁰⁰ and CR¹⁰⁰R¹⁰¹;
R¹⁰⁰ and R¹⁰¹ are independently selected from absent, hydrogen, halo, hydroxy, —C₁₋₆ alkyl and —C₁₋₆ alkoxy;
ring C is 5- to 14-membered heteroaryl or 5- to 14-membered partially unsaturated heterocyclic ring;
ring D is 3- to 6-membered monocyclic carbocyclic ring, 3- to 6-membered monocyclic heterocyclic ring, 7- to 12-membered bicyclic carbocyclic ring or 7- to 12-membered bicyclic heterocyclic ring;
M is selected from absent, CH₂, O, NH and S;
W is absent or —CR³¹R³²—;
each R^{A} is independently selected from hydrogen, halogen, —CN, —NO₂, ═O, C₁₋₆ alkyl, 3- to 14-membered saturated or partially unsaturated carbocyclic ring, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —P(═O)R¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or
two R^{A} together with the atoms to which they are attached to form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰;
each R^{B}, R^{C} and R^{L} are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;
R³¹ and R³² are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;
each R³⁰ is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;
R³, R⁴, R⁵, R¹³, R²⁶, R²⁷ and R²⁹ are independently selected from hydrogen, halogen, —CN, —NO₂, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OR⁶, —NR⁷R⁸, —SR⁹, —C(═O)OR¹⁰, —C(═O)NR¹¹R¹², —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;
R⁶, R⁷, R⁸, R⁹, R¹⁰, R¹¹, R¹², R¹⁴, R¹⁵, R¹⁶, R¹⁷, R¹⁸, R¹⁹, R²⁰, R²¹, R²², R²³, R²⁴, R²⁵ and R²⁸ are independently selected from hydrogen, hydroxyl, halogen, —CN, —NO₂, ═O, C₁₋₈ alkyl, C₁₋₆ alkoxy, C₁₋₆haloalkyl, C₃₋₈ cycloalkyl, 6- to 14-membered aryl, 5- to 14-membered heteroaryl, and 3- to 8-membered saturated or partially unsaturated heterocyclic ring;
m is selected from 0, 1, 2, 3, 4, 5 and 6;
n is selected from 0, 1, 2, 3 and 4;
p is selected from 0, 1, 2, 3 and 4;
q is selected from 0, 1, 2, 3 and 4;
r is selected from 1, 2, 3 and 4;
s is selected from 1, 2, 3 and 4.

8. (canceled)

9. (canceled)

10. (canceled)

11. (canceled)

12. (canceled)

13. (canceled)

14. (canceled)

15. (canceled)

16. (canceled)

17. The compound of claim 1, wherein the compound is of Formula IV, or a pharmaceutically acceptable salt, isomeride, stereoisomer, prodrug, chelate, non-covalent complex, or solvate thereof, 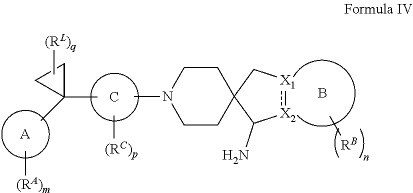
wherein,
ring A is 6- to 14-membered aryl, 5- to 14-membered heteroaryl, 5- to 15-membered partially unsaturated heterocyclic ring, or 5- to 15-membered partially unsaturated carbocyclic ring; wherein the heteroaryl, the heterocyclic ring having 1-4 heteroatoms independently selected from N, O, and S;
ring B is 6- to 10-membered aryl, 5- to 10-membered heteroaryl or 5- to 10-membered partially unsaturated heterocyclic ring;
X₁ and X₂ are independently selected from C and N;
ring C is 5- to 14-membered heteroaryl or 5- to 14-membered partially unsaturated heterocyclic ring;
each R^{A} is independently selected from hydrogen, halogen, —CN, —NO₂, ═O, C₁₋₆ alkyl, 3- to 14-membered saturated or partially unsaturated carbocyclic ring, 6- to 14-membered aryl, 5- to 14-membered heteroaryl, 3- to 14-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —P(═O)R¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one to four substituents independently selected from R³⁰; or
two R^{A} together with the atoms to which they are attached to form a 3- to 6-membered carbocyclic ring or 3- to 6-membered heterocyclic ring, wherein the 5- to 6-membered carbocyclic ring and 3- to 6-membered heterocyclic ring are optionally substituted with one to four substituents independently selected from R³⁰;
each R^{B}, R^{C} and R^{L} are independently selected from hydrogen, halogen, —CN, —NO₂, ═O, C₁₋₆ alkyl, —OR⁶, —NR⁷R⁸, and —SR⁹; wherein the C₁₋₆ alkyl is optionally substituted with one or more substituents independently selected from halogen, —CN, —NO₂, —OR⁶, and —NR⁷R⁸;
each R³⁰ is independently selected from hydrogen, halogen, —CN, —NO₂, ═O, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OC(═O)R³, —S(═O)R⁴, —C(═O)R⁵, —OR⁶, —NR⁷R⁸, —SR⁹, —C(═O)OR¹⁰, —C(═O)NR¹¹R¹², —S(═O)₂R¹³, —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, —NR²⁵C(═O)R²⁶, —OS(═O)₂R²⁷, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;
R³, R⁴, R⁵, R¹³, R²⁶, R²⁷ and R²⁹ are independently selected from hydrogen, halogen, —CN, —NO₂, C₁₋₆ alkyl, C₃₋₈ cycloalkyl, 6- to 14-membered aryl, 5- to 14-membered heteroaryl, 3- to 8-membered saturated or partially unsaturated heterocyclic ring, —OR⁶, —NR⁷R⁸, —SR⁹, —C(═O)OR¹⁰, —C(═O)NR¹¹R¹², —S(═O)₂OR¹⁴, —S(═O)₂NR¹⁵R¹⁶, —O(CH₂)ᵣOR¹⁷, —O(CH₂)ᵣNR¹⁸R¹⁹, —NR²⁰(CH₂)ₛNR²¹R²², —NR²³(CH₂)ₛOR²⁴, and —NR²⁸S(═O)₂R²⁹; wherein C₁₋₆ alkyl, C₃₋₈ cycloalkyl, 6- to 14-membered aryl, 5- to 14-membered heteroaryl, and 3- to 8-membered saturated or partially unsaturated heterocyclic ring are optionally substituted with one or more substituents independently selected from halogen, —CN, —NO₂, ═O, —OR⁶, —NR⁷R⁸, —SR⁹, C₃₋₈ cycloalkyl, 3- to 8-membered saturated or partially unsaturated heterocyclic ring, and —C₁₋₆ alkyl;
R⁶, R⁷, R⁸, R⁹, R¹⁰, R¹¹, R¹², R¹⁴, R¹⁵, R¹⁶, R¹⁷, R¹⁸, R¹⁹, R²⁰, R²¹, R²², R²³, R²⁴, R²⁵ and R²⁸ are independently selected from hydrogen, hydroxyl, halogen, —CN, —NO₂, ═O, C₁₋₈ alkyl, C₁₋₆ alkoxy, C₁₋₆haloalkyl, C₃₋₈ cycloalkyl, 6- to 14-membered aryl, 5- to 14-membered heteroaryl, and 3- to 8-membered saturated or partially unsaturated heterocyclic ring;
m is selected from 0, 1, 2, 3, 4, 5 and 6;
n is selected from 0, 1, 2, 3 and 4;
p is selected from 0, 1, 2, 3 and 4;
q is selected from 0, 1, 2, 3 and 4;
r is selected from 1, 2, 3 and 4;
s is selected from 1, 2, 3 and 4.

18. (canceled)

19. The compound of claim 1, wherein R^{L} is independently selected from hydrogen, F, Cl, Br, ═O, methyl and ethyl;
ring A is 6- to 14-membered aryl, 5- to 14-membered heteroaryl, 5- to 8-membered partially unsaturated monocyclic heterocyclic ring, 9- to 12-membered partially unsaturated bicyclic carbocyclic ring, 9- to 12-membered partially unsaturated bicyclic heterocyclic ring, 11- to 15-membered partially unsaturated tricyclic carbocyclic ring, or 11- to 15-membered partially unsaturated tricyclic heterocyclic ring;
m is selected from 0, 1 and 2; and
each R^{A} is independently selected from hydrogen, halogen, —CN, —NO₂, ═O, C₁₋₆ alkyl, 6- to 10-membered aryl, 5- to 10-membered heteroaryl, —C(═O)R⁵, —OR⁶, —NR⁷R⁸, —O(CH₂)ᵣOR¹⁷, —O(CH₂)ᵣNR¹⁸R¹⁹, —NR²O(CH₂)ₛNR²¹R²², —NR²³(CH₂)ₛOR²⁴, and —NR²⁵C(═O)R²⁶, wherein C₁₋₆ alkyl, 6- to 10-membered aryl, and 5- to 10-membered heteroaryl are optionally substituted with one to four substituents independently selected from R³⁰.

20. The compound of claim 1, wherein R^{L} is H, F, or Cl;
ring A is 6- to 14-membered aryl, 5- to 14-membered heteroaryl, 9- to 11-membered partially unsaturated bicyclic carbocyclic ring, or 9- to 11-membered partially unsaturated bicyclic heterocyclic ring; and
each R^{A} is independently selected from CH₃, F, CHF₂, CF₃, Cl, OCF₃, OCH₃, NH₂, CN, NH(CO)CH₂CH₃, OH, OCH₂CH₂OCH₃, OCHF₂, N(CH₃)₂, COCH₃, CH(CH₃)OH,

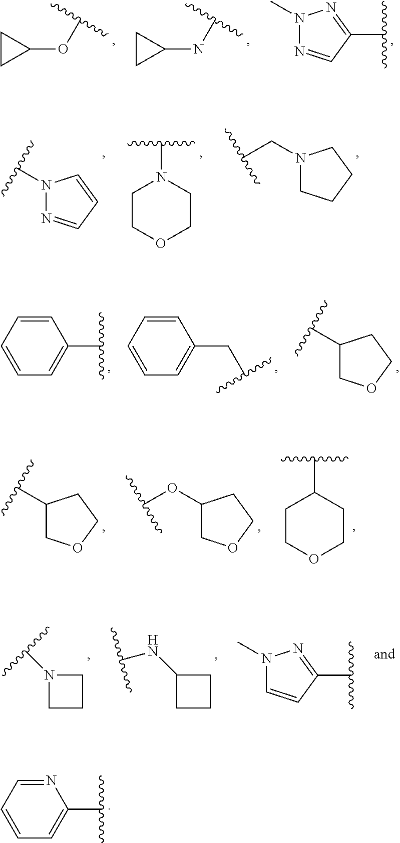

21. The compound of claim 1, wherein R^{L} is H;
ring A is 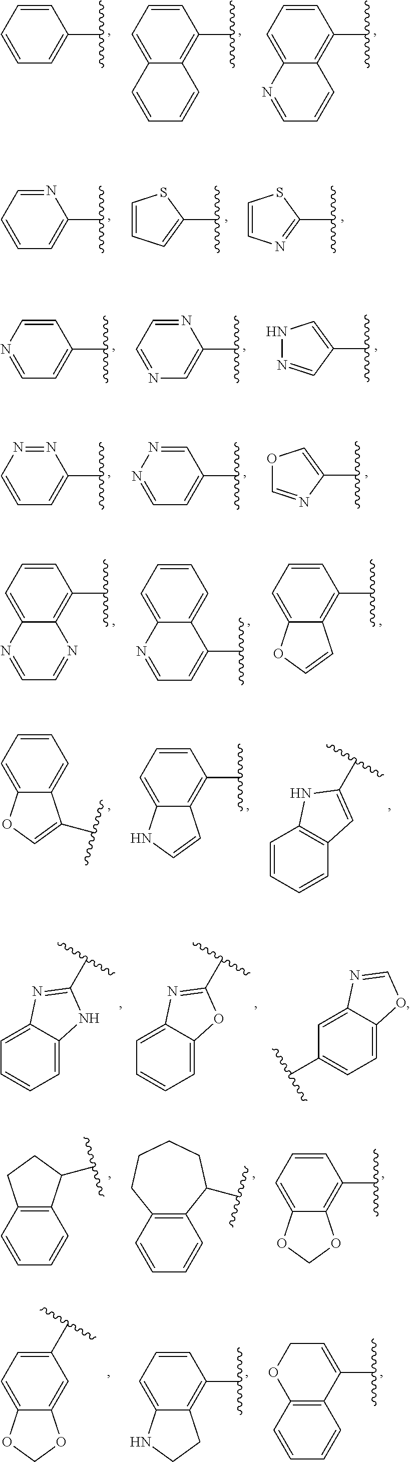 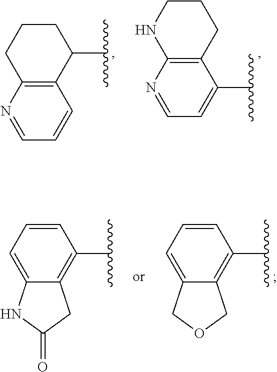 [SMILES: *C1CCCc2ncccc21 ; *c1ccnc2c1CCCN2 ; *c1cccc2c1CC(=O)N2 ; *c1cccc2c1COC2]
and
R^{A} is independently selected from hydrogen, CH₃, F, CF₃, Cl, Br, NH₂, CN, OH, COCH₃ and

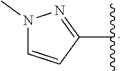 [SMILES: *c1ccn(C)n1]

22. (canceled)

23. (canceled)

24. (canceled)

25. The compound of claim 1, wherein ring A is 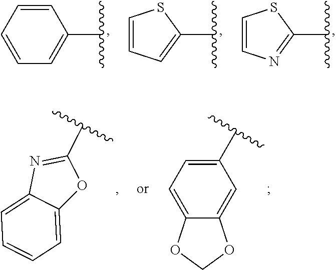
and
R^{A} is independently selected from H.

26. The compound of claim 1, wherein ring A is 6- to 14-membered aryl or 5- to 14-membered heteroaryl.

27. The compound of claim 1, wherein ring A is

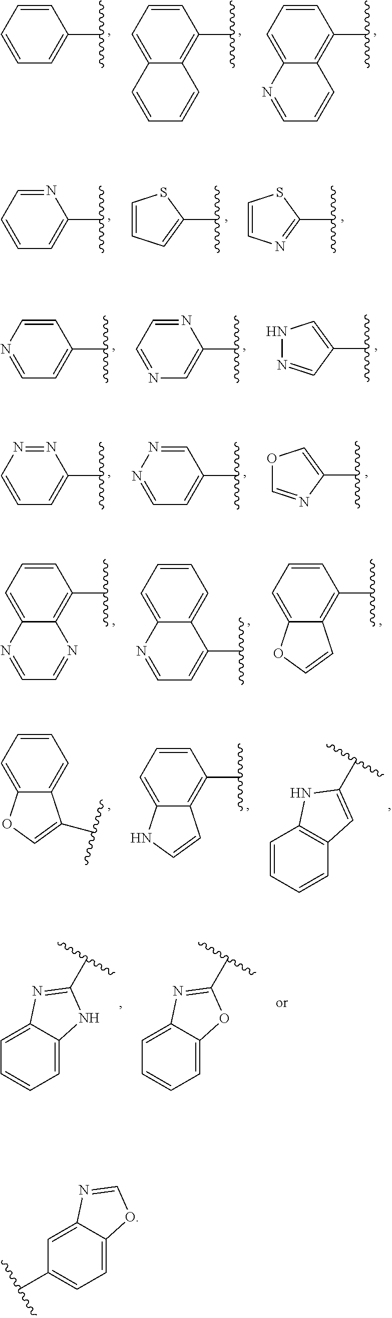

28. The compound of claim 1, wherein ring A is

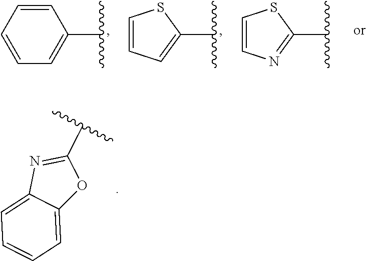

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. The compound of claim 1, wherein each R³⁰ is independently selected from hydrogen, halogen, —CN, —NO₂, ═O and C₁₋₆ alkyl,
p is selected from 1 or 2;
R^{C} is independently selected from hydrogen, ═O, and methyl;
ring C is 9- to 10-membered bicyclic heteroaryl, 9- to 14-membered partially unsaturated bicyclic heterocyclic ring, 12- to 14-membered tricyclic heteroaryl or 12- to 14-membered partially unsaturated tricyclic heterocyclic ring;
ring B is 6- to 10-membered aryl or 5- to 10-membered heteroaryl;
n is selected from 0, 1 and 2; and
each R^{B} is independently selected from hydrogen, halogen, —CN, —NO₂, ═O, and C₁₋₆ alkyl.

55. The compound of claim 1, wherein each R³⁰ is independently selected from hydrogen, F, Cl, Br, ═O, methyl and ethyl;
R^{C} is independently selected from hydrogen and ═O;
ring C is 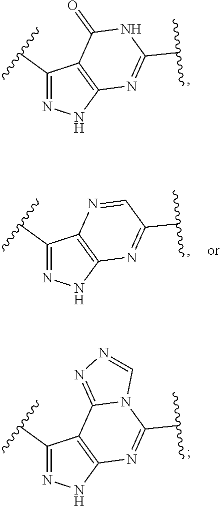 [SMILES: *c1nc2[nH]nc(*)c2c(=O)[nH]1 ; *c1cnc2c(*)n[nH]c2n1 ; *c1n[nH]c2nc(*)n3cnnc3c12]
ring B is 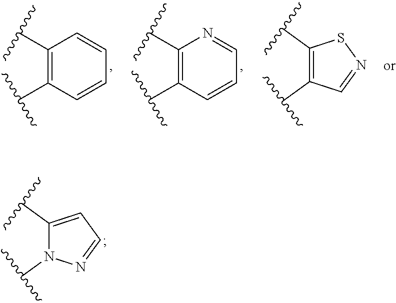 [SMILES: *c1ccccc1* ; *c1cccnc1* ; *c1cnsc1* ; *c1ccnn1*]
and
each R^{B} is independently selected from H, F, Cl, Br, ═O, methyl and ethyl.

56. The compound of claim 1, wherein R³⁰ is independently selected from H,
R^{C} is independently selected from hydrogen and ═O;
ring C is 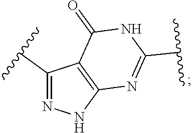 [SMILES: *c1nc2[nH]nc(*)c2c(=O)[nH]1]
ring B is 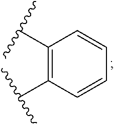 [SMILES: *c1ccccc1*]
and
R^{B} is H.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. The compound of claim 1, wherein M is CH₂; and W is absent.

73. (canceled)

74. The compound of claim 1, wherein the compound is:
(S)-6-(1-amino-1,3-dihydrospiro[indene-2,4′-piperidin]-1′-yl)-5-methyl-3-(1-phenylcyclopropyl)-1,5-dihydro-4H-pyrazolo[3,4-d]pyrimidin-4-one;
(S)-1′-(3-(1-phenylcyclopropyl)-1H-pyrazolo[3,4-b]pyrazin-6-yl)-1,3-dihydrospiro[indene-2,4′-piperidin]-1-amine;
(S)-1′-(9-(1-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
(S)-6-(1-amino-1,3-dihydrospiro[indene-2,4′-piperidin]-1′-yl)-3-(1-(3-chlorophenyl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(2,2-dimethyl-1-phenylcyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-fluorophenyl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(2,2-dichloro-1-phenylcyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(trifluoromethyl)phenyl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(2,2-difluoro-1-phenylcyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(m-tolyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-fluorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-chlorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2,2-difluorobenzo[d][1,3]dioxol-5-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(quinolin-6-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(trifluoromethyl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(trifluoromethoxy)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-methoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-methoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-methoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3,4-difluorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3,4-dichlorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-aminophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyridin-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(thiophen-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-chloropyridin-4-yl)cyclopropyl)-1,5-dihydro-4H-pyrazolo[3,4-d]pyrimidin-4-one;
(S)-4-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)picolinonitrile;
(S)-6-(1-amino-1,3-dihydrospiro[indene-2,4′-piperidin]-1′-yl)-3-(1-(2-(trifluoromethyl)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-methoxypyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(cyclopropylamino)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-cyclopropoxypyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-morpholinopyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(quinoxalin-6-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-phenylcyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(benzo[d][1,3]dioxol-5-yl)cyclopropyl)-1,5-dihydro-4H-pyrazolo[3,4-d]pyrimidin-4-one;
(S)-3-(1-(6-(1-amino-1,3-dihydrospiro[indene-2,4′-piperidin]-1′-yl)-4-oxo-4,5-dihydro-H-pyrazolo[3,4-d]pyrimidin-3-yl)cyclopropyl)benzonitrile;
(S)-6-(1-amino-1,3-dihydrospiro[indene-2,4′-piperidin]-1′-yl)-3-(1-(3-(1-methyl-1H-pyrazol-4-yl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(2-methyl-2H-1,2,3-triazol-4-yl)phenyl)cyclopropyl)-1,5-dihydro-4H-pyrazolo[3,4-d]pyrimidin-4-one;
(S)-3-(1-(3-(1H-pyrazol-1-yl)phenyl)cyclopropyl)-6-(1-amino-1,3-dihydrospiro[indene-2,4′-piperidin]-1′-yl)-1,5-dihydro-4H-pyrazolo[3,4-d]pyrimidin-4-one;
6-((S)-1-amino-1,3-dihydrospiro[indene-2,4′-piperidin]-1′-yl)-3-(1-(3-(tetrahydrofuran-3-yl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(tetrahydro-2H-pyran-4-yl)phenyl)cyclopropyl)-1,5-dihydro-4H-pyrazolo[3,4-d]pyrimidin-4-one;
ethyl (S)-(3-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)phenyl)carbamate;
(S)-6-(1-amino-1,3-dihydrospiro[indene-2,4′-piperidin]-1′-yl)-3-(1-(3-hydroxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-aminophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(2-methoxyethoxy)phenyl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(1-(3-((tetrahydrofuran-3-yl)oxy)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-amino-3-chloropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-fluoropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2,3-dichloropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2,3-dichlorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-chloro-3-hydroxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyrazin-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(difluoromethoxy)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(difluoromethoxy)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(dimethylamino)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(pyrrolidin-1-ylmethyl)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(1-phenyl-1H-pyrazol-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(1-benzyl-1H-pyrazol-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-fluoropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-amino-3-fluoropyridin-4-yl)cyclopropyl)-1,5-dihydro-4H-pyrazolo[3,4-d]pyrimidin-4-one;
(S)-3-(1-(1-acetyl-3,3-difluoroindolin-4-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-(dimethylamino)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-methoxypyridin-4-yl)cyclopropyl)-1,5-dihydro-4H-pyrazolo[3,4-d]pyrimidin-4-one;
(S)-3-(1-([1,1′-biphenyl]-3-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-morpholinopyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(azetidin-1-yl)-3-chloropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-(cyclobutylamino)pyridin-4-yl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(bicyclo[4.2.0]octa-1(6),2,4-trien-7-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(6-chloropyridazin-3-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(6-chloropyridazin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyridazin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-2-(cyclopropylamino)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-chloro-1-methyl-2-oxo-1,2-dihydropyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(naphthalen-1-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(quinolin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(5,6,7,8-tetrahydro-1,8-naphthyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(benzofuran-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(1-methyl-1H-indol-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-oxoindolin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(1,3-dihydroisobenzofuran-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(thiazol-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(oxazol-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-phenylpyridin-4-yl)cyclopropyl)-1,5-dihydro-4H-pyrazolo[3,4-d]pyrimidin-4-one;
(S)-3-(1-([2,2′-bipyridin]-4-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-(1-methyl-1H-pyrazol-3-yl)pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-methylpyridin-3-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-phenylcyclobutyl)-1,5-dihydro-4H-pyrazolo[3,4-d]pyrimidin-4-one;
(S)-6-(1-amino-1,3-dihydrospiro[indene-2,4′-piperidin]-1′-yl)-5-methyl-3-(1-phenylcyclobutyl)-1,5-dihydro-4H-pyrazolo[3,4-d]pyrimidin-4-one;
(S)-1′-(9-(1-phenylcyclobutyl)-7H-pyrazolo[4,3-e][1,2,4]triazolo[4,3-c]pyrimidin-5-yl)-1,3-dihydrospiro[indene-2,4′-piperidin]-1-amine;
(S)-6-(1-amino-1,3-dihydrospiro[indene-2,4′-piperidin]-1′-yl)-3-(1-(pyridin-2-yl)cyclobutyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(thiophen-2-yl)cyclobutyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-methoxyphenyl)cyclobutyl)-1,5-dihydro-4H-pyrazolo[3,4-d]pyrimidin-4-one;
(S)-6-(1-amino-1,3-dihydrospiro[indene-2,4′-piperidin]-1′-yl)-3-(3-phenyloxetan-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-phenylcyclopentyl)-1,5-dihydro-4H-pyrazolo[3,4-d]pyrimidin-4-one;
6-((S)-1-amino-1,3-dihydrospiro[indene-2,4′-piperidin]-1′-yl)-3-(3-phenyltetrahydrofuran-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-phenylcyclohexyl)-1,5-dihydro-4H-pyrazolo[3,4-d]pyrimidin-4-one;
(S)-6-(1-amino-1,3-dihydrospiro[indene-2,4′-piperidin]-1′-yl)-3-(4-phenyltetrahydro-2H-pyran-4-yl)-1,5-dihydro-4H-pyrazolo[3,4-d]pyrimidin-4-one;
6-((S)-1-amino-1,3-dihydrospiro[indene-2,4′-piperidin]-1′-yl)-3-(1-(2-methoxyphenyl)spiro[2.4]heptan-1-yl)-1,5-dihydro-4H-pyrazolo[3,4-d]pyrimidin-4-one;
6-((S)-1-amino-1,3-dihydrospiro[indene-2,4′-piperidin]-1′-yl)-3-((1S,2R)-2-phenylcyclopropyl)-1,5-dihydro-4H-pyrazolo[3,4-d]pyrimidin-4-one;
(S)-1′-(3-((1S,2R)-2-phenylcyclopropyl)-1H-pyrazolo[3,4-b]pyrazin-6-yl)-1,3-dihydrospiro[indene-2,4′-piperidin]-1-amine;
(S)-1′-(9-((1S,2R)-2-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
6-((S)-1-amino-1,3-dihydrospiro[indene-2,4′-piperidin]-1′-yl)-3-(5,6,7,8-tetrahydroquinolin-5-yl)-1,5-dihydro-4H-pyrazolo[3,4-d]pyrimidin-4-one;
6-((S)-1-amino-1,3-dihydrospiro[indene-2,4′-piperidin]-1′-yl)-3-(4-fluoro-3,3-dimethyl-2,3-dihydro-1H-inden-1-yl)-1,5-dihydro-4H-pyrazolo[3,4-d]pyrimidin-4-one;
6-((S)-1-amino-1,3-dihydrospiro[indene-2,4′-piperidin]-1′-yl)-3-(6,7,8,9-tetrahydro-5H-benzo[7]annulen-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(3,4-dihydronaphthalen-1-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(9-methyl-2,9-dihydro-1H-carbazol-4-yl)-1,5-dihydro-4H-pyrazolo[3,4-d]pyrimidin-4-one;
ethyl (S)-5-(6-(1-amino-1,3-dihydrospiro[indene-2,4′-piperidin]-1′-yl)-4-oxo-4,5-dihydro-1H-pyrazolo[3,4-d]pyrimidin-3-yl)-7,8-dihydroquinoline-3-carboxylate;
(S)-6-(1-amino-1,3-dihydrospiro[indene-2,4′-piperidin]-1′-yl)-3-(7-fluoro-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2H-pyrano[2,3-b]pyridin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6,7-dihydrobenzo[b]thiophen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-pentyl-6,7-dihydrobenzo[b]thiophen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6,7-dihydrobenzofuran-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-methyl-6,7-dihydro-1H-indol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-methyl-6,7-dihydro-2H-indazol-4-yl)-1,5-dihydro-4H-pyrazolo[3,4-d]pyrimidin-4-one;
(S)-5-(6-(1-amino-1,3-dihydrospiro[indene-2,4′-piperidin]-1′-yl)-4-oxo-4,5-dihydro-1H-pyrazolo[3,4-d]pyrimidin-3-yl)-7,8-dihydronaphthalene-2-carbonitrile;
(S)-6-(1-amino-1,3-dihydrospiro[indene-2,4′-piperidin]-1′-yl)-3-(4,4-difluoro-3,4-dihydronaphthalen-1-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8-fluoro-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6,7-dihydro-5H-benzo[7]annulen-9-yl)-1,5-dihydro-4H-pyrazolo[3,4-d]pyrimidin-4-one;
8-(6-(1-amino-1,3-dihydrospiro[indene-2,4′-piperidin]-1′-yl)-4-oxo-4,5-dihydro-1H-pyrazolo[3,4-d]pyrimidin-3-yl)-5,5-difluoro-5,6-dihydronaphthalene-2-carbonitrile;
6-(1-amino-1,3-dihydrospiro[indene-2,4′-piperidin]-1′-yl)-3-(8,8-dimethyl-7,8-dihydroquinolin-5-yl)-1,5-dihydro-4H-pyrazolo[3,4-d]pyrimidin-4-one;
6-(1-amino-1,3-dihydrospiro[indene-2,4′-piperidin]-1′-yl)-3-(5,6-dihydroimidazo[1,2-a]pyridin-8-yl)-1,5-dihydro-4H-pyrazolo[3,4-d]pyrimidin-4-one;
6-(1-amino-1,3-dihydrospiro[indene-2,4′-piperidin]-1′-yl)-3-(2,5,5-trimethyl-4,5-dihydrobenzo[d]thiazol-7-yl)-1,5-dihydro-4H-pyrazolo[3,4-d]pyrimidin-4-one;
6-(1-amino-1,3-dihydrospiro[indene-2,4′-piperidin]-1′-yl)-3-(2-amino-5,5-dimethyl-4,5-dihydrobenzo[d]thiazol-7-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-methyl-6,7-dihydro-TH-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2′H-spiro[cyclopropane-1,1′-naphthalen]-4′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7′H-spiro[cyclopropane-1,8′-quinolin]-5′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1H-isothiochromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6-chloro-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2,4-bis(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(3-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-chloro-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-methyl-4-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-cyclopropyl-4-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(4-(difluoromethyl)-2-methyl-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7,7-dimethyl-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7,7-dimethyl-2-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8,8-dimethyl-2-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7,7-dimethyl-2-(trifluoromethyl)-7,8-dihydroquinazolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2,7,7-trimethyl-7,8-dihydroquinazolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-cyclopropyl-7,7-dimethyl-7,8-dihydroquinazolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7,7-dimethyl-2-(methylamino)-7,8-dihydroquinazolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,6,6-trimethyl-6,7-dihydro-1H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6,6-dimethyl-1-(2,2,2-trifluoroethyl)-6,7-dihydro-1H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-cyclopropyl-6,6-dimethyl-6,7-dihydro-1H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2,6,6-trimethyl-6,7-dihydro-2H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6,6-dimethyl-2-(2,2,2-trifluoroethyl)-6,7-dihydro-2H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-cyclopropyl-6,6-dimethyl-6,7-dihydro-2H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(3-cyclopropyl-6,6-dimethyl-1-(2,2,2-trifluoroethyl)-6,7-dihydro-1H-indazol-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(3,3-dimethyl-3,4-dihydroacridin-1-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8,8-dimethyl-3,4,8,9-tetrahydro-1H-pyrano[3,4-b]quinolin-6-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-amino-5,5-dimethyl-4,5-dihydrobenzo[d]thiazol-7-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(3-bromo-7,7-dimethyl-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-chloro-7,7-dimethyl-7,8-dihydroquinolin-5-yl)-1,5-dihydro-4H-pyrazolo[3,4-d]pyrimidin-4-one;
(S)-5-(6-(1-amino-1,3-dihydrospiro[indene-2,4′-piperidin]-1′-yl)-4-oxo-4,5-dihydro-H-pyrazolo[3,4-d]pyrimidin-3-yl)-7,7-dimethyl-7,8-dihydroquinoline-2-carbonitrile;
(S)-5-(6-(1-amino-1,3-dihydrospiro[indene-2,4′-piperidin]-1′-yl)-4-oxo-4,5-dihydro-H-pyrazolo[3,4-d]pyrimidin-3-yl)-7,7-dimethyl-7,8-dihydroquinoline-3-carbonitrile;
(S)-6-(1-amino-1,3-dihydrospiro[indene-2,4′-piperidin]-1′-yl)-3-(3,7,7-trimethyl-7,8-dihydrocinnolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8,8-dimethyl-8,9-dihydro-[1,2,4]triazolo[4,3-a]quinazolin-6-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8,8-dimethyl-8,9-dihydro-[1,2,4]triazolo[3,4-b]quinazolin-6-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7-chloro-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7-(trifluoromethyl)-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(8,8-difluoro-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(spiro[indene-1,3′-oxetan]-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-methylspiro[azetidine-3,1′-inden]-3′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1H-inden-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-oxo-2H-chromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,1-difluoro-1H-inden-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,1-dimethyl-1H-inden-3-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,2-dihydroquinolin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-methyl-1,2-dihydroquinolin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,1-dimethyl-1,2-dihydroisoquinolin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,1-dioxido-2H-thiochromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1,1-dioxido-2H-benzo[e][1,2]thiazin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2,2-dioxido-1H-isothiochromen-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2,2-dioxido-1H-benzo[c][1,2]thiazin-4-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-methyl-2,2-dioxido-1H-benzo[c][1,2]thiazin-4-yl)-1,5-dihydro-4H-pyrazolo[3,4-d]pyrimidin-4-one;
6-((S)-1-amino-1,3-dihydrospiro[indene-2,4′-piperidin]-1′-yl)-3-((1S,2S)-2-phenylcyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(6-fluoro-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6-fluoro-2-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(2-chloro-6-fluoro-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6-fluoro-7,7-dimethyl-2-(trifluoromethyl)-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(6-fluoro-7,7-dimethyl-7,8-dihydroquinolin-5-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(7-methoxybenzofuran-3-yl)-1,5-dihydro-4H-pyrazolo[3,4-d]pyrimidin-4-one;
(S)-6-(1-amino-6-methoxy-1,3-dihydrospiro[indene-2,4′-piperidin]-1′-yl)-3-(1-phenylcyclopropyl)-1,5-dihydro-4H-pyrazolo[3,4-d]pyrimidin-4-one;
(S)-6-(4-amino-2-chloro-4,6-dihydrospiro[cyclopenta[d]thiazole-5,4′-piperidin]-1′-yl)-3-(1-phenylcyclopropyl)-1,5-dihydro-4H-pyrazolo[3,4-d]pyrimidin-4-one;
(S)-6-(1-amino-6-chloro-1,3-dihydrospiro[indene-2,4′-piperidin]-1′-yl)-3-(1-phenylcyclopropyl)-1,5-dihydro-4H-pyrazolo[3,4-d]pyrimidin-4-one;
(S)-6-(1-amino-6-fluoro-1,3-dihydrospiro[indene-2,4′-piperidin]-1′-yl)-3-(1-phenylcyclopropyl)-1,5-dihydro-4H-pyrazolo[3,4-d]pyrimidin-4-one;
(S)-6-(1-amino-6-(methylthio)-1,3-dihydrospiro[indene-2,4′-piperidin]-1′-yl)-3-(1-phenylcyclopropyl)-1,5-dihydro-4H-pyrazolo[3,4-d]pyrimidin-4-one;
(S)-1-amino-1′-(4-oxo-3-(1-phenylcyclopropyl)-4,5-dihydro-1H-pyrazolo[3,4-d]pyrimidin-6-yl)-1,3-dihydrospiro[indene-2,4′-piperidine]-6-carbonitrile;
(R)-6-(2-amino-2,3-dihydrospiro[indene-1,4′-piperidin]-1′-yl)-3-(1-phenylcyclopropyl)-1,5-dihydro-4H-pyrazolo[3,4-d]pyrimidin-4-one;
(S)-6-(5′-amino-5′,6′-dihydrospiro[piperidine-4,4′-pyrrolo[1,2-b]pyrazol]-1-yl)-3-(1-phenylcyclopropyl)-1,5-dihydro-4H-pyrazolo[3,4-d]pyrimidin-4-one;
(S)-6-(5-amino-5,7-dihydrospiro[cyclopenta[b]pyridine-6,4′-piperidin]-1′-yl)-3-(1-phenylcyclopropyl)-1,5-dihydro-4H-pyrazolo[3,4-d]pyrimidin-4-one;
(S)-1′-(3-(1-phenylcyclopropyl)-1H-pyrazolo[3,4-b]pyrazin-6-yl)-5,7-dihydrospiro[cyclopenta[b]pyridine-6,4′-piperidin]-5-amine;
(S)-6-chloro-1′-(3-(1-phenylcyclopropyl)-1H-pyrazolo[3,4-b]pyrazin-6-yl)-1,3-dihydrospiro[indene-2,4′-piperidin]-1-amine;
(S)-6-fluoro-1′-(3-(1-phenylcyclopropyl)-1H-pyrazolo[3,4-b]pyrazin-6-yl)-1,3-dihydrospiro[indene-2,4′-piperidin]-1-amine;
(S)-6-(methylthio)-1′-(3-(1-phenylcyclopropyl)-1H-pyrazolo[3,4-b]pyrazin-6-yl)-1,3-dihydrospiro[indene-2,4′-piperidin]-1-amine;
(S)-2-chloro-1′-(3-(1-phenylcyclopropyl)-1H-pyrazolo[3,4-b]pyrazin-6-yl)-4,6-dihydrospiro[cyclopenta[d]thiazole-5,4′-piperidin]-4-amine;
(S)-1-(3-(1-phenylcyclopropyl)-1H-pyrazolo[3,4-b]pyrazin-6-yl)-5′,6′-dihydrospiro[piperidine-4,4′-pyrrolo[1,2-b]pyrazol]-5′-amine;
(S)-1-(9-(1-phenylcyclopropyl)-7H-pyrazolo[4,3-e][1,2,4]triazolo[4,3-c]pyrimidin-5-yl)-5′,6′-dihydrospiro[piperidine-4,4′-pyrrolo[1,2-b]pyrazol]-5′-amine;
(S)-6-methoxy-1′-(9-(1-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
(S)-1′-(9-(1-phenylcyclopropyl)-7H-pyrazolo[4,3-e][1,2,4]triazolo[4,3-c]pyrimidin-5-yl)-5,7-dihydrospiro[cyclopenta[b]pyridine-6,4′-piperidin]-5-amine;
(S)-6-chloro-1′-(9-(1-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
(S)-6-fluoro-1′-(9-(1-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
(S)-6-(methylthio)-1′-(9-(1-phenylcyclopropyl)-7H-pyrazolo[4,3-e][1,2,4]triazolo[4,3-c]pyrimidin-5-yl)-1,3-dihydrospiro[indene-2,4′-piperidin]-1-amine;
(S)-2-chloro-1′-(9-(1-phenylcyclopropyl)-7H-pyrazolo[4,3-e][1,2,4]triazolo[4,3-c]pyrimidin-5-yl)-4,6-dihydrospiro[cyclopenta[d]thiazole-5,4′-piperidin]-4-amine;
(S)-6-(1-amino-1,3-dihydrospiro[indene-2,4′-piperidin]-1′-yl)-3-(2-phenylpropan-2-yl)-1,5-dihydro-4H-pyrazolo[3,4-d]pyrimidin-4-one;
6-((S)-1-amino-1,3-dihydrospiro[indene-2,4′-piperidin]-1′-yl)-3-(1-phenylpropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-cyclopropylphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyridin-4-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2-fluorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3,4-dimethoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-ethynylphenyl)cyclopropyl)-1,5-dihydro-4H-pyrazolo[3,4-d]pyrimidin-4-one;
(S)-3-(1-(3-acetylphenyl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(dimethylphosphoryl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(methylthio)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-(hydroxymethyl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(3-cyclopropoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-(trifluoromethyl)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-aminophenyl)cyclopropyl)-1,5-dihydro-4H-pyrazolo[3,4-d]pyrimidin-4-one;
ethyl (S)-(4-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)phenyl)carbamate;
(S)-6-(1-amino-1,3-dihydrospiro[indene-2,4′-piperidin]-1′-yl)-3-(1-(2,4-dimethoxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-(trifluoromethoxy)phenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(4-hydroxyphenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(2,4-dichlorophenyl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(thiophen-3-yl)cyclopropyl)-1,5-dihydro-4H-pyrazolo[3,4-d]pyrimidin-4-one;
(S)-4-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)benzonitrile;
(S)-5-(1-(6-(1-amino-1,3-dihydrospiro[indene-2,4′-piperidin]-1′-yl)-4-oxo-4,5-dihydro-1H-pyrazolo[3,4-d]pyrimidin-3-yl)cyclopropyl)-1,3,4-thiadiazole-2-carbonitrile;
(S)-3-(1-(1,3,4-thiadiazol-2-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-3-(1-(1,2,3-thiadiazol-4-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(5-methyl-1,2,3-thiadiazol-4-yl)cyclopropyl)-1,5-dihydro-4H-pyrazolo[3,4-d]pyrimidin-4-one;
6-((S)-1-amino-1,3-dihydrospiro[indene-2,4′-piperidin]-1′-yl)-3-(1-(1-oxidothiophen-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(oxazol-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(5-methyloxazol-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyrimidin-2-yl)cyclopropyl)-1,5-dihydro-4H-pyrazolo[3,4-d]pyrimidin-4-one;
(S)-3-(1-(2H-tetrazol-5-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(pyridin-3-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(6-methylpyridin-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(5-cyclopropyl-1,3,4-thiadiazol-2-yl)cyclopropyl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(benzofuran-3-yl)cyclopropyl)-1,5-dihydro-4H-pyrazolo[3,4-d]pyrimidin-4-one;
(S)-3-(1-(1H-benzo[d]imidazol-2-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(benzo[d]oxazol-2-yl)cyclopropyl)-1,5-dihydro-4H-pyrazolo[3,4-d]pyrimidin-4-one;
(S)-3-(1-(1H-1,2,3-triazol-4-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-3-(1-(1H-pyrrol-1-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-3-(1-(1H-pyrazol-1-yl)cyclopropyl)-6-(1-amino-1,3-dihydrospiro[indene-2,4′-piperidin]-1′-yl)-1,5-dihydro-4H-pyrazolo[3,4-d]pyrimidin-4-one;
(S)-6-(1-amino-1,3-dihydrospiro[indene-2,4′-piperidin]-1′-yl)-3-(1-(furan-2-yl)cyclopropyl)-1,5-dihydro-4H-pyrazolo[3,4-d]pyrimidin-4-one;
(R)-6-(1-amino-1,3-dihydrospiro[indene-2,4′-piperidin]-1′-yl)-3-(1-phenylcyclopropyl)-1,5-dihydro-4H-pyrazolo[3,4-d]pyrimidin-4-one;
(3S,4S)-3-methyl-8-(5-(1-phenylcyclopropyl)pyrazin-2-yl)-2-oxa-8-azaspiro[4.5]decan-4-amine;
3-((3S,4S)-4-amino-3-methyl-2-oxa-8-azaspiro[4.5]decan-8-yl)-6-(1-phenylcyclopropyl)pyrazine-2-carboxamide;
(3-((3S,4S)-4-amino-3-methyl-2-oxa-8-azaspiro[4.5]decan-8-yl)-6-(1-phenylcyclopropyl)pyrazin-2-yl)methanol;
(3-((3S,4S)-4-amino-3-methyl-2-oxa-8-azaspiro[4.5]decan-8-yl)-5-methyl-6-(1-phenylcyclopropyl)pyrazin-2-yl)methanol;
2-((3S,4S)-4-amino-3-methyl-2-oxa-8-azaspiro[4.5]decan-8-yl)-5-(1-phenylcyclopropyl)pyrimidin-4(3H)-one;
2-((3S,4S)-4-amino-3-methyl-2-oxa-8-azaspiro[4.5]decan-8-yl)-3-methyl-5-(1-phenylcyclopropyl)pyrimidin-4(3H)-one;
6-amino-2-((3S,4S)-4-amino-3-methyl-2-oxa-8-azaspiro[4.5]decan-8-yl)-3-methyl-5-(1-phenylcyclopropyl)pyrimidin-4(3H)-one;
(3S,4S)-8-(8-amino-9-(1-phenylcyclopropyl)-3,4-dihydro-2H-pyrimido[1,6-a]pyrimidin-6-yl)-3-methyl-2-oxa-8-azaspiro[4.5]decan-4-amine;
(3S,4S)-8-(5-amino-6-(1-phenylcyclopropyl)-2,3-dihydroimidazo[1,2-a]pyrimidin-7-yl)-3-methyl-2-oxa-8-azaspiro[4.5]decan-4-amine;
(3S,4S)-3-methyl-8-(3-(1-phenylcyclopropyl)-1H-pyrazolo[3,4-d]pyrimidin-6-yl)-2-oxa-8-azaspiro[4.5]decan-4-amine;
(3S,4S)-3-methyl-8-(3-(1-(thiophen-3-yl)cyclopropyl)-1H-pyrazolo[3,4-b]pyrazin-6-yl)-2-oxa-8-azaspiro[4.5]decan-4-amine;
(3S,4S)-3-methyl-8-(7-(1-phenylcyclopropyl)-5H-pyrrolo[2,3-b]pyrazin-3-yl)-2-oxa-8-azaspiro[4.5]decan-4-amine;
6-((3S,4S)-4-amino-3-methyl-2-oxa-8-azaspiro[4.5]decan-8-yl)-3-(1-phenylcyclopropyl)-1,5-dihydro-4H-pyrazolo[3,4-d]pyrimidin-4-one;
(S)-6-(4-amino-2-oxa-8-azaspiro[4.5]decan-8-yl)-3-(1-phenylcyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-5-methyl-3-(1-phenylcyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(pyrimidin-4-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(pyridin-4-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(pyridin-3-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(pyridin-2-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(thiazol-2-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(thiophen-2-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(oxazol-2-yl)cyclopropyl)-1,5-dihydro-4H-pyrazolo[3,4-d]pyrimidin-4-one;
3-(1-(1,3,4-thiadiazol-2-yl)cyclopropyl)-6-((3S,4S)-4-amino-3-methyl-2-oxa-8-azaspiro[4.5]decan-8-yl)-1,5-dihydro-4H-pyrazolo[3,4-d]pyrimidin-4-one;
(S)-6-(4-amino-2-oxa-8-azaspiro[4.5]decan-8-yl)-3-(1-(benzo[d]oxazol-2-yl)cyclopropyl)-1,5-dihydro-4H-pyrazolo[3,4-d]pyrimidin-4-one;
(S)-3-(1-(1H-benzo[d]imidazol-2-yl)cyclopropyl)-6-(4-amino-2-oxa-8-azaspiro[4.5]decan-8-yl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(benzo[d]oxazol-2-yl)cyclopropyl)-1,5-dihydro-4H-pyrazolo[3,4-d]pyrimidin-4-one;
3-(1-(1H-indol-2-yl)cyclopropyl)-6-((3S,4S)-4-amino-3-methyl-2-oxa-8-azaspiro[4.5]decan-8-yl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(benzo[d][1,3]dioxol-5-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(benzo[d]oxazol-5-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-fluoro-5-methoxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-fluoro-3-methoxyphenyl)cyclopropyl)-1,5-dihydro-4H-pyrazolo[3,4-d]pyrimidin-4-one;
3-(1-(6-((3S,4S)-4-amino-3-methyl-2-oxa-8-azaspiro[4.5]decan-8-yl)-4-oxo-4,5-dihydro-1H-pyrazolo[3,4-d]pyrimidin-3-yl)cyclopropyl)benzonitrile;
3-(1-(2-amino-3-chloropyridin-4-yl)cyclopropyl)-6-((3S,4S)-4-amino-3-methyl-2-oxa-8-azaspiro[4.5]decan-8-yl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-fluorophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-fluorophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2-fluorophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3,4-difluorophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-(trifluoromethyl)phenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-(trifluoromethyl)phenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2-(trifluoromethyl)phenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-aminophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(p-tolyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(m-tolyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(o-tolyl)cyclopropyl)-1,5-dihydro-4H-pyrazolo[3,4-d]pyrimidin-4-one;
ethyl (4-(1-(6-((3S,4S)-4-amino-3-methyl-2-oxa-8-azaspiro[4.5]decan-8-yl)-4-oxo-4,5-dihydro-1H-pyrazolo[3,4-d]pyrimidin-3-yl)cyclopropyl)phenyl)carbamate;
6-((3S,4S)-4-amino-3-methyl-2-oxa-8-azaspiro[4.5]decan-8-yl)-3-(1-(4-methoxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-methoxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2-methoxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-(trifluoromethoxy)phenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2,2-difluorobenzo[d][1,3]dioxol-5-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2,3-dichlorophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-hydroxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-hydroxyphenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(2,4-dichlorophenyl)cyclopropyl)-1,5-dihydro-4H-pyrazolo[3,4-d]pyrimidin-4-one;
3-(1-(3-(1H-pyrazol-1-yl)phenyl)cyclopropyl)-6-((3S,4S)-4-amino-3-methyl-2-oxa-8-azaspiro[4.5]decan-8-yl)-1,5-dihydro-4H-pyrazolo[3,4-d]pyrimidin-4-one;
4-(1-(6-((3S,4S)-4-amino-3-methyl-2-oxa-8-azaspiro[4.5]decan-8-yl)-4-oxo-4,5-dihydro-1H-pyrazolo[3,4-d]pyrimidin-3-yl)cyclopropyl)benzonitrile;
3-(1-(3-acetylphenyl)cyclopropyl)-6-((3R,4R)-4-amino-3-methyl-2-oxa-8-azaspiro[4.5]decan-8-yl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(3-bromophenyl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(4-methylthiazol-2-yl)cyclopropyl)-1,5-dihydro-4H-pyrazolo[3,4-d]pyrimidin-4-one;
6-((3S,4S)-4-amino-3-methyl-2-oxa-8-azaspiro[4.5]decan-8-yl)-3-(1-(6-methylpyridin-2-yl)cyclopropyl)-1,5-dihydro-4H-pyrazolo[3,4-d]pyrimidin-4-one;
6-((1R,2R)-1-amino-2-methyl-8-azaspiro[4.5]decan-8-yl)-3-(1-phenylcyclopropyl)-1,5-dihydro-4H-pyrazolo[3,4-d]pyrimidin-4-one;
(R)-6-(1-amino-8-azaspiro[4.5]decan-8-yl)-3-(1-phenylcyclopropyl)-1,5-dihydro-4H-pyrazolo[3,4-d]pyrimidin-4-one;
(R)-6-(3-amino-3H-spiro[benzofuran-2,4′-piperidin]-1′-yl)-3-(1-phenylcyclopropyl)-1,5-dihydro-4H-pyrazolo[3,4-d]pyrimidin-4-one;
(R)-1′-(3-(1-phenylcyclopropyl)-1H-pyrazolo[3,4-b]pyrazin-6-yl)-3H-spiro[benzofuran-2,4′-piperidin]-3-amine; or
(R)-1′-(9-(1-phenylcyclopropyl)-7H-pyrazolo[4,3-e][1,2,4]triazolo[4,3-c]pyrimidin-5-yl)-3H-spiro[benzofuran-2,4′-piperidin]-3-amine.

75. A pharmaceutical composition comprising a compound of claim 1, a pharmaceutically acceptable salt, isomeride, stereoisomer, prodrug, chelate, non-covalent complex, or solvate thereof, and at least one pharmaceutically acceptable carrier or excipient.

76. (canceled)

77. (canceled)

78. (canceled)

79. (canceled)

80. (canceled)

81. (canceled)

82. A method for treating and/or preventing a disease mediated by SHP2, said method comprising administering to a patient in need a compound of claim 1.

83. The method of claim 82, wherein the disease is cancer.

84. (canceled)

85. The method of claim 83, wherein the cancer is Noonan syndrome, leopard spot syndrome, juvenile myelomonocytic leukemia, neuroblastoma, melanoma, head and neck squamous cell carcinoma, acute myeloid leukemia, breast cancer, esophageal cancer, lung cancer, colon cancer, head cancer, gastric cancer, lymphoma, glioblastoma, and/or pancreatic cancer.
